(19) United States
(12) Patent Application Publication
(10) Pub. No.: US 2011/0052527 A1
(43) Pub. Date: Mar. 3, 2011

(54) IMIDAZOPYRIDINYL THIAZOLYL HISTONE DEACETYLASE INHIBITORS

(75) Inventors: Lawrence S. Melvin, JR., Longmont, CO (US); Michael Graupe, Pacifica, CA (US); Chandrasekar Venkataramani, Redwood City, CA (US)

(73) Assignee: Gilead Sciences, Inc., Foster City, CA (US)

(21) Appl. No.: 12/943,799

(22) Filed: Nov. 10, 2010

Related U.S. Application Data
(63) Continuation of application No. 12/146,894, filed on Jun. 26, 2008, now abandoned.
(60) Provisional application No. 60/946,276, filed on Jun. 26, 2007.
(60) Provisional application No. 61/051,190, filed on May 7, 2008.

(51) Int. Cl. A61K 38/19; C07D 471/04; A61K 31/437; A61K 31/675; A61K 31/655; A61K 33/24; A61K 31/519; A61K 31/52; A61K 31/513; A61K 31/7068; A61K 38/50; A61K 39/395; A61P 35/00; A61P 3/10; A61P 25/28; A61P 25/00; A61P 3/00

(52) U.S. Cl. 424/85.1; 546/121; 514/300; 514/110; 514/151; 424/649; 514/249; 514/263.32; 514/274; 514/49; 424/94.6; 424/133.1

(57) ABSTRACT

A compound of general Formula (I) having histone deacetylase (HDAC) and/or CDK inhibitory activity, a pharmaceutical composition comprising the compound, and a method useful to treat diseases using the compound.

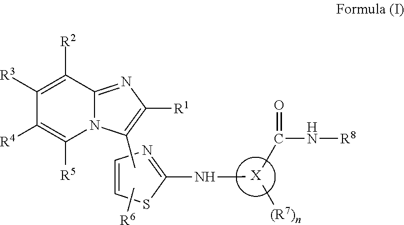

### CROSS REFERENCE TO RELATED APPLICATIONS

This application claims the benefit of U.S. provisional application Ser. No. 60/946,276 filed Jun. 26, 2007 and U.S. provisional application Ser. No. 61/051,190 filed May 7, 2008. The disclosure of these applications is hereby incorporated by reference.

### FIELD

The present invention generally relates to a compound having enzyme inhibitory activity, pharmaceutical compositions comprising the compound, and methods useful for treating diseases.

### BACKGROUND

Histones are protein components making up chromatin in association with DNA. Histones are subject to covalent modifications of various enzymes such as, for example, histone deacetylase (HDAC), histone methyltransferase (HMT) and histone acetyltransferase (HAT). Covalent modifications of core histones influence protein-protein interaction and protein access to DNA.

HDACs catalyze deacetylation of lysine residues on histones and other proteins. It is known that low levels of histone-acetylation are associated with repression of gene expression. Therefore, abnormal HDAC activities could destroy the delicate balance in cell regulation. The HDACs belong to four structurally and functionally different phylogenetic classes: class I (HDAC-1, -2, -3, and -8) compounds are closely related to yeast RPD3; class IIa (HDAC-4, -5, -7, and -9) and class IIb (HDAC-6 and -10) share domains with yeast HDAC-1; class IV, recently described (comprising HDAC-11), exhibits properties of both class I and class II HDACs. All the above HDACs are zinc dependent proteases. Class III HDACs have been identified on the basis of sequence similarity with Sir2, a yeast transcription repressor, and require the cofactor NAD⁺ for their deacetylase function. See, for example, Marielle Paris et al., Histone Deacetylase Inhibitors: From Bench to Clinic, JOURNAL OF MEDICINAL CHEMISTRY)(XXX, xxx, 000-000, published on the Web Feb. 5, 2008.

It has been reported that HDAC activities play an important role in a variety of human disease states. Accordingly, an HDAC inhibitor can provide therapeutic benefits to a broad range of patients. Due to the therapeutic significance, various types of HDAC inhibitors have been developed to date. See, for example, Moradeli et al., Histone Deacetylase Inhibitors: Latest Developments, Trends, and Prospects, CURR. MED. CHEM.: ANTI-CANCER AGENTS 5(5):529-560 (2005).

Cyclin-dependent kinases (CDKs) are protein kinase enzymes controlling transcription and mRNA processing for the regulation of the cell cycle. CDKs belong to a group of serine/threonine kinases phosphorylating proteins on serine and threonine amino acid residues. A CDK is activated by association with a cyclin forming a cyclin-dependent kinase complex. The CDK family has been identified to include at least 9 members, i.e., CDK1, CDK2, CDK3, CDK4, CDK5, CDK6, CDK7, CDK8 and CDK9, and CDKs pair with a specific cyclin in the various phases of the cell cycle for the progression. CDKs are considered a target for anti-cancer medication since the enzymes are major control switches for the cell cycle.

JP 2003-313126 discloses compounds that contain a thiazole ring attached to imidazopyridine and are said to be useful for treating tumors.

WO 2005/092899 mentions a series of compounds useful for inhibiting HDAC enzymatic activity where the compounds are amino or hydroxyl substituted aniline derivatives attached to various cyclic groups.

There is a continued need to develop new inhibitors to provide appropriate therapy for a variety of disease conditions implicated in HDAC and CDK activity.

### SUMMARY

In various embodiments, a compound having HDAC inhibitory activity, a composition comprising the compound, and a method useful to treat diseases arising from abnormal cell proliferation or differentiation are provided.

The compound is of Formula (I) or a pharmaceutically acceptable salt thereof:

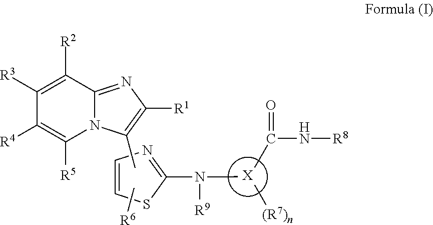

wherein
R¹, R², R³, R⁴ and R⁵ are independently selected from the group consisting of H, halo, nitro, cyano, hydroxy, hydroxyalkyl, haloalkyl, haloalkoxy, amino, aminoalkyl, azido, carboxy, carbamoyl, mercapto, sulphamoyl, C₁₋₁₀ alkyl, C₂₋₁₀ alkenyl, C₂₋₁₀ alkynyl, C₁₋₁₀ alkoxy, C₁₋₁₀ alkanoyl, C₁₋₁₀ alkanoyloxy, N—(C₁₋₁₀ alkyl)amino, N,N—(C₁₋₁₀ alkyl)₂-amino, C₁₋₁₀ alkanoylamino, N—(C₁₋₁₀ alkyl)carbamoyl, N,N—(C₁₋₁₀ alkyl)₂-carbamoyl, C₁₋₁₀ alkyl-S(O)ₐ wherein a is 0, 1 or 2, C₁₋₆ alkoxycarbonyl, NH₂—S(O)₂NH—, N—(C₁₋₁₀ alkyl)sulphamoyl, N,N—(C₁₋₁₀ alkyl)₂sulphamoyl, aryl, aryloxy, arylthio, heteroaryl, heteroaryloxy, cycloalkyl, cycloalkyloxy, heterocyclyl, heterocyclyl(C═O)—, heterocyclyloxy and heterocyclylthio; wherein each of R¹, R², R³, R⁴ and R⁵ is optionally substituted by one or more A where such an optional substitution is chemically feasible;
R⁶ is H, halo, nitro, cyano, trifluoromethyl, trifluoromethoxy, amino, carboxy, carbamoyl, sulphamoyl, C₁₋₁₀ alkyl, C₂₋₁₀ alkenyl, C₂₋₁₀ alkynyl, C₁₋₁₀ alkoxy, C₁₋₁₀ alkanoyl, N—(C₁₋₁₀ alkyl)amino, N,N—(C₁₋₁₀ alkyl)₂ amino, C₁₋₁₀ alkanoylamino, N—(C₁₋₁₀ alkyl)carbamoyl, N,N—(C₁₋₁₀ alkyl)₂ carbamoyl, C₁₋₁₀ alkyl-S(O)ₐ wherein a is 0, 1 or 2, NH₂—S(O)₂NH—, N—(C₁₋₁₀ alkyl)sulphamoyl or N,N—(C₁₋₁₀ alkyl)₂sulphamoyl; wherein R⁶ is optionally substituted by one or more B where such an optional substitution is chemically feasible;
X is phenyl, 5-membered heteroaryl, or 6-membered heteroaryl, wherein the heteroaryl contains one or more heteroatoms selected from N, S and O;
R⁷ represents one or more optional non-hydrogen substituents on ring X. When present, each R⁷ is independently selected from halo and methyl;
n is the number of non-hydrogen substituents R⁷ on the ring X and can be 0, 1, 2, 3, or 4. The maximum value of n depends on the nature of the ring X;
R⁸ is hydroxy, aryl or heteroaryl, wherein aryl or heteroaryl are substituted with —NH₂ or —OH and aryl or heteroaryl is optionally further substituted with one or more groups R¹⁰ selected from amino, halo, alkyl, cycloalkyl, heterocyclyl, aryl and heteroaryl;
R⁹ is H, alkyl, haloalkyl, aminoalkyl, cycloalkyl, heterocyclyl, aryl, heteroaryl, wherein R⁹ is optionally substituted by one or more D where such an optional substitution is chemically feasible;
A and B are independently selected from halo, nitro, cyano, hydroxy, hydroxyalkyl, haloalkyl, haloalkoxy, amino, azido, carboxy, carbamoyl, mercapto, sulphamoyl, C₁₋₁₀ alkyl, C₂₋₁₀ alkenyl, C₂₋₁₀ alkynyl, C₁₋₁₀ alkoxy, C₁₋₁₀ alkoxyalkyl, C₁₋₁₀ alkanoyl, C₁₋₁₀ alkanoyloxy, N—(C₁₋₁₀ alkyl)amino, N,N—(C₁₋₄₀ alkyl)₂ amino, C₁₋₁₀ alkanoylamino, N—(C₁₋₁₀ alkyl)carbamoyl, N,N—(C₁₋₁₀ alkyl)₂-carbamoyl, C₁₋₁₀ alkyl-S(O)ₐ wherein a is 0, 1 or 2, C₁₋₁₀ alkoxycarbonyl, N—(C₁₋₁₀ alkyl)sulphamoyl, N,N—(C₁₋₁₀ alkyl)₂sulphamoyl, H₂NS(O)₂NH—, N—(C₁₋₁₀ alkyl)NHS(O)₂NH—, N,N—(C₁₋₁₀ alkyl)₂NS(O)₂NH—, aryl, aryloxy, arylthio, heteroaryl, heteroaryloxy, cycloalkyl, cycloalkyloxy, heterocyclyl, heterocyclyl(C═O)—, heterocyclyloxy and heterocyclylthio; and
D is selected from halo, nitro, cyano, hydroxy, amino, azido, carboxy and mercapto.

Non-limiting examples of A and B include halo, alkyl, nitro, cyano, hydroxy, cycloalkyl, trifluoromethoxy, trifluoromethyl, trifluoroethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl, N-methyl-N-ethylsulphamoyl, aryl, heterocyclylcycloalkyl and heteroaryl.

In the definitions herein of R¹, R², R³, R⁴, R⁵, R⁶, A, and B, the carbon ranges for the groups alkyl, alkenyl, alkynyl, alkoxy, alkanoyl, alkanoyloxy, alkanoylamino, and the like include all ranges encompassed in the recited ranges C₁₋₁₀ and C₂₋₁₀. For example, in non-limiting fashion C₁₋₁₀ and C₂₋₁₀ include a disclosure of C₁₋₆ and C₁₋₃. In various embodiments, C₁₋₁₀ carbon-chain containing groups such as C₁₋₁₀ alkyl, C₂₋₁₀ alkenyl, C₂₋₁₀ alkynyl and so forth include the respective C₁₋₆ and C₁₋₃ shorter carbon-chains such as C₁₋₆ alkyl, C₁₋₃ alkyl, C₂₋₆ alkenyl, C₂₋₃ alkenyl, C₂₋₆ alkynyl and C₂₋₃ alkynyl.

In an embodiment, at least two of R², R³, R⁴, and R⁵ are H.

In an embodiment, R¹ is methyl and R⁶ is H.

In an embodiment, R⁹ is H.

In an embodiment when X is phenyl, n is 0; in another embodiment, n is 1; in another embodiment, n is 2.

In an embodiment, R⁸ is hydroxy and the compounds are characterized as hydroxamates. In another embodiment, R⁸ is substituted aryl or heteroaryl and the compounds are characterized as arylamides.

In an embodiment, X is phenyl. In various embodiments, the N—R⁹ and —C(O)NH—R⁸ groups are disposed on the phenyl in a 1,4-configuration, where N—R⁹ is considered as the 1-position.

In an embodiment, X is thiophene. In various embodiments, the N—R⁹ and —C(O)NH—R¹ groups are disposed on the thiophene in a 2,5-configuration, where N—R⁹ is considered as the 2-position (with the S atom of the thiophene ring taken as the 1-position).

In an embodiment, X is pyridine. In various embodiments, the N—R⁹ and —C(O)NH—R¹ groups are disposed on the pyridine in a 2,5-configuration, where N—R⁹ is considered as the 2-position, or in a 3,6-configuration, where N—R⁹ is considered as the 3-position (in all cases, the N atom of the pyridine ring is taken as the 1-position).

In the Tables that follow, examples are given with n=0 or n=1. When n=0, the entry in the R⁷ column reads H (hydrogen) to indicate that all substituents are hydrogen. When n=1, the entry in the R⁷ column gives the identity and position of the single non-hydrogen substituent.

Pharmaceutical compositions comprise an HDAC and/or CDK-inhibitory effective amount of one or more compounds described above and a pharmaceutically-acceptable carrier.

Methods of inhibiting or treating diseases arising from abnormal cell proliferation and differentiation comprise administering to a subject a therapeutically effective amount of one or more compounds described herein. Other methods involve co-therapies by administering one or more of the compounds together with other anti-cancer agents.

The compounds above are more fully described in the detailed description that follows.

### DETAILED DESCRIPTION

The following description is merely exemplary in nature and is not intended to limit the present disclosure, application, or uses.

### DEFINITIONS

“Alkenyl” refers to a straight or branched hydrocarbyl group with at least one site of unsaturation, i.e. a carbon-carbon, sp² double bond. In an embodiment, alkenyl has from 2 to 12 carbon atoms. In some embodiments, alkenyl is a C₂-C₁₀ alkenyl group or a C₂-C₆ alkenyl group. Examples of alkenyl group include, but are not limited to, ethylene or vinyl (—CH═CH₂), allyl (—CH₂CH═CH₂), cyclopentenyl (—C₅H₇), and 5-hexenyl (—CH₂CH₂CH₂CH₂CH═CH₂).

“Alkanoyl” is the group RC(O)—; “alkanoyloxy” is RC(O)O—; and “alkanoylamino” is RC(O)NR′—; where R is an alkyl group as defined herein, and R′ is hydrogen or alkyl. In various embodiments, R is a C₁-C₁₀ alkyl group or a C₁-C₆ alkyl group.

“Alkoxy” is RO— where R is alkyl. Non-limiting examples of alkoxy groups include methoxy, ethoxy and propoxy.

“Alkoxyalkyl” refers to an alkyl moiety substituted with an alkoxy group. Examples of alkoxyalkyl groups include methoxymethyl, methoxyethyl, methoxypropyl and ethoxyethyl.

“Alkoxycarbonyl” is ROC(O)—, where R is an alkyl group as defined herein. In various embodiments, R is a C₁-C₁₀ alkyl group or a C₁-C₆ alkyl group.

“Alkyl” refers to a straight or branched chain hydrocarbyl group. In an embodiment, alkyl has from 1 to 12 carbon atoms. In some embodiments, alkyl is a C₁-C₁₀ alkyl group or a C₁-C₆ alkyl group. Examples of alkyl groups include, but are not limited to, methyl, ethyl, propyl, isopropyl, butyl, isobutyl, t-butyl, pentyl, hexyl, heptyl, octyl, nonyl and decyl.

“Alkylamino” refers to an amino group substituted with one or more alkyl groups. “N-(alkyl)amino” is RNH- and “N,N-(alkyl)₂-amino” is R₂N—, where the R groups are alkyl as defined herein and are the same or different. In various embodiments, R is a C₁-C₁₀ alkyl group or a C₁-C₆ alkyl group. Examples of alkylamino groups include methylamino, ethylamino, propylamino, butylamino, dimethylamino, diethylamino, and methylethylamno.

“Alkylaminoalkyl” refers to an alkyl moiety substituted with an alkylamino group, wherein alkylamino is as defined herein. Examples of alkylaminoakyl groups include methylaminomethyl and ethylaminomethyl.

“Alkynyl” refers to a straight or branched carbon-chain group with at least one site of unsaturation, i.e. a carbon-carbon, sp triple bond. In an embodiment, alkynyl has from 2 to 12 carbon atoms. In some embodiments, alkynyl is a C₂-C₁₀ alkynyl group or a C₂-C₆ alkynyl group. Examples of alkynyl groups include acetylenic (—C≡CH) and propargyl (—CH₂C≡CH).

“Aryl” refers to any monocyclic or bicyclic carbon ring of up to 7 atoms in each ring, wherein at least one ring is aromatic, or an aromatic ring system of 5 to 14 carbons atoms which includes a carbocyclic aromatic group fused with a 5- or 6-membered cycloalkyl group. Examples of aryl groups include, but are not limited to, phenyl, naphthyl, tetrahydronaphthyl and indanyl.

“Aryloxy” is RO—, where R is aryl. “Arylthio” is RS—, where R is aryl.

“Carbamoyl” is the group NH₂—C(O)—; the nitrogen can be substituted with alkyl groups. N-(alkyl)carbamoyl is RNH—C(O)— and N,N-(alkyl)₂ carbamoyl is R₂N—C(O)—, where the R groups are alkyl as defined herein and are the same or different. In various embodiments, R is a C₁-C₁₀ alkyl group or a C₁-C₆ alkyl group.

“Cycloalkyl” is a hydrocarbyl group containing at least one saturated or partially unsaturated ring structure, and attached via a ring carbon. In various embodiments, it refers to a saturated or a partially unsaturated C₃-C₁₂ cyclic moiety, examples of which include cyclopropyl, cyclobutyl, cyclopentyl, cyclopentenyl, cyclohexyl, cyclohexenyl, cycloheptyl and cyclooctyl.

“Cycloalkyloxy” is RO—, where R is cycloalkyl.

“Cycloalkylalkyl” refers to an alkyl moiety substituted with a cycloalkyl group, wherein cycloalkyl is as defined herein. Examples of cycloalkylalkyl groups include cyclopropylmethyl, cyclobutylmethyl, cyclopentylethyl and cyclohexylmethyl.

“Dialkylamino” refers to an RR′N— group where R and R′ are independently alkyl as defined herein. Examples of dialkylamino groups include, but are not limited to, dimethylamino, diethylamino, methylethylamino and methylpropylamino. In various embodiments, R and R′ are independently C₁-C₁₀ alkyl or C₁-C₆ alkyl.

“Dialkylaminoalkyl” refers to an alkyl moiety substituted with a dialkylamino group, wherein dialkylamino is as defined herein. Examples of dialkylaminoalkyl groups include, but are not limited to, dimethylaminomethyl and diethylaminomethyl.

“Halo” refers to chloro (—Cl), bromo (—Br), fluoro (—F) or iodo (—I).

“Haloalkoxy” refers to an alkoxy group substituted with one or more halo groups and examples of haloalkoxy groups include, but are not limited to, —OCF₃, —OCHF₂ and —OCH₂F.

“Haloalkoxyalkyl” refers to an alkyl moiety substituted with a haloalkoxy group, wherein haloalkoxy is as defined herein. Examples of haloalkoxyalkyl groups include trifluoromethoxymethyl, trifluoroethoxymethyl and trifluoromethoxyethyl.

“Haloalkyl” refers to an alkyl moiety substituted with one or more halo groups. Examples of haloalkyl groups include —CF₃ and —CHF₂.

“Heterocyclyl” includes the heteroaryls defined below and refers to a saturated or partially unsaturated monocyclic, bicyclic or tricyclic group of 2 to 14 ring-carbon atoms and, in addition to ring-carbon atoms, 1 to 4 heteroatoms selected from P, N, O and S. In various embodiments the heterocyclic group is attached to another moiety through carbon or through a heteroatom, and is optionally substituted on carbon or a heteroatom. Examples of heterocyclyl include azetidinyl, benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrahydrothiopyranyl, tetrahydroisoquinolinyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof.

“Heterocyclyloxy” is RO—, where R is heterocyclyl.

“Heterocyclylthio” is RS—, where R is heterocyclyl.

“Heteroaryl” refers to a monocyclic, bicyclic or tricyclic ring having up to 7 atoms in each ring, wherein at least one ring is aromatic and contains from 1 to 4 heteroatoms in the ring selected from the group consisting of N, O and S, Non-limiting examples of heteroaryl include pyridyl, thienyl, furanyl, pyrimidyl, imidazolyl, pyranyl, pyrazolyl, thiazolyl, thiadiazolyl, isothiazolyl, oxazolyl, isoxazoyl, pyrrolyl, pyridazinyl, pyrazinyl, quinolinyl, isoquinolinyl, benzofuranyl, dibenzofuranyl, dibenzothiophenyl, benzothienyl, indolyl, benzothiazolyl, benzooxazolyl, benzimidazolyl, isoindolyl, benzotriazolyl, purinyl, thianaphthenyl and pyrazinyl. Attachment of heteroaryl can occur via an aromatic ring, or, if heteroaryl is bicyclic or tricyclic and one of the rings is not aromatic or contains no heteroatoms, through a non-aromatic ring or a ring containing no heteroatoms. “Heteroaryl” is also understood to include the N-oxide derivative of any nitrogen containing heteroaryl.

“Heteroaryloxy” is RO—, where R is heteroaryl.

“Hydroxyalkoxy” refers to an alkoxy group substituted with a hydroxyl group (—OH), wherein alkoxy is as defined herein. An example of hydroxyalkoxy is hydroxyethoxy.

“Hydroxyalkyl” refers to a linear or branched monovalent C₁-C₁₀ hydrocarbon group substituted with at least one hydroxy group and examples of hydroxyalkyl groups include, but are not limited to, hydroxymethyl, hydroxyethyl, hydroxypropyl and hydroxybutyl.

“Sulphamoyl” is NH₂—S(O)₂O—; “N-(alkyl)sulphamoyl” is RNH—S(O)₂O—; and “N,N-(alkyl)₂ sulphamoyl” is R₂N—S(O)₂O—, where the R groups are alkyl as defined herein and are the same or different. In various embodiments, R is a C₁-C₁₀ alkyl group or a C₁-C₆ alkyl group.

“Pharmaceutically-acceptable” means suitable for use in pharmaceutical preparations, generally considered as safe for such use, officially approved by a regulatory agency of a national or state government for such use, or being listed in the U.S. Pharmacopoeia or other generally recognized pharmacopoeia for use in animals, and more particularly in humans.

“Pharmaceutically-acceptable carrier” refers to a diluent, adjuvant, excipient, or carrier, or other ingredient which is pharmaceutically-acceptable and with which a compound of the invention is administered.

“Pharmaceutically-acceptable salt” refers to a salt which may enhance desired pharmacological activity. Examples of pharmaceutically-acceptable salts include acid addition salts formed with inorganic or organic acids, metal salts and amine salts. Examples of acid addition salts formed with inorganic acids include salts with hydrochloric acid, hydrobromic acid, sulfuric acid, nitric acid and phosphoric acid. Examples of acid addition salts formed with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxy-benzoyl)-benzoic acid, cinnamic acid, mandelic acid, methanesulfonic acid, ethanesulfonic acid, 1,2-ethanedisulfonic acid, 2-hydroxyethane-sulfonic acid, benzenesulfonic acid, p-chlorobenzenesulfonic acid, 2-naphthalenesulfonic acid, p-toluenesulfonic acid, camphorsulfonic acid, 4-methyl-bicyclo[2.2.2]oct-2-enel-carboxylic acid, gluco-heptonic acid, 4,4′-methylenebis(3-hydroxy-2-naphthoic) acid, 3-phenylpropionic acid, trimethyl-acetic acid, tertiary butylacetic acid, lauryl sulfuric acid, gluconic acid, glutamic acid, hydroxy-naphthoic acids, salicylic acid, stearic acid and muconic acid. Examples of metal salts include salts with sodium, potassium, calcium, magnesium, aluminum, iron, and zinc ions. Examples of amine salts include salts with ammonia and organic nitrogenous bases strong enough to form salts with carboxylic acids.

“Therapeutically-effective amount” refers to an amount of a compound that, when administered to a subject for treating a disease, is sufficient to effect treatment for the disease. “Therapeutically effective amount” can vary depending on the compound, the disease and its severity, the age, the weight, etc. of the subject to be treated.

Embraced herein, where applicable, are permissible isomers such as tautomers, racemates, enantiomers, diastereomers, atropisomers, configurational isomers of double bonds (E- and/or Z-), cis- and trans-configurations in ring substitution patterns, and isotopic variants.

In one embodiment, the invention provides a compound of Formula (I) or a pharmaceutically acceptable salt thereof, wherein R¹, R², R³, R⁴ and R⁵ are independently selected from the group consisting of H, halo, nitro, cyano, hydroxy, hydroxyalkyl, haloalkyl, haloalkoxy, amino, azido, carboxy, carbamoyl, mercapto, sulphamoyl, alkyl, C₂₋₆ alkenyl, C₂₋₆ alkynyl, C₁₋₆ alkoxy, C₁₋₆ alkanoyl, C₁₋₆ alkanoyloxy, N—(C₁₋₆ alkyl)amino, N,N—(C₁₋₆ alkyl)₂ amino, C₁₋₆ alkanoylamino, N—(C₁₋₆ alkyl)carbamoyl, N,N—(C₁₋₆ alkyl)₂-carbamoyl, C₁₋₆ alkyl-S(O)ₐ wherein a is 0, 1 or 2, C₁₋₆alkoxycarbonyl, NH₂—S(O)₂NH—, N—(C₁₋₆ alkyl)sulphamoyl, N,N—(C₁₋₆ alkyl)₂sulphamoyl, aryl, aryloxy, arylthio, heteroaryl, heteroaryloxy, cycloalkyl, cycloalkyloxy, heterocyclyl, heterocyclyl(C═O)—, heterocyclyloxy and heterocyclylthio; wherein each of R¹, R², R³, R⁴ and R⁵ is optionally substituted by one or more A where such an optional substitution is chemically feasible; R⁶ is H, halo, nitro, cyano, trifluoromethyl, trifluoromethoxy, amino, carboxy, carbamoyl, sulphamoyl, C₁₋₃ alkyl, C₂₋₃ alkenyl, C₂₋₃ alkynyl, C₁₋₃ alkoxy, C₁₋₃ alkanoyl, N—(C₁₋₃ alkyl)amino, N,N—(C₁₋₂ alkyl)₂ amino, C₁₋₃ alkanoylamino, N—(C₁₋₃ alkyl)carbamoyl, N,N—(C₁₋₂ alkyl)₂ carbamoyl, C₁₋₃ alkyl-S(O)ₐ wherein a is 0, 1 or 2, NH₂—S(O)₂NH—, N—(C₁₋₃ alkyl)sulphamoyl or N,N—(C₁₋₃ alkyl)₂sulphamoyl; wherein R⁶ is optionally substituted by one or more B where such an optional substitution is chemically feasible; X is phenyl, 5-membered heteroaryl, or 6-membered heteroaryl, wherein the heteroaryl contains one or more heteroatoms selected from N, S and O; R⁷ and n are as defined above; R⁸ is hydroxy, aryl or heteroaryl, wherein aryl or heteroaryl are substituted with —NH₂ or —OH and aryl or heteroaryl is optionally further substituted with one or more groups R¹⁰ selected from amino, halo, alkyl, cycloalkyl, heterocyclyl, aryl and heteroaryl; R⁹ is H, alkyl, haloalkyl, aminoalkyl, cycloalkyl, heterocyclyl, aryl, heteroaryl, wherein R⁹ is optionally substituted by one or more D where such an optional substitution is chemically feasible; A and B are independently selected from halo, nitro, cyano, hydroxy, hydroxyalkyl, haloalkyl, haloalkoxy, amino, azido, carboxy, carbamoyl, mercapto, sulphamoyl, C₁₋₆ alkyl, C₂₋₆ alkenyl, C₂₋₆ alkynyl, C₁₋₆ alkoxy, C₁₋₆ alkanoyl, C₁₋₆ alkanoyloxy, N—(C₁₋₆ alkyl)amino, N,N—(C₁₋₆ alkyl)₂ amino, C₁₋₆ alkanoylamino, N—(C₁₋₆ alkyl)carbamoyl, N,N—(C₁₋₆ alkyl)₂-carbamoyl, C₁₋₆ alkyl-S(O)ₐ wherein a is 0, 1 or 2, C₁₋₆ alkoxycarbonyl, N—(C₁₋₆ alkyl)sulphamoyl, N,N—(C₁₋₆ alkyl)₂sulphamoyl, H₂NS(O)₂NH—, N—(C₁₋₆ alkyl)NH S(O)₂NH—, N,N—(C₁₋₆ alkyl)₂N S(O)₂NH—, aryl, aryloxy, arylthio, heteroaryl, heteroaryloxy, cycloalkyl, cycloalkyloxy, heterocyclyl, heterocyclyl(C═O)—, heterocyclyloxy and heterocyclylthio; and D is selected from halo, nitro, cyano, hydroxy, amino, azido, carboxy and mercapto.

In one embodiment, the invention provides a compound of Formula (I) or a pharmaceutically acceptable salt thereof:

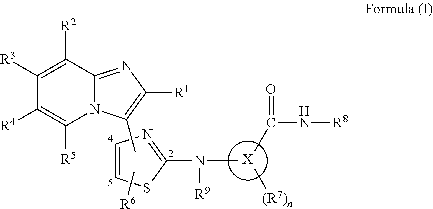

wherein R¹, R², R³, R⁴, R⁵, R⁶, R⁷, R⁸, R⁹ and X are as defined in various embodiments above. In a particular embodiment, R⁹ is H.

A compound of Formula (I) contains a divalent thiazole ring linking a substituted or unsubstituted imidazopyridine ring to an amino-containing group —NR⁹—X—CONH—R⁸. The thiazole ring is also substituted by R⁶. Formula (I) indicates that the attachment of substituents on the thiazole ring is variable. For example, the imidazopyridine ring and any R⁶ can be attached to carbon atoms 4- and 5-drawn in Formula (I). When R⁶ is hydrogen, it is conventional to call the thiazole divalent to account for attachment of the imidazopyridine ring and the amino-containing group. In particular embodiments, compounds are selected from those of Formula (I-a), Formula (I-b), Formula (I-c), Formula (I-d) and Formula (I-e), with substituents defined as in Formula (I).

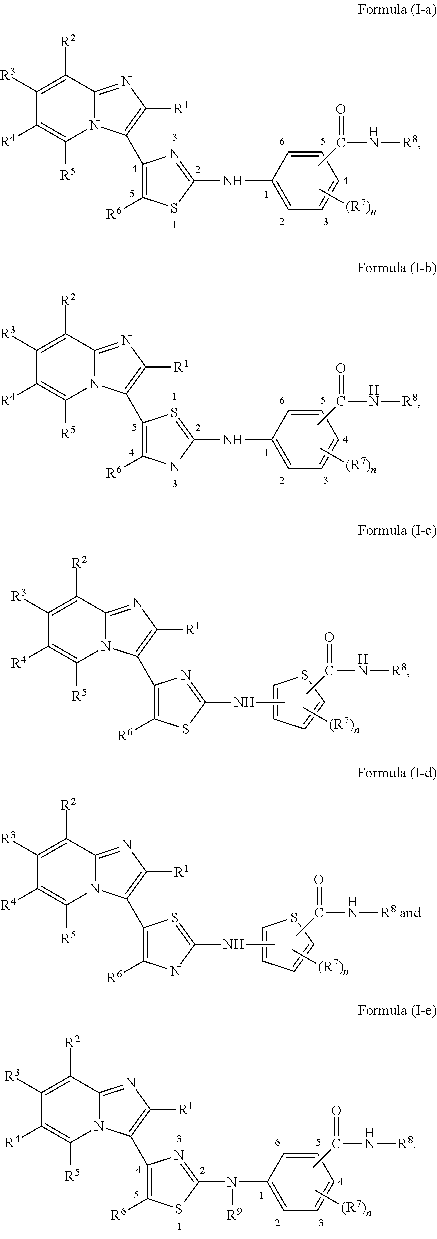

Compounds described herein are useful to inhibit HDACs and/or CDKs. In one embodiment, therefore, a compound of the invention is used in inhibiting HDAC and/or CDK enzymes such as, for example, mammalian HDAC and/or CDK. More specifically, a compound of the invention can be used to treat or inhibit HDAC and/or CDK-mediated diseases or abnormalities.

In an embodiment of the compounds, one or more (including all) of the substituents R¹, R², R³, R⁴, R⁵, R⁶, R⁷ and R⁸ are further limited as follows:

R¹ is selected from the group consisting of hydrogen, methyl, ethyl, propyl, methoxy, ethoxy, methoxymethyl, ethoxyethyl, propoxyethyl, methoxyethoxy, trifluoromethyl, hydroxyethoxy, dimethylamino, diethylamino, dimethylaminomethyl, diethylaminomethyl, dimethylaminoethoxy, trifluoromethoxymethyl, trifluoroethoxymethyl, benzyl, phenylethyl, trifluoromethylphenylethyl, phenoxymethyl, fluorophenoxymethyl, phenylethylaminomethyl, benzylaminomethyl, morpholinylmethyl, morpholinylethoxy, imidazolylmethyl, triazinylmethyl, piperidinylmethyl, piperidinyloxy, trifluoromethylpiperidinylmethyl, pyridinyloxymethyl, pyridinylmethoxy, tetrahydropyrazinyloxy, methylpiperazinylmethyl, pyrrolidinylmethyl and pyrrolidinylethoxy;

R², R³, R⁴, and R⁵ are independently selected from hydrogen, chloro, fluoro, bromo, methyl, ethyl, propyl, methoxy, ethoxy, carboxy, cyano, methoxymethyl, ethoxyethyl, propoxyethyl, methoxyethoxy, trifluoromethyl, hydroxyethoxy, dimethylamino, diethylamino, dimethylaminomethyl, dimethylaminoethyl, diethylaminomethyl, dimethylaminoethoxy, trifluoromethoxymethyl, trifluoroethoxymethyl, 3-oxetanoxy, trifluoro ethylaminomethyl, N-methyl-N-methoxyethyl-aminoethyl, cyclopropanylmethyl, cyclobutoxy, 1-cyclopropanylethoxy, cyclopropanylmethylaminomethyl, 4-methylpiperazin-1-carbonyl, isoindolin-2-yl, N-methoxyethylcarb amoyl, N-(morpholin-4-yl)-ethylcarbamoyl, dimethylamino ethylamino, methylcarboxy, N,N-dimethylamino ethylcarbamoyl, benzyl, phenylethyl, trifluoromethylphenylethyl, phenoxymethyl, fluorophenoxymethyl, phenylethylaminomethyl, benzylaminomethyl, triazinylmethyl, piperidinylmethyl, piperidinyloxy, trifluoromethylpiperidinylmethyl, pyridinyloxymethyl, pyridinylmethoxy, tetrahydropyrazinyloxy, methylpiperazinylmethyl, pyrrolidin-1-yl, pyrrolidin-2-yl, pyrrolidin-3-yl, pyrrolidin-1-ylmethyl, pyrrolidin-2-ylmethyl, pyrrolidin-3-ylmethyl, pyrrolidin-1-ylethoxy, pyrrolidin-2-ylethoxy, pyrrolidin-3-ylethoxy, imidazol-1-ylmethyl, imidazol-2-ylmethyl, imidazol-4-ylmethyl, imidazolidin-1-yl, imidazolidin-2-yl, imidazolidin-4-yl, imidazolidin-1-ylmethyl, imidazolidin-2-ylmethyl, imidazolidin-4-ylmethyl, imidazolin-1-yl, imidazolin-2-yl, imidazolin-4-yl, pyrazolidin-1-yl, pyrazolidin-3-yl, pyrazolidin-4-yl, pyrazolin-1-yl, pyrazolin-3-yl, pyrazolin-4-yl, piperidin-1-yl, piperidin-2-yl, piperidin-3-yl, piperidin-4-yl, piperidin-1-ylmethyl, piperidin-2-ylmethyl, piperidin-3-ylmethyl, piperidin-4-ylmethyl, piperazin-1-yl, piperazin-2-yl, piperazin-3-yl, morpholin-2-yl, morpholin-3-yl, morpholin-4-yl, morpholin-2-ylmethyl, morpholin-3-ylmethyl, morpholin-4-ylmethyl, morpholin-2-ylethoxy, morpholin-3-ylethoxy and morpholin-4-ylethoxy; in an embodiment, at least two of R², R³, R⁴, and R⁵ are H;

R⁶ is H, methyl, ethyl, bromo or trifluoromethyl;

X is phenyl or 5-membered heteroaryl;

R⁷ is independently fluoro, chloro, bromo, or methyl and n is 0, 1 or 2; and

R⁸ is hydroxyl, aryl or heteroaryl, wherein aryl or heteroaryl are substituted with —NH₂ or —OH at a ring position adjacent to attachment of the —CONH-moiety, and R⁸ is optionally further substituted with one or more groups R¹⁰ selected from amino, halo, alkyl, alicyclyl, heterocyclyl and aryl.

In particular embodiments, R⁸ is hydroxy,

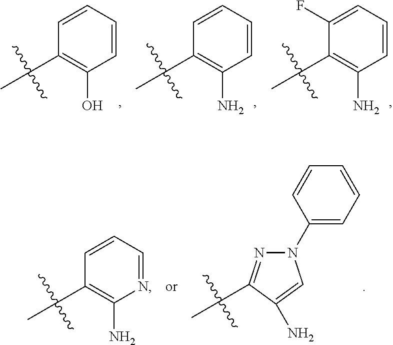

In various embodiments, the NH linker to thiazole and —CONHR⁸ moiety are disposed about the phenyl ring of Formula (I-a) or (I-b) in either a 1,3-(meta) or a 1,4-(para) configuration. R⁷ can be attached to any ring position of the phenyl ring which is not occupied by the NH linker and —CONHR⁸ moiety and such attachment includes 1,2-(ortho), 1,3-(meta) and 1,4-(para) configurations wherein the NH linker is at position 1. In the Tables that follow, ortho-, meta- and para-configurations of R⁷ mean attachment to positions 2, 3, and 4 of the phenyl ring as shown in Formulas (I-a) and (I-b), respectively. Where R⁷ is an ortho-substitution (i.e., position 2), meta-CONHR⁸ moiety is intended to be at position 5.

In one embodiment, the invention provides a compound of Formula (I-a) and a pharmaceutically acceptable salt thereof:

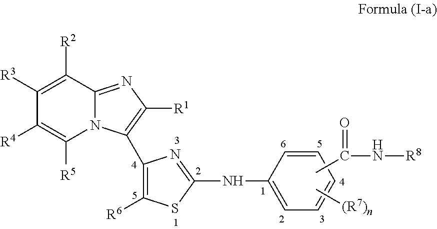

wherein R¹, R², R³, R⁴, R⁵, R⁶, R⁷ and R⁸ are as defined above for various aspects of Formula (I).

In an embodiment of Formula (I-a), R¹, R², R³, R⁴ and R⁵ are H; R⁶ is H, alkyl or haloalkyl; R⁷ is independently fluoro, chloro, bromo, or methyl and n is 0, 1 or 2; and R⁸ is hydroxyl, aryl or heteroaryl, wherein aryl or heteroaryl are substituted with —NH₂ or —OH at a ring position adjacent to attachment of the —CONH-moiety, and R⁸ is optionally further substituted with one or more groups selected from amino, halo, alkyl, alicyclyl, heterocyclyl and aryl.

Non-limiting examples of such compounds include the following compounds and pharmaceutically acceptable salts thereof:

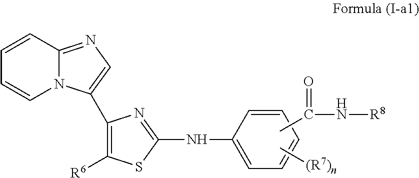

| Compound | | | —CONHR⁸ | |
|---|---|---|---|---|
| No. | R⁶ | R⁷ | attachment | R⁸ |
| a1-01 | H | H | para | —OH |
| a1-02 | H | H | meta | —OH |
| a1-03 | —CH₃ | H | para | —OH |
| a1-04 | —CH₃ | H | meta | —OH |
| a1-05 | H | H | para | 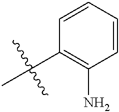 |
| a1-06 | H | H | meta | 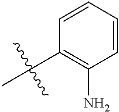 |
| a1-07 | —CH₃ | H | para | 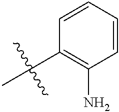 |
| a1-08 | —CH₃ | H | meta | 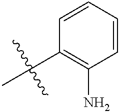 |
| a1-09 | H | H | para | 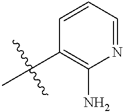 |
| a1-10 | H | H | meta | 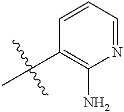 |
| a1-11 | —CH₃ | H | para | 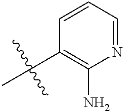 |
| a1-12 | —CH₃ | H | meta | 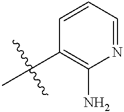 |
| a1-13 | H | H | para | 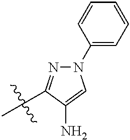 |
| a1-14 | H | H | meta | 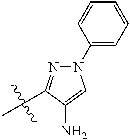 |
| a1-15 | —CH₃ | H | para | 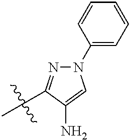 |
| a1-16 | —CH₃ | H | meta | 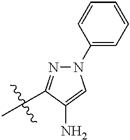 |
| a1-17 | H | H | para | 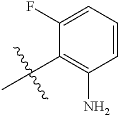 |
| a1-18 | H | H | meta | 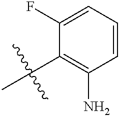 |
| a1 -19 | —CH₃ | H | para | 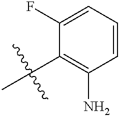 |
| a1-20 | —CH₃ | H | meta | 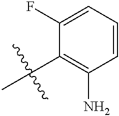 |
| a1 -21 | H | ortho-F | para | 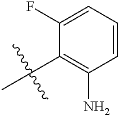 |
| a1-22 | H | ortho-F | meta | 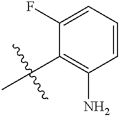 |
| a1-23 | —CH₃ | ortho-F | para | 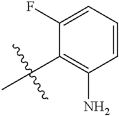 |
| a1-24 | —CH₃ | ortho-F | meta | 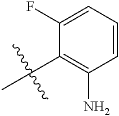 |

In another embodiment of Formula (I-a), R¹ is methyl; R², R³, R⁴ and R⁵ are H; R⁶ is H, alkyl or haloalkyl; R⁷ is fluoro, chloro, bromo, or methyl and n is 0 or 1; and R⁸ is hydroxyl, aryl or heteroaryl, wherein aryl or heteroaryl are substituted with —NH₂ or —OH at a ring position adjacent to attachment of the —CONH-moiety, and R⁸ is optionally substituted with one or more groups R¹⁰ selected from amino, halo, alkyl, alicyclyl, heterocyclyl and aryl.

Non-limiting examples of such compounds include the following compounds and pharmaceutically acceptable salts thereof:

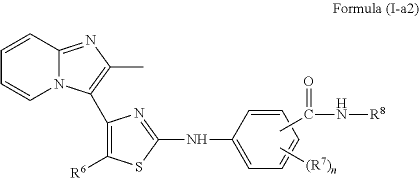

| Compound | | | —CONHR⁸ | |
|---|---|---|---|---|
| No. | R⁶ | R⁷ | attachment | R⁸ |
| a2-01 | H | H | para | —OH |
| a2-02 | H | H | meta | —OH |
| a2-03 | —CH₃ | H | para | —OH |
| a2-04 | —CH₃ | H | meta | —OH |
| a2-05 | H | H | para | 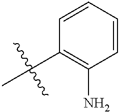 |
| a2-06 | H | H | meta | 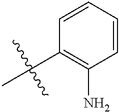 |
| a2-07 | —CH₃ | H | para | 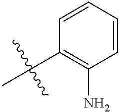 |
| a2-08 | —CH₃ | H | meta | 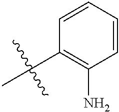 |
| a2-09 | H | H | para | 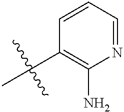 |
| a2-10 | H | H | meta | 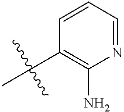 |
| a2-11 | —CH₃ | H | para | 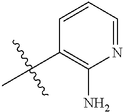 |
| a2-12 | —CH₃ | H | meta | 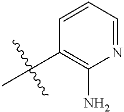 |
| a2-13 | H | H | para | 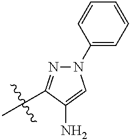 |
| a2-14 | H | H | meta | 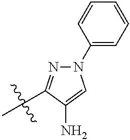 |
| a2-15 | —CH₃ | H | para | 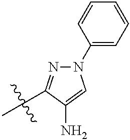 |
| a2-16 | —CH₃ | H | meta | 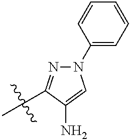 |
| a2-17 | H | H | para | 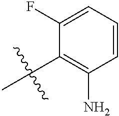 |
| a2-18 | H | H | meta | 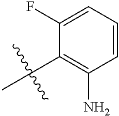 |
| a2-19 | —CH₃ | H | para | 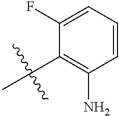 |
| a2-20 | —CH₃ | H | meta | 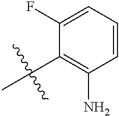 |
| a2-21 | H | ortho-F | para | 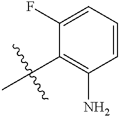 |
| a2-22 | H | ortho-F | meta | 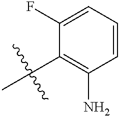 |
| a2-23 | —CH₃ | ortho-F | para | 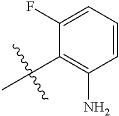 |
| a2-24 | —CH₃ | ortho-F | meta | 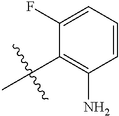 |

In another embodiment of Formula (I-a), at least two of R¹, R², R³, R⁴ and R⁵ are H, and each non-hydrogen R¹, R², R³, R⁴ and R⁵ is independently selected from, chloro, fluoro, bromo, methyl, ethyl, propyl, methoxy, ethoxy, carboxy, cyano, methoxymethyl, ethoxyethyl, propoxyethyl, methoxyethoxy, trifluoromethyl, hydroxyethoxy, dimethylamino, diethylamino, dimethylaminomethyl, dimethylamino ethyl, diethylaminomethyl, dimethylaminoethoxy, trifluoromethoxymethyl, trifluoroethoxymethyl, 3-oxetanoxy, trifluoroethylaminomethyl, N-methyl-N-methoxyethyl-aminoethyl, cyclopropanylmethyl, cyclobutoxy, 1-cyclopropanylethoxy, cyclopropanylmethylaminomethyl, 4-methylpiperazin-1-carbonyl, isoindolin-2-yl, N-methoxyethylcarbamoyl, N-(morpholin-4-yl)-ethylcarbamoyl, dimethylaminoethylamino, methylcarboxy, N,N-dimethylaminoethylcarbamoyl, benzyl, phenylethyl, trifluoromethylphenylethyl, phenoxymethyl, fluorophenoxymethyl, phenylethylaminomethyl, benzylaminomethyl, triazinylmethyl, piperidinylmethyl, piperidinyloxy, trifluoromethylpiperidinylmethyl, pyridinyloxymethyl, pyridinylmethoxy, tetrahydropyrazinyloxy, methylpiperazinylmethyl, pyrrolidin-1-yl, pyrrolidin-2-yl, pyrrolidin-3-yl, pyrrolidin-1-ylmethyl, pyrrolidin-2-ylmethyl, pyrrolidin-3-ylmethyl, pyrrolidin-1-ylethoxy, pyrrolidin-2-ylethoxy, pyrrolidin-3-ylethoxy, imidazol-1-ylmethyl, imidazol-2-ylmethyl, imidazol-4-ylmethyl, imidazolidin-1-yl, imidazolidin-2-yl, imidazolidin-4-yl, imidazolidin-1-ylmethyl, imidazolidin-2-ylmethyl, imidazolidin-4-ylmethyl, imidazolin-1-yl, imidazolin-2-yl, imidazolin-4-yl, pyrazolidin-1-yl, pyrazolidin-3-yl, pyrazolidin-4-yl, pyrazolin-1-yl, pyrazolin-3-yl, pyrazolin-4-yl, piperidin-1-yl, piperidin-2-yl, piperidin-3-yl, piperidin-4-yl, piperidin-1-ylmethyl, piperidin-2-ylmethyl, piperidin-3-ylmethyl, piperidin-4-ylmethyl, piperazin-1-yl, piperazin-2-yl, piperazin-3-yl, morpholin-2-yl, morpholin-3-yl, morpholin-4-yl, morpholin-2-ylmethyl, morpholin-3-ylmethyl, morpholin-4-ylmethyl, morpholin-2-ylethoxy, morpholin-3-ylethoxy and morpholin-4-ylethoxy; R⁶ is H, alkyl or haloalkyl; R⁷ is fluoro, chloro, bromo, or methyl and n is 0 or 1; and R⁸ is hydroxyl, aryl or heteroaryl, wherein aryl or heteroaryl are substituted with —NH₂ or —OH at a ring position adjacent to attachment of the —CONH-moiety, and R⁸ is optionally further substituted with one or more groups R¹⁰ selected from amino, halo, alkyl, alicyclyl, heterocyclyl and aryl.

Non-limiting examples of such compounds include the following compounds and pharmaceutically acceptable salts thereof:

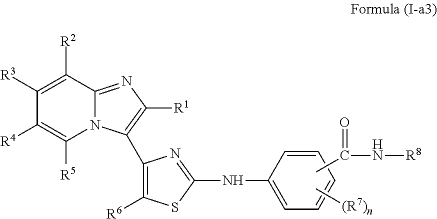

| Com- | | | | | | | | | |
|---|---|---|---|---|---|---|---|---|---|
| pound | | | | | | | | —CONHR⁸ | |
| No. | R¹ | R² | R³ | R⁴ | R⁵ | R⁶ | R⁷ | attachment | R⁸ |
| a3-01 | —CH₃ | —Cl | H | H | H | H | H | para | —OH |
| a3-02 | —CH₃ | H | —Cl | H | H | H | H | para | —OH |
| a3-03 | —CH₃ | H | H | —Cl | H | H | H | para | —OH |
| a3-04 | —CH₃ | H | H | H | —Cl | H | H | para | —OH |
| a3-05 | —CH₃ | —Cl | H | H | H | H | H | meta | —OH |
| a3-06 | —CH₃ | H | —Cl | H | H | H | H | meta | —OH |
| a3-07 | —CH₃ | H | H | —Cl | H | H | H | meta | —OH |
| a3-08 | —CH₃ | H | H | H | —Cl | H | H | meta | —OH |
| a3-09 | —CH₃ | —Cl | H | H | H | H | H | para | 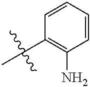 |
| a3-10 | —CH₃ | H | —Cl | H | H | H | H | para | 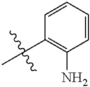 |
| a3-11 | —CH₃ | H | H | —Cl | H | H | H | para | 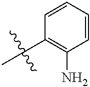 |
| a3-12 | —CH₃ | H | H | H | —Cl | H | H | para | 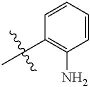 |
| a3-13 | —CH₃ | —Cl | H | H | H | H | H | para | 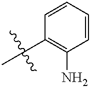 |
| a3-14 | —CH₃ | H | —Cl | H | H | H | H | para | 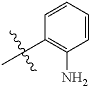 |
| a3-15 | —CH₃ | H | H | —Cl | H | H | H | para | 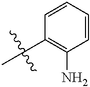 |
| a3-16 | —CH₃ | H | H | H | —Cl | H | H | para | 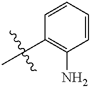 |
| a3-17 | —CH₃ | —Cl | H | H | H | H | H | para | 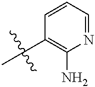 |
| a3-18 | —CH₃ | H | —Cl | H | H | H | H | para | 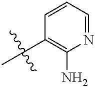 |
| a3-19 | —CH₃ | H | H | —Cl | H | H | H | para | 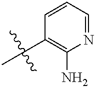 |
| a3-20 | —CH₃ | H | H | H | —Cl | H | H | para | 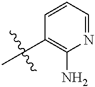 |
| a3-21 | —CH₃ | —Cl | H | H | H | H | H | para | 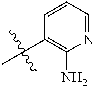 |
| a3-22 | —CH₃ | H | —Cl | H | H | H | H | para | 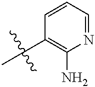 |
| a3-23 | —CH₃ | H | H | —Cl | H | H | H | para | 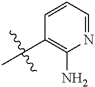 |
| a3-24 | —CH₃ | H | H | H | —Cl | H | H | para | 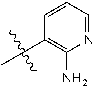 |
| a3-25 | —CH₃ | —CF₃ | H | H | H | H | H | para | —OH |
| a3-26 | —CH₃ | H | —CF₃ | H | H | H | H | para | —OH |
| a3-27 | —CH₃ | H | H | —CF₃ | H | H | H | para | —OH |
| a3-28 | —CH₃ | H | H | H | —CF₃ | H | H | para | —OH |
| a3-29 | —CH₃ | —CF₃ | H | H | H | H | H | para | 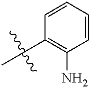 |
| a3-30 | —CH₃ | H | —CF₃ | H | H | H | H | para | 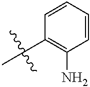 |
| a3-31 | —CH₃ | H | H | —CF₃ | H | H | H | para | 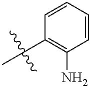 |
| a3-32 | —CH₃ | H | H | H | —CF₃ | H | H | para | 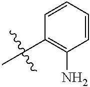 |
| a3-33 | —CH₃ | —OCH₃ | H | H | H | H | H | para | —OH |
| a3-34 | —CH₃ | H | —OCH₃ | H | H | H | H | para | —OH |
| a3-35 | —CH₃ | H | H | —OCH₃ | H | H | H | para | —OH |
| a3-36 | —CH₃ | H | H | H | —OCH₃ | H | H | para | —OH |
| a3-37 | —CH₃ | —OCH₃ | H | H | H | H | H | para | 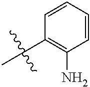 |
| a3-38 | —CH₃ | H | —OCH₃ | H | H | H | H | para | 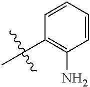 |
| a3-39 | —CH₃ | H | H | —OCH₃ | H | H | H | para | 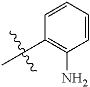 |
| a3-40 | —CH₃ | H | H | H | —OCH₃ | H | H | para | 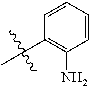 |
| a3-41 | H | 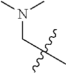 | H | H | H | H | H | para | —OH |
| a3-42 | H | H | 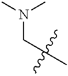 | H | H | H | H | para | —OH |
| a3-43 | H | H | H | 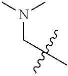 | H | H | H | para | —OH |
| a3-44 | H | H | H | H | 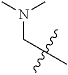 | H | H | para | —OH |
| a3-45 | H | 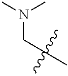 | H | H | H | H | H | para | 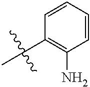 |
| a3-46 | H | H | 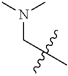 | H | H | H | H | para | 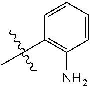 |
| a3-47 | H | H | H | 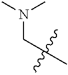 | H | H | H | para | 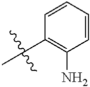 |
| a3-48 | H | H | H | H | 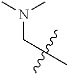 | H | H | para | 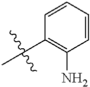 |
| a3-45 | H | 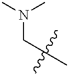 | H | H | H | H | ortho- F | para | 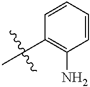 |
| a3-46 | H | H | 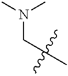 | H | H | H | ortho- F | para | 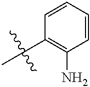 |
| a3-47 | H | H | H | 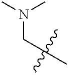 | H | H | ortho- F | para | 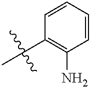 |
| a3-48 | H | H | H | H | 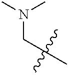 | H | ortho- F | para | 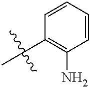 |
| a3-49 | H | 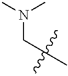 | H | H | H | H | ortho- F | para | 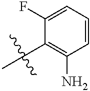 |
| a3-50 | H | H | 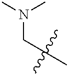 | H | H | H | ortho- F | para | 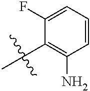 |
| a3-51 | H | H | H | 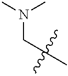 | H | H | ortho- F | para | 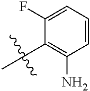 |
| a3-52 | H | H | H | H | 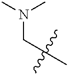 | H | ortho- F | para | 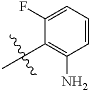 |
| a3-53 | H | 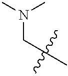 | H | H | H | H | H | para | 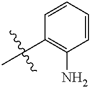 |
| a3-54 | H | H | 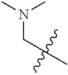 | H | H | H | H | para | 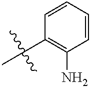 |
| a3-55 | H | H | H | 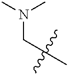 | H | H | H | para | 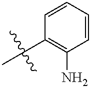 |
| a3-56 | H | H | H | H | 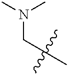 | H | H | para | 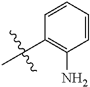 |
| a3-57 | H | 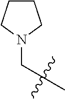 | H | H | H | H | H | para | 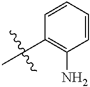 |
| a3-58 | H | H | 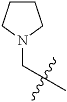 | H | H | H | H | para | 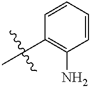 |
| a3-59 | H | H | H | 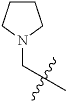 | H | H | H | para | 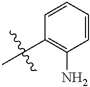 |
| a3-60 | H | H | H | H | 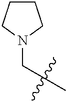 | H | H | para | 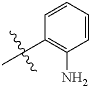 |
| a3-61 | H | 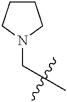 | H | H | H | H | ortho- F | para | 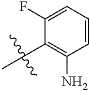 |
| a3-62 | H | H | 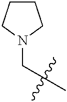 | H | H | H | ortho- F | para | 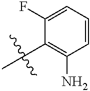 |
| a3-63 | H | H | H | 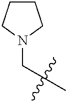 | H | H | ortho- F | para | 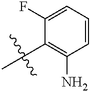 |
| a3-64 | H | H | H | H | 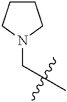 | H | ortho- F | para | 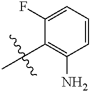 |
| a3-65 | H | 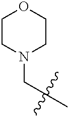 | H | H | H | H | H | para | 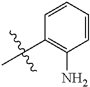 |
| a3-66 | H | H | 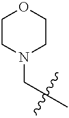 | H | H | H | H | para | 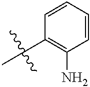 |
| a3-67 | H | H | H | 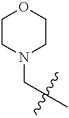 | H | H | H | para | 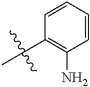 |
| a3-68 | H | H | H | H | 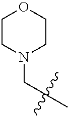 | H | H | para | 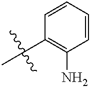 |
| a3-69 | H | 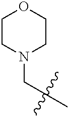 | H | H | H | H | ortho- F | para | 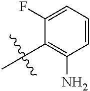 |
| a3-70 | H | H | 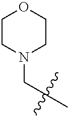 | H | H | H | ortho- F | para | 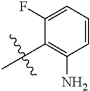 |
| a3-71 | H | H | H | 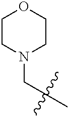 | H | H | ortho- F | para | 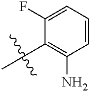 |
| a3-72 | H | H | H | H | 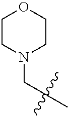 | H | ortho- F | para | 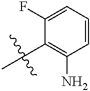 |
| a3-73 | H | 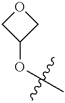 | H | H | H | H | H | para | 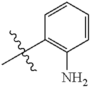 |
| a3-74 | H | H | 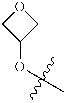 | H | H | H | H | para | 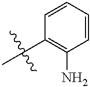 |
| a3-75 | H | H | H | 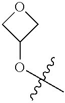 | H | H | H | para | 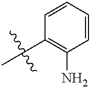 |
| a3-76 | H | H | H | H | 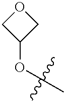 | H | H | para | 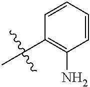 |
| a3-77 | H | 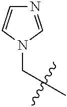 | H | H | H | H | H | para | 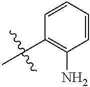 |
| a3-78 | H | H | 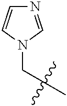 | H | H | H | H | para | 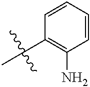 |
| a3-79 | H | H | H | 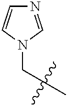 | H | H | H | para | 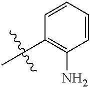 |
| a3-80 | H | H | H | H | 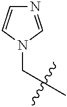 | H | H | para | 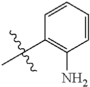 |
| a3-81 | H | 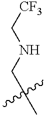 | H | H | H | H | H | para | 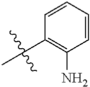 |
| a3-82 | H | H | 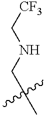 | H | H | H | H | para | 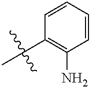 |
| a3-83 | H | H | H | 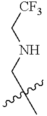 | H | H | H | para | 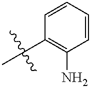 |
| a3-84 | H | H | H | H | 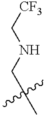 | H | H | para | 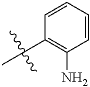 |
| a3-85 | H | 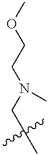 | H | H | H | H | H | para | 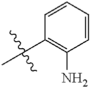 |
| a3-86 | H | H | 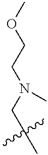 | H | H | H | H | para | 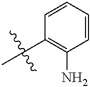 |
| a3-87 | H | H | H | 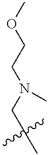 | H | H | H | para | 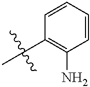 |
| a3-88 | H | H | H | H | 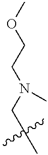 | H | H | para | 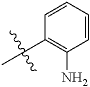 |
| a3-89 | H | 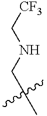 | H | H | H | H | H | para | 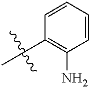 |
| a3-90 | H | H | 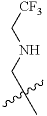 | H | H | H | H | para | 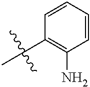 |
| a3-91 | H | H | H | 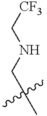 | H | H | H | para | 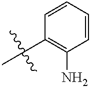 |
| a3-92 | H | H | H | H | 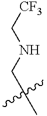 | H | H | para | 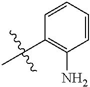 |
| a3-93 | H | 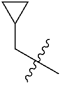 | H | H | H | H | H | para | 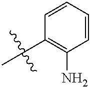 |
| a3-94 | H | H | 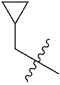 | H | H | H | H | para | 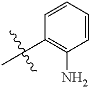 |
| a3-95 | H | H | H | 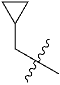 | H | H | H | para | 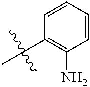 |
| a3-96 | H | H | H | H | 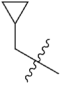 | H | H | para | 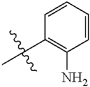 |
| a3-97 | H | 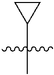 | H | H | H | H | H | para | 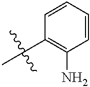 |
| a3-98 | H | H | 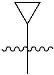 | H | H | H | H | para | 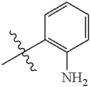 |
| a3-99 | H | H | H | 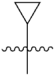 | H | H | H | para | 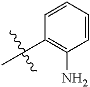 |
| a3-100 | H | H | H | H | 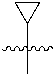 | H | H | para | 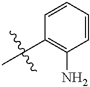 |
| a3-101 | H | 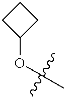 | H | H | H | H | H | para | 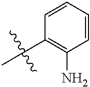 |
| a3-102 | H | H | 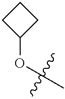 | H | H | H | H | para | 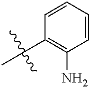 |
| a3-103 | H | H | H | 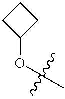 | H | H | H | para | 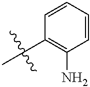 |
| a3-104 | H | H | H | H | 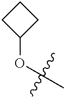 | H | H | para | 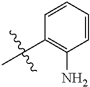 |
| a3-105 | H | 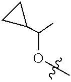 | H | H | H | H | H | para | 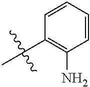 |
| a3-106 | H | H | 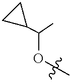 | H | H | H | H | para | 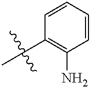 |
| a3-107 | H | H | H | 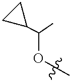 | H | H | H | para | 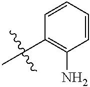 |
| a3-108 | H | H | H | H | 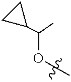 | H | H | para | 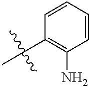 |
| a3-109 | H | 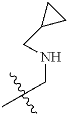 | H | H | H | H | H | para | 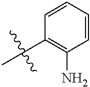 |
| a3-110 | H | H | 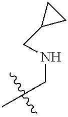 | H | H | H | H | para | 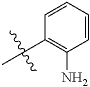 |
| a3-111 | H | H | H | 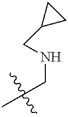 | H | H | H | para | 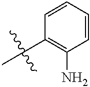 |
| a3-112 | H | H | H | H | 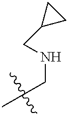 | H | H | para | 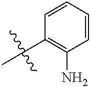 |
| a3-113 | H | 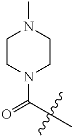 | H | H | H | H | H | para | 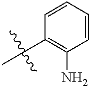 |
| a3-114 | H | H | 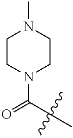 | H | H | H | H | para | 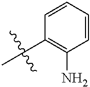 |
| a3-115 | H | H | H | 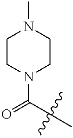 | H | H | H | para | 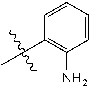 |
| a3-116 | H | H | H | H | 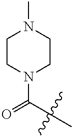 | H | H | para | 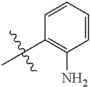 |
| a3-117 | H | 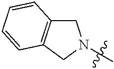 | H | H | H | H | H | para | 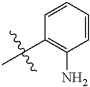 |
| a3-118 | H | H | 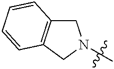 | H | H | H | H | para | 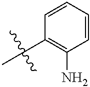 |
| a3-119 | H | H | H | 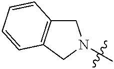 | H | H | H | para | 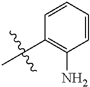 |
| a3-120 | H | H | H | H | 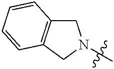 | H | H | para | 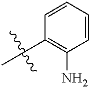 |
| a3-121 | —CH₃ | —F | H | H | H | H | H | para | —OH |
| a3-122 | —CH₃ | —F | H | H | H | H | H | para | 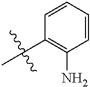 |
| a3-123 | —CH₃ | H | H | —Br | H | H | H | para | —OH |
| a3-124 | —CH₃ | H | H | —Br | H | H | H | para | 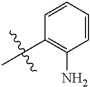 |
| a3-125 | —CH₃ | H | 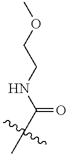 | H | H | H | H | para | —OH |
| a3-126 | —CH₃ | H | 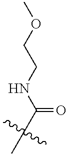 | H | H | H | H | para | 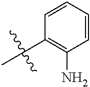 |
| a3-127 | —CH₃ | H | H | 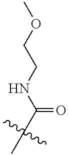 | H | H | H | para | —OH |
| a3-128 | —CH₃ | H | H | 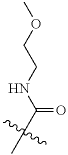 | H | H | H | para | 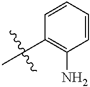 |
| a3-129 | —CH₃ | H | 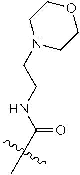 | H | H | H | H | para | —OH |
| a3-130 | —CH₃ | H | 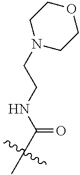 | H | H | H | H | para | 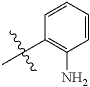 |
| a3-131 | —CH₃ | H | H | 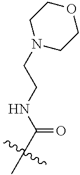 | H | H | H | para | —OH |
| a3-132 | —CH₃ | H | H | 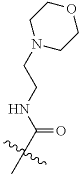 | H | H | H | para | 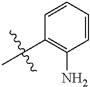 |
| a3-133 | —CH₃ | H | 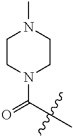 | H | H | H | H | para | —OH |
| a3-134 | —CH₃ | H | 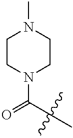 | H | H | H | H | para | 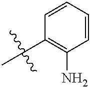 |
| a3-135 | —CH₃ | H | H | 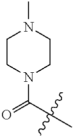 | H | H | H | para | —OH |
| a3-136 | —CH₃ | H | H | 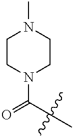 | H | H | H | para | 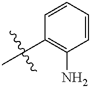 |
| a3-137 | —CH₃ | H | 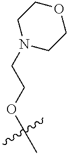 | H | H | H | H | para | —OH |
| a3-138 | —CH₃ | H | 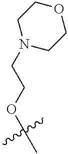 | H | H | H | H | para | 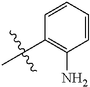 |
| a3-139 | —CH₃ | H | 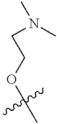 | H | H | H | H | para | —OH |
| a3-140 | —CH₃ | H | 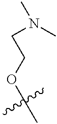 | H | H | H | H | para | 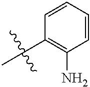 |
| a3-141 | —CH₃ | H | 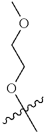 | H | H | H | H | para | —OH |
| a3-142 | —CH₃ | H | 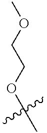 | H | H | H | H | para | 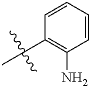 |
| a3-143 | —CH₃ | H | H | 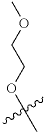 | H | H | H | para | —OH |
| a3-144 | —CH₃ | H | H | 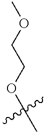 | H | H | H | para | 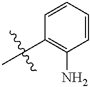 |
| a3-145 | —CH₃ | H | 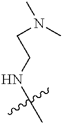 | H | H | H | H | para | —OH |
| a3-146 | —CH₃ | H | 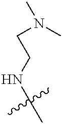 | H | H | H | H | para | 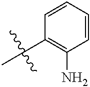 |
| a3-147 | —CH₃ | H | H | —OCH₃ | H | H | H | para | —OH |
| a3-148 | —CH₃ | H | H | —OCH₃ | H | H | H | para | 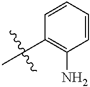 |
| a3-149 | H | H | —OCH₃ | H | H | H | H | para | —OH |
| a3-150 | H | H | —OCH₃ | H | H | H | H | para | 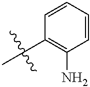 |
| a3-151 | —CH₃ | H | 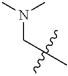 | H | H | H | H | para | —OH |
| a3-152 | —CH₃ | H | 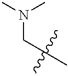 | H | H | H | H | para | 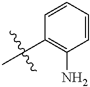 |
| a3-153 | —CF₃ | H | H | H | H | H | H | para | —OH |
| a3-154 | —CF₃ | H | H | H | H | H | H | para | 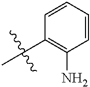 |
| a3-155 | —CH₃ | H | —CN | H | H | H | H | para | —OH |
| a3-156 | —CH₃ | H | —CN | H | H | H | H | para | 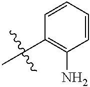 |
| a3-157 | —CH₃ | H | 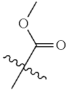 | H | H | H | H | para | —OH |
| a3-158 | —CH₃ | H | 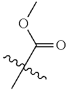 | H | H | H | H | para | 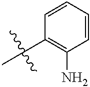 |
| a3-159 | —CH₃ | H | —COOH | H | H | H | H | para | —OH |
| a3-160 | —CH₃ | H | —COOH | H | H | H | H | para | 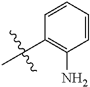 |
| a3-161 | —CH₃ | H | H | H | H | H | ortho- | para | —OH |
| | | | | | | | F | | |
| a3-162 | —CH₃ | H | H | H | H | H | meta- | para | —OH |
| | | | | | | | F | | |
| a3-163 | —CH₃ | H | H | H | H | H | ortho- F | para | 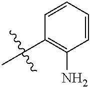 |
| a3-164 | —CH₃ | H | H | H | H | H | meta- F | para | 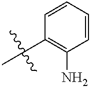 |
| a3-165 | —CH₃ | H | 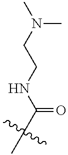 | H | H | H | H | para | —OH |
| a3-166 | —CH₃ | H | 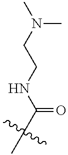 | H | H | H | H | para | 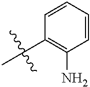 |
| a3-167 | H | H | H | H | H | F | H | para | —OH |
| a3-168 | H | H | H | H | H | F | H | para | 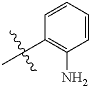 |

In one embodiment, the invention provides a compound of Formula (I-b) and a pharmaceutically acceptable salt thereof:

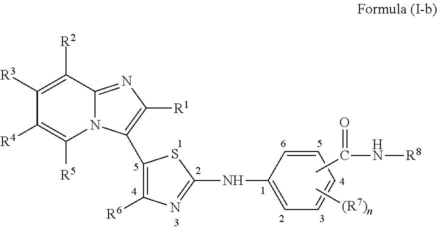

wherein R¹, R², R³, R⁴, R⁵, R⁶, R⁷ and R⁸ are as defined for various aspects of Formulae (I) and (I-a) above.

In an embodiment of Formula (I-b), R¹, R², R³, R⁴ and R⁵ are H; R⁶ is H, alkyl or haloalkyl; R⁷ is fluoro, chloro, bromo, or methyl and n is 0, or 1; and R⁸ is hydroxyl, aryl or heteroaryl, wherein aryl or heteroaryl are substituted with —NH₂ or —OH at a ring position adjacent to attachment of the —CONH-moiety, and R⁸ is optionally substituted with one or more groups R¹⁰ selected from amino, halo, alkyl, alicyclyl, heterocyclyl and aryl.

Non-limiting examples of such compounds include the following compounds and pharmaceutically acceptable salts thereof:

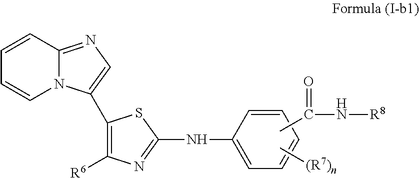

| Com- | | | | |
|---|---|---|---|---|
| pound | | | —CONHR⁸ | |
| No. | R⁶ | R⁷ | attachment | R⁸ |
| b1-01 | H | H | para | —OH |
| b1-02 | H | H | meta | —OH |
| b1-03 | —CH₃ | H | para | —OH |
| b1-04 | —CH₃ | H | meta | —OH |
| b1-05 | H | H | para | 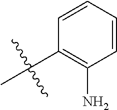 |
| b1-06 | H | H | meta | 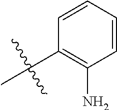 |
| b1-07 | —CH₃ | H | para | 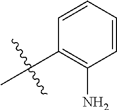 |
| b1-08 | —CH₃ | H | meta | 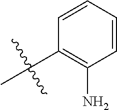 |
| b1-09 | H | H | para | 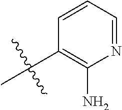 |
| b1-10 | H | H | meta | 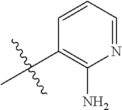 |
| b1-11 | —CH₃ | H | para | 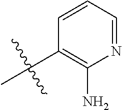 |
| b1-12 | —CH₃ | H | meta | 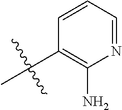 |
| b1-13 | H | H | para | 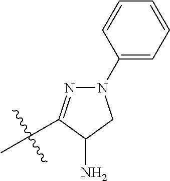 |
| b1-14 | H | H | meta | 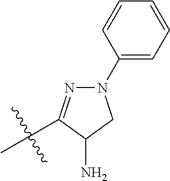 |
| b1-15 | —CH₃ | H | para | 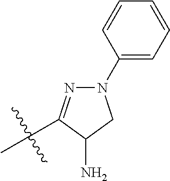 |
| b1-16 | —CH₃ | H | meta | 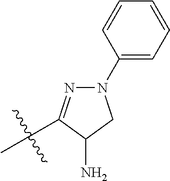 |
| b1-17 | H | H | para | 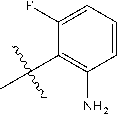 |
| b1-18 | H | H | meta | 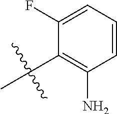 |
| b1-19 | —CH₃ | H | para | 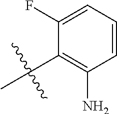 |
| b1-20 | —CH₃ | H | meta | 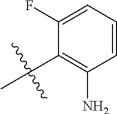 |
| b1-21 | H | ortho-F | para | 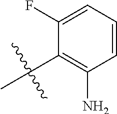 |
| b1-22 | H | ortho-F | meta | 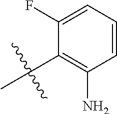 |
| b1-23 | —CH₃ | ortho-F | para | 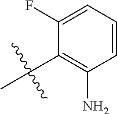 |
| b1-24 | —CH₃ | ortho-F | meta | 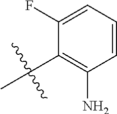 |

In other embodiment of Formula (I-b), R¹ is methyl; R², R³, R⁴ and R⁵ are H; R⁶ is H, alkyl or haloalkyl; R⁷ is fluoro, chloro, bromo, or methyl and n is 0 or 1; and R⁸ is hydroxyl, aryl or heteroaryl, wherein aryl or heteroaryl are substituted with —NH₂ or —OH at a ring position adjacent to attachment of the —CONH-moiety, and R⁸ is optionally substituted with one or more groups R¹⁰ selected from amino, halo, alkyl, alicyclyl, heterocyclyl and aryl.

Non-limiting examples of such compounds include the following compounds and pharmaceutically acceptable salts thereof:

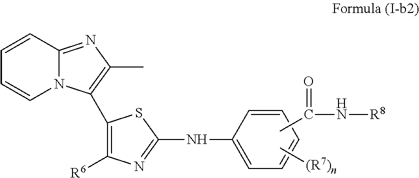

| Com- | | | | |
|---|---|---|---|---|
| pound | | | —CONHR⁸ | |
| No. | R⁶ | R⁷ | attachment | R⁸ |
| b2-01 | H | H | para | —OH |
| b2-02 | H | H | meta | —OH |
| b2-03 | —CH₃ | H | para | —OH |
| b2-04 | —CH₃ | H | meta | —OH |
| b2-05 | H | H | para | 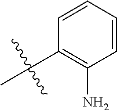 |
| b2-06 | H | H | meta | 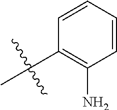 |
| b2-07 | —CH₃ | H | para | 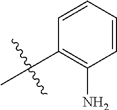 |
| b2-08 | —CH₃ | H | meta | 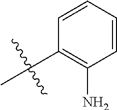 |
| b2-09 | H | H | para | 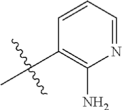 |
| b2-10 | H | H | meta | 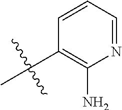 |
| b2-11 | —CH₃ | H | para | 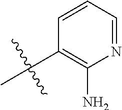 |
| b2-12 | —CH₃ | H | meta | 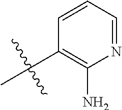 |
| b2-13 | H | H | para | 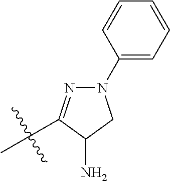 |
| b2-14 | H | H | meta | 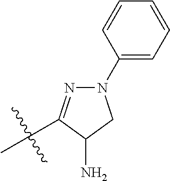 |
| b2-15 | —CH₃ | H | para | 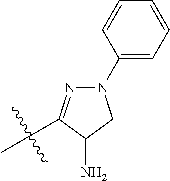 |
| b2-16 | —CH₃ | H | meta | 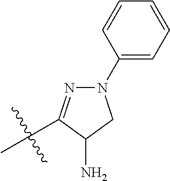 |
| b2-17 | H | H | para | 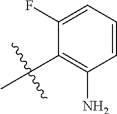 |
| b2-18 | H | H | meta | 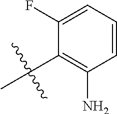 |
| b2-19 | —CH₃ | H | para | 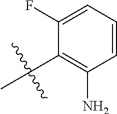 |
| b2-20 | —CH₃ | H | meta | 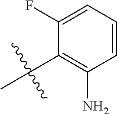 |
| b2-21 | H | ortho-F | para | 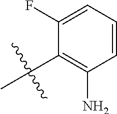 |
| b2-22 | H | ortho-F | meta | 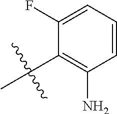 |
| b2-23 | —CH₃ | ortho-F | para | 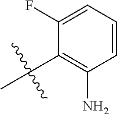 |
| b2-24 | —CH₃ | ortho-F | meta | 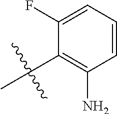 |

In another embodiment of Formula (I-b), at least two of R¹, R², R³, R⁴ and R⁵ are H and each non-hydrogen R¹, R², R³, R⁴ and R⁵ is independently selected from chloro, fluoro, bromo, methyl, ethyl, propyl, methoxy, ethoxy, carboxy, cyano, methoxymethyl, ethoxyethyl, propoxyethyl, methoxyethoxy, trifluoromethyl, hydroxyethoxy, dimethylamino, diethylamino, dimethylaminomethyl, dimethylamino ethyl, diethylaminomethyl, dimethylaminoethoxy, trifluoromethoxymethyl, trifluoroethoxymethyl, 3-oxetanoxy, trifluoroethylaminomethyl, N-methyl-N-methoxyethyl-aminoethyl, cyclopropanylmethyl, cyclobutoxy, 1-cyclopropanylethoxy, cyclopropanylmethylaminomethyl, 4-methylpiperazin-1-carbonyl, isoindolin-2-yl, N-methoxyethylcarbamoyl, N-(morpholin-4-yl)-ethylcarbamoyl, dimethylaminoethylamino, methylcarboxy, N,N-dimethylaminoethylcarbamoyl, benzyl, phenylethyl, trifluoromethylphenylethyl, phenoxymethyl, fluorophenoxymethyl, phenylethylaminomethyl, benzylaminomethyl, triazinylmethyl, piperidinylmethyl, piperidinyloxy, trifluoromethylpiperidinylmethyl, pyridinyloxymethyl, pyridinylmethoxy, tetrahydropyrazinyloxy, methylpiperazinylmethyl, pyrrolidin-1-yl, pyrrolidin-2-yl, pyrrolidin-3-yl, pyrrolidin-1-ylmethyl, pyrrolidin-2-ylmethyl, pyrrolidin-3-ylmethyl, pyrrolidin-1-ylethoxy, pyrrolidin-2-ylethoxy, pyrrolidin-3-ylethoxy, imidazol-1-ylmethyl, imidazol-2-ylmethyl, imidazol-4-ylmethyl, imidazolidin-1-yl, imidazolidin-2-yl, imidazolidin-4-yl, imidazolidin-1-ylmethyl, imidazolidin-2-ylmethyl, imidazolidin-4-ylmethyl, imidazolin-1-yl, imidazolin-2-yl, imidazolin-4-yl, pyrazolidin-1-yl, pyrazolidin-3-yl, pyrazolidin-4-yl, pyrazolin-1-yl, pyrazolin-3-yl, pyrazolin-4-yl, piperidin-1-yl, piperidin-2-yl, piperidin-3-yl, piperidin-4-yl, piperidin-1-ylmethyl, piperidin-2-ylmethyl, piperidin-3-ylmethyl, piperidin-4-ylmethyl, piperazin-1-yl, piperazin-2-yl, piperazin-3-yl, morpholin-2-yl, morpholin-3-yl, morpholin-4-yl, morpholin-2-ylmethyl, morpholin-3-ylmethyl, morpholin-4-ylmethyl, morpholin-2-ylethoxy, morpholin-3-ylethoxy and morpholin-4-ylethoxy; R⁶ is H, alkyl or haloalkyl; R⁷ is independently fluoro, chloro, bromo, or methyl and n is 0 or 1; and R⁸ is hydroxyl, aryl or heteroaryl, wherein aryl or heteroaryl are substituted with —NH₂ or —OH and R⁸ is optionally substituted with one or more groups R¹⁰ selected from amino, halo, alkyl, alicyclyl, heterocyclyl and aryl.

Non-limiting examples of such compounds include the following compounds and pharmaceutically acceptable salts thereof:

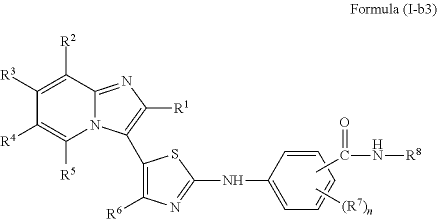

| Com- | | | | | | | | | |
|---|---|---|---|---|---|---|---|---|---|
| pound | | | | | | | | —CONHR⁸ | |
| No. | R¹ | R² | R³ | R⁴ | R⁵ | R⁶ | R⁷ | attachment | R⁸ |
| b3-01 | —CH₃ | —Cl | H | H | H | H | H | para | —OH |
| b3-02 | —CH₃ | H | -Cl | H | H | H | H | para | —OH |
| b3-03 | —CH₃ | H | H | —Cl | H | H | H | para | —OH |
| b3-04 | —CH₃ | H | H | H | —Cl | H | H | para | —OH |
| b3-05 | —CH₃ | —Cl | H | H | H | H | H | meta | —OH |
| b3-06 | —CH₃ | H | —Cl | H | H | H | H | meta | —OH |
| b3-07 | —CH₃ | H | H | —Cl | H | H | H | meta | —OH |
| b3-08 | —CH₃ | H | H | H | —Cl | H | H | meta | —OH |
| b3-09 | —CH₃ | —Cl | H | H | H | H | H | para | 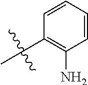 |
| b3-10 | —CH₃ | H | —Cl | H | H | H | H | para | 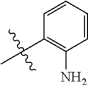 |
| b3-11 | —CH₃ | H | H | —Cl | H | H | H | para | 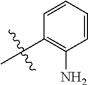 |
| b3-12 | —CH₃ | H | H | H | —Cl | H | H | para | 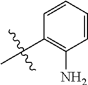 |
| b3-13 | —CH₃ | —Cl | H | H | H | H | H | para | 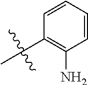 |
| b3-14 | —CH₃ | H | —Cl | H | H | H | H | para | 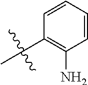 |
| b3-15 | —CH₃ | H | H | —Cl | H | H | H | para | 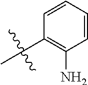 |
| b3-16 | —CH₃ | H | H | H | —Cl | H | H | para | 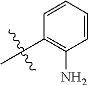 |
| b3-17 | —CH₃ | —Cl | H | H | H | H | H | para | 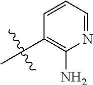 |
| b3-18 | —CH₃ | H | —Cl | H | H | H | H | para | 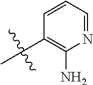 |
| b3-19 | —CH₃ | H | H | —Cl | H | H | H | para | 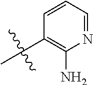 |
| b3-20 | —CH₃ | H | H | H | —Cl | H | H | para | 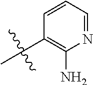 |
| b3-21 | —CH₃ | —Cl | H | H | H | H | H | para | 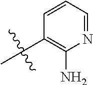 |
| b3-22 | —CH₃ | H | —Cl | H | H | H | H | para | 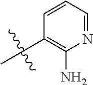 |
| b3-23 | —CH₃ | H | H | —Cl | H | H | H | para | 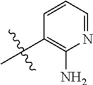 |
| b3-24 | —CH₃ | H | H | H | —Cl | H | H | para | 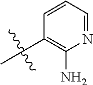 |
| b3-25 | —CH₃ | —CF₃ | H | H | H | H | H | para | —OH |
| b3-26 | —CH₃ | H | —CF₃ | H | H | H | H | para | —OH |
| b3-27 | —CH₃ | H | H | —CF₃ | H | H | H | para | —OH |
| b3-28 | —CH₃ | H | H | H | —CF₃ | H | H | para | —OH |
| b3-29 | —CH₃ | —CF₃ | H | H | H | H | H | para | 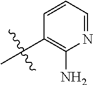 |
| b3-30 | —CH₃ | H | —CF₃ | H | H | H | H | para | 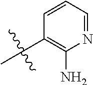 |
| b3-31 | —CH₃ | H | H | —CF₃ | H | H | H | para | 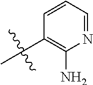 |
| b3-32 | —CH₃ | H | H | H | —CF₃ | H | H | para | 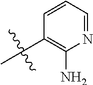 |
| b3-33 | —CH₃ | —OCH₃ | H | H | H | H | H | para | —OH |
| b3-34 | —CH₃ | H | —OCH₃ | H | H | H | H | para | —OH |
| b3-35 | —CH₃ | H | H | —OCH₃ | H | H | H | para | —OH |
| b3-36 | —CH₃ | H | H | H | —OCH₃ | H | H | para | —OH |
| b3-37 | —CH₃ | —OCH₃ | H | H | H | H | H | para | 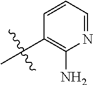 |
| b3-38 | —CH₃ | H | —OCH₃ | H | H | H | H | para | 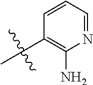 |
| b3-39 | —CH₃ | H | H | —OCH₃ | H | H | H | para | 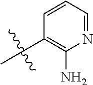 |
| b3-40 | —CH₃ | H | H | H | —OCH₃ | H | H | para | 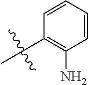 |
| b3-41 | —CH₃ | 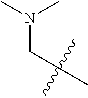 | H | H | H | H | H | para | —OH |
| b3-41 | —CH₃ | H | 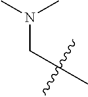 | H | H | H | H | para | —OH |
| b3-43 | —CH₃ | H | H | 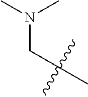 | H | H | H | para | —OH |
| b3-44 | —CH₃ | H | H | H | 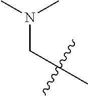 | H | H | para | —OH |
| b3-45 | —CH₃ | 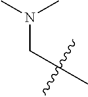 | H | H | H | H | H | para | 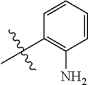 |
| b3-46 | —CH₃ | H | 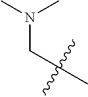 | H | H | H | H | para | 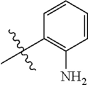 |
| b3-47 | —CH₃ | H | H | 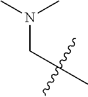 | H | H | H | para | 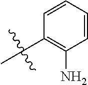 |
| b3-48 | —CH₃ | H | H | H | 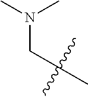 | H | H | para | 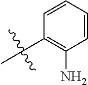 |
| b3-45 | —CH₃ | 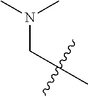 | H | H | H | H | ortho- F | para | 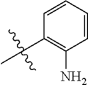 |
| b3-46 | —CH₃ | H | 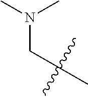 | H | H | H | ortho- F | para | 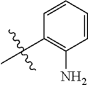 |
| b3-47 | —CH₃ | H | H | 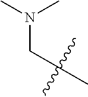 | H | H | ortho- F | para | 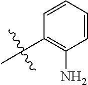 |
| b3-48 | —CH₃ | H | H | H | 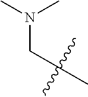 | H | ortho- F | para | 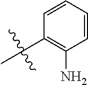 |
| b3-49 | —CH₃ | 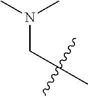 | H | H | H | H | ortho- F | para | 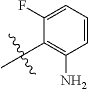 |
| b3-50 | —CH₃ | H | 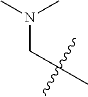 | H | H | H | ortho- F | para | 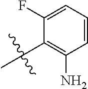 |
| b3-51 | —CH₃ | H | H | 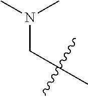 | H | H | ortho- F | para | 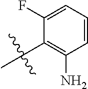 |
| b3-52 | —CH₃ | H | H | H | 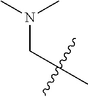 | H | ortho- F | para | 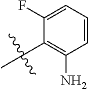 |
| b3-53 | —CH₃ | 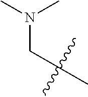 | H | H | H | H | H | para | 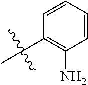 |
| b3-54 | —CH₃ | H | 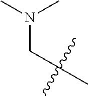 | H | H | H | H | para | 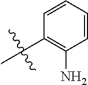 |
| b3-55 | —CH₃ | H | H | 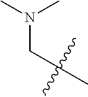 | H | H | H | para | 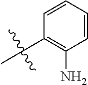 |
| b3-56 | —CH₃ | H | H | H | 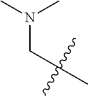 | H | H | para | 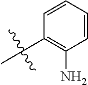 |
| b3-57 | —CH₃ | 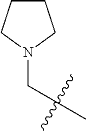 | H | H | H | H | H | para | 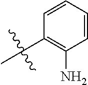 |
| b3-58 | —CH₃ | H | 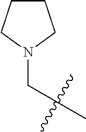 | H | H | H | H | para | 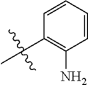 |
| b3-59 | —CH₃ | H | H | 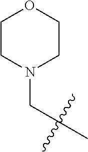 | H | H | H | para | 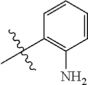 |
| b3-60 | —CH₃ | H | H | H | 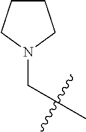 | H | H | para | 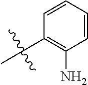 |
| b3-61 | —CH₃ | 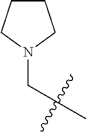 | H | H | H | H | ortho- F | para | 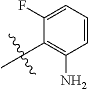 |
| b3-62 | —CH₃ | H | 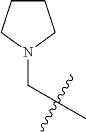 | H | H | H | ortho- F | para | 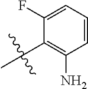 |
| b3-63 | —CH₃ | H | H | 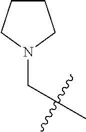 | H | H | ortho- F | para | 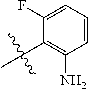 |
| b3-64 | —CH₃ | H | H | H | 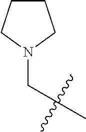 | H | ortho- F | para | 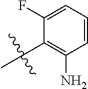 |
| b3-65 | —CH₃ | 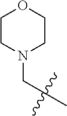 | H | H | H | H | H | para | 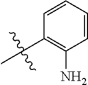 |
| b3-66 | —CH₃ | H | 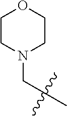 | H | H | H | H | para | 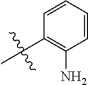 |
| b3-67 | —CH₃ | H | H | 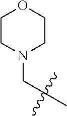 | H | H | H | para | 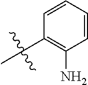 |
| b3-68 | —CH₃ | H | H | H | 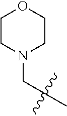 | H | H | para | 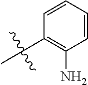 |
| b3-69 | —CH₃ | 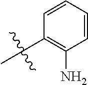 | H | H | H | H | ortho- F | para | 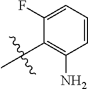 |
| b3-70 | | H | 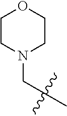 | H | H | H | ortho- F | para | 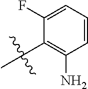 |
| b3-71 | —CH₃ | H | H | 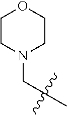 | H | H | ortho- F | para | 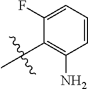 |
| b3-72 | —CH₃ | H | H | H | 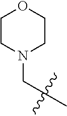 | H | ortho- F | para | 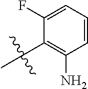 |
| b3-73 | H | 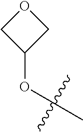 | H | H | H | H | H | para | 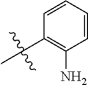 |
| b3-74 | H | H | 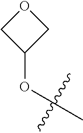 | H | H | H | H | para | 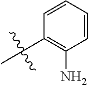 |
| b3-75 | H | H | H | 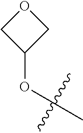 | H | H | H | para | 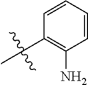 |
| b3-76 | H | H | H | H | 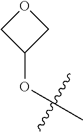 | H | H | para | 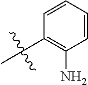 |
| b3-77 | H | 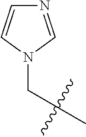 | H | H | H | H | H | para | 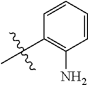 |
| b3-78 | H | H | 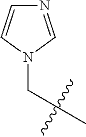 | H | H | H | H | para | 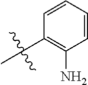 |
| b3-79 | H | H | H | 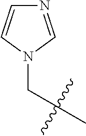 | H | H | H | para | 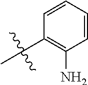 |
| b3-80 | H | H | H | H | 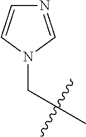 | H | H | para | 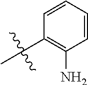 |
| b3-81 | H | 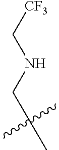 | H | H | H | H | H | para | 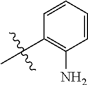 |
| b3-82 | H | H | 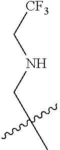 | H | H | H | H | para | 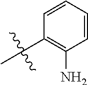 |
| b3-83 | H | H | H | 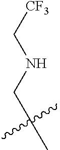 | H | H | H | para | 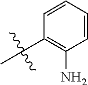 |
| b3-84 | H | H | H | H | 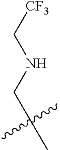 | H | H | para | 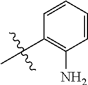 |
| b3-85 | H | 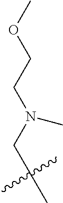 | H | H | H | H | H | para | 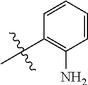 |
| b3-86 | H | H | 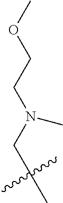 | H | H | H | H | para | 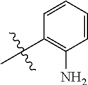 |
| b3-87 | H | H | H | 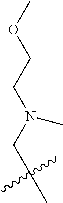 | H | H | H | para | 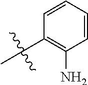 |
| b3-88 | H | H | H | H | 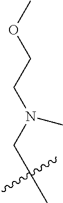 | H | H | para | 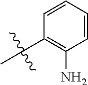 |
| b3-89 | H | 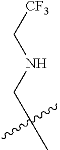 | H | H | H | H | H | para | 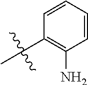 |
| b3-90 | H | H | 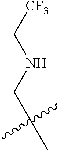 | H | H | H | H | para | 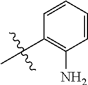 |
| b3-91 | H | H | H | 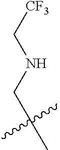 | H | H | H | para | 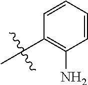 |
| b3-92 | H | H | H | H | 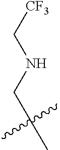 | H | H | para | 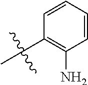 |
| b3-93 | H | 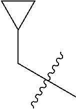 | H | H | H | H | H | para | 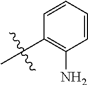 |
| b3-94 | H | H | 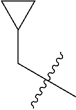 | H | H | H | H | para | 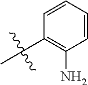 |
| b3-95 | H | H | H | 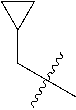 | H | H | H | para | 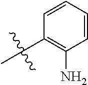 |
| b3-96 | H | H | H | H | 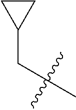 | H | H | para | 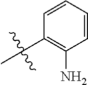 |
| b3-97 | H | 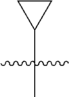 | H | H | H | H | H | para | 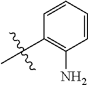 |
| b3-98 | H | H | 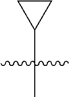 | H | H | H | H | para | 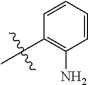 |
| b3-99 | H | H | H | 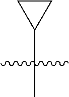 | H | H | H | para | 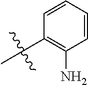 |
| b3-100 | H | H | H | H | 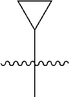 | H | H | para | 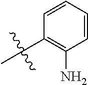 |
| b3-101 | H | 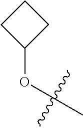 | H | H | H | H | H | para | 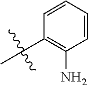 |
| b3-102 | H | H | 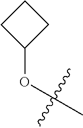 | H | H | H | H | para | 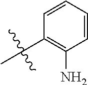 |
| b3-103 | H | H | H | 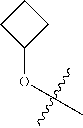 | H | H | H | para | 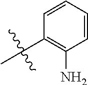 |
| b3-104 | H | H | H | H | 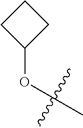 | H | H | para | 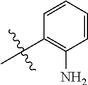 |
| b3-105 | H | 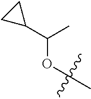 | H | H | H | H | H | para | 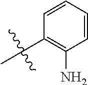 |
| b3-106 | H | H | 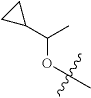 | H | H | H | H | para | 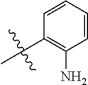 |
| b3-107 | H | H | H | 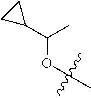 | H | H | H | para | 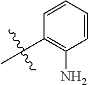 |
| b3-108 | H | H | H | H | 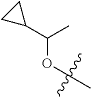 | H | H | para | 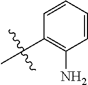 |
| b3-109 | H | 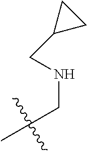 | H | H | H | H | H | para | 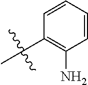 |
| b3-110 | H | H | 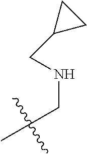 | H | H | H | H | para | 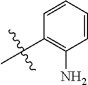 |
| b3-111 | H | H | H | 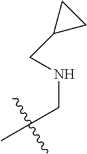 | H | H | H | para | 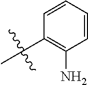 |
| b3-112 | H | H | H | H | 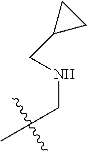 | H | H | para | 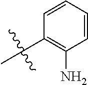 |
| b3-113 | H | 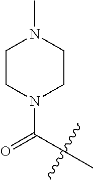 | H | H | H | H | H | para | 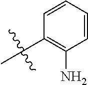 |
| b3-114 | H | H | 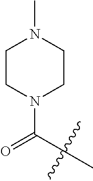 | H | H | H | H | para | 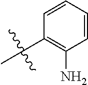 |
| b3-115 | H | H | H | 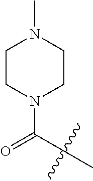 | H | H | H | para | 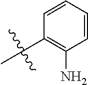 |
| b3-116 | H | H | H | H | 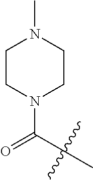 | H | H | para | 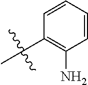 |
| b3-117 | H | 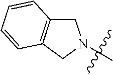 | H | H | H | H | H | para | 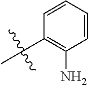 |
| b3-118 | H | H | 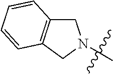 | H | H | H | H | para | 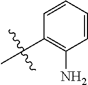 |
| b3-119 | H | H | H | 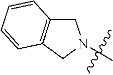 | H | H | H | para | 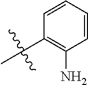 |
| b3-120 | H | H | H | H | 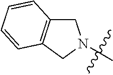 | H | H | para | 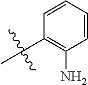 |
| b3-121 | —CH₃ | —F | H | H | H | H | H | para | —OH |
| b3-122 | —CH₃ | —F | H | H | H | H | H | para | 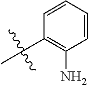 |
| b3-123 | —CH₃ | H | H | —Br | H | H | H | para | —OH |
| b3-124 | —CH₃ | H | H | —Br | H | H | H | para | 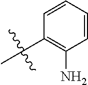 |
| b3-125 | —CH₃ | H | 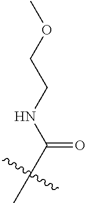 | H | H | H | H | para | —OH |
| b3-126 | —CH₃ | H | 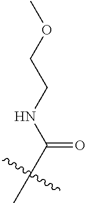 | H | H | H | H | para | 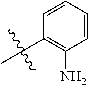 |
| b3-127 | —CH₃ | H | H | 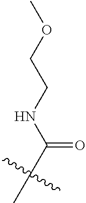 | H | H | H | para | —OH |
| b3-128 | —CH₃ | H | H | 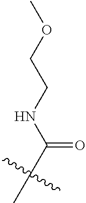 | H | H | H | para | 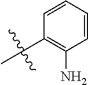 |
| b3-129 | —CH₃ | H | 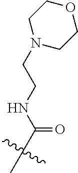 | H | H | H | H | para | —OH |
| b3-130 | —CH₃ | H | 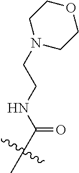 | H | H | H | H | para | 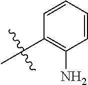 |
| b3-131 | —CH₃ | H | H | 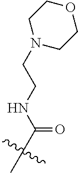 | H | H | H | para | —OH |
| b3-132 | —CH₃ | H | H | 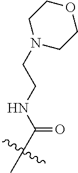 | H | H | H | para | 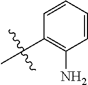 |
| b3-133 | —CH₃ | H | 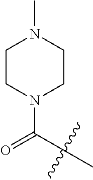 | H | H | H | H | para | —OH |
| b3-134 | —CH₃ | H | 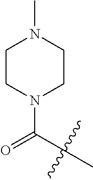 | H | H | H | H | para | 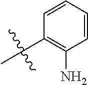 |
| b3-135 | —CH₃ | H | H | 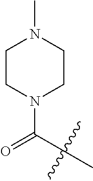 | H | H | H | para | —OH |
| b3-136 | —CH₃ | H | H | 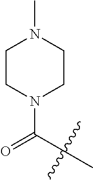 | H | H | H | para | 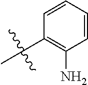 |
| b3-137 | —CH₃ | H | 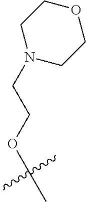 | H | H | H | H | para | —OH |
| b3-138 | —CH₃ | H | 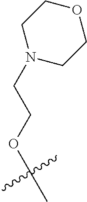 | H | H | H | H | para | 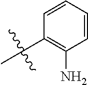 |
| b3-139 | —CH₃ | H | 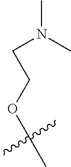 | H | H | H | H | para | —OH |
| b3-140 | —CH₃ | H | 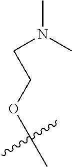 | H | H | H | H | para | 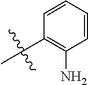 |
| b3-141 | —CH₃ | H | 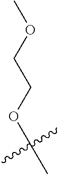 | H | H | H | H | para | —OH |
| b3-142 | —CH₃ | H | 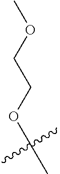 | H | H | H | H | para | 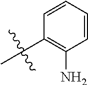 |
| b3-143 | —CH₃ | H | H | 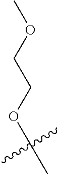 | H | H | H | para | —OH |
| b3-144 | —CH₃ | H | H | 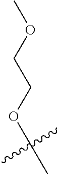 | H | H | H | para | 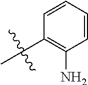 |
| b3-145 | —CH₃ | H | 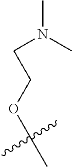 | H | H | H | H | para | —OH |
| b3-146 | —CH₃ | H | 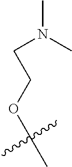 | H | H | H | H | para | 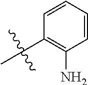 |
| b3-147 | —CH₃ | H | H | —OCH₃ | H | H | H | para | —OH |
| b3-148 | —CH₃ | H | H | —OCH₃ | H | H | H | para | 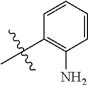 |
| b3-149 | H | H | —OCH₃ | H | H | H | H | para | —OH |
| b3-150 | H | H | —OCH₃ | H | H | H | H | para | 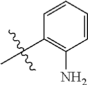 |
| b3-151 | —CH₃ | H | 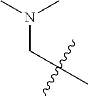 | H | H | H | H | para | —OH |
| b3-152 | | H | 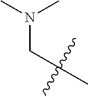 | H | H | H | H | para | 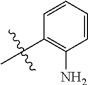 |
| b3-153 | —CF₃ | H | H | H | H | H | H | para | —OH |
| b3-154 | —CF₃ | H | H | H | H | H | H | para | 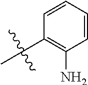 |
| b3-155 | —CH₃ | H | —CN | H | H | H | H | para | —OH |
| b3-156 | —CH₃ | H | —CN | H | H | H | H | para | 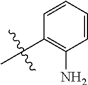 |
| b3-157 | —CH₃ | H | 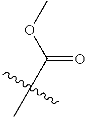 | H | H | H | H | para | —OH |
| b3-158 | —CH₃ | H | 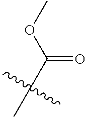 | H | H | H | H | para | 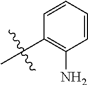 |
| b3-159 | —CH₃ | H | —COOH | H | H | H | H | para | —OH |
| b3-160 | —CH₃ | H | —COOH | H | H | H | H | para | 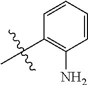 |
| b3-161 | —CH₃ | H | H | H | H | H | ortho- F | para | —OH |
| b3-162 | —CH₃ | H | H | H | H | H | meta- F | para | —OH |
| b3-163 | —CH₃ | H | H | H | H | H | ortho- F | para | 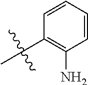 |
| b3-164 | —CH₃ | H | H | H | H | H | meta- F | para | 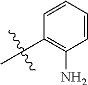 |
| b3-165 | —CH₃ | H | 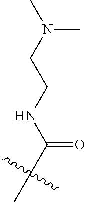 | H | H | H | H | para | —OH |
| b3-166 | —CH₃ | H | 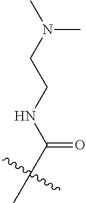 | H | H | H | H | para | 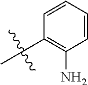 |
| b3-167 | H | H | H | H | H | F | H | para | —OH |
| b3-168 | H | H | H | H | H | F | H | para | 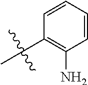 |

In one embodiment, the invention provides a compound of Formula (I-c) and a pharmaceutically acceptable salt thereof:

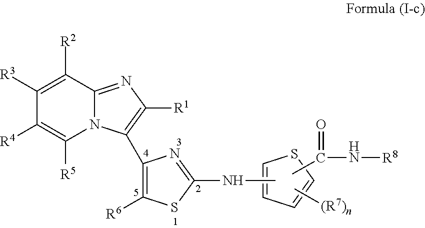

wherein R¹, R², R³, R⁴, R⁵, R⁶, R⁷ and R⁸ are as defined above for various aspects of Formulae (I), (I-a), and (I-b).

In an embodiment of Formula (I-c), R¹, R², R³, R⁴ and R⁵ are H; R⁶ is H, alkyl or haloalkyl; R⁷ is fluoro, chloro, bromo, or methyl and n is 0 or 1; and R⁸ is hydroxyl, aryl or heteroaryl, wherein aryl or heteroaryl are substituted with —NH₂ or —OH at a ring position adjacent to attachment of the —CONH-moiety, and R⁸ is optionally substituted with one or more groups R¹⁰ selected from amino, halo, alkyl, alicyclyl, heterocyclyl and aryl.

Non-limiting examples of such compounds include the following compounds and pharmaceutically acceptable salts thereof:

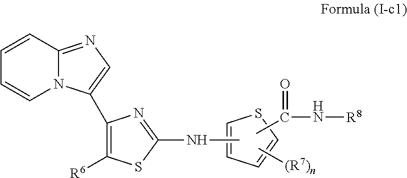

In various embodiments, the NH linker and the CONHR⁸ group are disposed in a 2,4- or a 2,5-configuration about the thiophene ring, with the optional R⁷ groups occupying the other positions. The Table lists compounds of Formula (I-c1) that in one embodiment have a 2,5-configuration on the thiophene and in another have a 2,4-configuration on the thiophene; in the compounds of the Table, n=0 and this is indicated by a listing of “H” under the R⁷ column.

| Compound No. | R⁶ | R⁷ | R⁸ |
|---|---|---|---|
| c1-01 | H | H | —OH |
| c1-02 | —CH₃ | H | —OH |
| c1-03 | H | H | 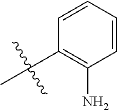 |
| c1-04 | —CH₃ | H | 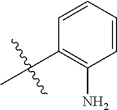 |
| c1-05 | H | H | 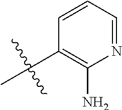 |
| c1-06 | —CH₃ | H | 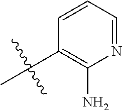 |
| c1-07 | H | H | 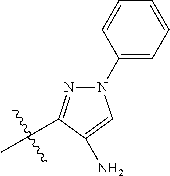 |
| c1-08 | —CH₃ | H | 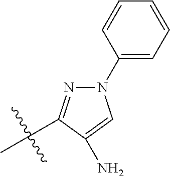 |
| c1-09 | H | H | 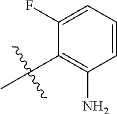 |
| c1-10 | —CH₃ | H | 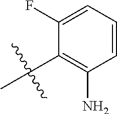 |

In other embodiment of Formula (I-c), R¹ is methyl; R², R³, R⁴ and R⁵ are H; R⁶ is H, alkyl or haloalkyl; R⁷ is fluoro, chloro, bromo, or methyl and n is 0 or 1; and R⁸ is hydroxyl, aryl or heteroaryl, wherein aryl or heteroaryl are substituted with —NH₂ or —OH at a ring position adjacent to attachment of the —CONH-moiety and R⁸ is optionally substituted with one or more groups selected from amino, halo, alkyl, alicyclyl, heterocyclyl and aryl.

Non-limiting examples of such compounds include the following compounds and pharmaceutically acceptable salts thereof:

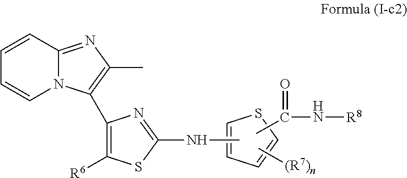

The following Table lists compounds of Formula (I-c2) that in one embodiment have a 2,5-configuration on the thiophene and in another have a 2,4-configuration on the thiophene; in the compounds of the Table, n=0 and this is indicated by a listing of “H” under the R⁷ column.

| Compound No. | R⁶ | R⁷ | R⁸ |
|---|---|---|---|
| c2-01 | H | H | —OH |
| c2-02 | —CH₃ | H | —OH |
| c2-03 | H | H | 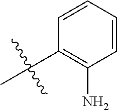 |
| c2-04 | —CH₃ | H | 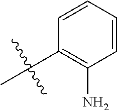 |
| c2-05 | H | H | 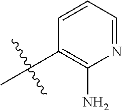 |
| c2-06 | —CH₃ | H | 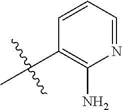 |
| c2-07 | H | H | 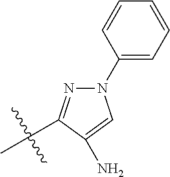 |
| c2-08 | —CH₃ | H | 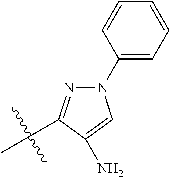 |
| c2-09 | H | H | 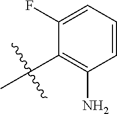 |
| c2-10 | —CH₃ | H | 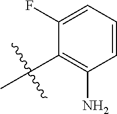 |

In another embodiment of Formula (I-c), at least two of R¹, R², R³, R⁴ and R⁵ are H and each non-hydrogen R¹, R², R³, R⁴ and R⁵ is independently selected from chloro, fluoro, bromo, methyl, ethyl, propyl, methoxy, ethoxy, carboxy, cyano, methoxymethyl, ethoxyethyl, propoxyethyl, methoxyethoxy, trifluoromethyl, hydroxyethoxy, dimethylamino, diethylamino, dimethylaminomethyl, dimethylamino ethyl, diethylaminomethyl, dimethylaminoethoxy, trifluoromethoxymethyl, trifluoroethoxymethyl, 3-oxetanoxy, trifluoroethylaminomethyl, N-methyl-N-methoxyethyl-aminoethyl, cyclopropanylmethyl, cyclobutoxy, 1-cyclopropanylethoxy, cyclopropanylmethylaminomethyl, 4-methylpiperazin-1-carbonyl, isoindolin-2-yl, N-methoxyethylcarbamoyl, N-(morpholin-4-yl)-ethylcarbamoyl, dimethylaminoethylamino, methylcarboxy, N,N-dimethylaminoethylcarbamoyl, benzyl, phenylethyl, trifluoromethylphenylethyl, phenoxymethyl, fluorophenoxymethyl, phenylethylaminomethyl, benzylaminomethyl, triazinylmethyl, piperidinylmethyl, piperidinyloxy, trifluoromethylpiperidinylmethyl, pyridinyloxymethyl, pyridinylmethoxy, tetrahydropyrazinyloxy, methylpiperazinylmethyl, pyrrolidin-1-yl, pyrrolidin-2-yl, pyrrolidin-3-yl, pyrrolidin-1-ylmethyl, pyrrolidin-2-ylmethyl, pyrrolidin-3-ylmethyl, pyrrolidin-1-ylethoxy, pyrrolidin-2-ylethoxy, pyrrolidin-3-ylethoxy, imidazol-1-ylmethyl, imidazol-2-ylmethyl, imidazol-4-ylmethyl, imidazolidin-1-yl, imidazolidin-2-yl, imidazolidin-4-yl, imidazolidin-1-ylmethyl, imidazolidin-2-ylmethyl, imidazolidin-4-ylmethyl, imidazolin-1-yl, imidazolin-2-yl, imidazolin-4-yl, pyrazolidin-1-yl, pyrazolidin-3-yl, pyrazolidin-4-yl, pyrazolin-1-yl, pyrazolin-3-yl, pyrazolin-4-yl, piperidin-1-yl, piperidin-2-yl, piperidin-3-yl, piperidin-4-yl, piperidin-1-ylmethyl, piperidin-2-ylmethyl, piperidin-3-ylmethyl, piperidin-4-ylmethyl, piperazin-1-yl, piperazin-2-yl, piperazin-3-yl, morpholin-2-yl, morpholin-3-yl, morpholin-4-yl, morpholin-2-ylmethyl, morpholin-3-ylmethyl, morpholin-4-ylmethyl, morpholin-2-ylethoxy, morpholin-3-ylethoxy and morpholin-4-ylethoxy; R⁶ is H, alkyl or haloalkyl; R⁷ is fluoro, chloro, bromo, or methyl and n is 0 or 1; and R⁸ is hydroxyl, aryl or heteroaryl, wherein aryl or heteroaryl are substituted with —NH₂ or —OH and R⁸ is optionally substituted with one or more groups R¹⁰ selected from amino, halo, alkyl, alicyclyl, heterocyclyl, and aryl.

Non-limiting examples of such compounds include the following compounds and pharmaceutically acceptable salts thereof:

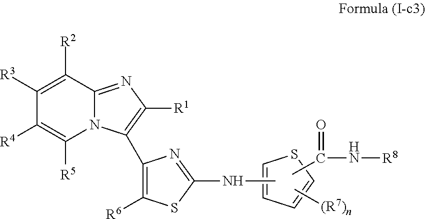

The Table discloses compounds of Formula (I-c3) that in one embodiment have a 2,5-configuration on the thiophene and in another have a 2,4-configuration on the thiophene. To illustrate, the row labeled as “reference No. c3-01” discloses two thiophene HDAC compounds and their pharmaceutically acceptable salts. The first compound contains the R¹-R⁸ substituents of the c3-01 row on a compound of Formula (I-c3) where the —NH— and the —C(O)NHR⁸ are disposed about the thiophene ring in a 2,5-configuration, with the S atom taken as position 1. The second compound (and salts) embraced by Reference No. c3-01 has the same substituents R¹-R⁸, but the —NH— and the —C(O)NHR⁸ are disposed about the thiophene ring in a 2,4-configuration.

In the compounds of the Table, n=0 or n=1. When n=0, by convention this is indicated by a listing of “H” under the R⁷ column. When n=1, the substituent listed in the R⁷ column is attached to one of the two “free” positions on the thiophene ring not occupied by the —NH— or —C(O)NHR⁸ groups. When the Reference No. discloses a 2,5-substituted thiophene, the substituent R⁷ is on the 3-position in a first embodiment and on the 4-position in a second embodiment. Similarly, when the Reference No. discloses a 2,4-substituted thiophene, the substituent R⁷ is on the 3-position in a first embodiment and on the 5-position in a second embodiment. This is indicated in the Table (Reference No. c3-145 through c3-148) by a parenthetical mention of the particular thiophene configuration below the reference no. Thus to illustrate, each of Reference No. c3-145 (hydroxamate) and c3-147 (arylamide) embraces both the 3-fluoro-2,5-thiophendiyl and the 4-fluoro-2,5-thiophendiyl species of the respective compound, while each of c3-146 and c3-148 embraces the 3-fluoro-2,4-thiophendiyl and 5-fluoro-2,4-thiophendiyl species of the respective hydroxamate and arylamide.

Table of compounds of Formula I-c3

| Reference | | | | | | | | |
|---|---|---|---|---|---|---|---|---|
| No. | R¹ | R² | R³ | R⁴ | R⁵ | R⁶ | R⁷ | R⁸ |
| c3-01 | —CH₃ | —Cl | H | H | H | H | H | —OH |
| c3-02 | —CH₃ | H | —Cl | H | H | H | H | —OH |
| c3-03 | —CH₃ | H | H | —Cl | H | H | H | —OH |
| c3-04 | —CH₃ | H | H | H | —Cl | H | H | —OH |
| c3-05 | —CH₃ | —Cl | H | H | H | H | H | 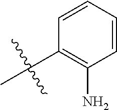 |
| c3-06 | —CH₃ | H | —Cl | H | H | H | H | 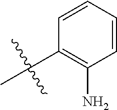 |
| c3-07 | —CH₃ | H | H | —Cl | H | H | H | 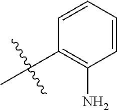 |
| c3-08 | —CH₃ | H | H | H | —Cl | H | H | 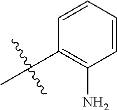 |
| c3-09 | —CH₃ | —Cl | H | H | H | H | H | 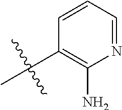 |
| c3-10 | —CH₃ | H | —Cl | H | H | H | H | 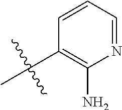 |
| c3-11 | —CH₃ | H | H | —Cl | H | H | H | 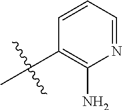 |
| c3-12 | —CH₃ | H | H | H | —Cl | H | H | 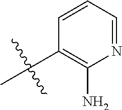 |
| c3-13 | —CH₃ | —CF₃ | H | H | H | H | H | —OH |
| c3-14 | —CH₃ | H | —CF₃ | H | H | H | H | —OH |
| c3-15 | —CH₃ | H | H | —CF₃ | H | H | H | —OH |
| c3-16 | —CH₃ | H | H | H | —CF₃ | H | H | —OH |
| c3-17 | —CH₃ | —CF₃ | H | H | H | H | H | 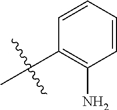 |
| c3-18 | —CH₃ | H | —CF₃ | H | H | H | H | 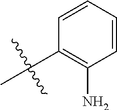 |
| c3-19 | —CH₃ | H | H | —CF₃ | H | H | H | 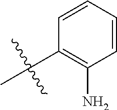 |
| c3-20 | —CH₃ | H | H | H | —CF₃ | H | H | 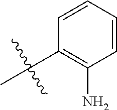 |
| c3-21 | —CH₃ | —OH | H | H | H | H | H | —OH |
| c3-22 | —CH₃ | H | —OH | H | H | H | H | —OH |
| c3-23 | —CH₃ | H | H | —OH | H | H | H | —OH |
| c3-24 | —CH₃ | H | H | H | —OH | H | H | —OH |
| c3-25 | —CH₃ | —OH | H | H | H | H | H | 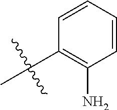 |
| c3-26 | —CH₃ | H | —OH | H | H | H | H | 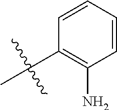 |
| c3-27 | —CH₃ | H | H | —OH | H | H | H | 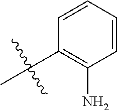 |
| c3-28 | —CH₃ | H | H | H | —OH | H | H | 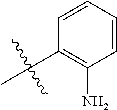 |
| c3-29 | —CH₃ | 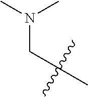 | H | H | H | H | H | —OH |
| c3-30 | —CH₃ | H | 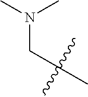 | H | H | H | H | —OH |
| c3-31 | —CH₃ | H | H | 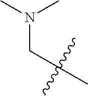 | H | H | H | —OH |
| c3-32 | —CH₃ | H | H | H | 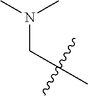 | H | H | —OH |
| c3-33 | —CH₃ | 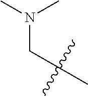 | H | H | H | H | H | 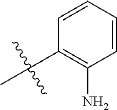 |
| c3-34 | —CH₃ | H | 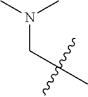 | H | H | H | H | 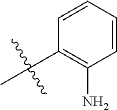 |
| c3-35 | —CH₃ | H | H | 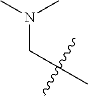 | H | H | H | 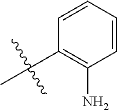 |
| c3-36 | —CH₃ | H | H | H | 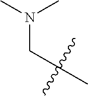 | H | H | 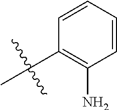 |
| c3-37 | —CH₃ | 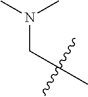 | H | H | H | H | H | 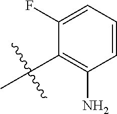 |
| c3-38 | —CH₃ | H | 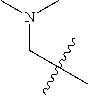 | H | H | H | H | 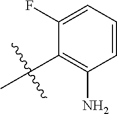 |
| c3-39 | —CH₃ | H | H | 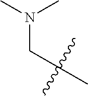 | H | H | H | 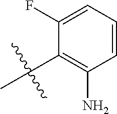 |
| c3-40 | —CH₃ | H | H | H | 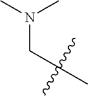 | H | H | 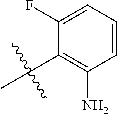 |
| c3-41 | —CH₃ | 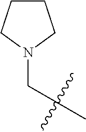 | H | H | H | H | H | 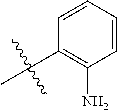 |
| c3-42 | —CH₃ | H | 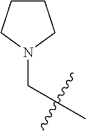 | H | H | H | H | 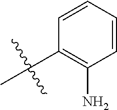 |
| c3-43 | —CH₃ | H | H | 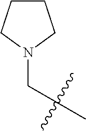 | H | H | H | 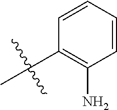 |
| c3-44 | —CH₃ | H | H | H | 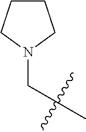 | H | H | 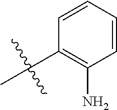 |
| c3-45 | —CH₃ | 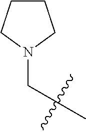 | H | H | H | H | H | 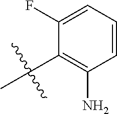 |
| c3-46 | —CH₃ | H | 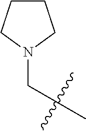 | H | H | H | H | 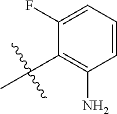 |
| c3-47 | —CH₃ | H | H | 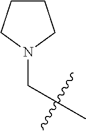 | H | H | H | 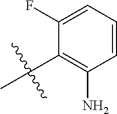 |
| c3-48 | —CH₃ | H | H | H | 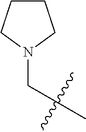 | H | H | 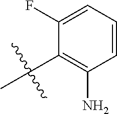 |
| c3-49 | —CH₃ | 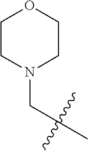 | H | H | H | H | H | 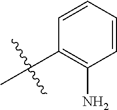 |
| c3-50 | —CH₃ | H | 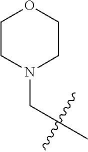 | H | H | H | H | 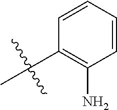 |
| c3-51 | —CH₃ | H | H | 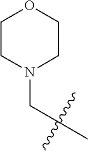 | H | H | H | 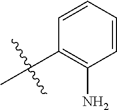 |
| c3-52 | —CH₃ | H | H | H | 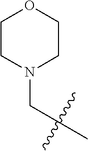 | H | H | 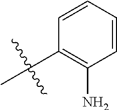 |
| c3-53 | —CH₃ | 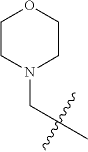 | H | H | H | H | H | 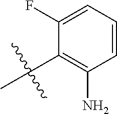 |
| c3-54 | —CH₃ | H | 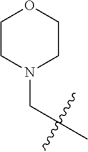 | H | | H | H | 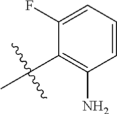 |
| c3-55 | —CH₃ | H | H | 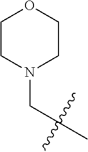 | H | H | H | 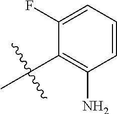 |
| c3-56 | —CH₃ | H | H | H | 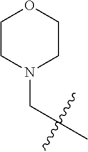 | H | H | 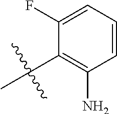 |
| c3-57 | H | 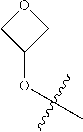 | H | H | H | H | H | 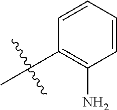 |
| c3-58 | H | H | 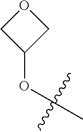 | H | H | H | H | 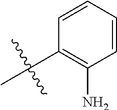 |
| c3-59 | H | H | H | 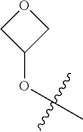 | H | H | H | 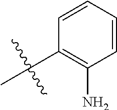 |
| c3-60 | H | H | H | H | 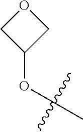 | H | H | 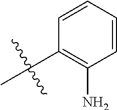 |
| c3-61 | H | 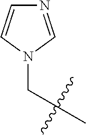 | H | H | H | H | H | 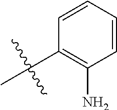 |
| c3-62 | H | H | 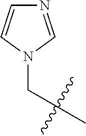 | H | H | H | H | 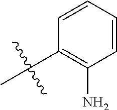 |
| c3-63 | H | H | H | 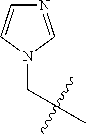 | H | H | H | 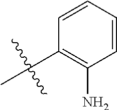 |
| c3-64 | H | H | H | H | 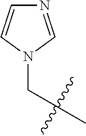 | H | H | 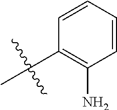 |
| c3-65 | H | 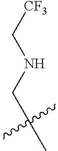 | H | H | H | H | H | 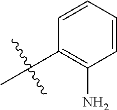 |
| c3-66 | H | H | 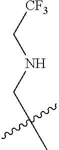 | H | H | H | H | 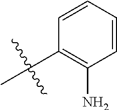 |
| c3-67 | H | H | H | 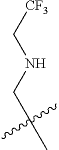 | H | H | H | 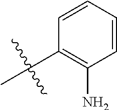 |
| c3-68 | H | H | H | H | 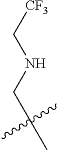 | H | H | 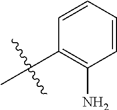 |
| c3-69 | H | 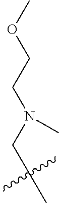 | H | H | H | H | H | 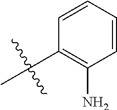 |
| c3-70 | H | H | 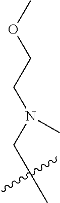 | H | H | H | H | 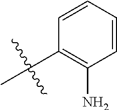 |
| c3-71 | H | H | H | 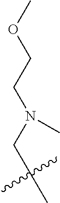 | H | H | H | 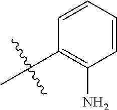 |
| c3-72 | H | H | H | H | 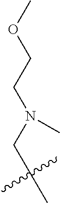 | H | H | 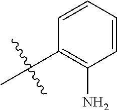 |
| c3-73 | H | 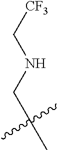 | H | H | H | H | H | 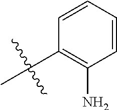 |
| c3-74 | H | H | 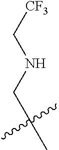 | H | H | H | H | 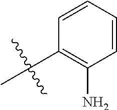 |
| c3-75 | H | H | H | 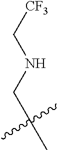 | H | H | H | 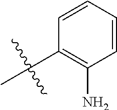 |
| c3-76 | H | H | H | H | 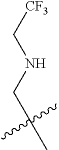 | H | H | 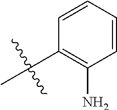 |
| c3-77 | H | 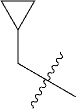 | H | H | H | H | H | 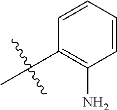 |
| c3-78 | H | H | 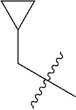 | H | H | H | H | 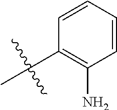 |
| c3-79 | H | H | H | 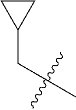 | H | H | H | 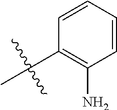 |
| c3-80 | H | H | H | H | 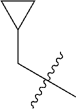 | H | H | 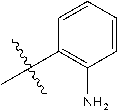 |
| c3-81 | H | 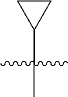 | H | H | H | H | H | 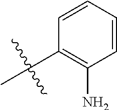 |
| c3-82 | H | H | 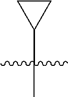 | H | H | H | H | 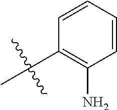 |
| c3-83 | H | H | H | 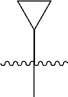 | H | H | H | 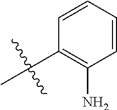 |
| c3-84 | H | H | H | H | 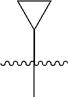 | H | H | 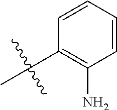 |
| c3-85 | H | 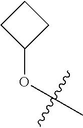 | H | H | H | H | H | 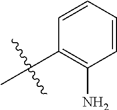 |
| c3-86 | H | H | 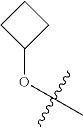 | H | H | H | H | 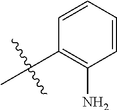 |
| c3-87 | H | H | H | 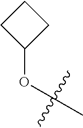 | H | H | H | 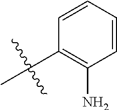 |
| c3-88 | H | H | H | H | 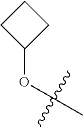 | H | H | 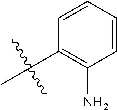 |
| c3-89 | H | 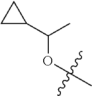 | H | H | H | H | H | 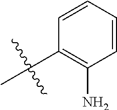 |
| c3-90 | H | H | 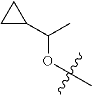 | H | H | H | H | 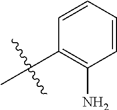 |
| c3-91 | H | H | H | 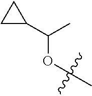 | H | H | H | 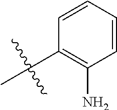 |
| c3-92 | H | H | H | H | 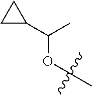 | H | H | 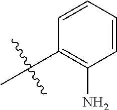 |
| c3-93 | H | 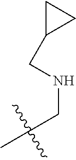 | H | H | H | H | H | 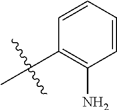 |
| c3-94 | H | H | 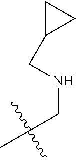 | H | H | H | H | 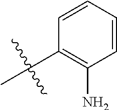 |
| c3-95 | H | H | H | 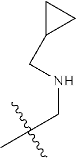 | H | H | H | 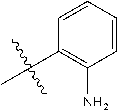 |
| c3-96 | H | H | H | H | 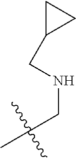 | H | H | 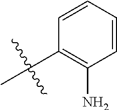 |
| c3-97 | H | 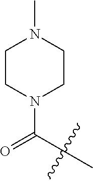 | H | H | H | H | H | 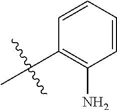 |
| c3-98 | H | H | 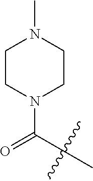 | H | H | H | H | 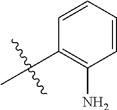 |
| c3-99 | H | H | H | 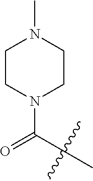 | H | H | H | 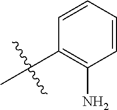 |
| c3-100 | H | H | H | H | 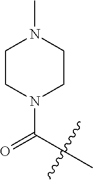 | H | H | 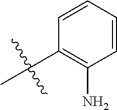 |
| c3-101 | H | 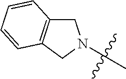 | H | H | H | H | H | 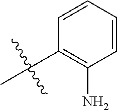 |
| c3-102 | H | H | 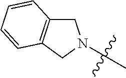 | H | H | H | H | 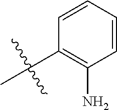 |
| c3-103 | H | H | H | 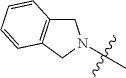 | H | H | H | 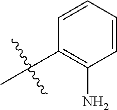 |
| c3-104 | H | H | H | H | 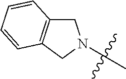 | H | H | 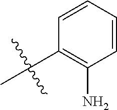 |
| c3-105 | —CH₃ | —F | H | H | H | H | H | —OH |
| c3-106 | —CH₃ | —F | H | H | H | H | H | 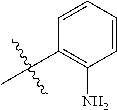 |
| c3-107 | —CH₃ | H | H | —Br | H | H | H | —OH |
| c3-108 | —CH₃ | H | H | —Br | H | H | H | 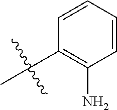 |
| c3-109 | —CH₃ | H | 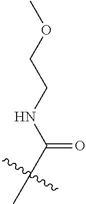 | H | H | H | H | —OH |
| c3-110 | —CH₃ | H | 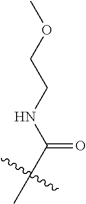 | H | H | H | H | 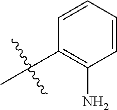 |
| c3-111 | —CH₃ | H | H | 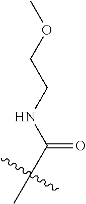 | H | H | H | —OH |
| c3-112 | —CH₃ | H | H | 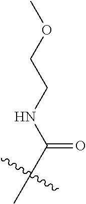 | H | H | H | 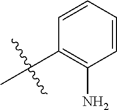 |
| c3-113 | —CH₃ | H | 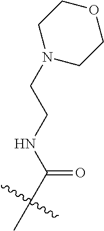 | H | H | H | H | —OH |
| c3-114 | —CH₃ | H | 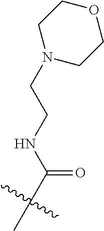 | H | H | H | H | 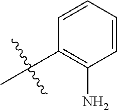 |
| c3-115 | —CH₃ | H | H | 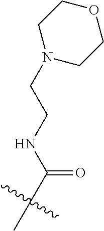 | H | H | H | —OH |
| c3-116 | —CH₃ | H | H | 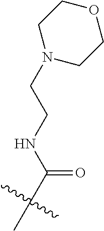 | H | H | H | 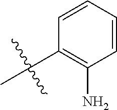 |
| c3-117 | —CH₃ | H | 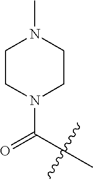 | H | H | H | H | —OH |
| c3-118 | —CH₃ | H | 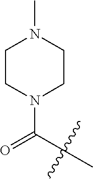 | H | H | H | H | 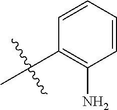 |
| c3-119 | —CH₃ | H | H | 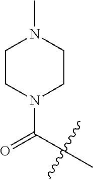 | H | H | H | —OH |
| c3-120 | —CH₃ | H | H | 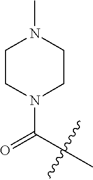 | H | H | H | 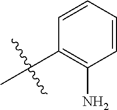 |
| c3-121 | —CH₃ | H | 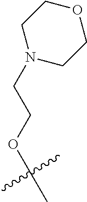 | H | | H | H | —OH |
| c3-122 | —CH₃ | H | 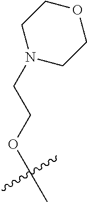 | H | H | H | H | 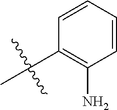 |
| c3-123 | —CH₃ | H | 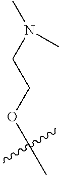 | H | H | H | H | —OH |
| c3-124 | —CH₃ | H | 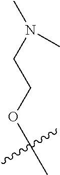 | H | H | H | H | 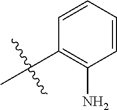 |
| c3-125 | —CH₃ | H | 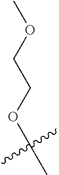 | H | H | H | H | —OH |
| c3-126 | —CH₃ | H | 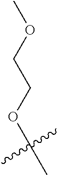 | H | H | H | H | 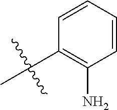 |
| c3-127 | —CH₃ | H | H | 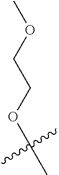 | H | H | H | —OH |
| c3-128 | —CH₃ | H | H | 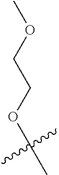 | H | H | H | 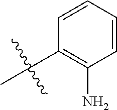 |
| c3-129 | —CH₃ | H | 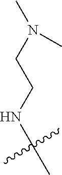 | H | H | H | H | —OH |
| c3-130 | —CH₃ | H | 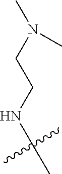 | H | H | H | H | 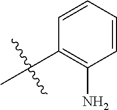 |
| c3-131 | —CH₃ | H | H | —OCH₃ | H | H | H | —OH |
| c3-132 | —CH₃ | H | H | —OCH₃ | H | H | H | 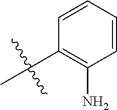 |
| c3-133 | H | H | —OCH₃ | H | H | H | H | —OH |
| c3-134 | H | H | —OCH₃ | H | H | H | H | 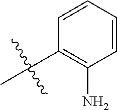 |
| c3-135 | —CH₃ | H | 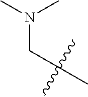 | H | H | H | H | —OH |
| c3-136 | —CH₃ | H | 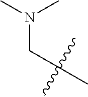 | H | H | H | H | 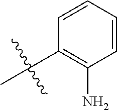 |
| c3-137 | —CF₃ | H | H | H | H | H | H | —OH |
| c3-138 | —CF₃ | H | H | H | H | H | H | 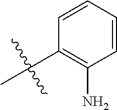 |
| c3-139 | —CH₃ | H | —CN | H | H | H | H | —OH |
| c3-140 | —CH₃ | H | —CN | H | H | H | H | 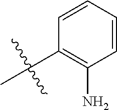 |
| c3-141 | —CH₃ | H | 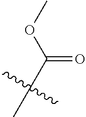 | H | H | H | H | —OH |
| c3-142 | —CH₃ | H | 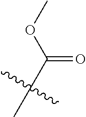 | H | H | H | H | 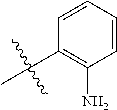 |
| c3-143 | —CH₃ | H | —COOH | H | H | H | H | —OH |
| c3-144 | —CH₃ | H | —COOH | H | H | H | H | 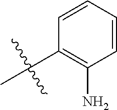 |
| c3-145 | —CH₃ | H | H | H | H | H | F* | —OH |
| (2,5-thiophene) | | | | | | | | |
| c3-146 | —CH₃ | H | H | H | H | H | F* | —OH |
| (2,4-thiophene) | | | | | | | | |
| c3-147 (2,5-thiophene) | —CH₃ | H | H | H | H | H | F* | 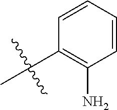 |
| c3-148 (2,4-thiophene) | —CH₃ | H | H | H | H | H | F* | 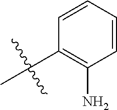 |
| c3-149 | —CH₃ | H | 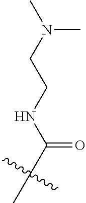 | H | H | H | H | —OH |
| c3-150 | —CH₃ | H | 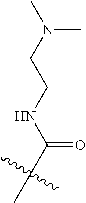 | H | H | H | H | 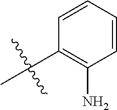 |
| c3-151 | H | H | H | H | H | F | H | —OH |
| c3-152 | H | H | H | H | H | F | H | 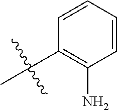 |

*Each of Reference No. c3-145 (hydroxamate) and c3-147 (arylamide) embraces both the 3-fluoro-2,5-thiophendiyl and the 4-fluoro-2,5-thiophendiyl species of the respective compound, while each of c3-146 and c3-148 embraces both the 3-fluoro-2,4-thiophendiyl and 5-fluoro-2,4-thiophendiyl species of the respective hydroxamate and arylamide.

In one embodiment, the invention provides a compound of Formula (I-c) and a pharmaceutically acceptable salt thereof:

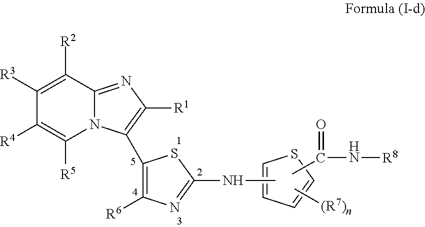

wherein R¹, R², R³, R⁴, R⁵, R⁶, R⁷ and R⁸ are as defined for any of Formulae (I), (I-a), (I-b), and (I-c) above.

In an embodiment of Formula (I-d), R¹, R², R³, R⁴ and R⁵ are H; R⁶ is H, alkyl or haloalkyl; R⁷ is fluoro, chloro, bromo, or methyl and n is 0 or 1; and R⁸ is hydroxyl, aryl or heteroaryl, wherein aryl or heteroaryl are substituted with —NH₂ or —OH at a ring position adjacent to attachment of the —CONH-moiety is optionally substituted with one or more groups R¹⁰ selected from amino, halo, alkyl, alicyclyl, heterocyclyl and aryl.

Non-limiting examples of such compounds include the following compounds and pharmaceutically acceptable salts thereof:

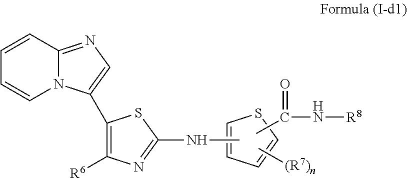

The Table lists compounds of Formula (I-d1) that in one embodiment have a 2,5-configuration on the thiophene and in another have a 2,4-configuration on the thiophene; in the compounds of the Table, n=0, which is indicated by a listing of “H” under the R⁷ column.

| Compound No. | R⁶ | R⁷ | R⁸ |
|---|---|---|---|
| d1-01 | H | H | —OH |
| d1-02 | —CH₃ | H | —OH |
| d1-03 | H | H | 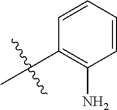 |
| d1-04 | —CH₃ | H | 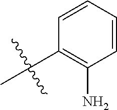 |
| d1-05 | H | H | 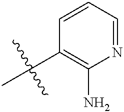 |
| d1-06 | —CH₃ | H | 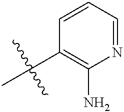 |
| d1-07 | H | H | 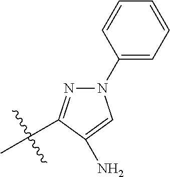 |
| d1-08 | —CH₃ | H | 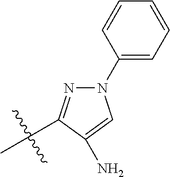 |
| d1-09 | H | H | 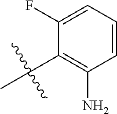 |
| d1-10 | —CH₃ | H | 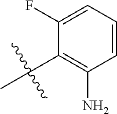 |

In another embodiment of Formula (I-d), R¹ is methyl; R², R³, R⁴ and R⁵ are H; R⁶ is H, alkyl or haloalkyl; R⁷ is fluoro, chloro, bromo, or methyl and n is 0 or 1; and R⁸ is hydroxyl, aryl or heteroaryl, wherein aryl or heteroaryl are substituted with —NH₂ or —OH at a ring position adjacent to attachment of the —CONH-moiety and R⁸ is optionally substituted with one or more groups selected from amino, halo, alkyl, alicyclyl, heterocyclyl and aryl.

Non-limiting examples of such compounds include the following compounds and pharmaceutically acceptable salts thereof:

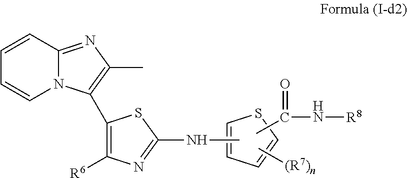

The Table lists compounds of Formula (I-d2) that in one embodiment have a 2,5-configuration on the thiophene and in another have a 2,4-configuration on the thiophene; in the compounds of the Table, n=0, which is indicated by a listing of “H” under the R⁷ column.

| Compound No. | R⁶ | R⁷ | R⁸ |
|---|---|---|---|
| d2-01 | H | H | —OH |
| d2-02 | —CH₃ | H | —OH |
| d2-03 | H | H | 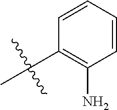 |
| d2-04 | —CH₃ | H | 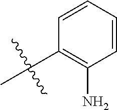 |
| d2-05 | H | H | 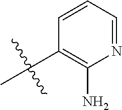 |
| d2-06 | —CH₃ | H | 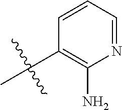 |
| d2-07 | H | H | 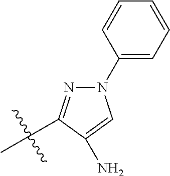 |
| d2-08 | —CH₃ | H | 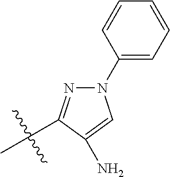 |
| d2-09 | H | H | 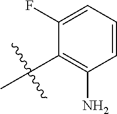 |
| d2-10 | —CH₃ | H | 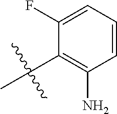 |

In another embodiment of Formula (I-d), at least two of R¹, R², R³, R⁴ and R⁵ are H and each non-hydrogen R¹, R², R³, R⁴ and R⁵ is independently selected from chloro, fluoro, bromo, methyl, ethyl, propyl, methoxy, ethoxy, carboxy, cyano, methoxymethyl, ethoxyethyl, propoxyethyl, methoxyethoxy, trifluoromethyl, hydroxyethoxy, dimethylamino, diethylamino, dimethylaminomethyl, dimethylamino ethyl, diethylaminomethyl, dimethylaminoethoxy, trifluoromethoxymethyl, trifluoroethoxymethyl, 3-oxetanoxy, trifluoroethylaminomethyl, N-methyl-N-methoxyethyl-aminoethyl, cyclopropanylmethyl, cyclobutoxy, 1-cyclopropanylethoxy, cyclopropanylmethylaminomethyl, 4-methylpiperazin-1-carbonyl, isoindolin-2-yl, N-methoxyethylcarbamoyl, N-(morpholin-4-yl)-ethylcarbamoyl, dimethylaminoethylamino, methylcarboxy, N,N-dimethylaminoethylcarbamoyl, benzyl, phenylethyl, trifluoromethylphenylethyl, phenoxymethyl, fluorophenoxymethyl, phenylethylaminomethyl, benzylaminomethyl, triazinylmethyl, piperidinylmethyl, piperidinyloxy, trifluoromethylpiperidinylmethyl, pyridinyloxymethyl, pyridinylmethoxy, tetrahydropyrazinyloxy, methylpiperazinylmethyl, pyrrolidin-1-yl, pyrrolidin-2-yl, pyrrolidin-3-yl, pyrrolidin-1-ylmethyl, pyrrolidin-2-ylmethyl, pyrrolidin-3-ylmethyl, pyrrolidin-1-ylethoxy, pyrrolidin-2-ylethoxy, pyrrolidin-3-ylethoxy, imidazol-1-ylmethyl, imidazol-2-ylmethyl, imidazol-4-ylmethyl, imidazolidin-1-yl, imidazolidin-2-yl, imidazolidin-4-yl, imidazolidin-1-ylmethyl, imidazolidin-2-ylmethyl, imidazolidin-4-ylmethyl, imidazolin-1-yl, imidazolin-2-yl, imidazolin-4-yl, pyrazolidin-1-yl, pyrazolidin-3-yl, pyrazolidin-4-yl, pyrazolin-1-yl, pyrazolin-3-yl, pyrazolin-4-yl, piperidin-1-yl, piperidin-2-yl, piperidin-3-yl, piperidin-4-yl, piperidin-1-ylmethyl, piperidin-2-ylmethyl, piperidin-3-ylmethyl, piperidin-4-ylmethyl, piperazin-1-yl, piperazin-2-yl, piperazin-3-yl, morpholin-2-yl, morpholin-3-yl, morpholin-4-yl, morpholin-2-ylmethyl, morpholin-3-ylmethyl, morpholin-4-ylmethyl, morpholin-2-ylethoxy, morpholin-3-ylethoxy and morpholin-4-ylethoxy; R⁶ is H, alkyl or haloalkyl; R⁷ is independently fluoro, chloro, bromo, or methyl and n is 0 or 1; and R⁸ is hydroxyl, aryl or heteroaryl, wherein aryl or heteroaryl are substituted with —NH₂ or —OH and R⁸ is optionally substituted with one or more groups selected from amino, halo, alkyl, alicyclyl, heterocyclyl and aryl.

Non-limiting examples of such compounds include the following compounds and pharmaceutically acceptable salts thereof:

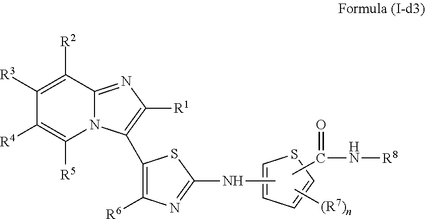

The Table discloses compounds of Formula (I-d3) that in one embodiment have a 2,5-configuration on the thiophene and in another have a 2,4-configuration on the thiophene. To illustrate, the row labeled as “reference No. d3-01” discloses two thiophene HDAC compounds and their pharmaceutically acceptable salts. The first compound contains the R¹-R⁸ substituents of the c3-01 row on a compound of Formula (I-d3) where the —NH— and the —C(O)NHR⁸ are disposed about the thiophene ring in a 2,5-configuration, with the S atom taken as position 1. The second compound (and salts) embraced by Reference No. c3-01 has the same substituents R¹-R⁸, but the —NH— and the —C(O)NHR⁸ are disposed about the thiophene ring in a 2,4-configuration.

In the compounds of the Table, n=0 or n=1. When n=0, by convention this is indicated by a listing of “H” under the R⁷ column. When n=1, the substituent listed in the R⁷ column is attached to one of the two “free” positions on the thiophene ring not occupied by the —NH— or —C(O)NHR⁸ groups. When the Reference No. discloses a 2,5-substituted thiophene, the substituent R⁷ is on the 3-position in a first embodiment and on the 4-position in a second embodiment. Similarly, when the Reference No. discloses a 2,4-substituted thiophene, the substituent R⁷ is on the 3-position in a first embodiment and on the 5-position in a second embodiment. This is indicated in the Table (Reference No. d3-145 through d3-148) by a parenthetical mention of the particular thiophene configuration below the reference no. Thus to illustrate, each of Reference No. d3-145 (hydroxamate) and d3-147 (arylamide) embraces both the 3-fluoro-2,5-thiophendiyl and the 4-fluoro-2,5-thiophendiyl species of the respective compound, while each of d3-146 and d3-148 embraces the 3-fluoro-2,4-thiophendiyl and 5-fluoro-2,4-thiophendiyl species of the respective hydroxamate and arylamide.

Compounds of Formula I-d3:

| Reference No. | R¹ | R² | R³ | R⁴ | R⁵ | R⁶ | R⁷ | R⁸ |
|---|---|---|---|---|---|---|---|---|
| d3-01 | —CH₃ | —Cl | H | H | H | H | H | —OH |
| d3-02 | —CH₃ | H | —Cl | H | H | H | H | —OH |
| d3-03 | —CH₃ | H | H | —Cl | H | H | H | —OH |
| d3-04 | —CH₃ | H | H | H | —Cl | H | H | —OH |
| d3-05 | —CH₃ | —Cl | H | H | H | H | H | 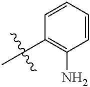 |
| d3-06 | —CH₃ | H | —Cl | H | H | H | H | 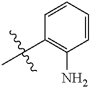 |
| d3-07 | —CH₃ | H | H | —Cl | H | H | H | 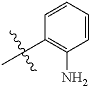 |
| d3-08 | —CH₃ | H | H | H | —Cl | H | H | 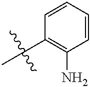 |
| d3-09 | —CH₃ | —Cl | H | H | H | H | H | 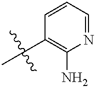 |
| d3-10 | —CH₃ | H | —Cl | H | H | H | H | 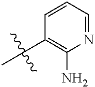 |
| d3-11 | —CH₃ | H | H | —Cl | H | H | H | 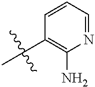 |
| d3-12 | —CH₃ | H | H | H | —Cl | H | H | 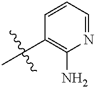 |
| d3-13 | —CH₃ | —CF₃ | H | H | H | H | H | —OH |
| d3-14 | —CH₃ | H | —CF₃ | H | H | H | H | —OH |
| d3-15 | —CH₃ | H | H | —CF₃ | H | H | H | —OH |
| d3-16 | —CH₃ | H | H | H | —CF₃ | H | H | —OH |
| d3-17 | —CH₃ | —CF₃ | H | H | H | H | H | 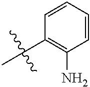 |
| d3-18 | —CH₃ | H | —CF₃ | H | H | H | H | 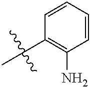 |
| d3-19 | —CH₃ | H | H | —CF₃ | H | H | H | 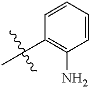 |
| d3-20 | —CH₃ | H | H | H | —CF₃ | H | H | 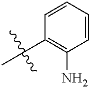 |
| d3-21 | —CH₃ | —OH | H | H | H | H | H | —OH |
| d3-22 | —CH₃ | H | —OH | H | H | H | H | —OH |
| d3-23 | —CH₃ | H | H | —OH | H | H | H | —OH |
| d3-24 | —CH₃ | H | H | H | —OH | H | H | —OH |
| d3-25 | —CH₃ | —OH | H | H | H | H | H | 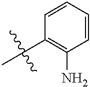 |
| d3-26 | —CH₃ | H | —OH | H | H | H | H | 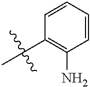 |
| d3-27 | —CH₃ | H | H | —OH | H | H | H | 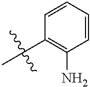 |
| d3-28 | —CH₃ | H | H | H | —OH | H | H | 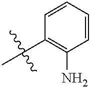 |
| d3-29 | —CH₃ | 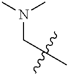 | H | H | H | H | H | —OH |
| d3-30 | —CH₃ | H | 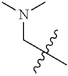 | H | H | H | H | —OH |
| d3-31 | —CH₃ | H | H | 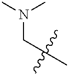 | H | H | H | —OH |
| d3-32 | —CH₃ | H | H | H | 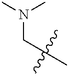 | H | H | —OH |
| d3-33 | —CH₃ | 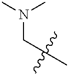 | H | H | H | H | H | 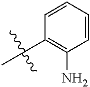 |
| d3-34 | —CH₃ | H | 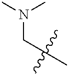 | H | H | H | H | 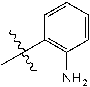 |
| d3-35 | —CH₃ | H | H | 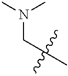 | H | H | H | 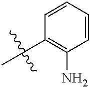 |
| d3-36 | —CH₃ | H | H | H | 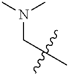 | H | H | 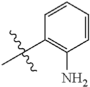 |
| d3-37 | —CH₃ | 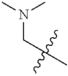 | H | H | H | H | H | 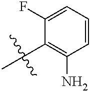 |
| d3-38 | —CH₃ | H | 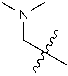 | H | H | H | H | 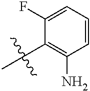 |
| d3-39 | —CH₃ | H | H | 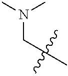 | H | H | H | 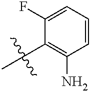 |
| d3-40 | —CH₃ | H | H | H | 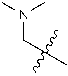 | H | H | 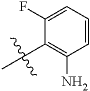 |
| d3-41 | —CH₃ | 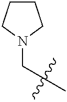 | H | H | H | H | H | 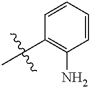 |
| d3-42 | —CH₃ | H | 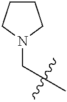 | H | H | H | H | 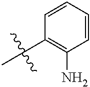 |
| d3-43 | —CH₃ | H | H | 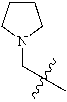 | H | H | H | 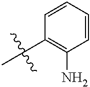 |
| d3-44 | —CH₃ | H | H | H | 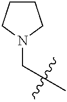 | H | H | 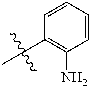 |
| d3-45 | —CH₃ | 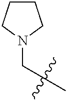 | H | H | H | H | H | 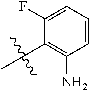 |
| d3-46 | —CH₃ | H | 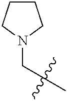 | H | H | H | H | 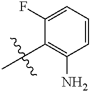 |
| d3-47 | —CH₃ | H | H | 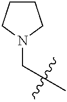 | H | H | H | 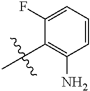 |
| d3-48 | —CH₃ | H | H | H | 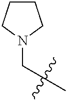 | H | H | 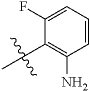 |
| d3-49 | —CH₃ | 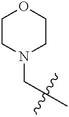 | H | H | H | H | H | 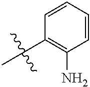 |
| d3-50 | —CH₃ | H | 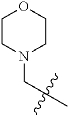 | H | H | H | H | 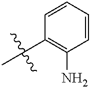 |
| d3-51 | —CH₃ | H | H | 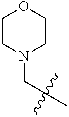 | H | H | H | 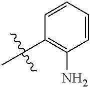 |
| d3-52 | —CH₃ | H | H | H | 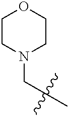 | H | H | 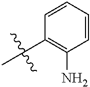 |
| d3-53 | —CH₃ | 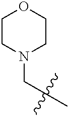 | H | H | H | H | H | 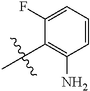 |
| d3-54 | —CH₃ | H | 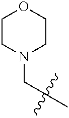 | H | H | H | H | 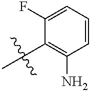 |
| d3-55 | —CH₃ | H | H | 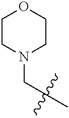 | H | H | H | 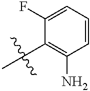 |
| d3-56 | —CH₃ | H | H | H | 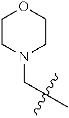 | H | H | 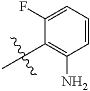 |
| d3-57 | H | 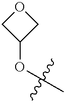 | H | H | H | H | H | 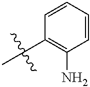 |
| d3-58 | H | H | 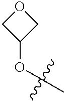 | H | H | H | H | 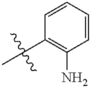 |
| d3-59 | H | H | H | 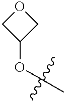 | H | H | H | 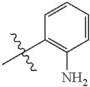 |
| d3-60 | H | H | H | H | 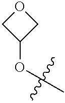 | H | H | 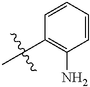 |
| d3-61 | H | 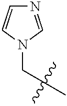 | H | H | H | H | H | 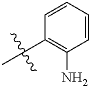 |
| d3-62 | H | H | 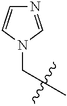 | H | H | H | H | 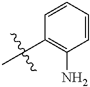 |
| d3-63 | H | H | H | 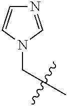 | H | H | H | 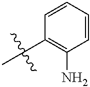 |
| d3-64 | H | H | H | H | 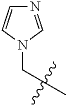 | H | H | 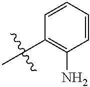 |
| d3-65 | H | 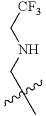 | H | H | H | H | H | 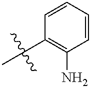 |
| d3-66 | H | H | 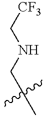 | H | H | H | H | 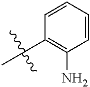 |
| d3-67 | H | H | H | 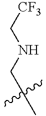 | H | H | H | 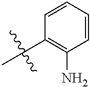 |
| d3-68 | H | H | H | H | 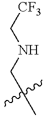 | H | H | 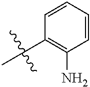 |
| d3-69 | H | 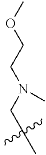 | H | H | H | H | H | 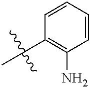 |
| d3-70 | H | H | 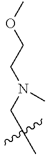 | H | H | H | H | 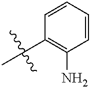 |
| d3-71 | H | H | H | 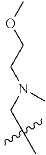 | H | H | H | 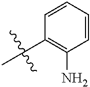 |
| d3-72 | H | H | H | H | 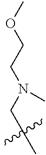 | H | H | 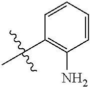 |
| d3-73 | H | 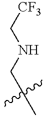 | H | H | H | H | H | 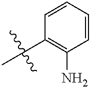 |
| d3-74 | H | H | 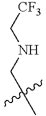 | H | H | H | H | 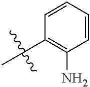 |
| d3-75 | H | H | H | 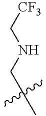 | H | H | H | 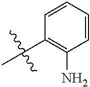 |
| d3-76 | H | H | H | H | 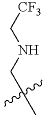 | H | H | 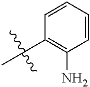 |
| d3-77 | H | 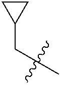 | H | H | H | H | H | 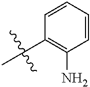 |
| d3-78 | H | H | 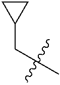 | H | H | H | H | 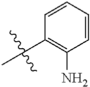 |
| d3-79 | H | H | H | 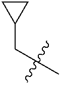 | H | H | H | 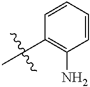 |
| d3-80 | H | H | H | H | 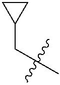 | H | H | 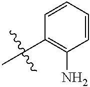 |
| d3-81 | H | 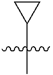 | H | H | H | H | H | 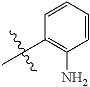 |
| d3-82 | H | H | 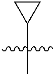 | H | H | H | H | 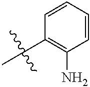 |
| d3-83 | H | H | H | 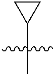 | H | H | H | 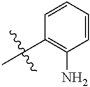 |
| d3-84 | H | H | H | H | 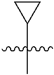 | H | H | 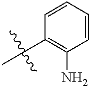 |
| d3-85 | H | 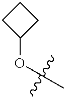 | H | H | H | H | H | 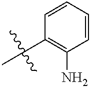 |
| d3-86 | H | H | 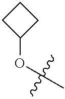 | H | H | H | H | 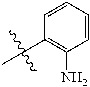 |
| d3-87 | H | H | H | 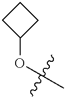 | H | H | H | 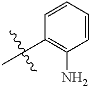 |
| d3-88 | H | H | H | H | 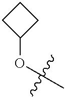 | H | H | 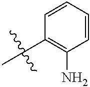 |
| d3-89 | H | 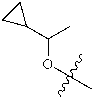 | H | H | H | H | H | 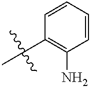 |
| d3-90 | H | H | 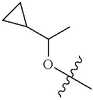 | H | H | H | H | 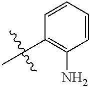 |
| d3-91 | H | H | H | 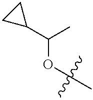 | H | H | H | 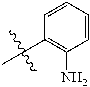 |
| d3-92 | H | H | H | H | 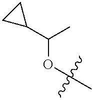 | H | H | 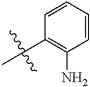 |
| d3-93 | H | 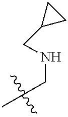 | H | H | H | H | H | 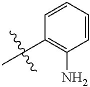 |
| d3-94 | H | H | 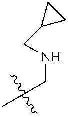 | H | H | H | H | 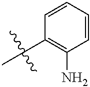 |
| d3-95 | H | H | H | 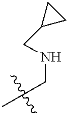 | H | H | H | 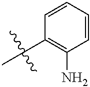 |
| d3-96 | H | H | H | H | 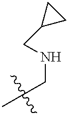 | H | H | 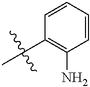 |
| d3-97 | H | 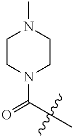 | H | H | H | H | H | 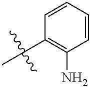 |
| d3-98 | H | H | 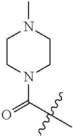 | H | H | H | H | 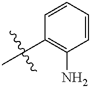 |
| d3-99 | H | H | H | 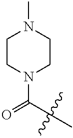 | H | H | H | 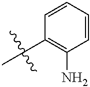 |
| d3-100 | H | H | H | H | 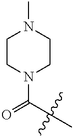 | H | H | 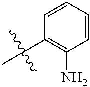 |
| d3-101 | H | 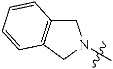 | H | H | H | H | H | 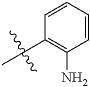 |
| d3-102 | H | H | 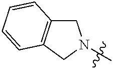 | H | H | H | H | 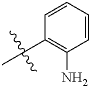 |
| d3-103 | H | H | H | 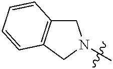 | H | H | H | 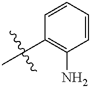 |
| d3-104 | H | H | H | H | 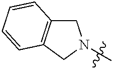 | H | H | 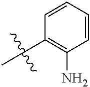 |
| d3-105 | —CH₃ | —F | H | H | H | H | H | —OH |
| d3-106 | —CH₃ | —F | H | H | H | H | H | 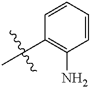 |
| d3-107 | —CH₃ | H | H | —Br | H | H | H | —OH |
| d3-108 | —CH₃ | H | H | —Br | H | H | H | 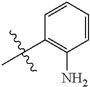 |
| d3-109 | —CH₃ | H | 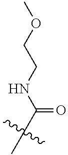 | H | H | H | H | —OH |
| d3-110 | —CH₃ | H | 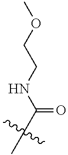 | H | H | H | H | 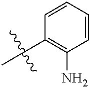 |
| d3-111 | —CH₃ | H | H | 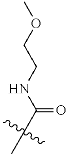 | H | H | H | —OH |
| d3-112 | —CH₃ | H | H | 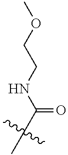 | H | H | H | 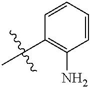 |
| d3-113 | —CH₃ | H | 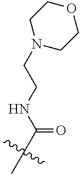 | H | H | H | H | —OH |
| d3-114 | —CH₃ | H | 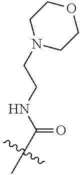 | H | H | H | H | 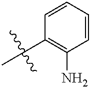 |
| d3-115 | —CH₃ | H | H | 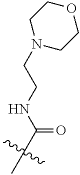 | H | H | H | —OH |
| d3-116 | —CH₃ | H | H | 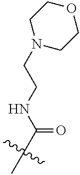 | H | H | H | 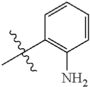 |
| d3-117 | —CH₃ | H | 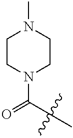 | H | H | H | H | —OH |
| d3-118 | —CH₃ | H | 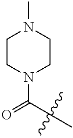 | H | H | H | H | 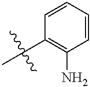 |
| d3-119 | —CH₃ | H | H | 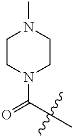 | H | H | H | —OH |
| d3-120 | —CH₃ | H | H | 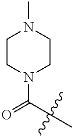 | H | H | H | 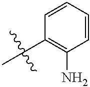 |
| d3-121 | —CH₃ | H | 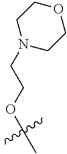 | H | H | H | H | —OH |
| d3-122 | —CH₃ | H | 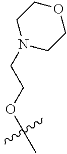 | H | H | H | H | 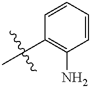 |
| d3-123 | —CH₃ | H | 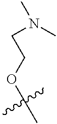 | H | H | H | H | —OH |
| d3-124 | —CH₃ | H | 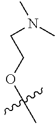 | H | H | H | H | 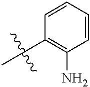 |
| d3-125 | —CH₃ | H | 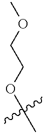 | H | H | H | H | —OH |

| | | | | | | | | |
|---|---|---|---|---|---|---|---|---|
| d3-126 | —CH₃ | H | 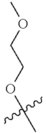 | H | H | H | H | 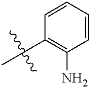 |

| | | | | | | | | |
|---|---|---|---|---|---|---|---|---|
| d3-127 | —CH₃ | H | H | 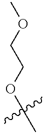 | H | H | H | —OH |
| d3-128 | —CH₃ | H | H | 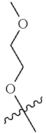 | H | H | H | 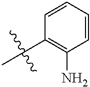 |
| d3-129 | —CH₃ | H | 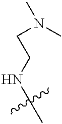 | H | H | H | H | —OH |
| d3-130 | —CH₃ | H | 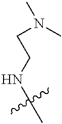 | H | H | H | H | 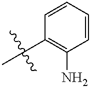 |
| d3-131 | —CH₃ | H | H | —OCH₃ | H | H | H | —OH |
| d3-132 | —CH₃ | H | H | —OCH₃ | H | H | H | 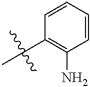 |
| d3-133 | H | H | —OCH₃ | H | H | H | H | —OH |
| d3-134 | H | H | —OCH₃ | H | H | H | H | 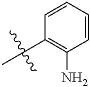 |
| d3-135 | —CH₃ | H | 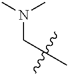 | H | H | H | H | —OH |
| d3-136 | —CH₃ | H | 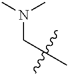 | H | H | H | H | 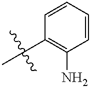 |
| d3-137 | —CF₃ | H | H | H | H | H | H | —OH |
| d3-138 | —CF₃ | H | H | H | H | H | H | 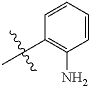 |
| d3-139 | —CH₃ | H | —CN | H | H | H | H | —OH |
| d3-140 | —CH₃ | H | —CN | H | H | H | H | 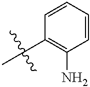 |
| d3-141 | —CH₃ | H | 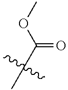 | H | H | H | H | —OH |
| d3-142 | —CH₃ | H | 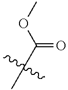 | H | H | H | H | 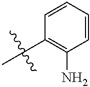 |
| d3-143 | —CH₃ | H | —COOH | H | H | H | H | —OH |
| d3-144 | —CH₃ | H | —COOH | H | H | H | H | 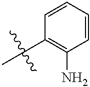 |
| d3-145 | —CH₃ | H | H | H | H | H | F * | —OH |
| (2,5-thiophene) | | | | | | | | |
| d3-146 | —CH₃ | H | H | H | H | H | F * | —OH |
| (2,4-thiophene) | | | | | | | | |
| d3-147 (2,5-thiophene) | —CH₃ | H | H | H | H | H | F * | 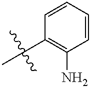 |
| d3-148 (2,4-thiophene) | —CH₃ | H | H | H | H | H | F * | 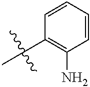 |
| d3-149 | —CH₃ | H | 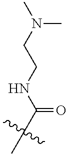 | H | H | H | H | —OH |
| d3-150 | —CH₃ | H | 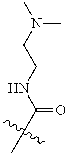 | H | H | H | H | 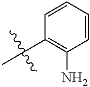 |
| d3-151 | H | H | H | H | H | F | H | —OH |
| d3-152 | H | H | H | H | H | F | H | 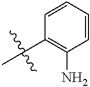 |

* Each of Reference No. d3-145 (hydroxamate) and d3-147 (arylamide) embraces both the 3-fluoro-2,5-thiophendiyl and the 4-fluoro-2,5-thiophendiyl species of the respective compound, while each of d3-146 and d3-148 embraces both the 3-fluoro-2,4-thiophendiyl and 5-fluoro-2,4-thiophendiyl species of the respective hydroxamate and arylamide.

*Each of Reference No. d3-145 (hydroxamate) and d3-147 (arylamide) embraces both the 3-fluoro-2,5-thiophendiyl and the 4-fluoro-2,5-thiophendiyl species of the respective compound, while each of d3-146 and d3-148 embraces both the 3-fluoro-2,4-thiophendiyl and 5-fluoro-2,4-thiophendiyl species of the respective hydroxamate and arylamide.

In one embodiment, the invention provides a compound of Formula (I-e) and a pharmaceutically acceptable salt thereof:

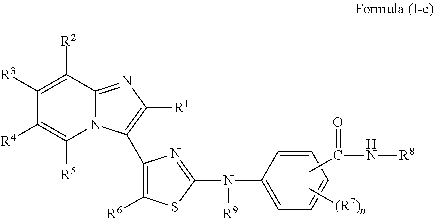

wherein R¹, R², R³, R⁴, R⁵, R⁶, R⁷ and R⁸ are as defined above for various aspects of Formula (I) and R⁹ is a non-hydrogen substitutent.

In an embodiment of Formula (I-e), R¹, R², R³, R⁴ and R⁵ are independently selected from the group consisting of hydrogen, chloro, fluoro, bromo, methyl, ethyl, propyl, methoxy, ethoxy, carboxy, cyano, methoxymethyl, ethoxyethyl, propoxyethyl, methoxyethoxy, trifluoromethyl, hydroxyethoxy, dimethylamino, diethylamino, dimethylaminomethyl, dimethylaminoethyl, diethylaminomethyl, dimethylaminoethoxy, trifluoromethoxymethyl, trifluoroethoxymethyl, 3-oxetanoxy, trifluoroethylaminomethyl, N-methyl-N-methoxyethyl-aminoethyl, cyclopropanylmethyl, cyclobutoxy, 1-cyclopropanylethoxy, cyclopropanylmethylaminomethyl, 4-methylpiperazin-1-carbonyl, isoindolin-2-yl, N-methoxyethylcarbamoyl, N-(morpholin-4-yl)-ethylcarbamoyl, dimethylaminoethylamino, methylcarboxy, N,N-dimethylaminoethylcarbamoyl, benzyl, phenylethyl, trifluoromethylphenylethyl, phenoxymethyl, fluorophenoxymethyl, phenylethylaminomethyl, benzylaminomethyl, triazinylmethyl, piperidinylmethyl, piperidinyloxy, trifluoromethylpiperidinylmethyl, pyridinyloxymethyl, pyridinylmethoxy, tetrahydropyrazinyloxy, methylpiperazinylmethyl, pyrrolidin-1-yl, pyrrolidin-2-yl, pyrrolidin-3-yl, pyrrolidin-1-ylmethyl, pyrrolidin-2-ylmethyl, pyrrolidin-3-ylmethyl, pyrrolidin-1-ylethoxy, pyrrolidin-2-ylethoxy, pyrrolidin-3-ylethoxy, imidazol-1-ylmethyl, imidazol-2-ylmethyl, imidazol-4-ylmethyl, imidazolidin-1-yl, imidazolidin-2-yl, imidazolidin-4-yl, imidazolidin-1-ylmethyl, imidazolidin-2-ylmethyl, imidazolidin-4-ylmethyl, imidazolin-1-yl, imidazolin-2-yl, imidazolin-4-yl, pyrazolidin-1-yl, pyrazolidin-3-yl, pyrazolidin-4-yl, pyrazolin-1-yl, pyrazolin-3-yl, pyrazolin-4-yl, piperidin-1-yl, piperidin-2-yl, piperidin-3-yl, piperidin-4-yl, piperidin-1-ylmethyl, piperidin-2-ylmethyl, piperidin-3-ylmethyl, piperidin-4-ylmethyl, piperazin-1-yl, piperazin-2-yl, piperazin-3-yl, morpholin-2-yl, morpholin-3-yl, morpholin-4-yl, morpholin-2-ylmethyl, morpholin-3-ylmethyl, morpholin-4-ylmethyl, morpholin-2-ylethoxy, morpholin-3-ylethoxy and morpholin-4-ylethoxy; R⁶ is H, alkyl or haloalkyl; each R⁷ is independently fluoro, chloro, bromo, or methyl and n is 0, 1 or 2; R⁸ is hydroxyl, aryl or heteroaryl, wherein aryl or heteroaryl are substituted with —NH₂ or —OH at a ring position adjacent to attachment of the —CONH-moiety, and R⁸ is optionally further substituted with one or more groups selected from amino, halo, alkyl, alicyclyl, heterocyclyl and aryl; and R⁹ is a non-hydrogen substitutent selected from alkyl, haloalkyl and aminoalkyl.

In various embodiments, the groups R¹, R², R³, R⁴, R⁵, R⁶, R⁷ and R⁸ are selected to have the same combination of substituents given in the tables for Compounds a1-1 to a1-24, a2-1 to a2-24 and a3-1 to a3-168 and R⁹ is methyl, ethyl, trifluoromethyl or trifluoroethyl. Non-limiting examples of such compounds include the following compounds and pharmaceutically acceptable salts thereof:

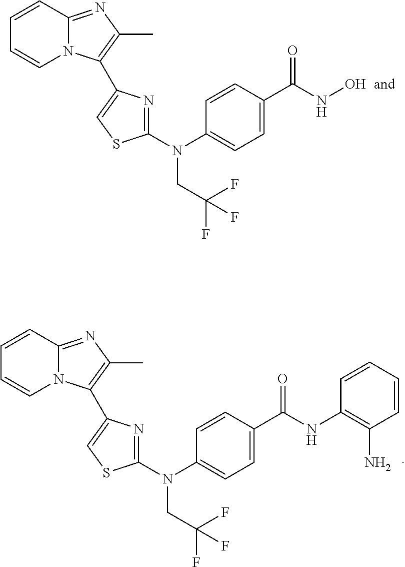

In yet another embodiment, the invention provides a compound of Formula (II) or a pharmaceutically acceptable salt thereof:

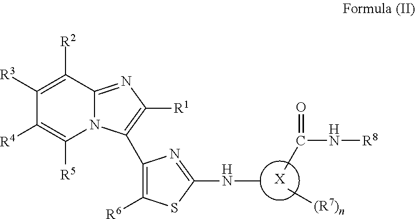

wherein R¹ is selected from the group consisting of H, methyl, ethyl, trifluoromethyl, dimethylaminomethyl, morpholinylmethyl and pyrrolidinylmethyl; at least two of R², R³, R⁴ and R⁵ are H, and the others (i.e., any that are non-hydrogen) are independently selected from the group consisting of hydroxyl, methyl, methoxy, chloro, fluoro, trifluoromethyl, dimethylaminomethyl, morpholinylmethyl and pyrrolidinylmethyl; R⁶ is H or methyl; X is phenyl, 5-membered heteroaryl, or 6-membered heteroaryl, wherein the heteroaryl contains one or more heteroatoms selected from N, S and O; R⁷ when present is halo (e.g., fluoro, bromo, or chloro) and n is 0 or 1; and R⁸ is hydroxyl, aryl or heteroaryl, wherein aryl or heteroaryl are substituted with —NH₂ or —OH at a ring position adjacent to attachment of the —CONH-moiety, and R⁸ is optionally substituted with one or more groups R¹⁰ selected from amino, halo, alkyl, alicyclyl, heterocyclyl and aryl.

Examples of such compounds include:

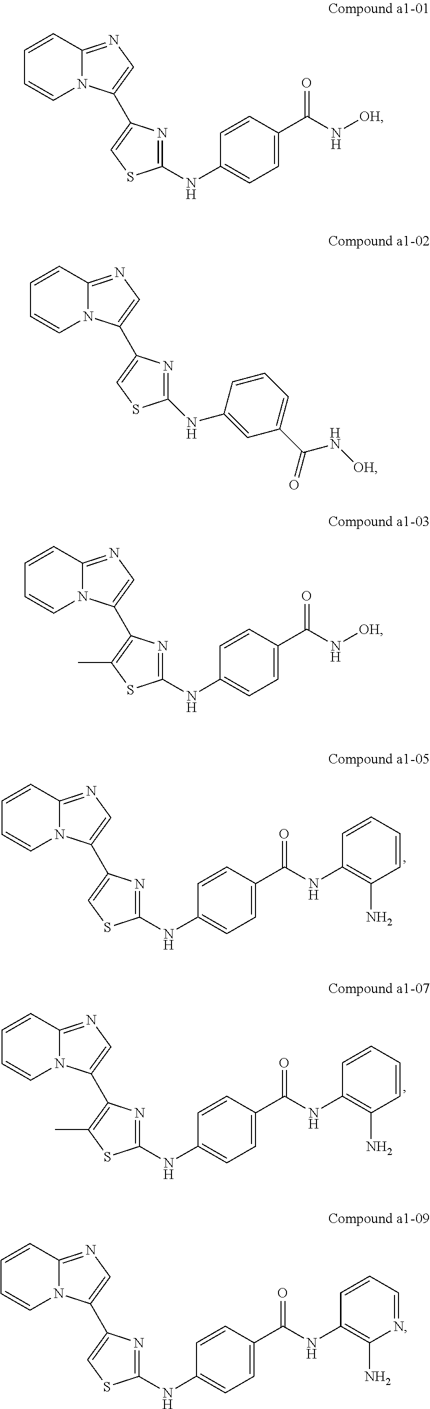

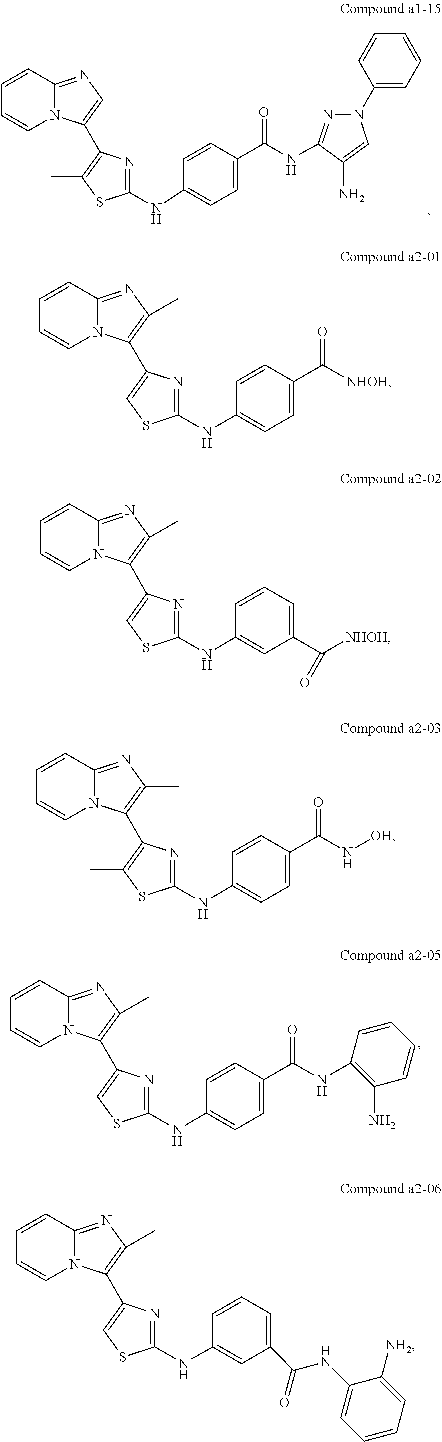

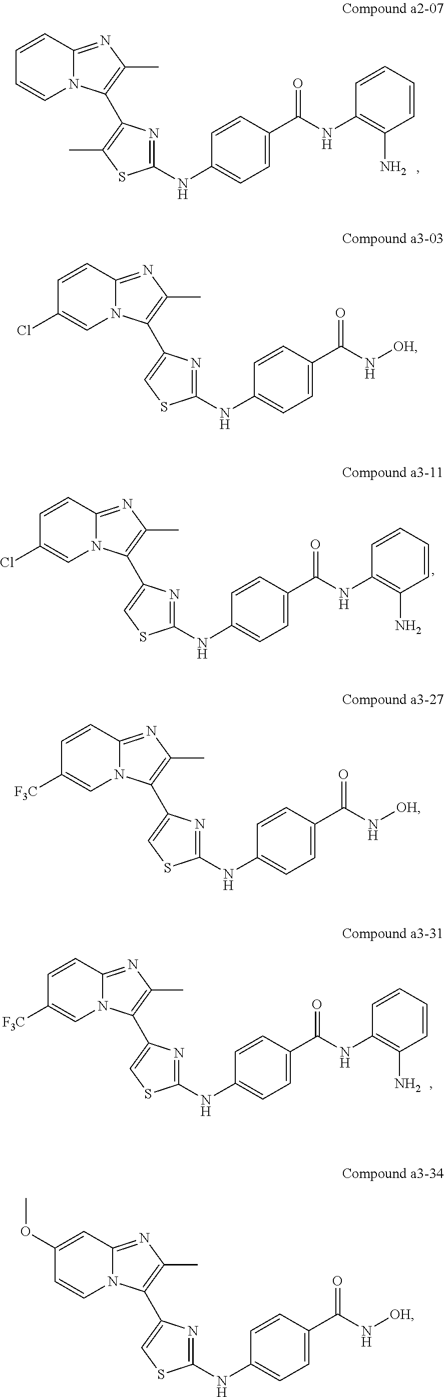

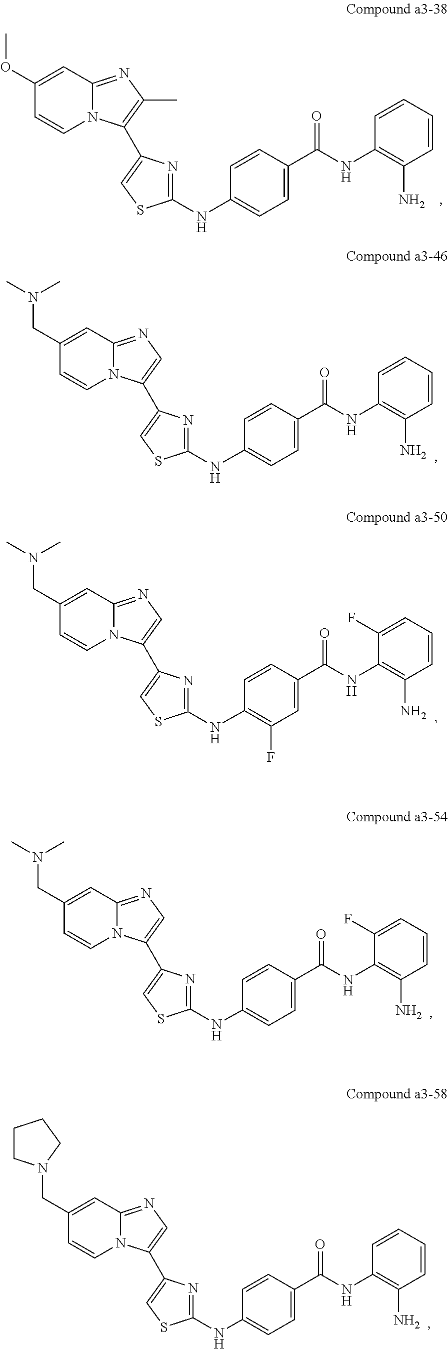

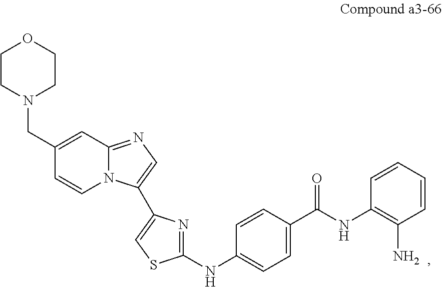

and pharmaceutically acceptable salts thereof.

### Compound Preparation

A compound of the present invention such as those of Formulas (I), (I-a), (I-b), (I-c), and (I-d) can be prepared according to the schemes described below, but it shall be appreciated that modifications of the illustrated process or other process can also be used. Schemes A, B, and C illustrate a method to prepare a compound of

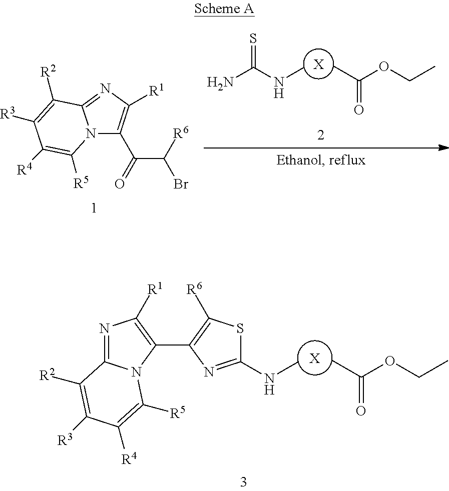

Bromoketone compound 1 is dissolved in a solvent such as ethanol to prepare a solution. Thioureido compound 2 containing an X aromatic group (for clarity the group X in the synthetic schemes is given without the R⁷ group that is attached in the compounds) is added to the solution, and the mixture is refluxed. The solvent is removed under vacuum, and the residue is diluted with ether and then stirred. The solid is filtered and dried under vacuum to yield Compound 3, containing the thiazole ring formed from the reaction of 1 and 2.

In various embodiments, compound 3 is converted to hydroxamates or arylamides of Formula (I). Scheme B below illustrates synthesis of hydroxamates and scheme C illustrates synthesis of benzamides (where the group R⁸ is a substituted aryl ring) from intermediate compound 3.

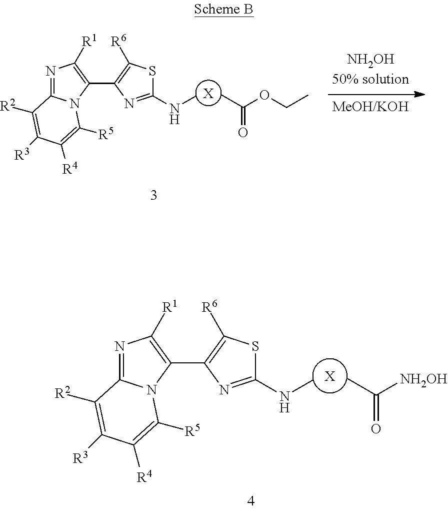

In an illustrative synthesis, compound 3 is dissolved in a solvent such as a mixture of methanol and dichloromethane and the mixture is stirred to prepare a solution. NH₂OH is added to the stirred solution slowly. After stirring, NaOH is added dropwise and brought to room temperature and stirred. The volatiles are evaporated under vacuum, diluted with water, and cooled. The pH of the solution is adjusted to about 7 using HCl and stirred. The resulting solid is filtered, washed with water and dried under vacuum to afford Compound 4 containing a hydroxamate group —NH₂OH.

In scheme C, the intermediate ester compound 3 is converted to an arylamide compound, illustrated by compound 5, wherein T stands for NH₂ or OH and R¹⁰ is selected from amino, halo, alkyl, cycloalkyl, heterocyclyl, aryl and heteroaryl.

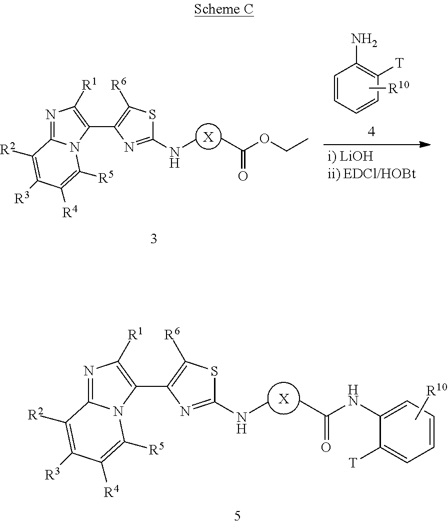

Ester compound 3 is converted to the free carboxylic acid, and is then reacted with substituted aniline 4 to yield an arylamide of formula 5. For example, LiOH is added to a stirred solution of 3 in a mixture of solvents. The volatiles are removed under vacuum, and the residue is diluted with water and acidified to pH about 3. The resulting solids are filtered, washed with water and dried under vacuum to furnish a carboxylic acid intermediate. The intermediate is dissolved in a solvent such as dimethylformamide (DMF) and the mixture is stirred to prepare a solution. To the stirred solution is added 1-ethyl-3-(3′-dimethylaminopropyl)carbodiimide hydrochloride (EDCI) followed by hydroxybenzotriazole (HOBt). After stirring, diisopropyl ethyl amine is added and stirred. Then substituted aniline 4 (representative of substituted aryl or heteroaryl) is added, and the reaction mixture is stirred. The solvent is removed under vacuum. The residue is diluted with water and stirred. The resulting solids are filtered and purified through column chromatography to provide benzamide 5.

Bromoketone 1 can be synthesized by several pathways, depending on the substitution pattern of R¹, R², R³, R⁴, R⁵, and R⁶ and the availability of starting materials.

A first synthetic route begins with the reaction of an aminopyridine 2′ with a chlorodiketone 1′ to make an acyl imidazopyridine 3′, which is brominated to bromoketone 1. Typical starting materials and reaction conditions are illustrated in Scheme D.

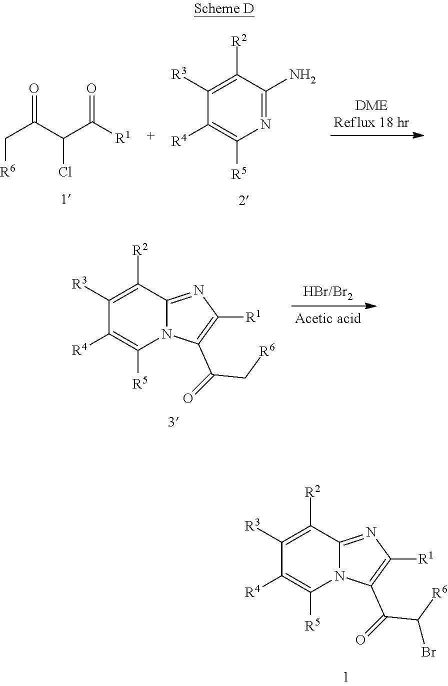

A second route to bromoketone 1 is given in Scheme E, where the imidazopyridine is formed first and is then acylated and brominated.

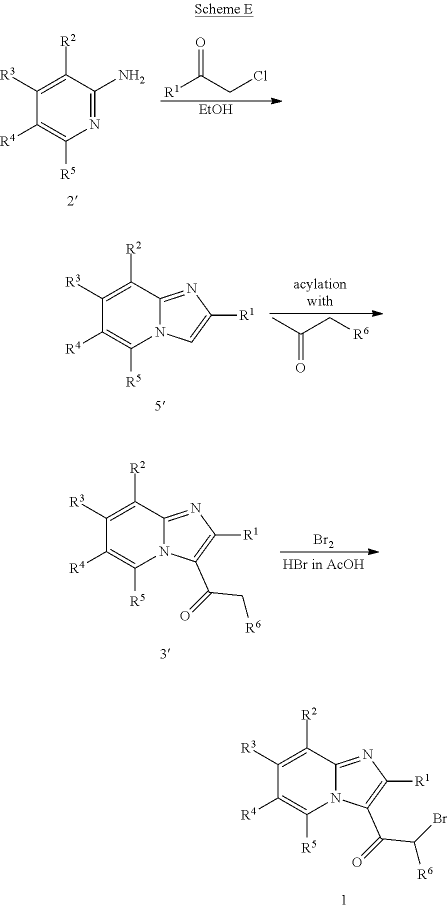

In Scheme E, the imidazo ring is elaborated first, and then subjected to acylation to add the ketone side chain and group R⁶, both of which will become part of the thiazole in subsequent synthetic steps. In one sense, this affords more flexibility in the choices of R¹ and R⁶ than does Scheme D. At the same time, the reaction of aminopyridine 2′ with chloroketone or chloroaldehyde 4′ occurs under similar conditions as in Scheme D, and is permissive of the same broad range of substituents R², R³, R⁴, and R⁵ on the aminopyridine starting material 2′.

The reactions and starting materials for Schemes A, B, C, D, and E are generally known from the literature or represent applications of well known chemical transformations, such as Friedel-Crafts type acylation and the like. Illustrative conditions are also given in the Examples.

Compounds of Formula (I-b) and (I-d) can be prepared according to Scheme F. Thioureido compound 2 undergoes ring elaboration to thiazole 7, followed by bromination to compound 8. Bromo compound 8 is alkylated with a suitable imidazopyridyl derivative 9 to give intermediate 10, which is converted to hydroxamate 11 via Scheme B or to benzamide 12 by Scheme C.

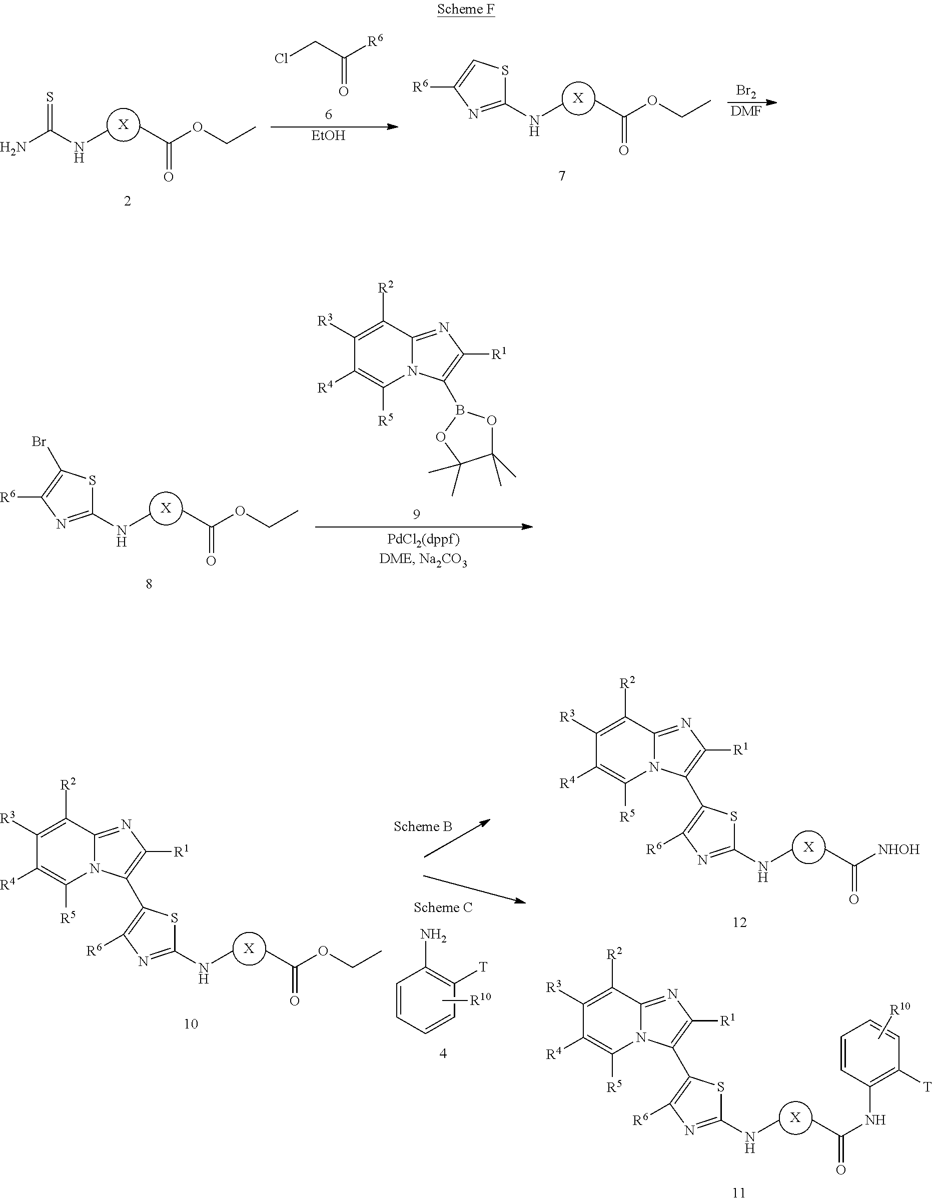

Compounds with X=thiophene can be made according to Scheme G.

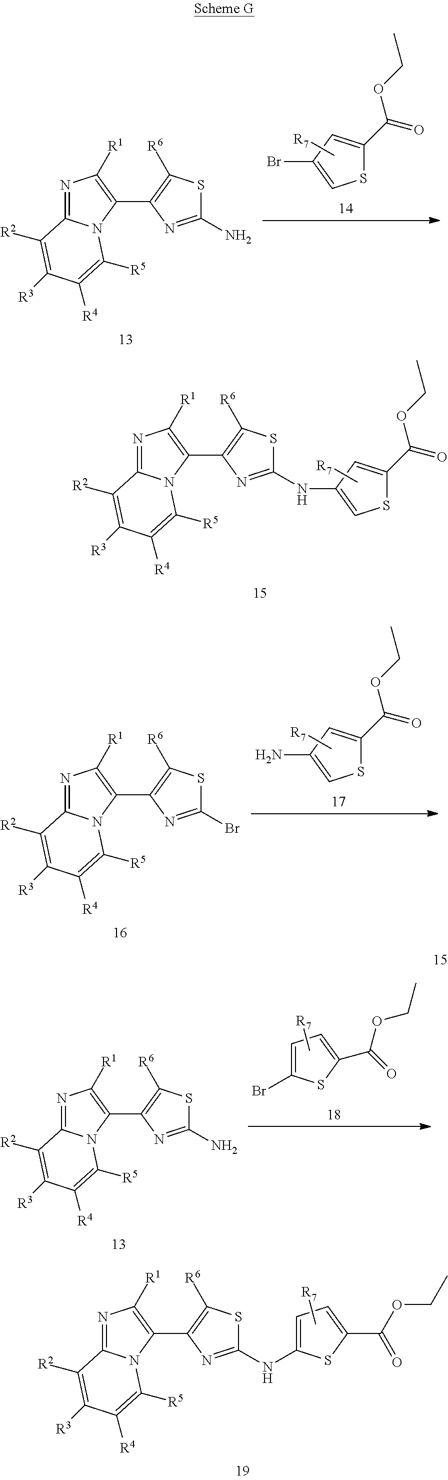

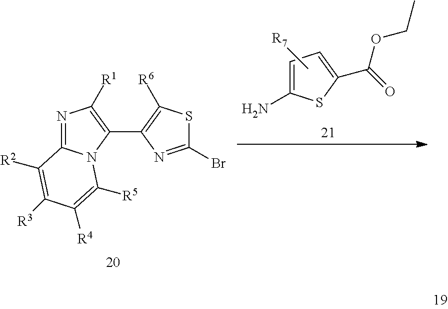

Aminothiazole 13 is coupled with bromothiophene 14 or 18 to produce thiophene compounds 15 or 19. Similarly, compounds 15 and 19 can be synthesized from the reaction of bromothiazoles 16 or 20 with aminothiophenes 17 or 21. Compounds 15 and 19 in turn are converted to hydroxamates according to Scheme B or to arylamides according to Scheme C.

The compounds of the present invention inhibit histone deacetylase and/or CDK and are useful to treat or ameliorate diseases mediated directly or indirectly by HDAC and/or CDK. Therefore, another aspect of the present invention is to provide a pharmaceutical composition comprising an effective amount of one or more compounds as described above.

In one embodiment of the invention, a pharmaceutical composition is provided comprising, in addition to one or more compounds described herein, at least one pharmaceutically-acceptable diluent, adjuvant, excipient, or carrier. The composition can take any suitable form for the desired route of administration. Where the composition is to be administered orally, any suitable orally deliverable dosage form can be used, including without limitation tablets, capsules (solid- or liquid-filled), powders, granules, syrups and other liquids, elixirs, inhalants, troches, lozenges, and solutions. Injectable compositions or iv infusions are also provided in the form of solutions, suspensions, and emulsions.

A pharmaceutical composition according to the present invention may contain one or more additional therapeutic agents, for example, to increase the efficacy or decrease the side effects. In some embodiments, accordingly, a pharmaceutical composition further contains one or more additional therapeutic agents selected from active ingredients useful to treat or inhibit diseases mediated directly or indirectly by HDAC and/or CDK. Examples of such active ingredients are, without limitation, agents to treat or inhibit cancer, Huntington's disease, cystic fibrosis, liver fibrosis, renal fibrosis, pulmonary fibrosis, skin fibrosis, Rheumatoid arthritis, diabetes, stroke, amyotrophic lateral sclerosis, cardiac hypertrophy, congestive heart failure, or Alzheimer's disease.

In an embodiment, an additional therapeutic agent to be included is an anti-cancer agent. Examples of an anti-cancer agent include, but are not limited to, alkylating agents such as cyclophosphamide, dacarbazine, and cisplatin; antimetabolites such as methotrexate, mercaptopurine, thioguanine, fluorouracil, and cytarabine; plant alkaloids such as vinblastine, and paclitaxel; antitumor antibiotics such as doxorubicin, bleomycin, and mitomycin; hormones/antihormones such as prednisone, tamoxifen, and flutamide; other types of anticancer agents such as asparaginase, rituximab, trastuzumab, imatinib, retinoic acid and derivatives, colony-stimulating factors, amifostine, camptothecin, topotecan, thalidomide analogs such as lenalidomide, CDK inhibitor and other HDAC inhibitor such as histone deacetylase 1 inhibitors, histone deacetylase 2 inhibitors, histone deacetylase 3 inhibitors, histone deacetylase 4 inhibitors, histone deacetylase 5 inhibitors, histone deacetylase 6 inhibitors, histone deacetylase 7 inhibitors, histone deacetylase 8 inhibitors, histone deacetylase 9 inhibitors, histone deacetylase 10 inhibitors, and histone deacetylase 11 inhibitors.

Yet another aspect of the present invention is to provide a method of inhibiting or treating diseases arising from abnormal cell proliferation and/or differentiation in animal, comprising administering to said animal a therapeutically effective amount of one or more compounds according to the present invention. In one embodiment, the method of inhibiting or treating disease comprises administering to an animal a composition comprising an effective amount of one or more compounds of the invention and a pharmaceutically-acceptable carrier. The composition to be administered may further contain a therapeutic agent such as anti-cancer agent.

A method of the present invention is particularly suitable for use with humans, but may be used with other animals, particularly mammals, such as, for example, non-human primates, companion animals, farm animals, laboratory animals, and wild and zoo animals.

A method of the present invention is particularly useful to treat diseases mediated directly or indirectly by HDAC and/or CDK since the compounds of the present invention have inhibitory activity against those molecules. In some embodiments, therefore, a method of the present invention is used in inhibiting or treating HDAC- and/or CDK-mediated diseases. Examples of such disease include, but are not limited to, cell proliferative diseases such as cancer, autosomal dominant disorders such as Huntington's disease, genetic related metabolic disorder such as cystic fibrosis, fibrosis such as liver fibrosis, renal fibrosis, pulmonary fibrosis and skin fibrosis, autoimmune diseases such as Rheumatoid arthritis, diabetes, acute and chronic neurological diseases such as stroke, amyotrophic lateral sclerosis, hypertrophy such as cardiac hypertrophy, heart failure (or congestive heart failure), and Alzheimer's disease.

In an embodiment, a method according to the present invention is applied to a patient with cancer, cystic fibrosis, or pulmonary fibrosis. In some embodiments, a method using a compound according to the present invention is used to treat or inhibit a cancer selected from bladder cancer, breast cancer, colon and rectal cancer, endometrial cancer, kidney (renal cell) cancer, leukemia, lung cancer, melanoma, non-Hodgkin's lymphoma, pancreatic cancer, prostate cancer, skin cancer (non-melanoma), and thyroid cancer.

### EXAMPLES

The following examples are merely illustrative, and do not limit this disclosure in any way.

### Example 1

### N-Hydroxy-3-[4-(2-methyl-imidazo[1,2-a]pyridin-3-yl)-thiazol-2-ylamino]-benzamide

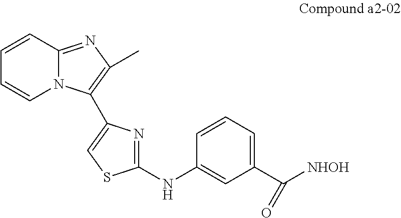

To a stirred solution of 2-aminopyridine (1.035 g, 11 mmol) in 1,2-dimethoxy ethane (5 mL) was added sodium bicarbonate (0.924 g, 11 mmol) and 3-chloro-2,3-pentane dione (2 g, 14.8 mmol). The heterogeneous reaction mixture was refluxed for about 16 hours, and cooled to room temperature. The volatiles were removed under vacuum. The residue was diluted with water (50 mL) and extracted with DCM (3×50 mL). The organic layer was dried over anhydrous Na₂SO₄, filtered and concentrated under vacuum. The crude product was purified by column chromatography (SiO₂). The product was eluted with ethyl acetate/hexane (6:4) to provide 1-(2-Methyl-imidazo[1,2-a]pyridin-3-yl)-ethanone (1 g, 38% yield based on the diketone-1, 54% based on the aminopyridine-2) as a solid. Bromine (0.163 mL, 3.1 mmol) was added to 1-(2-Methyl-imidazo[1,2-a]pyridin-3-yl)-ethanone (500 mg, 2.8 mmol) in 33% HBr in acetic acid (w/v, 5 mL) at 0° C., and the mixture was stirred for about 1.5 hours at room temperature. The reaction mixture was diluted with diethyl ether (50 mL) and stirred for about 30 minutes. The resulting solid was filtered, washed with ether (2×10 mL) to furnish 2-bromo-1-(2-methyl-imidazo[1,2-a]pyridin-3-yl)-ethanone (Compound (1)) (581 mg, 80%) as a solid. To Compound (1) (2.5 g, 9.88 mmol) in ethanol (25 mL), 3-thioureido-benzoic acid ethyl ester (Compound (2)) (2.213 g; 9.88 mmol) was added and the mixture was refluxed for overnight. Ethanol was removed under vacuum, and then residue was diluted with ether (75 mL) and stirred for 30 minutes. The solid was filtered and dried under vacuum to yield 3-[4-(2-Methyl-imidazo[1,2-a]pyridin-3-yl)-thiazol-2-ylamino]-benzoic acid ethyl ester (Compound (3)) (2.8 g, 75%) as a solid. To a stirred solution of Compound (3) (300 mg, 0.79 mmol) in methanol:dichloromethane (15 mL:6 mL) at 0° C. was added 50% aqueous NH₂OH (6 mL) slowly. After stirring for about 10 minutes at 0° C., NaOH (240 mg dissolved in 1.5 mL water) was added dropwise, brought to room temperature after about 15 minutes and stirred for about 3 hours. The volatiles were evaporated under vacuum below 35° C., diluted with water (6 mL), and cooled to 0° C. The pH was adjusted to about 7 using 2N HCl and stirred for about 30 minutes. The resulting solid was filtered, washed with water (75 mL) and dried under vacuum for about 5 hours to afford the title compound (150 mg; 51%) as an off-white solid.

MS m/z 366.0 (M⁺+1), MP 170.4° C., ¹H NMR (DMSO-D₆, 200 MH_{z}) δ 2.52 (s, 3H), 7.00 (dd, 1H), 7.10 (s, 1H), 7.2-7.4 (m, 3H), 7.53 (d, 1H), 7.67 (d, 1H), 8.20 (s, 1H), 9.03 (s, OH), 9.05 (d, 1H), 10.54 (s, NH) and 11.18 (s, NH).

### Example 2

### N-(2-Amino-phenyl)-3-[4-(2-methyl-imidazo[1,2-a]pyridin-3-yl)-thiazol-2-ylamino]-benzamide

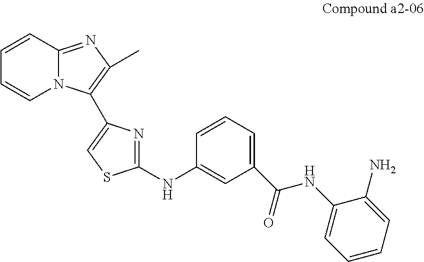

To a stirred solution of Compound (3) of Example 1 (1 g, 2.64 mmol) in a mixture of solvents methanol:THF:water (6.5 mL:6.5 mL:3.5 mL) was added LiOH (monohydrate) (332 mg, 7.93 mmol). The mixture was stirred overnight at room temperature. The volatiles were removed under vacuum, and the residue was diluted with water (5 mL) and acidified with 2N HCl to pH about 3 at 0° C. The resulting solids were filtered, washed with water (50 mL) and dried under vacuum to furnish Compound (4) (600 mg, 65%) as a solid. To a stirred solution of N-Hydroxy-3-[4-(2-methyl-imidazo[1,2-a]pyridin-3-yl)-thiazol-2-ylamino]-benzamide (Compound (4)) (300 mg, 0.85 mmol) in DMF (5 mL) at 0° C. was added EDCI-HCl (361 mg, 1.88 mmol) followed by HOBt (115 mg, 0.85 mmol). After stirring for about 10 minutes, diisopropyl ethyl amine (0.4 mL, 2.12 mmol) was added and stirred for about 15 minutes. Then 1,2-phenyl diamine (92.5 mg, 0.85 mmol) was added and the reaction mixture was stirred overnight at room temperature. The solvent (DMF) was removed under vacuum below 45° C. The residue was diluted with water (15 mL) and stirred for about 15 minutes. The resulting solids were filtered and purified through column chromatography using DCM:meOH as eluent to provide the title compound.

MS m/z 440.8 (M⁺+1), MP 194.9-197° C., ¹H NMR (DMSO-D₆, 200 MH_{z}) δ 2.52 (s, 3H), 4.92 (bs, NH_{z}), 6.60 (s, 1H), 6.79 (d, 1H), 6.83-7.05 (m, 2H), 7.1-7.05 (m, 3H), 7.4-7.6 (m, 3H), 7.73 (d, 1H), 8.33 (s, 1H), 9.02 (d, 1H), 9.62 (s, NH) and 10.58 (s, NH).

### Example 3

### N-Hydroxy-4-[4-(2-methyl-imidazo[1,2-a]pyridin-3-yl)-thiazol-2-ylamino]-benzamide

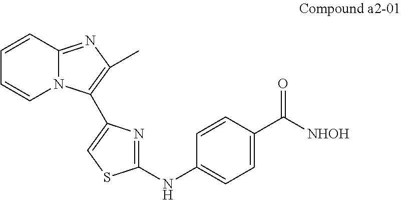

The same procedure as that of Example 1 was used, except that 4-thioureido-benzoic acid ethyl ester was used as Compound (2). Yield of the title compound was 51%.

MS m/z 366.0 (M⁺+1), MP 188.3° C., ¹H NMR (DMSO-D₆, 200 MH_{z}) δ 2.52 (s, 3H), 6.89 (dd, 1H), 7.18 (s, 1H), 7.29 (dd, 1H), 7.57 (d, 1H), 7.65-7.8 (m, 4H), 8.90 (s, 1H), 8.93 (s, OH), 10.7 (s, NH) and 11.08 (bs, NH).

### Example 4

### N-(2-Amino-phenyl)-4-[4-(2-methyl-imidazo[1,2-a]pyridin-3-yl)-thiazol-2-ylamino]-benzamide

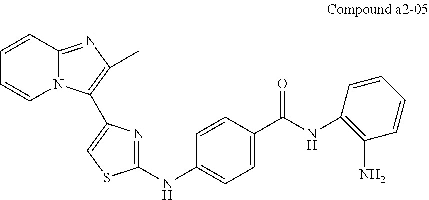

The same procedure as that of Example 2 was used, except that 4-thioureido-benzoic acid ethyl ester was used as Compound (2). Yield of the title compound was 41%.

MS m/z 441.0 (M⁺+1), MP 161.1-165.4° C., ¹H NMR (DMSO-D₆, 200 MH_{z}) δ 2.53 (s, 3H), 4.95 (bs, NH₂), 6.59 (dd, 1H), 6.77 (d, 1H), 6.9-7.05 (m, 2H), 7.15 (d, 1H), 7.19 (s, 1H), 7.30 (dd, 1H), 7.55 (d, 1H), 7.73 (d, 2H), 7.98 (d, 2H), 8.93 (d, 1H), 9.52 (s, NH) and 10.75 (s, NH).

Examples 5-21 were prepared using a procedure similar to those described in Examples 1-4.

### Example 5

### N-Hydroxy-4-[5-methyl-4-(2-methyl-imidazo[1,2-a]pyridin-3-yl)-thiazol-2-ylamino]-benzamide

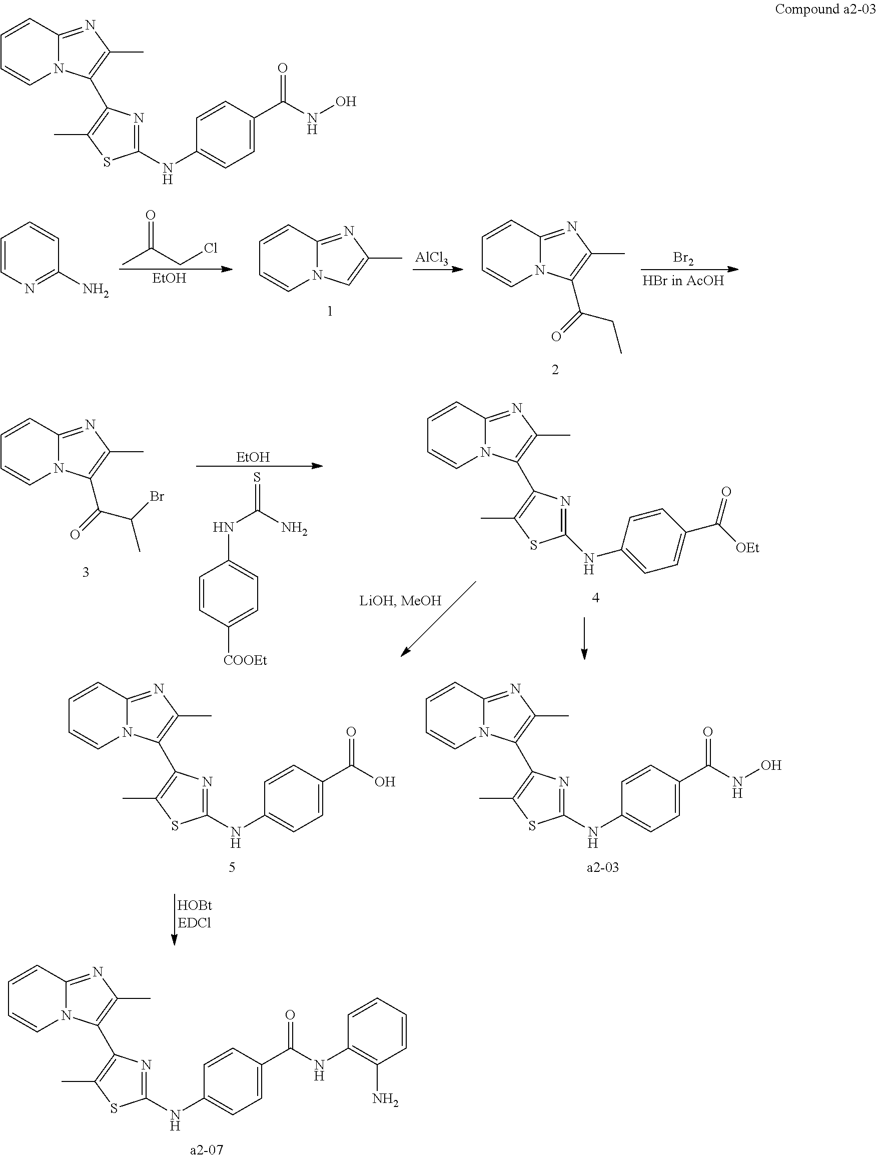

Preparation of 2-methyl-H-imidazo[1,2-a]pyridine (1): To a solution of 2-aminopyridine (20 g, 0.2 mol) in ethanol (100 mL) was added chloroacetone (21.6 g, 0.23 mol) drop wise at room temperature under inert atmosphere. The reaction mixture heated at reflux temperature for 16 hours upon completion of starting material (by TLC), ethanol was evaporated under reduced pressure, resulting residue was diluted in DCM (600 mL) and washed with saturated NaHCO₃ solution (2×100 mL), water (100 mL) and brine (150 mL). The organic layer was dried over anhydrous Na₂SO₄ and evaporated under vacuum. Crude material was purified by silica gel column chromatography eluting with 5% MeOH/DCM to afford pure Int-1 (16 g, 57%) as brown color liquid. ¹H NMR (200 MHz, CDCl₃): δ 8.04 (d, J=7.0 Hz, 1H), 7.52 (d, J=9.0 Hz, 1H), 7.33 (s, 1H), 7.15-7.06 (m, 1H), 6.72 (t, J=6.6 Hz, 1H), 2.46 (s, 3H). Mass (m/z): 133.1 [M⁺+1].

Preparation of 1-(2-methyl-H-imidazo[1,2-a]pyridin-3-yl)propan-1-one (2): To Int-1 (15 g, 0.11 mole) in polyphosphoric acid (20 mL) was added propionic anhydride (30.0 mL, 0.22 mole) slowly at room temperature. The reaction mixture was heated at 80° C. for 5 hours. The reaction mixture was cooled to room temperature and poured into ice water (400 mL) very slowly. Aqueous layer was extracted with EtOAc (3×300 mL), combined organic layers were washed with water (200 mL) and brine (200 mL). Organic layer was dried over anhydrous Na₂SO₄ and evaporated under vacuum. Crude material was purified over silica gel column chromatography eluting with 25% EtOAc/hexane to afford pure Int-2 (4.5 g, 21%) as brown color solid. ¹H NMR (200 MHz, CDCl₃): δ 9.80 (d, J=6.0 Hz, 1H), 7.66 (d, J=7.0 Hz, 1H), 7.49-7.39 (m, 1H), 7.05- (t, J=6.6 Hz, 1H), 2.96 (q, 2H), 2.81 (s, 3H), 1.28 (t, J=7.4 Hz, 3H). Mass (m/z): 189.1 [M⁺+1].

Preparation of 2-bromo-1-(2-methyl-H-imidazol[1,2-a]pyridin-3-yl)propan-1-one (3): To a stirred suspension of Int-2 (3.0 g, 1.5 mmol) in HBr in AcOH solution (33% w/v, 42 mL) at 0° C. was added bromine (2.5 g, 1.5 mmol) drop wise for 10 minutes. The reaction mixture was allowed to room temperature and stirred for 4 hours. Upon complete consumption of starting material (by TLC), reaction mixture was diluted with diethyl ether (20 mL) and stirred for 15 minutes. The precipitated solid was filtered, washed with diethyl ether (2×5 mL) and dried under vacuum to provide bromo-compound-3 (3.3 g, 78%) as solid. ¹H NMR (200 MHz, CDCl₃): δ 9.89 (d, J=6.6 Hz, 1H), 8.49 (d, J=6.9 Hz, 1H), 8.02 (t, J=7.1 Hz, 1H), 7.60 (d, J=6.6 Hz, 1H), 5.02 (q, 1H), 3.21 (s, 3H), 2.12 (d, J=7.1 Hz, 3H). Mass (m/z): 267.0 [M⁺+1].

Preparation of ethyl-4-(5-methyl-4-(2-methyl-H-imidazo[1,2-a]pyridin-3-yl)thiazol-2-ylamino)benzoate (4): To a solution of bromo compound-3 (2.0 g, 7.5 mmol) in ethanol (20 mL) was added 1-(4-Ethoxy carbonylphenyl)-2-thiourea (1.6 g, 7.5 mmol) at room temperature under N₂ atm and the mixture was stirred at reflux temperature for 16 hours. Reaction mixture was cooled to room temperature and stirred to for 15 minutes. The precipitated solid was filtered, washed with cold ethanol (5 mL) and dried under vacuum to provide ester-4 (2.4 g, 82%) as off white solid. ¹H NMR (200 MHz, DMSO-d₆): δ 10.8 (s, 1H), 8.62 (d, J=7.0 Hz, 1H), 7.80-7.86 (m, 2H), 7.88 (d, J=8.8 Hz, 2H), 7.70 (d, J=8.8 Hz, 2H), 7.54-7.48 (m, 1H) 4.25 (q, 2H), 2.47 (s, 3H), 2.30 (s, 3H), 1.27 (t, J=7.0 Hz, 3H). Mass (m/z): 392.9 [M⁺+1].

Preparation of 4-(5-methyl-4-(2-methyl-H-imidazo[1,2-a]pyridin-3-yl)thiazol-2-ylamino)-N-hydroxybenzamide (Compound a2-03): To a stirred solution of ester-4 (0.3 g, 0.76 mmol) in methanol (15 mL) and DCM (6 mL) was added hydroxylamine 50 wt % solution in water (6.0 mL) at 0° C. After being stirred for 10 min at same temperature, aqueous NaOH solution (0.24 g, 6.1 mmol) in water (1.5 mL) was added to the reaction mixture at 0° C. The reaction mixture allowed warming to room temperature and stirred for 16 hours. Volatiles were evaporated under vacuum, resulting residue was neutralized (pH˜7) using 2 N HCl at 0° C. and stirred for 10 minutes. The precipitated solid was filtered, washed with water (2×3 mL) and dried under vacuum. Crude material was washed with 10% MeOH/DCM (10 mL) to afford pure Compound a2-03 (0.2 g, 69%) as off white solid.

¹H NMR (200 MHz, DMSO-d₆): δ 11.0 (bs, 1H), 10.4 (s, 1H), 8.86 (bs, 1H), 8.21 (d, J=7.0 Hz, 1H), 7.69-7.51 (m, 5H), 7.26 (t, J=6.8 Hz, 1H), 6.89 (t, J=7.0 Hz, 1H), 2.31 (s, 3H), 2.22 (s, 3H). ¹³C NMR (125 MHz, DMSO-d₆): δ 164.0, 160.2, 144.1, 143.4, 141.6, 135.3, 128.0, 125.2, 124.9, 124.5, 121.7, 116.1, 115.9, 115.2, 111.9, 14.3, 11.8. Mass (m/z): 379.9 [M⁺+1]. MP: 200.5° C.

### Example 6

### N-(2-Amino-phenyl)-4-[5-methyl-4-(2-methyl-imidazo[1,2-a]pyridin-3-yl)-thiazol-2-ylamino]-benzamide

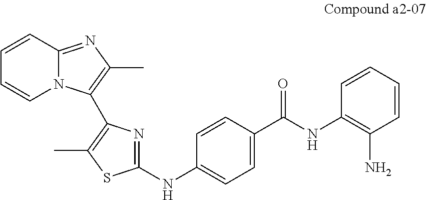

Preparation of ethyl-4-(5-methyl-4-(2-methyl-H-imidazo[1,2-a]pyridin-3-yl)thiazol-2-ylamino)benzoic acid (5): To a solution of ester-4 of Example 5 (1.5 g, 3.8 mmol) in methanol (15 mL) and THF (15 mL) was added lithium hydroxide (0.48 g, 11.4 mmol) at room temperature followed by water (7 mL). Resulting mixture was stirred at room temperature for 24 hours. Upon completion of starting material (by TLC), volatiles were evaporated under vacuum, resulting residue was diluted with water (5 mL) and acidified to pH˜6 using 2 N HCl at 0° C. The precipitated solid was filtered, washed with water (2×4 mL) and dried under vacuum to provide acid-5 (1.0 g, 72%) as off white solid. ¹H NMR (200 MHz, DMSO-D₆): δ 12.2 (bs, 1H), 10.7 (s, 1H), 8.43 (d, J=7.0 Hz, 1H), 7.85-7.77 (m, 3H), 7.70-7.63 (m, 3H), 7.22 (t, J=6.8 Hz, 1H), 2.40 (s, 3H), 2.26 (s, 3H). Mass (m/z): 364.9 [M⁺+1]. MP: 288.6° C.

Preparation of 4-(5-methyl-4-(2-methyl-H-imidazo[1,2-a]pyridin-3-yl)thiazol-2-ylamino)-N-(2-aminophenyl)benzamide (Compound a2-07): To a stirred suspension of acid-5 (0.6 g, 1.6 mmol) in DMF (8 mL) were added HOBt (0.22 g, 1.6 mmol), EDCI (0.69 g, 3.6 mmol), N-Ethyldiisopropylamine (0.53 g, 4.1 mmol) followed by o-Phenylenediamine (0.17 g, 1.6 mmol) at 0° C. under inert atmosphere. The reaction mixture was allowed to warm to room temperature and stirring was continued for 16 hours. Reaction mixture was poured into ice water (100 mL) and stirred for 15 minutes. The precipitated solid was filtered, washed with water (2×5 mL) and dried under vacuum. Crude material was purified over silica gel column chromatography eluting with 5% MeOH/DCM to afford Compound a2-07 (0.40 g, 54%) as off white solid.

¹H NMR (200 MHz, DMSO-d₆): δ 10.5 (s, 1H), 9.47 (s, 1H), 8.24 (d, J=6.6 Hz, 1H), 7.93 (d, J=8.4 Hz, 2H), 7.69 (d, J=8.4 Hz, 2H), 7.56 (d, J=9.2 Hz, 1H), 7.27-7.11 (m, 2H), 6.97-6.90 (m, 2H), 6.77 (d, J=8.0 Hz, 1H), 6.60-6.55 (m, 1H), 4.83 (bs, 2H), 2.32 (s, 3H), 2.23 (s, 3H). ¹³C NMR (125 MHz, DMSO-d₆): δ 164.5, 160.1, 144.0, 143.6, 142.9, 141.5, 135.2, 128.9, 126.4, 126.1, 125.1, 124.4, 123.5, 121.6, 116.0, 115.7, 115.1, 111.7, 14.3, 11.7. Mass (m/z): 454.9 [M⁺+1]. MP: 250.1° C.

### Example 7

### 4-[4-(6-Chloro-2-methyl-imidazo[1,2-a]pyridin-3-yl)-thiazol-2-ylamino]-N-hydroxy-benzamide

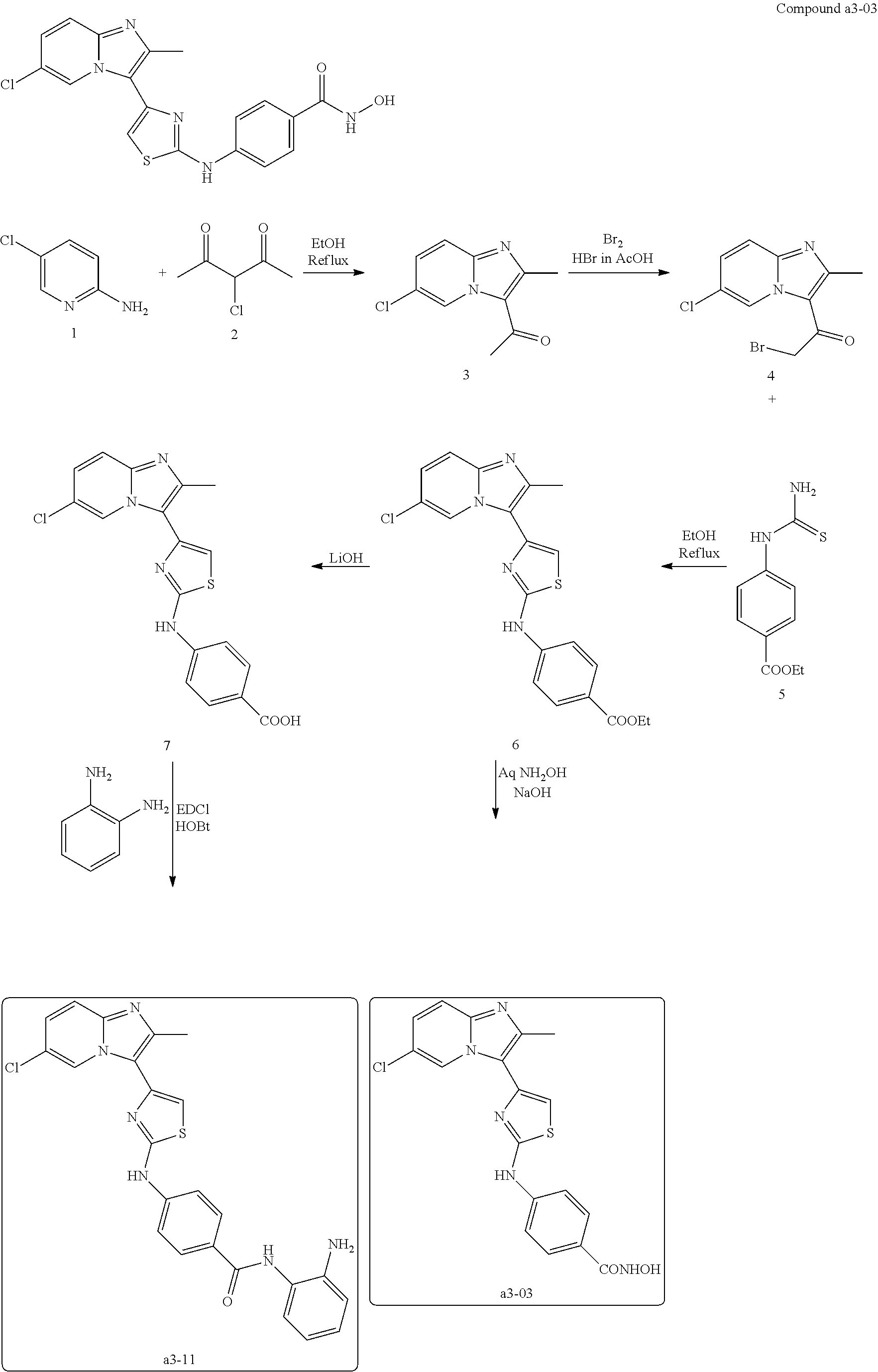

Preparation of Int-3: A mixture of SM-1 (5.0 g, 39.0 mmol) and SM-2 (4.4 mL, 39.0 mmol) in ethanol (30 mL) was stirred under reflux for 12 hours. The reaction mixture was concentrated under vacuum to obtain crude mass which was purified by column chromatography using EtOAc and hexane (40:60) to furnish Int-3 (2.0 g, 25%) as a yellow solid.

Preparation of Int-4: To Int-3 (3.0 g, 14.0 mmol) in HBr in acetic acid (33%, 60 mL) at 0° C. was added Br₂ (4.61 g, 28.8 mmol) drop wise and the mixture was stirred at room temperature for 4 hours. The reaction mixture was diluted with ether (25 mL) and stirred for 15 minutes. The solid precipitated was filtered, washed with a mixture of EtOH and ether (25 mL, 7:3) and dried under vacuum to afford Bromo-compound 4 (3.55 g, 86%) as an off white solid.

Preparation of Int-6: A mixture of bromo-Int-4 (1.5 g, 5.2 mmol) and thiourea-5 (1.17 g, 5.2 mmol) in ethanol (60 mL) was stirred under reflux for over night. Cooled the reaction mixture to room temperature, the precipitated solid was filtered, washed with ethanol (2×20 mL) and dried under vacuum to afford pure Int-6 (1.85 g, 86%) as a white solid.

Preparation of Compound a3-03: To a solution of ester-6 (0.5 g, 1.2 mmol) in methanol (25 mL) and DCM (10 mL) was added hydroxylamine (50% w/v solution in water (15 mL) and stirred for 10 minutes. After cooling to 0° C., aqueous NaOH (388 mg, 9.7 mmol in 2.5 mL water) was added and the mixture was stirred at room temperature over night. The reaction mixture was concentrated under reduced pressure, the residue was neutralized with 2N HCl at 0° C. (pH˜7), the precipitated solid was filtered, washed with water and dried under vacuum to afford pure Compound a3-03 (0.36 g, 74%). ¹H NMR (200 MHz, DMSO-d₆): δ 11.09 (bs, 1H), 10.75 (bs, 1H), 9.23 (s, 1H), 8.91 (bs, 1H), 7.78-7.58 (m, 4H), 7.35 (d, J=7.6 Hz, 1H), 7.21 (s, 1H) and 2.52 (s, 3H). ¹³C NMR (125 MHz, DMSO-d₆): δ 15.0, 105.9, 116.1, 116.8, 117.0, 119.0, 123.8, 124.9, 125.3, 128.0, 139.5, 141.6, 142.6, 143.2, 162.9, 163.9. Mass (m/z): 399 [M⁺+1]; Melting Point: 234° C.

### Example 8

### N-(2-Amino-phenyl)-4-[4-(6-chloro-2-methyl-imidazo[1,2-a]pyridin-3-yl)-thiazol-2-ylamino]-benzamide

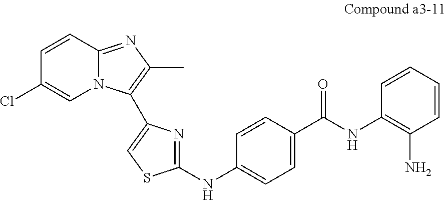

Preparation of Int-acid (7): To a stirred suspension of Int-6 of Example 7, (0.7 g, 1.6 mmol) in a mixture of THF (14 mL), water (7 mL), and methanol (3 mL) was added LiOH (0.213 g, 5.0 mmol) and the mixture was stirred at room temperature over night. The reaction mixture was concentrated under reduced pressure, diluted with water (20 mL) and acidified with 2 N HCl. The precipitated solid was filtered, washed with water (2×20 mL) and dried under vacuum to afford pure acid-7 (0.4 g, 61%).

Preparation of Compound a3-11: To the solution of acid-7 (0.5 g, 1.3 mmol) in DMF (10 mL) at 0° C. was added EDCI (0.547 g, 2.8 mmol), HOBt (0.175 g, 1.3 mmol), DIPEA (0.419 g, 3.2 mmol) followed by o-phenylenediamin-8 (0.140 g, 1.3 mmol) and the mixture was stirred at room temperature overnight. The reaction mixture was poured into ice water (20 mL) and stirred for 5 min, the precipitated solid was filtered, washed with water (2×20 mL) and dried under vacuum to afford Compound a3-11 (0.26 g, 42%). ¹H NMR (200 MHz, DMSO-d₆): δ 10.79 (s, 1H), 9.55 (bs, 1H), 9.22 (s, 1H), 7.99 (d, J=8.4 Hz, 2H), 7.73 (d, J=8.8 Hz, 2H), 7.61 (d, J=9.6 Hz, 1H), 7.33 (d, J=7.4 Hz, 1H), 7.24 (s, 1H), 7.15 (d, J=6.8 Hz, 1H), 6.95 (t, J=7.4 Hz, 1H), 6.77 (d, J=7.0, 1H), 6.58 (t, J=8.0 Hz, 1H), 4.88 (bs, 2H) and 2.55 (s, 3H). ¹³C NMR (125 MHz, DMSO-D₆): δ 15.1, 106.0, 115.9, 116.1, 116.2, 116.9, 117.1, 119.0, 123.6, 123.7, 124.8, 126.2, 126.5, 127.1, 129.0, 139.6, 141.7, 142.8, 142.9, 143.4, 162.9, 164.5. Mass (m/z): 474 (M⁺+1). Melting Point: 194° C.

### Example 9

### N-Hydroxy-4-[4-(2-methyl-6-trifluoromethyl-imidazo[1,2-a]pyridin-3-yl)-thiazol-2-ylamino]-benzamide

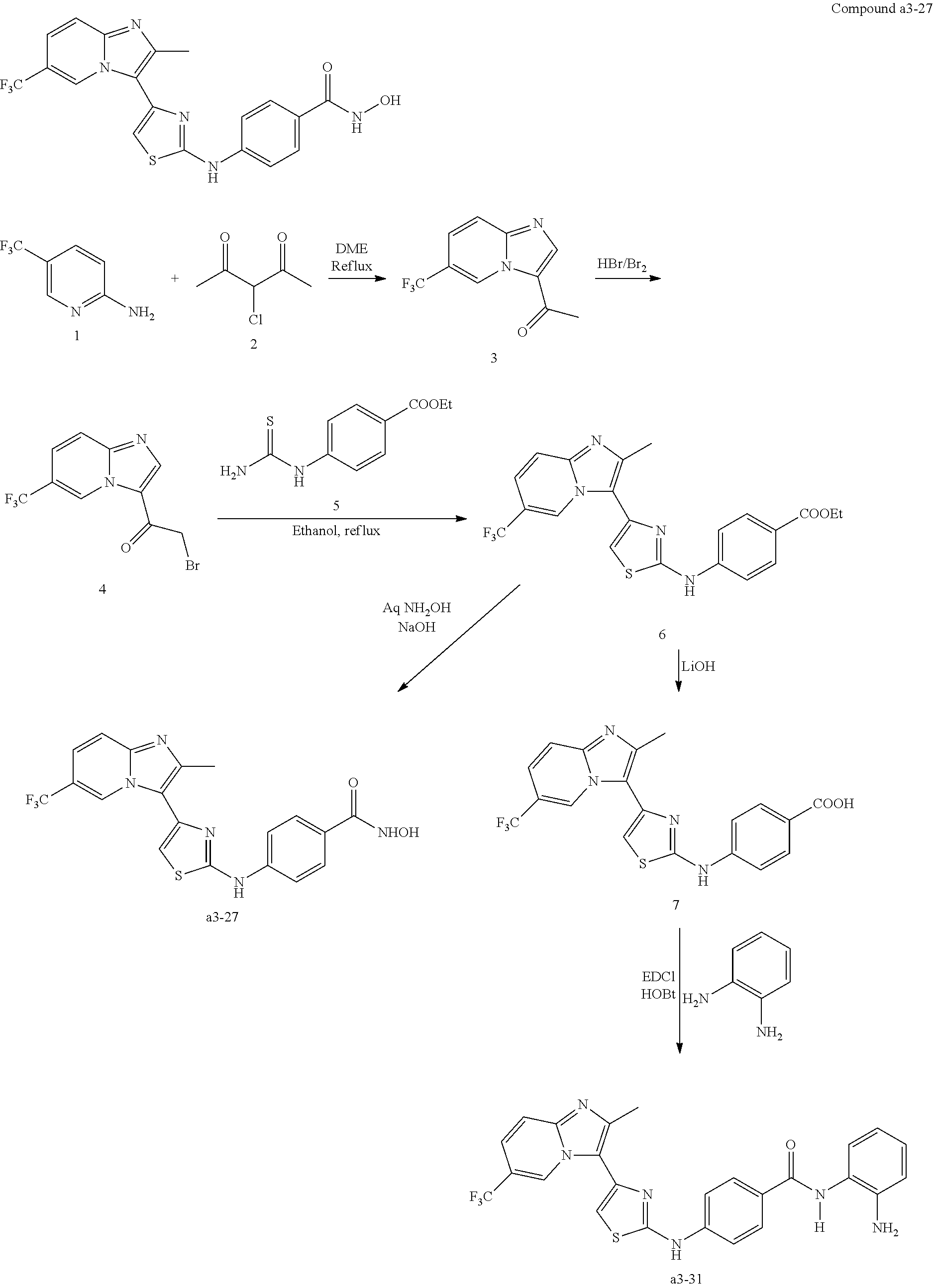

Preparation of 1-(6-(trifluoromethyl)-2-methylH-imidazo[1,2-a]pyridin-3-yl)ethanone (3): To a stirred solution of SM-1 (4.0 g, 24 mmol) in DME (20 ml) was added 3-chloro-2,4-pentadione (2, 3.5 mL, 29 mmol) drop wise at room temperature under inert condition. The mixture was refluxed for overnight, cooled to room temperature, concentrated under reduced pressure. The residue was diluted with EtOAc, washed with 1N HCl and water, dried over Na₂SO₄, filtered and concentrated under reduced pressure and purified by column chromatography using 20% EtOAc:Hexane to obtain pure Int-3 (1.0 g, 16%).

Preparation of 2-bromo-1-(6-(trifluoromethyl)-2-methylH-imidazo[1,2-a]pyridin-3-yl)ethanone (4): To a stirred solution of Int-3 (1.25 g, 5.1 mmol) in HBr in AcOH (10 mL, 33% w/v) at 0° C. was added Br₂ (0.29 mL, 5.6 mmol) drop wise. The mixture was stirred for further 3 hours at the same temperature. Then ether (80 mL) was added to the reaction mixture and stirred at room temperature for 30 minutes. The precipitated solid was filtered, washed with ether and dried under vacuum to obtain pure Int-4 (1.0 g, 60%).

Preparation of ethyl 4-(4-(6-(trifluoromethyl)-2-methylH-imidazo[1,2-a]pyridin-3-yl)thiazol-2-ylamino)benzoate (6): A mixture of Bromo-compound 4 (1.0 g, 3.11 mmol) and thiourea-5 (0.697 g, 3.11 mmol) in EtOH (25 mL) was refluxed for overnight. The reaction mixture was cooled to room temperature; the resulting solid was filtered, washed with ether and dried under vacuum to provide pure Int-6 (0.9 g, 65%).

Preparation of 4-(4-(6-(trifluoromethyl)-2-methylH-imidazo[1,2-a]pyridin-3-yl)thiazol-2-ylamino)-N-hydroxybenzamide (Compound a3-27): To a cooled solution of ester-6 (0.6 g, 1.3 mmol) in MeOH:DCM (42 mL, 5:2) was added aqueous NH₂OH (12 mL, 50% solution) slowly and the mixture was stirred for 15 minutes. Then aqueous NaOH (0.48 g in 3 mL water) was added and the reaction mixture was stirred at room temperature for overnight. The reaction mixture was concentrated under reduced pressure, the residue was diluted with water (30 mL), pH was adjusted to ˜7.0 using 2N HCl, the precipitated solid was filtered, washed with water, and dried under vacuum to obtain crude product which was further washed with 30% MeOH:DCM to furnish the pure Compound a3-27 (0.4 g, 75%). ¹H NMR (200 MHz, DMSO-d₆): δ 2.58 (s, 3H), 7.27 (s, 1H) 7.52 (m, 1H), 7.78-7.70 (m, 5H), 8.91 (bs, 1H) 9.63 (s, 1H), 10.76 (s, 1H) and 11.15 (bs, 1H). ¹³C NMR (125 MHz, DMSO-d₆): δ 15.3, 106.5, 114.4, 114.6, 116.1, 117.3, 117.8, 119.6, 122.9, 125.1, 125.4, 128.0, 139.4, 143.0, 143.1, 143.7, 163.2, and 164.0. Mass (m/z): 433 (M⁺+1). MP: 183.2° C.

### Example 10

### N-(2-Amino-phenyl)-4-[4-(2-methyl-6-trifluoromethyl-imidazo[1,2-a]pyridin-3-yl)-thiazol-2-ylamino]-benzamide

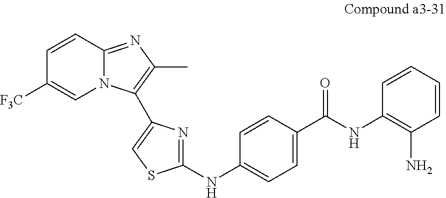

Preparation of 4-(4-(6-(trifluoromethyl)-2-methylH-imidazo[1,2-a]pyridin-3-yl)thiazol-2-ylamino)benzoic acid (7): To a stirred solution of ester-6 of Example 9 (1.8 g, 4.0 mmol) in a mixture of MeOH:THF:H₂O (45 mL, 2:2:1), was added LiOH (0.85 g, 20 mmol) and stirred at room temperature for overnight. The reaction mixture was concentrated under reduced pressure, diluted with water (30 mL) and acidified with 2N HCl to pH˜5 at 0° C. The precipitated solid was filtered, washed with water and dried under vacuum to obtain pure Acid-7 (1.15 g, 68%).

Preparation of 4-(4-(6-(trifluoromethyl)-2-methylH-imidazo[1,2-a]pyridin-3-yl)thiazol-2-ylamino)-N-(2-aminophenyl)benzamide (Compound a3-31): To a stirred solution of Acid-7 (0.6 g, 1.4 mmol) in DMF (12 mL) at 0° C. were added HOBt (0.19 g, 1.4 mmol), EDCI (0.6 g, 3.1 mmol), DIPEA (0.65 mL, 3.5 mmol) and o-phenylene diamine (0.15 g, 1.4 mmol) sequentially. The mixture was stirred under inert atmosphere at room temperature for overnight. Water (30 mL) was added to the reaction mixture, the precipitated solid was filtered, washed with water, dried under vacuum and washed with 30% MeOH:DCM to yield the pure Compound a3-31 (328 mg, 45%). ¹H NMR (200 MHz, DMSO-d₆): δ 4.88 (bs, 2H), 6.61 (t, J=7.6 Hz, 1H), 6.77 (d, J=7.6 Hz, 1H), 6.93 (t, J=7.4 Hz, 1H), 7.17 (d, J=7.6 Hz, 1H), 7.31 (d, J=4.8 Hz, 1H), 7.62 (s, 1H), 8.01-7.82 (m, 5H), 8.58 (bs, 1H), 9.51 (s, 1H) and 10.66 (s, 1H). ¹³C NMR (125 MHz, DMSO-d₆): δ 107.5, 115.8, 116.1, 116.2, 120.3, 122.6, 123.6, 126.1, 126.4, 126.8, 129.0, 137.1, 142.8, 143.6, 149.3, 150.3, 151.9, 162.7 and 164.7. Mass (m/z): 387 (M⁺+1). MP: 243.8° C.

### Example 11

### N-(2-Amino-phenyl)-4-[4-(7-methoxy-2-methyl-imidazo[1,2-a]pyridin-3-yl)-thiazol-2-ylamino]-benzamide

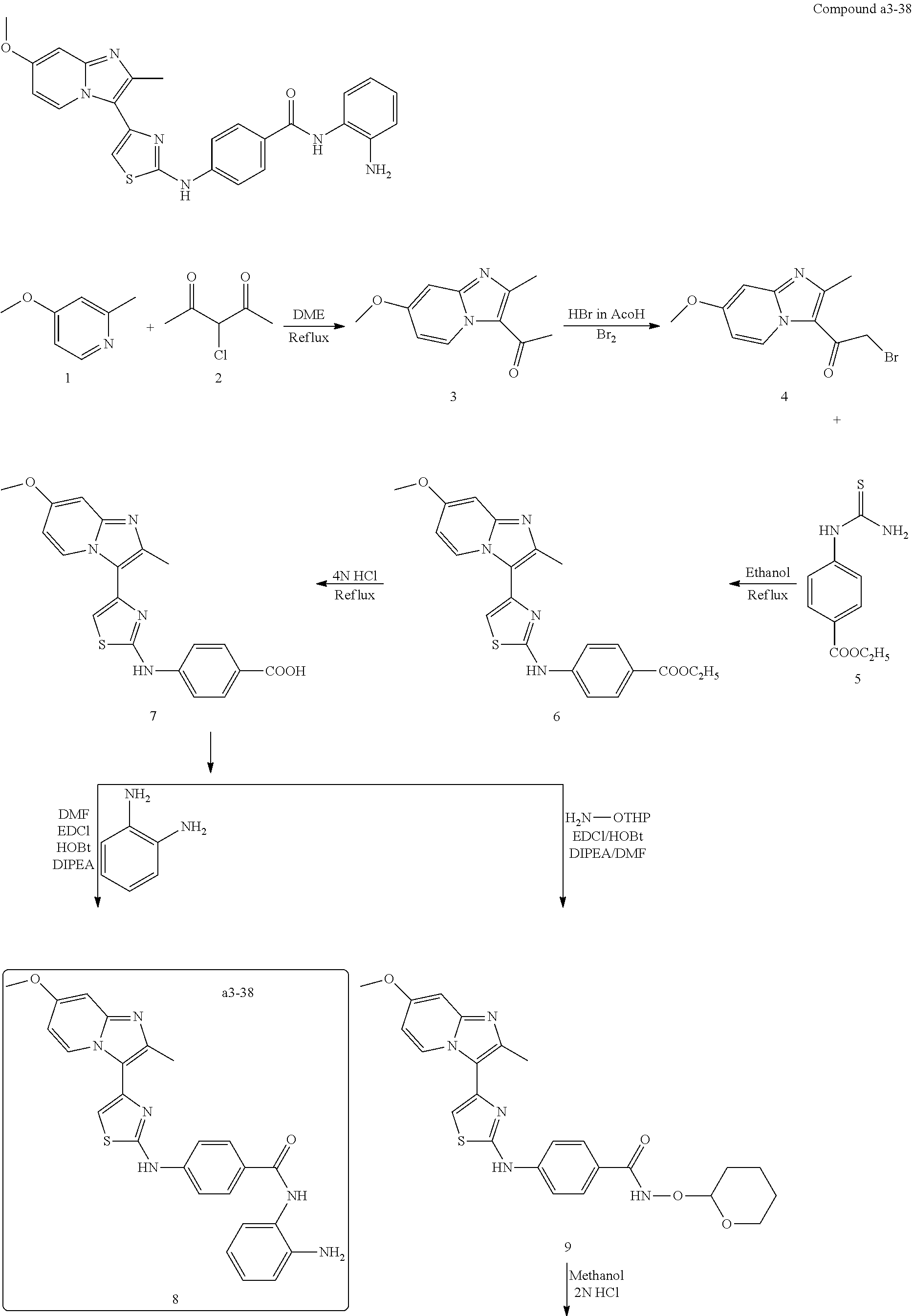

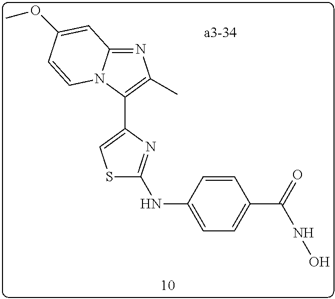

Preparation of 1-(7-methoxy-2-methylH-imidazo[1,2-a]pyridin-3-yl)ethanone (3): To a solution of int-1 (3.0 g, 24.19 mmol) in DMF (30 mL) was added int-2 (4.5 g, 33.8 mmol) and stirred under reflux for 24 hours. The reaction mixture was concentrated under vacuum to obtain crude mass which was purified by column chromatography eluting with ethyl acetate to afford Int-3 (2.0 g, 41%). ¹H NMR (200 MHz, CDCl₃): δ 9.55 (d, J=7.6 Hz, 1H), 6.9 (d, J=2.2 Hz, 1H), 6.67 (q, J=3.0 Hz, 1H), 3.89 (s, 3H), 2.74 (s, 3H) and 2.58 (s, 3H). Mass (m/z): 205 (M⁺+1).

Preparation of 2-bromo-1-(7-methoxy-2-methylH-imidazo[1,2-a]pyridin-3-yl)ethanone (4): To a solution of int-3 (2.0 g, 9.8 mmol) in HBr in acetic acid (60 mL) was added bromine (1.56 g, 9.8 mmol) drop wise at 0° C. and stirred at room temperature for 4 hours. The reaction mixture was diluted with diethyl ether (50 mL), stirred for 30 minutes; the precipitated solid was filtered and dried under vacuum to afford Int-4 (2.5 g, 90%) as a white solid. ¹H NMR (200 MHz, CDCl₃): δ 9.63 (d, J=7.8 Hz, 1H), 7.61 (d, J=3.0 Hz, 1H), 7.14 (q, J=2.6 Hz, 1H), 4.40 (s, 2H), 4.08 (s, 3H) and 3.07 (s, 3H). Mass (m/z): 282 (M⁺+1).

Preparation of ethyl 4-(4-(7-methoxy-2-methylH-imidazo[1,2-a]pyridin-3-yl)thiazol-2-ylamino)benzoate (6): To a solution of int-4 (2.5 g, 8.86 mmol) in ethanol (30 mL) was added int-5 (2.0 g, 8.92 mmol) at room temperature and stirred under reflux for 16 hours. The reaction mixture was cooled to room temperature, the precipitated solid was filtered and washed with ether (15 mL), dried under vacuum to gave int-6 (3.0 g, 83%) as a white solid. ¹H NMR (200 MHz, DMSO-d₆): δ 10.86 (s, 1H), 8.76 (d, J=7.4 Hz, 1H), 7.92 (d, J=8.8 Hz, 2H), 7.73 (q, J=8.8 Hz, 2H), 7.17 (s, 1H), 6.97 (d, J=2.2 Hz, 1H), 6.80 (q, J=7.0 Hz, 1H), 4.26 (q, J=7.0 Hz, 2H), 3.87 (s, 3H), and 1.29 (t, J=7.0 Hz, 3H). Mass (m/z): 408 (M⁺+1).

Preparation of 4-(4-(7-methoxy-2-methylH-imidazo[1,2-a]pyridin-3-yl)thiazol-2-ylamino)benzoic acid (7): A suspension of int-6 (1.7 g, 4.16 mmol) in 4N HCl (100 mL) was refluxed for 4 hours. The reaction mixture was cooled to room temperature, the precipitated solid was filtered, washed with water (25 mL) and dried under vacuum to afford acid-7 (1.4 g, 88%) as off white solid. ¹H NMR (200 MHz, DMSO-d₆): δ 14.6 (bs, 1H), 11.14 (s, 1H), 8.95 (d, J=8.4 Hz, 1H), 7.89 (d, J=8.8 Hz, 2H), 7.72 (d, J=8.8 Hz, 2H), 7.48 (s, 1H), 7.27-7.25 (m, 2H), 4.01 (s, 3H), and 2.59 (s, 3H). Mass (m/z): 480.8 (M⁺+1)

Preparation of 4-(4-(7-methoxy-2-methylH-imidazo[1,2-a]pyridin-3-yl)thiazol-2-ylamino)-N-(2-aminophenyl)benzamide (8): To the suspension of acid-7 (0.7 g, 1.84 mmol) in DMF (8 mL) was added EDCI.HCl (0.77 g, 4.05 mmol), HOBt (0.25 g, 1.85 mmol) and DIPEA (0.6 g, 4.65 mmol) at 0° C. followed by the o-phenylene diamine (0.2 g, 1.85 mmol) and stirred at room temperature for overnight. The reaction mixture was diluted with water (80 mL) stirred for 30 minutes and the precipitated solid was filtered, washed with water (25 mL) and dried under vacuum to obtain crude compound which was purified by column chromatography eluting with DCM and methanol (97:3) to afford Compound a3-38 (0.45 g, 52%) as pink solid. ¹H NMR (200 MHz, DMSO-d₆): δ 10.71 (s, 1H), 9.51 (s, 1H), 8.79 (d, J=7.8 Hz, 1H), 7.98 (d, J=8.6 Hz, 2H), 7.73 (d, J=8.4 Hz, 2H), 7.16-7.09 (m, 2H), 7.07-6.94 (m, 2H), 6.78-6.70 (m, 2H), and 6.65-6.58 (m, 2H). ¹³C-NMR (125 MHz, DMSO-d₆): δ 168.0, 164.68, 162.75, 157.38, 144.89, 143.58, 143.08, 140.82, 140.23, 129.13, 126.94, 126.50, 126.26, 123.61, 116.28, 116.14, 115.91, 115.44, 106.34, 104.91, 94.16, 55.61, 14.80; Mass (m/z): 470.7 (M⁺+1), Melting Point: 159.7° C.

### Example 12

### N-Hydroxy-4-[4-(7-methoxy-2-methyl-imidazo[1,2-a]pyridin-3-yl)-thiazol-2-ylamino]-benzamide

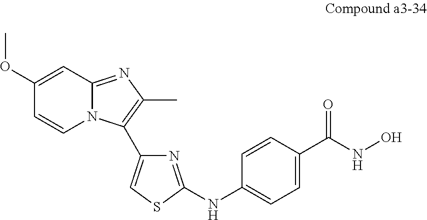

Preparation of 4-(4-(7-methoxy-2-methylH-imidazo[1,2-a]pyridin-3-yl)thiazol-2-ylamino)-N-(tetrahydro-2H-pyran-2-yloxy)benzamide (9): To the solution of acid-7 of Example 11 (0.450 g, 1.18 mmol) in DMF (8 mL) was added EDCI.HCl (0.5 g, 2.6 mmol), HOBt (0.16 g, 1.18 mmol) and DIPEA (0.38 g, 2.96 mmol) followed by the H₂N-OTHP (0.138 g, 1.18 mmol) at 0° C., and stirred overnight at room temperature. The reaction mixture was diluted with water (50 mL) stirred for 30 minutes and the solid precipitated was filtered, washed with water (25 mL) and dried under vacuum to obtain crude compound which was purified by column chromatography eluting pure compound with DCM and methanol (98:2) to afford the THP protected final compound 9 (0.4 g, 70%) as pink solid. ¹H NMR (200 MHz, DMSO-d₆): δ 11.46 (s, 1H), 10.71 (s, 1H), 8.78 (d, J=7.8 Hz, 1H), 7.72 (q, J=9.2 Hz, 4H), 7.14 (s, 1H), 6.97 (s, 1H), 6.82-6.77 (m, 1H), 4.96 (s, 1H), 4.10-4.04 (m, 1H), 3.87 (s, 3H), 3.53-3.47 (m, 1H), 2.49 (s, 3H), and 1.7-1.53 (m, 6H). Mass (m/z): 479.7 (M⁺+1).

Preparation of 4-(4-(7-methoxy-2-methylH-imidazo[1,2-a]pyridin-3-yl)thiazol-2-ylamino)-N-hydroxybenzamide (10): To the solution of compound 9 (0.4 g, 0.83 mmol) in methanol (12 mL) was added 2N HCl (2 mL) at 0° C. and stirred over night at room temperature. The solid precipitated was filtered, washed with methanol (5 mL) and dried under vacuum to afford Compound a3-34 (0.27 g, 82%) as white solid. ¹H NMR (200 MHz, DMSO-d₆): δ 11.05 (s, 1H), 10.09 (s, 1H), 8.97 (d, J=8.0 Hz, 1H), 7.70 (q, J=7.0 Hz, 4H), 7.45 (s, 1H), 7.25-7.22 (m, 2H), 4.01 (s, 3H) and 2.58 (s, 3H). ¹³C NMR (125 MHz, DMSO-d₆): δ 163.93, 163.60, 162.65, 143.14, 140.45, 135.96, 130.34, 128.78, 128.07, 125.31, 116.86, 116.19, 110.33, 110.05, 91.04, 56.95, 10.81; Mass (m/z): 495.8 (M⁺+1); Melting Point: 233.4° C.

### Example 13

### N-Hydroxy-4-(4-imidazo[1,2-a]pyridin-3-yl-thiazol-2-ylamino)-benzamide

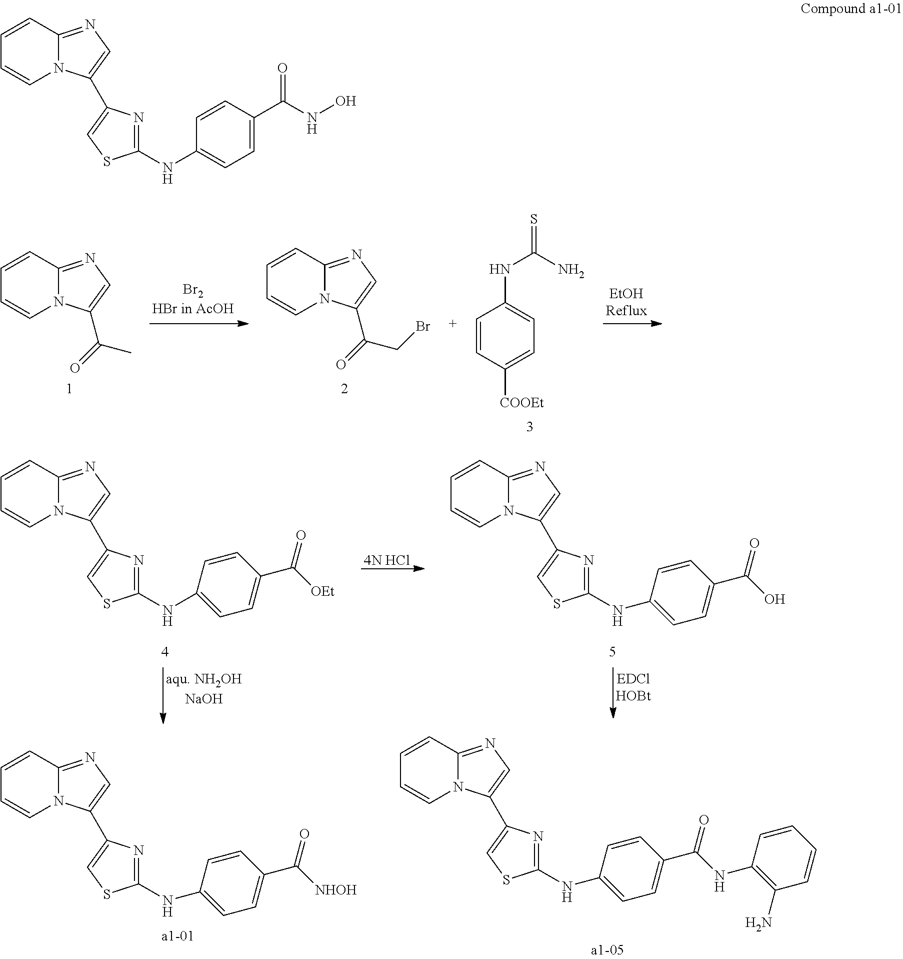

Preparation of 2-bromo-1-(H-imidazo[1,2-a]pyridine-3-yl)ethanone (2): To a stirred suspension of compound 1 (1.0 g, 6.2 mmol) in HBr in AcOH (15 mL, 33% w/v) was added bromine (0.32 mL, 6.2 mmol) drop wise at 0° C. under inert atmosphere and stirring was continued for 5 hours at 0° C. and 2 hours at room temperature. The Reaction mixture was diluted with ether (25 mL) and stirred for 10 minutes. The precipitated solid was filtered, washed with ether (3×10 mL), dried under vacuum. This solid was dissolved in water (25 mL) and was extracted with EtOAc (3×50 mL). The organic extracts were washed with water (30 mL) and brine (30 mL), dried over anhydrous Na₂SO₄ and evaporated under vacuum to provide the bromo-Int-2 (0.7 g, 47%) as brown solid. ¹H NMR (200 MHz, DMSO-d₆): δ 9.63 (d, J=7.0 Hz, 1H), 8.46 (s, 1H), 7.84 (d, J=8.8 Hz, 1H), 7.61-7.53 (m, 1H), 7.16 (t, J=6.8 Hz, 1H), 4.38 (s, 3H). Mass (m/z): 240.8 [M⁺+1].

Preparation of ethyl-4-(4-(H-imidazo[1,2-a]pyridin-3-yl)thiazol-2-ylamino)benzoate (4): To a stirred solution of bromo-Int-2 (1.0 g, 4.1 mmol) in ethanol (10 mL) was added thiourea-3 (0.93 g, 4.1 mmol) at room temperature under N₂ atmosphere and the mixture was stirred at reflux temperature for 16 hours. After consumption of starting material (by TLC), ethanol was evaporated under reduced pressure and the residue was washed with ether (10 mL) which afforded pure ester-4 (1.3 g, 86%) as off white solid. ¹H NMR (200 MHz, DMSO-d₆): δ 11.01 (s, 1H), 9.39 (d, J=7.0 Hz, 1H), 8.66 (s, 1H), 8.02-7.94 (m, 4H), 7.79-7.61 (m, 4H), 4.32-4.22 (q, 2H), 1.30 (t, J=7.0 Hz, 3H). Mass (m/z): 364.9 [M⁺+1].

Preparation of 4-(4-(H-imidazo[1,2-a]pyridine-3-yl)thiazol-2-ylamino)-N-hydroxy benzamide (Compound a1-01): To a stirred suspension of ester-4 (0.6 g, 1.6 mmol) in methanol (30 mL) and DCM (12 mL) was added hydroxylamine 50 wt % solution in water (12 mL) at 0° C. After being stirred for 10 minutes at same temperature, aqueous NaOH (0.48 g, 12.1 mmol in 3.0 ml water) was added to the reaction mixture at 0° C. The reaction mixture was allowed to warm to room temperature and stirred for 4 hours. Volatiles were evaporated under vacuum, resulting residue was diluted with water (10 mL) and neutralized (pH˜7) using 2 N HCl at 0° C. and stirred for 10 minutes. The precipitated solid was filtered, washed with water (2×5 mL) and dried under vacuum to afford Compound a1-01 (0.3 g, 50%) as off white solid. ¹H NMR (200 MHz, DMSO-d₆): δ 11.05 (bs, 1H), 10.70 (bs, 1H), 9.12 (d, J=7.0 Hz, 1H), 8.93 (bs, 1H), 8.02 (s, 1H), 7.94-7.64 (m, 5H), 7.36-7.28 (m, 2H), 7.07 (t, 6.6 Hz, 1H). ¹³NMR (125 MHz, DMSO-d₆): 163.9, 163.1, 145.2, 143.2, 140.1, 133.0, 128.0, 126.0, 125.2, 124.4, 120.5, 117.3, 116.2, 112.8, 103.7. Mass (m/z): 351.9 [M⁺+1]. MP: 209.9° C.

### Example 14

### N-Hydroxy-3-(4-imidazo[1,2-a]pyridin-3-yl-thiazol-2-ylamino)-benzamide

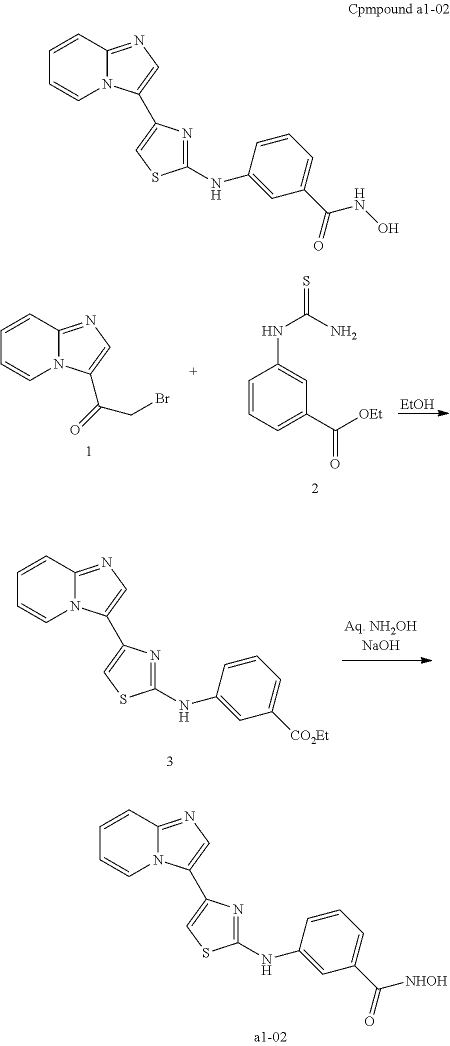

Preparation of ethyl-3-(4-(H-imidazo[1,2-a]pyridin-3-yl)thiazol-2-ylamino) benzoate (3): To a stirred solution of bromo-compound 1 (0.4 g, 1.6 mmol) in ethanol (8.0 mL) was added thiourea-2 (0.37 g, 1.6 mmol) at room temperature under N₂ atmosphere and the mixture was stirred at reflux temperature for 16 hours. After complete consumption of starting precursor (by TLC), ethanol was evaporated under reduced pressure and the crude material was washed with ether (10 mL) to afford ester-3 (0.5 g, 83%) as off white solid. ¹H NMR (200 MHz, DMSO-d₆): δ 10.8 (s, 1H), 9.49 (d, J=7.0 Hz, 1H), 8.66 (s, 1H), 8.55 (s, 1H), 7.94-8.05 (m, 2H), 7.81 (d, J=6.8 Hz, 1H), 7.67-7.46 (m, 4H), 4.32-4.22 (q, 2H), 1.30 (t, J=7.0 Hz, 3H). Mass (m/z): 364.9 [M⁺+1].

Preparation of 3-(4-(H-imidazo[1,2-a]pyridin-3-yl)thiazol-2-ylamino)-N-hydroxy benzamide (Compound a1-02): To a stirred suspension of ester-3 (0.7 g, 1.9 mmol) in methanol (35 mL) and DCM (14 mL) was added hydroxylamine 50 wt % solution in water (14 mL) at 0° C. After being stirred for 10 minutes at the same temperature, aqueous NaOH (0.57 g, 14.4 mmol in 3.5 mL water) was added to the reaction mixture at 0° C. The reaction mixture was allowed to warm up to room temperature and stirred for 4 hours. Volatiles were evaporated under vacuum, resulting residue was diluted with water (15 mL) and neutralized (pH˜7) using 2 N HCl at 0° C. and stirred for 10 minutes. The precipitated solid was filtered, washed with water (2×5 mL) and dried under vacuum to afford Compound a1-02 (0.37 g, 55%) as off white solid. ¹H NMR (200 MHz, DMSO-d₆): δ 10.5 (bs, 1H), 9.31 (d, J=7.4 Hz, 1H), 8.34 (s, 1H), 8.05 (s, 1H), 7.67-7.59 (m, 2H), 7.43-7.33 (m, 4H), 7.11 (t, J=6.8 Hz, 1H). ¹³C NMR (125 MHz, DMSO-d₆): 164.2, 163.5, 145.1, 141.0, 140.2, 133.9, 132.8, 128.9, 126.5, 124.4, 120.5, 119.3, 117.1, 115.7, 113.0, 102.8, Mass (m/z): 351.9 [M⁺+1]. MP: 184.1° C.

### Example 15

### N-(2-Amino-phenyl)-4-(4-imidazo[1,2-a]pyridin-3-yl-thiazol-2-ylamino)-benzamide

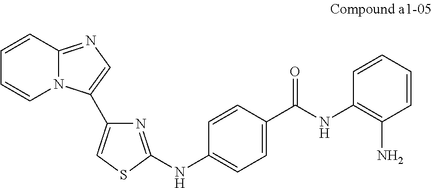

Preparation of ethyl-4-(4-(H-imidazo[1,2-a]pyridin-3-yl)thiazol-2-ylamino)benzoic acid (5): A mixture of ester-4 of Example 13 (0.4 g, 1.0 mmol) and 4N HCl (10 mL) was stirred at 95° C. for 4 hours. After complete consumption of starting material (by TLC), the reaction mixture was cooled to room temperature and stirred for 20 minutes. The precipitated solid was filtered, washed with water (5×5 mL) and dried under vacuum to afford acid-5 (0.26 g, 70%) as white solid. ¹H NMR (200 MHz, DMSO-d₆): δ 11.0 (s, 1H), 9.29 (d, J=7.0 Hz, 1H), 8.42 (s, 1H), 7.96-7.88 (m, 3H), 7.78-7.69 (m, 3H), 7.57 (s, 1H), 7.43 (t, J=6.6 Hz, 1H). Mass (m/z): 336.9 [M⁺+1].

Preparation of 4-(4-(H-imidazo[1,2-a]pyridin-3-yl)thiazol-2-ylamino)-N-(2-aminophenyl)benzamide (Compound a1-05): To a stirred suspension of acid-5 (0.6 g, 1.7 mmol) in DMF (10 mL) were added HOBt (0.24 g, 1.7 mmol), EDCI (0.75 g, 3.9 mmol), o-Phenylenediamine (0.19 g, 1.7 mmol) and N-Ethyldiisopropylamine (0.57 g, 4.4 mmol) at 0° C. under inert atmosphere. The reaction mixture was allowed to warm to room temperature and stirring was continued for 16 hours. Reaction mixture was diluted with water (40 mL) and stirred for 15 minutes. The precipitated solid was filtered, washed with water (3×10 mL), dried under vacuum and finally purified by column chromatography (SiO₂) eluting with 4% MeOH/DCM to afford Compound a1-05 (0.30 g, 40%) as off white solid. ¹H NMR (200 MHz, DMSO-d₆): δ 10.7 (s, 1H), 9.54 (s, 1H), 9.57 (d, J=7.0 Hz, 1H), 8.04 (d, J=10.6 Hz, 3H), 7.78-7.65 (m, 3H), 7.38-7.28 (m, 2H), 7.17-7.05 (m, 2H), 6.99-6.91 (m, 1H), 6.79 (d, J=8.0 Hz, 1H), 6.58 (t, J=7.4 Hz, 1H), 4.87 (bs, 2H). ¹³C NMR (125 MHz, DMSO-d₆): 164.6, 163.1, 145.2, 143.5, 142.9, 140.2, 133.0, 129.1, 127.0, 126.4, 126.1, 126.0, 124.4, 123.6, 120.5, 117.3, 116.2, 116.1, 115.9, 112.7, 103.7, Mass (m/z): 427.0 [M⁺+1]. MP: 229.4° C.

### Example 16

### N-(2-Amino-pyridin-3-yl)-4-(4-imidazo[1,2-a]pyridin-3-yl-thiazol-2-ylamino)-benzamide

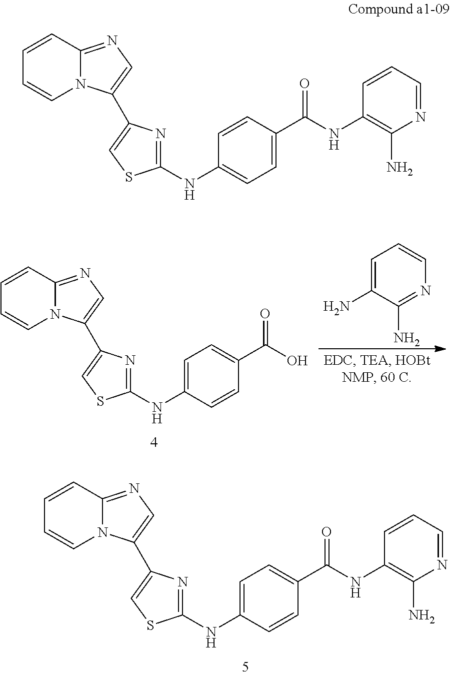

Preparation of N-(2-Amino-pyridin-3-yl)-4-(4-imidazo[1,2-a]pyridin-3-yl-thiazol-2-ylamino)-benzamide (Compound a1-09) —Compound 4 was prepared following the procedure of Example 13. Compound 4 (20 mg, 0.059 mmol), pyridine-2,3-diamine (9.7 mg, 0.088 mmol), EDC (22.81 mg, 0.118 mmol), and 1-hydroxybenzotriazole (8.04 mg, 0.059 mmol) were dissolved in NMP (3 ml) and stirred for 30 minutes. 20 μl of DIPEA was added and the solution was stirred at 60° C. for 4 hours. The product was precipitated out with water and a saturated aqueous solution of NaHCO₃. The solids were purified by HPLC to yield the compound 5. MS: m/z 428 (M+H⁺)

### Example 17

### N-(2-Amino-phenyl)-4-(4-imidazo[1,2-a]pyridin-3-yl-5-methyl-thiazol-2-ylamino)-benzamide

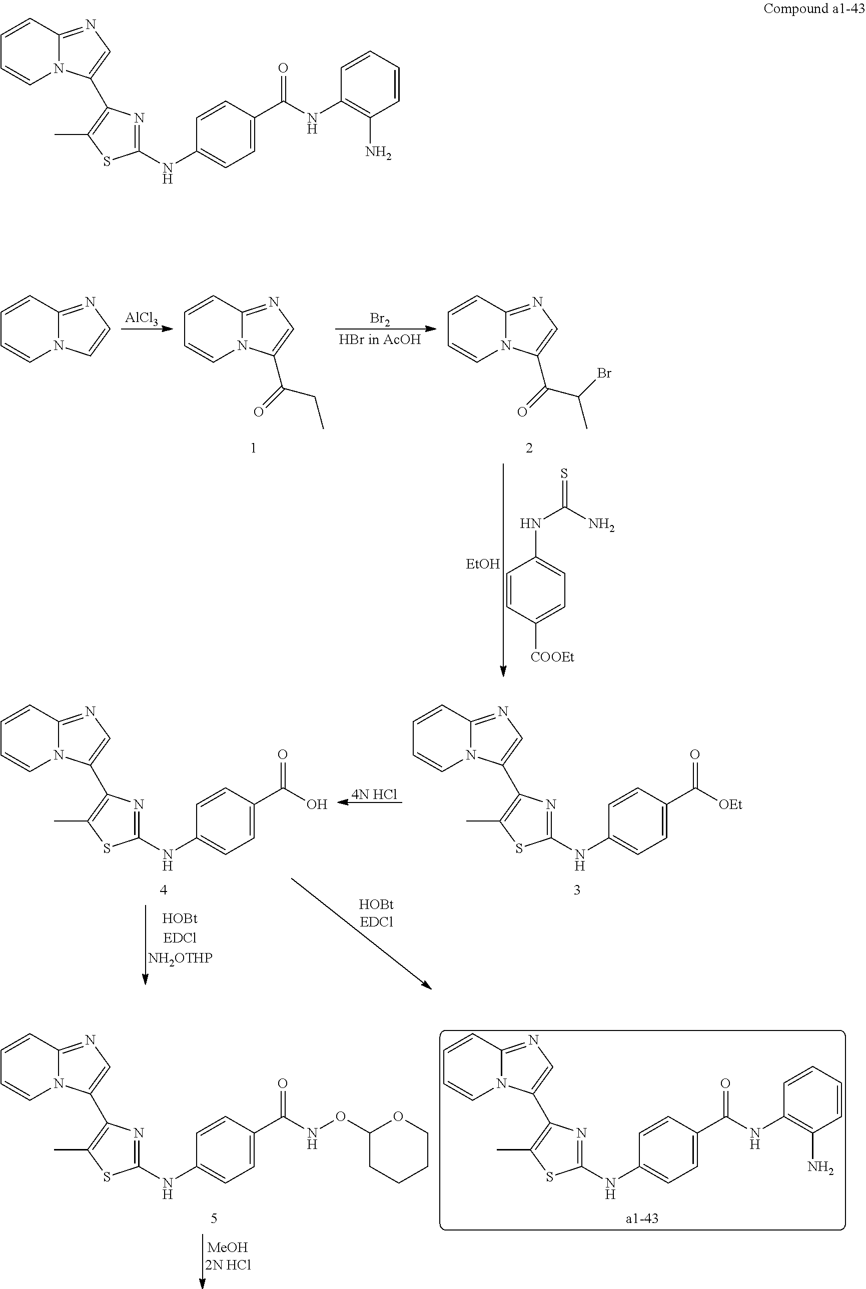

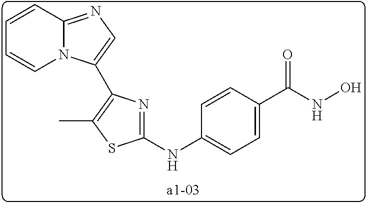

Preparation of 1-(H-imidazo[1,2-a]pyridin-3-yl)propan-1-one (1): A mixture of imidazo[1,2-a]pyridine (5 g, 0.04 mole), propionic anhydride (11 g, 0.084 mole) and AlCl₃ (14 g, 0.1 mole) was irradiated at 65° C. in CEM-Discover microwave for 7 minutes. Reaction mixture was cooled to room temperature and poured into ice water (100 mL), extracted with DCM (2×200 mL). Organic extracts were washed with water (100 mL), brine (100 mL) and dried over anhydrous Na₂SO₄ and evaporated under vacuum. Crude material was purified over silica gel column chromatography eluting with 25% EtOAc/hexane to afford pure Int-1 (1.35 g, 20%) as brown color solid. [The aqueous layer was basified (using aqueous NaOH) and extracted with EtOAc to recover unreacted starting material (2.5 g)]. ¹H NMR (200 MHz, CDCl₃): δ 9.69 (d, J=7.0 Hz, 1H), 8.36 (s, 1H), 7.79 (d, J=9.2 Hz, 1H), 7.49 (t, J=8.4 Hz, 1H), 7.08 (t, J=7.0 Hz, 1H), 3.00 (q, 2H), 1.30 (t, J=7.4 Hz, 3H). Mass (m/z): 175.2 [M⁺+1].

Preparation of 2-bromo-1-(H-imidazol[1,2-a]pyridin-3-yl)propan-1-one (2): To a stirred suspension of Int-1 (1.5 g, 8.6 mmol) in HBr in AcOH solution 33% w/v (21 mL) was added bromine (1.37 g, 8.6 mmol) drop wise at 0° C. After addition, the reaction mixture was stirred at room temperature for 4 hours. Upon complete consumption of starting material (by TLC), reaction mixture was diluted with diethyl ether (60 mL) and stirred for 15 minutes. The precipitated solid was filtered, washed with diethyl ether (2×10 mL), dried under vacuum to provide bromo-2 (1.7 g, 80%) as solid. ¹H NMR (200 MHz, DMSO-d₆): δ 9.61 (d, J=6.8 Hz, 1H), 9.09 (s, 1H), 8.03-7.86 (m, 2H), 7.57-7.48 (m, 1H), 5.77 (q, 1H), 1.83 (d, J=6.6 Hz, 3H). Mass (m/z): 254.8 [M⁺+1].

Preparation of ethyl-4-(4-(H-imidazo[1,2-a]pyridin-3-yl)-5-methylthiazol-2-ylamino)benzoate (3): To a stirred suspension of bromo-2 (2 g, 7.9 mmol) in ethanol (20 mL) was added 1-(4-Ethoxy carbonylphenyl)-2-thiourea (1.7 g, 7.9 mmol) at room temperature under N₂ atm and the mixture was heated at reflux temperature for 16 hours. Reaction mixture was cooled to room temperature and stirred to for 15 minutes. The precipitated solid was filtered, washed with cold ethanol (5 mL) and dried under vacuum to provide ester-3 (2.5 g, 83%) as off white solid. ¹H NMR (200 MHz, DMSO-d₆): δ 10.8 (s, 1H), 9.07 (d, J=6.6 Hz, 1H), 8.47 (s, 1H), 8.02-7.85 (m, 4H), 7.72 (d, J=6.8 Hz, 2H), 7.58 (t, J=5.2 Hz, 1H), 4.27 (q, 2H), 2.47 (s, 3H), 1.28 (t, J=7.4 Hz, 3H). Mass (m/z): 379.1 [M⁺+1].

Preparation of 4-(4-(H-imidazo[1,2-a]pyridine-3-yl)-5-methylthiazol-2-ylamino)benzoic acid (4): A mixture of ester-3 (0.8 g, 2.1 mmol) and 4N HCl (16 mL) was heated at 95° C. for 4 hours. After complete consumption of starting material (by TLC), the reaction was mixture cooled to room temperature and stirred for 10 minutes. The precipitated solid was filtered, washed with water (3×5 mL) and dried under vacuum to afford acid-4 (0.57 g, 77%) as white solid. ¹H NMR (200 MHz, DMSO-d₆): δ 12.5 (bs, 1H), 10.9 (s, 1H), 9.06 (d, J=6.6 Hz, 1H), 8.43 (s, 1H), 8.04-7.85 (m, 4H), 7.71 (d, J=8.6 Hz, 2H), 7.60-7.53 (m, 1H), 2.38 (s, 3H). ¹³C NMR (125 MHz, DMSO-d₆): 166.9, 160.4, 144.7, 140.0, 132.6, 132.1, 130.7, 128.0, 124.3, 123.1, 122.9, 120.0, 116.8, 116.0, 113.0, 11.4. Mass (m/z): 350.9 [M⁺+1]. MP: 234.1° C.

Preparation of 4-(4-(H-imidazol[1,2-a]pyridine-3-yl)-5-methylthiazol-2-ylamino)-N-(2-aminophenyl)benzamide (Compound a1-43): To a stirred suspension of acid-4 (0.6 g, 1.7 mmol) in DMF (8 mL) were added HOBt (0.23 g, 1.7 mmol), EDCI (0.72 g, 3.7 mmol), o-Phenylenediamine (0.18 g, 1.7 mmol) followed by N-Ethyldiisopropylamine (0.5 g, 4.2 mmol) at 0° C. under inert atmosphere. The reaction mixture was allowed to warm to room temperature and stirring was continued for 16 hours. Reaction mixture was poured into ice water (60 mL) and stirred for 15 minutes. The precipitated solid was filtered, washed with water (3×5 mL) and dried under vacuum. Crude material was purified over silica gel column chromatography using 4% MeOH/DCM to afford Compound a1-43 (0.26 g, 35%) as off white solid. ¹H NMR (200 MHz, DMSO-d₆): δ 10.5 (s, 1H), 9.50 (s, 1H), 8.89 (d, J=7.0 Hz, 1H), 7.98 (d, J=8.8 Hz, 2H), 7.84 (s, 1H), 7.71-7.65 (m, 3H), 7.36-7.29 (m, 1H), 7.15 (d, J=7.6 Hz, 1H), 7.06-6.90 (m, 2H), 6.74 (d, J=7.6 Hz, 1H), 6.58 (t, J=7.6 Hz, 1H), 4.87 (bs, 2H), 2.37 (s, 3H). ¹³C NMR (125 MHz, DMSO-d₆): δ 164.7, 159.9, 144.8, 143.7, 143.0, 135.5, 133.4, 129.0, 126.8, 126.5, 126.3, 126.2, 124.7, 123.7, 119.5, 119.0, 117.2, 116.3, 116.2, 115.8, 112.4, 11.5. Mass (m/z): 441.0 [M⁺+1]. MP: 186.5° C.

### Example 18

### N-Hydroxy-4-(4-imidazo[1,2-a]pyridin-3-yl-5-methyl-thiazol-2-ylamino)-benzamide

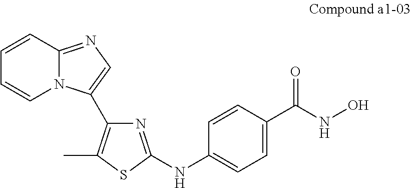

Preparation of 4-(4-(H-imidazol[1,2-a]pyridine-3-yl)-5-methylthiazol-2-ylamino)-N-(tetrahydro-2H-pyran-2-yloxy)benzamide (5): To a stirred suspension of acid-4 of Example 17 (0.7 g, 2.0 mmol) in DMF (8 mL) were added HOBt (0.27 g, 2.0 mmol), EDCI (0.84 g, 4.4 mmol), N-Ethyldiisopropylamine (0.64 g, 5.0 mmol) followed by NH₂OTHP (0.23 g, 2.0 mmol) at 0° C. under inert atmosphere. The reaction mixture was allowed to warm to room temperature and stirring was continued for 16 hours. Reaction mixture was poured into ice water (80 mL) and stirred for 10 minutes. The precipitated solid was filtered, washed with water (2×5 mL) and dried under vacuum. Crude material was purified over silica gel column chromatography eluting with 5% MeOH/DCM to afford Int-5 (0.53 g, 60%) as pale yellow solid. ¹H NMR (200 MHz, DMSO-d₆): δ 11.4 (s, 1H), 10.62 (s, 1H), 8.76 (d, J=7.0 Hz, 1H), 7.73 (s, 1H), 7.69-7.57 (m, 5H), 7.35 (d, J=7.0 Hz, 1H), 7.01 (t, J=6.6 Hz, 1H), 4.90 (s, 1H), 3.95 (bs, 1H), 3.49 (bs, 1H), 2.36 (s, 3H), 1.67 (bs, 2H), 1.48 (bs, 2H), 1.14 (s, 2H). Mass (m/z): 449.8 [M⁺+1].

Preparation of 4-(4-(H-imidazol[1,2-a]pyridin-3-yl)-5-methylthiazol-2-ylamino)-N-hydroxybenzamide (Compound a1-03): To a solution of Int-5 (0.4 g, 0.89 mmol) in methanol (7 mL) was added 2 N HCl (0.4 mL) at 0° C. and stirring was continued for 16 hours at room temperature. The precipitated solid was filtered, washed with dichloromethane (5 mL) and dried under vacuum to provide Compound a1-03 (0.26 g, 78%) as off white solid. ¹H NMR (200 MHz, DMSO-d₆): δ 11.0 (bs, 1H), 10.7 (s, 1H), 9.08 (d, J=7.0 Hz, 1H), 8.47 (s, 1H), 8.05-7.99 (m, 2H), 7.73-7.56 (m, 5H), 2.37 (s, 3H). ¹³C NMR (125 MHz, DMSO-d₆): δ 163.9, 160.6, 143.2, 139.7, 133.2, 131.8, 128.2, 128.0, 125.1, 124.3, 122.3, 120.1, 117.1, 116.1, 112.8, 11.3. Mass (m/z): 365.8 [M⁺+1]. MP: 262.6° C.

### Example 19

### N-(4-Amino-1-phenyl-1H-pyrazol-3-yl)-4-(4-imidazo[1,2-a]pyridin-3-yl-5-methyl-thiazol-2-ylamino)-benzamide

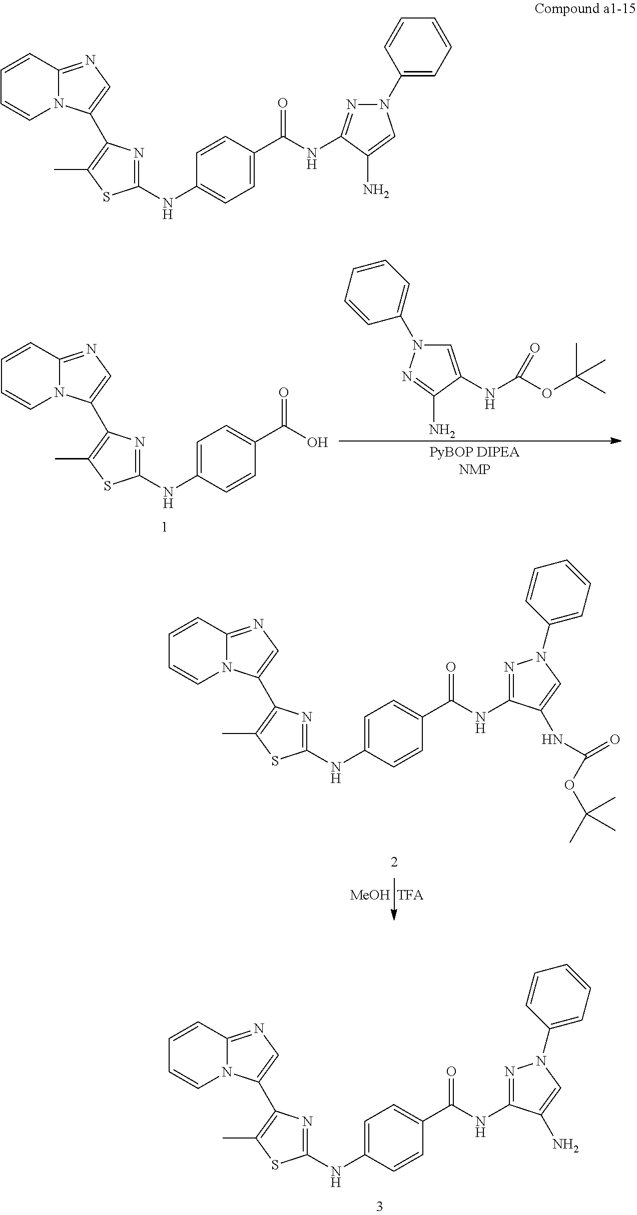

Preparation of {3-[4-(4-Imidazo[1,2-a]pyridin-3-yl-5-methyl-thiazol-2-ylamino)-benzoylamino]-1-phenyl-1H-pyrazol-4-yl}-carbamic acid tert-butyl ester (Compound 2)—A solution of Compound 1 (prepared using the procedure as shown in paragraph [00202]; 0.05 g, 0.14 mmol), tert-butyl 3-amino-1-phenyl-1H-pyrazol-4-ylcarbamate (which can be synthesized, for example, via procedure described in WO2007/087129) (58.71 mg, 0.21 mmol) and PyBOP (74.28 mg, 0.14 mmol) in NMP (3 ml) was stirred for 30 minutes and then EDIPA (49 μl) was added. The mixture was stirred at 70° C. for 8 hours. The product was precipitated out by adding water and a saturated solution of aqueous NaHCO₃ and was used for next step without further purification.

Preparation of N-(4-Amino-1-phenyl-1H-pyrazol-3-yl)-4-(4-imidazo[1,2-a]pyridin-3-yl-5-methyl-thiazol-2-ylamino)-benzamide (Compound 3)-Compound 2 was dissolved in DCM (2 ml) and then TFA was added (1 ml) at room temperature. The reaction mixture was stirred for 2 hours until reaction was done. The reaction mixture was evaporated and purified by HPLC to yield the Compound 3. ¹H-NMR (DMSO) δ: 8.85 (d, J=7.2 Hz, 1H), 8.10-7.92 (m, 2H), 7.90-7.75 (m, 2H), 7.70-7.61 (m, 7H), 7.40 (t, J=7.6 Hz, 2H), 7.31 (t, J=6.8 Hz, 1H), 7.15 (t, J=7.6 Hz, 1H), 7.01 (t, J=7.2 Hz, 1H), 4.03 (s, 2H), 2.42 (s, 3H). MS: m/z 507 (M+H⁺)

### Example 20

### N-(2-Amino-phenyl)-4-(5-imidazo[1,2-a]pyridin-3-yl-thiazol-2-ylamino)-benzamide

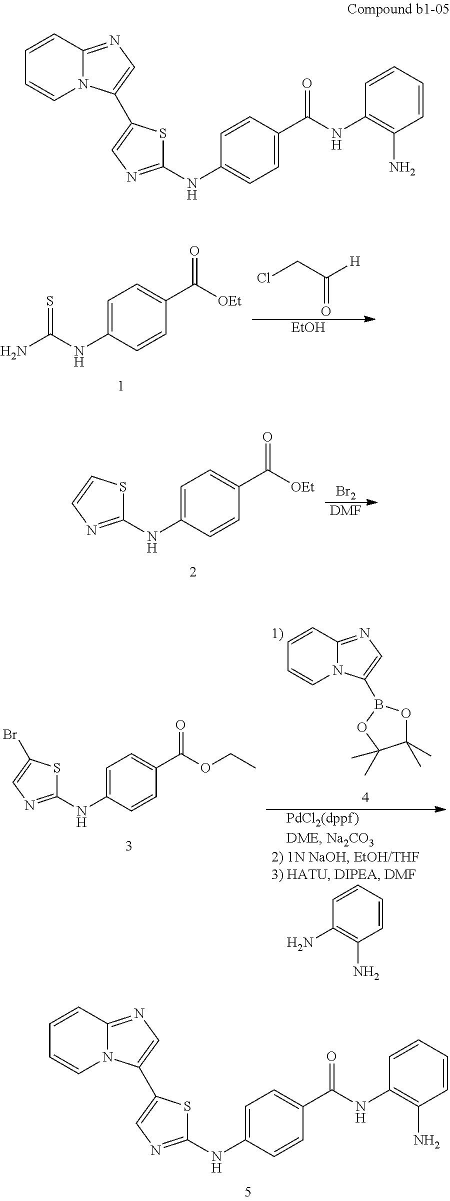

Preparation of Ethyl-4-(thiazol-2-ylamino)benzoate (2): To a mixture of 1-(4-Ethoxy carbonylphenyl)-2-thiourea (1) (3.7 g, 16.5 mmol) in ethanol (25 mL) was added 50% aqueous solution of chloroacetaldehyde (13.0 mL, 82.5 mmol). The reaction mixture was then heated at reflux temperature. After 1 hour, reaction mixture was cooled to room temperature and concentrated in vacuo. Saturated NaHCO₃ was slowly added to the oily residue until CO₂ evolution ceased. The resulting solid was filtered and washed with water and dried to give the title compound. ¹H NMR (400 MHz, dmso-d6): δ 10.61 (s, 1H); 7.89 (d, J=8.8 Hz, 1H); 7.74 (d, J=8.8 Hz, 1H); 7.32 (d, J=4.0 Hz, 1H); 7.01 (d, J=4.0 Hz, 1H), 4.25 (q, 2H); 1.28 (t, J=7.2 Hz, 1H), MS found for C₁₂H₁₂N₂O₂S (m/z): 249.1 [M⁺+1].

Preparation of Ethyl-4-(5-bromothiazol-2-ylamino)benzoate (3): To the mixture of Ethyl-4-(thiazol-2-ylamino)benzoate (2) (2 g, 8.1 mmol) in DMF (25 mL) was added 1M solution of bromine in DMF (8.1 mL, 8.1 mmol). After 20 minutes, reaction mixture was poured into hexanes/DCM mixture (˜4:1, 50 mL). The resulting solid was filtered and washed with ether and dried to give the title compound. ¹H NMR (400 MHz, dmso-d6): δ 10.9 (brs, 1H); 7.87 (d, J=8.8 Hz, 1H); 7.74 (d, J=8.8 Hz, 1H); 7.34 (s, 1H), 4.25 (q, 2H); 1.25 (t, J=7.2 Hz, 1H), MS found for C₁₂H₁₁N₂O₂SBr (m/z): 329.0 [M⁺+1].

Preparation of 4-(5-(H-imidazo[1,2-a]pyridin-3-yl)thiazol-2-ylamino)-N-(2-aminophenyl)benzamide (5): A mixture of 3-(4,4,5,5-tetramethyl-1,3,2-dioxaborolan-2-yl)H-imidazo[1,2-a]pyridine (215 mgs, 0.88 mmol), ethyl-4-(5-bromothiazol-2-ylamino)benzoate (3) (240 mgs, 0.73 mmol), 2.0M sodium carbonate solution (0.73 mL, 1.5 mmol), PdCl₂(dppf) (30 mgs, 0.037 mmol), DME (3 mL) was heated in microwave (Emry's Optimizer) at 120° C. for 20 minutes. The reaction mixture was then poured into DCM and washed with water (2×), brine (1×) and concentrated. The crude ethyl ester from above was diluted with ethanol (6 mL), THF (6 mL) followed by aqueous sodium hydroxide (1.0 M, 7.3 mL, 7.3 mmol) and heated to 65° C. for 3 hours. The reaction mixture was then cooled to room temperature, diluted with water and washed with ether (3×). The aqueous layer was then acidified and concentrated and used for next step. To the above crude carboxylic acid in DMF (12 mL), was added HATU (361 mgs, 0.95 mmol), 1,2-phenylenediamine (95 mgs, 0.876 mmol) and DIPEA (0.4 mL, 2.2 mmol) and stirred overnight. The reaction mixture was then concentrated and diluted with water and acetonitrile and directly purified by preparative HPLC affording the title compound as tan solid, after lyophilization. MS found for C₂₃H₁₈N₆OS as (M+H)⁺ 427.2. ¹H NMR (400 MHz, dmso-d₆): δ 10.96 (brs, 1H); 9.56 (s, 1H); 9.01 (d, J=6.8 Hz, 1H); 8.56 (d, J=2.4 Hz, 1H); 8.49 (d, J=2.4 Hz, 1H); 8.13 (m, 2H), 7.98 (d, J=8.8 Hz, 2H); 7.77 (d, J=8.8 Hz, 2H); 7.76 (s, 1H); 7.66 (t, J=6.8 Hz, 1H); 7.15 (d, J=7.6 Hz, 1H); 6.97 (t, J=8 Hz, 1H); 6.78 (d, J=8 Hz, 1H); 6.61 (t, J=8.8 Hz, 1H); 4.86 (brs, 1H).

### Example 21

### Example 21a

### N-(2-Amino-phenyl)-4-[[4-(2-methyl-imidazo[1,2-a]pyridin-3-yl)-thiazol-2-yl]-(2,2,2-trifluoro-ethyl)-amino]-benzamide

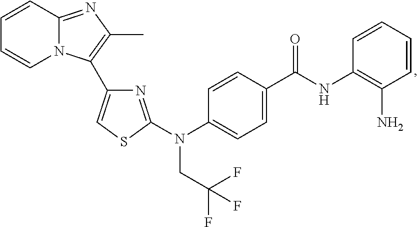

### Example 21b

### N-Hydroxy-4-[[4-(2-methyl-imidazo[1,2-a]pyridin-3-yl)-thiazol-2-yl]-(2,2,2-trifluoro-ethyl)-amino]-benzamide

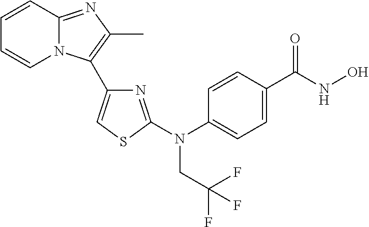

This Example demonstrates how to prepare compounds where R⁹ is other than hydrogen. Intermediate 3 is alkylated with an appropriate iodo compound to introduce R⁹.

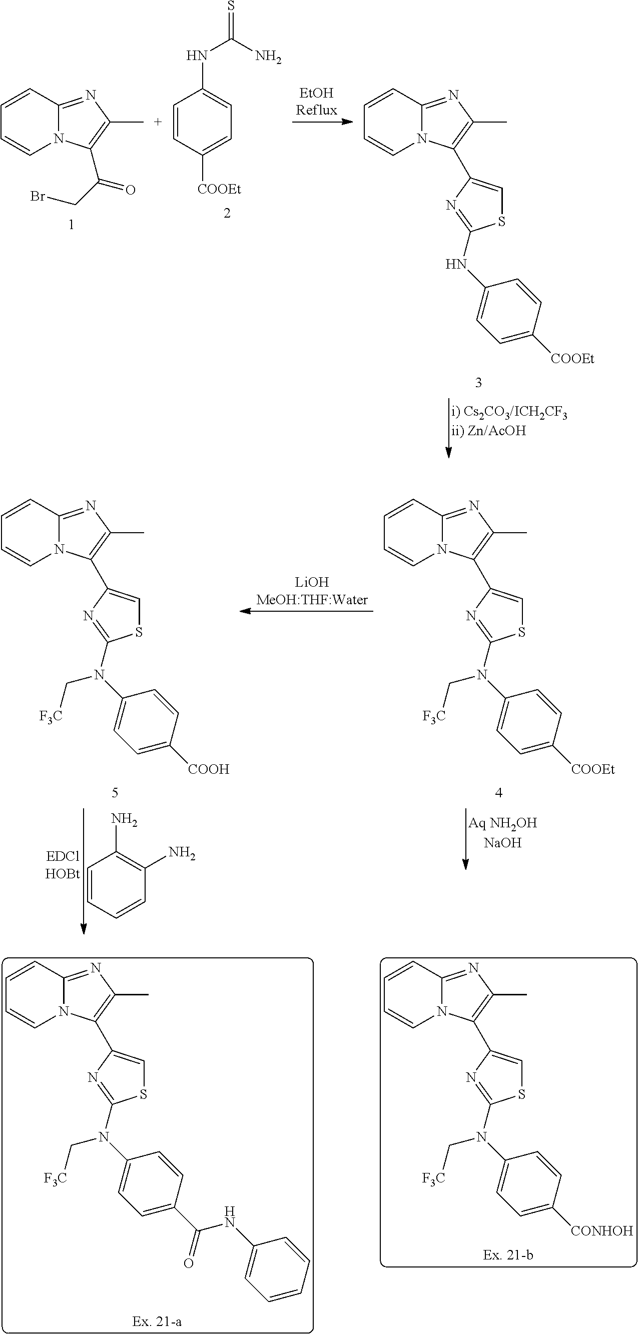

Preparation of Int-3: A mixture of previously prepared Bromo-Int-1 (2.0 g, 7.9 mmol) and thiourea-2 (1.8 g, 7.9 mmol) were refluxed in ethanol (100 mL) for overnight. The reaction mixture was cooled to room temperature, the precipitated solids were filtered, washed with ethanol (2×20 mL) and dried under vacuum to afford Int-3 (2.3 g, 77%).

Preparation of Int-4: To a solution of Int-3 (3.0 g, 7.93 mmol) in DMF (10 mL) was added Cs₂CO₃ (6.44 g, 19.84 mmol) and the mixture was stirred for 20 minutes. Then 2-iodo-1,1,1-trifluoroethane (3.9 mL, 39.68 mmol) was added and the mixture was stirred at 80° C. for 10 hours. The reaction mixture was poured into ice cold water (50 mL) and extracted with EtOAc (120 mL). The organic layer was washed with water (2×60 mL), dried over Na₂SO₄, filtered and evaporated under vacuum to obtain crude dark oil which was purified by column chromatography using EtOAc:Hexane (80:20) to afford a mixture of the desired product and N-vinylic-bi-product (1.6 g) [almost single spot on TLC but mixture by HPLC and ¹H NMR].

The N-vinylic by-product was removed from the desired product as follows:

To the above mixture (1.6 g) in acetic acid (13 mL) was added Zn dust (0.44 g, 6.95 mmol) at room temperature and the mixture was stirred for 1 hour [during which the N-vinylic bi-product was converted to Int-3]. The mixture was poured into cold water (30 mL), basified carefully with solid NaHCO₃ and stirred for 5 minutes, extracted with EtOAc (70 mL). The organic layer was washed with water (50 mL), dried over Na₂SO₄, filtered, evaporated under vacuum and purified by column chromatography using EtOAc:Hexane (80:20) to provide pure N-alkylated product-4 (0.7 g, 19%).

### Preparation of Example 21-b

To a cooled solution of ester-4 (0.50 g, 1.0 mmol) in methanol (25 mL) and DCM (10 mL) was added aqueous hydroxyl amine (10 ml, 50% w/v solution) and stirred for 10 minutes Then, aqueous NaOH (0.40 g, 13.8 mmol in 2.5 mL water) was added and the mixture was stirred at room temperature for 3 hours The reaction mixture was concentrated under reduced pressure, neutralized with 1 N HCl at 0° C. (pH˜7), the precipitated solid was filtered, washed with water and dried under vacuum to afford the hydroxamic acid Example 21-b (0.34 g, 70%) as a white powder. ¹H NMR (200 MHz, DMSO-d₆): δ 11.45 (bs, 1H), 9.30 (bs, 1H), 8.91 (d, J=7.0 Hz, 1H), 7.89 (d, J=8.8 Hz, 2H), 7.67 (d, J=8.4 Hz, 2H), 7.53 (d, J=9.2 Hz, 1H), 7.26 (t, J=7.0 Hz, 1H), 7.07 (s, 1H), 6.93 (t, J=6.6 Hz, 1H), 4.95 (q, J=9.6 Hz, 2H) and 2.49 (s, 3H). ¹³C NMR (125 MHz, DMSO-d₆): δ 14.9, 106.6, 111.9, 116.0, 116.1, 123.6, 124.4, 125.7, 125.9, 126.4, 128.8, 132.2, 140.3, 141.9, 143.5, 145.9, 163.1, 169.0. Mass (m/z): 447 (M⁺+1]; Melting Point: 169° C.

Preparation of Acid-5: To a stirred solution of ester-4 (0.7 g, 1.52 mmol) in a mixture of THF (6.5 mL), water (3 mL) methanol (2 mL) was added LiOH,H₂O (0.19 g, 4.56 mmol) and the mixture was stirred at room temperature for overnight. The reaction mixture was concentrated under reduced pressure and acidified with 2N HCl (pH˜5). The precipitated solids were filtered, washed with water (20 mL), dried under vacuum to furnish the acid-5 (0.51 g, 77%).

### Preparation of Example 21-a

To a stirred solution of acid-5 (0.58 g, 1.34 mmol) in DMF (10 mL) at 0° C. was added EDCI (0.57 g, 2.9 mmol), HOBt (0.18 g, 1.3 mmol) and DIPEA (0.58 mL, 3.3 mmol). After stirring for 15 minutes at 0° C., o-phenylenediamine (0.145 g, 1.3 mmol) was added. The reaction mixture was stirred at room temperature overnight. The reaction mixture was concentrated under reduced pressure, diluted with water (20 mL) and stirred for 30 minutes. The resulting precipitates were filtered, washed with water (20 mL), dried under vacuum and finally purified by column chromatography using 3% MeOH in DCM to provide Example 21-a (0.4 g, 57%). ¹H NMR (200 MHz, DMSO-d₆): δ 2.49 (s, 3H), 4.97 (m, 2H), 6.59 (t, J=7.4 Hz, 1H), 6.77 (d, J=8.0 Hz, 1H), 6.95 (m, 2H), 7.09 (s, 1H), 7.27 (m, 2H), 7.54 (d, J=9.2 Hz, 1H), 7.74 (d, J=6.8 Hz, 2H), 8.13 (d, J=8.4.0, 2H), 8.93 (d, J=6.6 Hz, 1H) 9.76 (s, 1H). ¹³C NMR (125 MHz, DMSO-d₆): δ 14.9, 106.7, 112.0, 116.0, 116.1, 116.2, 123.0, 124.5, 125.7, 126.0, 126.2, 126.6, 126.8, 129.9, 133.9, 140.3, 141.9, 143.3, 143.5, 146.2, 164.4, 169.0. Mass (m/z): 522 [M⁺+1]; Melting Point: 183.1° C.

### Example 22

### Biological Assays

HDAC inhibitory activity of the compound of Example 1 was measured by two types of assays in which HDAC 1 and 6 were used as a target molecule. The first assay was carried out without preincubation after addition of the enzyme. The test compound was suspended in and titrated in DMSO. It was then spotted into a 384-well test plate. The enzyme, HDAC 1 or 6, was diluted in assay buffer containing 25 mM Tris-HCl (pH 8.0), 137 mM NaCl, 2.7 mM KCl, and 0.01% Tween-20 and added to the pre-spotted compound. The peptide substrate containing a fluorophore/quencher pair was diluted in the same assay buffer and added to the compound/enzyme mix initiating the reaction. The reaction incubated at room temperature for about 45 minutes. A concentrated developer solution was diluted in the assay buffer, and added to the reaction. The reaction was incubated at room temperature for about 15 minutes and relative fluorescence was read on an instrument reader.

The second assay is similar to the first assay described above, except that preincubation is carried out for about 3 hours after the enzyme is introduced. The test compound was suspended in, and titrated in DMSO. It was then spotted into a 384-well test plate. The enzyme, HDAC 1 or 6, was diluted in the same assay buffer as used in the previous assay and added to the pre-spotted compound. The enzyme/compound mix was incubated at room temperature for about 3 hours. The peptide substrate containing a fluorophore/quencher pair was diluted in the assay buffer and added to the compound/enzyme mix initiating the reaction. The reaction incubated at room temperature for 45 minutes. A concentrated developer solution was diluted in the assay buffer, and added to the reaction. The reaction was incubated at room temperature for about 15 minutes and relative fluorescence was read on an instrument reader.

The following table shows IC₅₀ data for the compound tested with the protocols described above.

Table 1. IC₅₀ of HDAC inhibitor compound

TABLE 1
IC₅₀ of HDAC inhibitor compound

| | HDAC 1 inhibitory activity | | HDAC 6 inhibitory activity | | |
|---|---|---|---|---|---|
| | (IC₅₀ [μM]) | | (IC₅₀ [μM]) | | |
| | No | 3-hour | No | 3-hour | CDK2 |
| Compound | preincubation | preincubation | preincubation | preincubation | IC₅₀ |
| Example 1 | 1.08 | 0.0927 | 0.318 | 0.490 | |
| Example 2 | 3.26 | 42 | >50 | >50 | |
| Example 3 | 0.080 | 0.0535 | 0.0467 | 0.0558 | |
| Example 4 | 0.313 | 0.080 | >50 | >50 | |
| Example 5 | | 0.0539 | | | 4.7 |
| Example 6 | | 0.0215 | | | >40 |
| Example 7 | | 0.022 | | | |
| Example 8 | | 0.0510 | | | >20 |
| Example 9 | | 0.1540 | | | |
| Example 10 | | 0.0590 | | | |
| Example 11 | | 0.0569 | | | >40 |
| Example 12 | | 0.0626 | | | |
| Example 13 | 0.0855 | 0.0290 | 0.0185 | 0.017 | 4.1 |
| Example 14 | 5.4 | 0.1140 | 0.183 | | 22.05 |
| Example 15 | | 0.0370 | | >10 | 4.7 |
| Example 16 | | 9.1300 | | | >40 |
| Example 17 | | 0.0246 | | | 4.385 |
| Example 18 | | 0.0467 | | | 2.67 |
| Example 19 | | 6.0300 | | | >40 |
| Example 20 | | 0.048 | | | >40 |
| Example 21a | | 0.147 | | | 0.74 |
| Example 21b | | 5.58 | | | >40 |

The results indicate that the compounds have inhibitory activity against HDAC and/or CDK and thus can be useful to treat or inhibit diseases caused by abnormal activities of HDAC and/or CDK.

All patents and publications cited herein are incorporated by reference into this application in their entirety.

What is claimed is:

1. A compound selected from those of Formula (I) and pharmaceutically acceptable salts thereof: 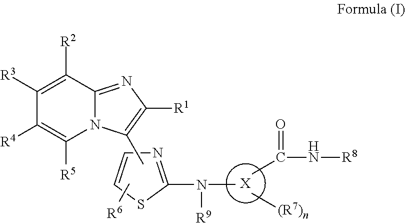
wherein
R¹, R², R³, R⁴ and R⁵ are independently selected from the group consisting of H, halo, nitro, cyano, hydroxy, hydroxyalkyl, haloalkyl, haloalkoxy, amino, aminoalkyl, azido, carboxy, carbamoyl, mercapto, sulphamoyl, C₁₋₁₀ alkyl, C₂₋₁₀ alkenyl, C₂₋₁₀ alkynyl, C₁₋₁₀ alkoxy, C₁₋₁₀ alkanoyl, C₁₋₁₀ alkanoyloxy, N—(C₁₋₁₀ alkyl)amino, N,N—(C₁₋₁₀ alkyl)₂-amino, C₁₋₁₀ alkanoylamino, N—(C₁₋₁₀ alkyl)carbamoyl, alkyl)₂-carbamoyl, C₁₋₁₀ alkyl-S(O)ₐ wherein a is 0, 1 or 2, C₁₋₁₀ alkoxycarbonyl, NH₂—S(O)₂NH—, N—(C₁₋₁₀ alkyl)sulphamoyl, N,N—(C₁₋₁₀ alkyl)₂sulphamoyl, aryl, aryloxy, arylthio, heteroaryl, heteroaryloxy, cycloalkyl, cycloalkyloxy, heterocyclyl, heterocyclyl(C═O)—, heterocyclyloxy and heterocyclylthio; wherein each of R¹, R², R³, R⁴ and R⁵ is optionally substituted by one or more A where such an optional substitution is chemically feasible;
R⁶ is H, halo, nitro, cyano, trifluoromethyl, trifluoromethoxy, amino, carboxy, carbamoyl, sulphamoyl, C₁₋₁₀ alkyl, C₂₋₁₀ alkenyl, C₂₋₁₀ alkynyl, C₁₋₁₀ alkoxy, C₁₋₁₀ alkanoyl, N—(C₁₋₁₀ alkyl)amino, N,N—(C₁₋₁₀ alkyl)₂ amino, C₁₋₁₀ alkanoylamino, N—(C₁₋₁₀ alkyl)carbamoyl, N,N—(C₁₋₁₀ alkyl)₂ carbamoyl, C₁₋₁₀ alkyl-S(O)ₐ wherein a is 0, 1 or 2, NH₂—S(O)₂NH—, N—(C₁₋₁₀ alkyl)sulphamoyl or N,N—(C₁₋₁₀ alkyl)₂sulphamoyl; wherein R⁶ is optionally substituted by one or more B where such an optional substitution is chemically feasible;
X is phenyl, 5-membered heteroaryl, or 6-membered heteroaryl, wherein the heteroaryl contains one or more heteroatoms selected from N, S and O;
R⁷ represents one or more non-hydrogen substituents selected from halo and methyl;
n is 0, 1, 2, 3, or 4;
R⁸ is hydroxy, aryl or heteroaryl, wherein aryl or heteroaryl are substituted with —NH₂ or —OH and aryl or heteroaryl is optionally further substituted with one or more groups R¹⁰ selected from amino, halo, alkyl, cycloalkyl, heterocyclyl, aryl and heteroaryl;
R⁹ is H, alkyl, haloalkyl, aminoalkyl, cycloalkyl, heterocyclyl, aryl, heteroaryl, wherein R⁹ is optionally substituted by one or more D where such an optional substitution is chemically feasible;
A and B are independently selected from halo, nitro, cyano, hydroxy, hydroxyalkyl, haloalkyl, haloalkoxy, amino, azido, carboxy, carbamoyl, mercapto, sulphamoyl, C₁₋₁₀ alkyl, C₂₋₁₀ alkenyl, C₂₋₁₀ alkynyl, C₁₋₁₀ alkoxy, C₁₋₁₀ alkoxyalkyl, C₁₋₁₀ alkanoyl, C₁₋₁₀ alkanoyloxy, N—(C₁₋₁₀ alkyl)amino, N,N—(C₁₋₁₀ alkyl)₂-amino, C₁₋₁₀ alkanoylamino, N—(C₁₋₁₀ alkyl)carbamoyl, N,N—(C₁₋₁₀ alkyl)₂-carbamoyl, C₁₋₁₀ alkyl-S(O)ₐ wherein a is 0, 1 or 2, C₁₋₁₀ alkoxycarbonyl, N—(C₁₋₁₀ alkyl)sulphamoyl, N,N—(C₁₋₁₀ alkyl)₂sulphamoyl, H₂NS(O)₂NH—, N-(C₁₋₁₀ alkyl)NHS(O)₂NH—, N,N—(C₁₋₁₀ alkyl)₂NS(O)₂NH—, aryl, aryloxy, arylthio, heteroaryl, heteroaryloxy, cycloalkyl, cycloalkyloxy, heterocyclyl, heterocyclyl(C═O)—, heterocyclyloxy and heterocyclylthio; and
D is selected from halo, nitro, cyano, hydroxy, amino, azido, carboxy and mercapto.

2. The compound according to claim 1, wherein
R¹ is selected from the group consisting of hydrogen, methyl, ethyl, propyl, methoxy, ethoxy, methoxymethyl, ethoxyethyl, propoxyethyl, methoxyethoxy, trifluoromethyl, hydroxyethoxy, dimethylamino, diethylamino, dimethylaminomethyl, diethylaminomethyl, dimethylaminoethoxy, trifluoromethoxymethyl, trifluoroethoxymethyl, benzyl, phenylethyl, trifluoromethylphenylethyl, phenoxymethyl, fluorophenoxymethyl, phenylethylaminomethyl, benzylaminomethyl, morpholinylmethyl, morpholinylethoxy, imidazolylmethyl, triazinylmethyl, piperidinylmethyl, piperidinyloxy, trifluoromethylpiperidinylmethyl, pyridinyloxymethyl, pyridinylmethoxy, tetrahydropyrazinyloxy, methylpiperazinylmethyl, pyrrolidinylmethyl and pyrrolidinylethoxy;
at least two of R², R³, R⁴, and R⁵ are hydrogen and each non-hydrogen R², R³, R⁴, and R⁵ is selected from chloro, fluoro, bromo, methyl, ethyl, propyl, methoxy, ethoxy, carboxy, cyano, methoxymethyl, ethoxyethyl, propoxyethyl, methoxyethoxy, trifluoromethyl, hydroxyethoxy, dimethylamino, diethylamino, dimethylaminomethyl, dimethylamino ethyl, diethylaminomethyl, dimethylaminoethoxy, trifluoromethoxymethyl, trifluoroethoxymethyl, 3-oxetanoxy, trifluoroethylaminomethyl, N-methyl-N-methoxyethyl-aminoethyl, cyclopropanylmethyl, cyclobutoxy, 1-cyclopropanylethoxy, cyclopropanylmethylaminomethyl, 4-methylpiperazin-1-carbonyl, isoindolin-2-yl, N-methoxyethylcarbamoyl, N-(morpholin-4-yl)-ethylcarbamoyl, dimethylaminoethylamino, methylcarboxy, N,N-dimethylaminoethylcarbamoyl, benzyl, phenylethyl, trifluoromethylphenylethyl, phenoxymethyl, fluorophenoxymethyl, phenylethylaminomethyl, benzylaminomethyl, triazinylmethyl, piperidinylmethyl, piperidinyloxy, trifluoromethylpiperidinylmethyl, pyridinyloxymethyl, pyridinylmethoxy, tetrahydropyrazinyloxy, methylpiperazinylmethyl, pyrrolidin-1-yl, pyrrolidin-2-yl, pyrrolidin-3-yl, pyrrolidin-1-ylmethyl, pyrrolidin-2-ylmethyl, pyrrolidin-3-ylmethyl, pyrrolidin-1-ylethoxy, pyrrolidin-2-ylethoxy, pyrrolidin-3-ylethoxy, imidazol-1-ylmethyl, imidazol-2-ylmethyl, imidazol-4-ylmethyl, imidazolidin-1-yl, imidazolidin-2-yl, imidazolidin-4-yl, imidazolidin-1-ylmethyl, imidazolidin-2-ylmethyl, imidazolidin-4-ylmethyl, imidazolin-1-yl, imidazolin-2-yl, imidazolin-4-yl, pyrazolidin-1-yl, pyrazolidin-3-yl, pyrazolidin-4-yl, pyrazolin-1-yl, pyrazolin-3-yl, pyrazolin-4-yl, piperidin-1-yl, piperidin-2-yl, piperidin-3-yl, piperidin-4-yl, piperidin-1-ylmethyl, piperidin-2-ylmethyl, piperidin-3-ylmethyl, piperidin-4-ylmethyl, piperazin-1-yl, piperazin-2-yl, piperazin-3-yl, morpholin-2-yl, morpholin-3-yl, morpholin-4-yl, morpholin-2-ylmethyl, morpholin-3-ylmethyl, morpholin-4-ylmethyl, morpholin-2-ylethoxy, morpholin-3-ylethoxy and morpholin-4-ylethoxy;
R⁶ is H, methyl, ethyl, bromo or trifluoromethyl; and
X is phenyl or 5-membered heteroaryl.

3. The compound of claim 1 selected from those of Formula (I-a) and pharmaceutically acceptable salts thereof:

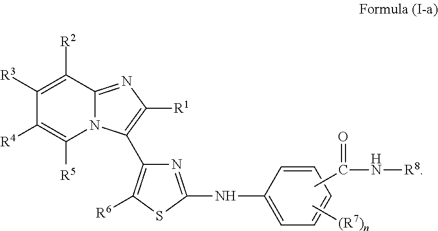

4. The compound of claim 3, wherein R¹, R², R³, R⁴ and R⁵ are H; R⁶ is H, alkyl or haloalkyl; each R⁷ is independently fluoro, chloro, bromo, or methyl; n is 0, 1 or 2; and R⁸ is hydroxyl, aryl or heteroaryl, wherein aryl or heteroaryl are substituted with —NH₂ or —OH at a ring position adjacent to attachment of the —CONH-moiety and R⁸ is optionally further substituted with one or more groups R¹⁰ selected from amino, halo, alkyl, cycloalkyl, heterocyclyl, aryl, and heteroaryl.

5. The compound of claim 4 which is selected from the group consisting of: 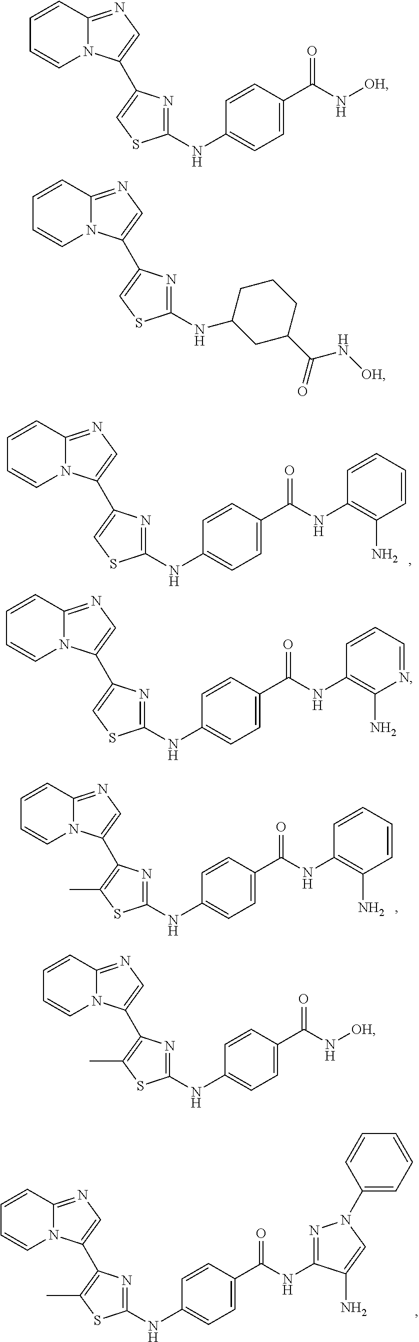 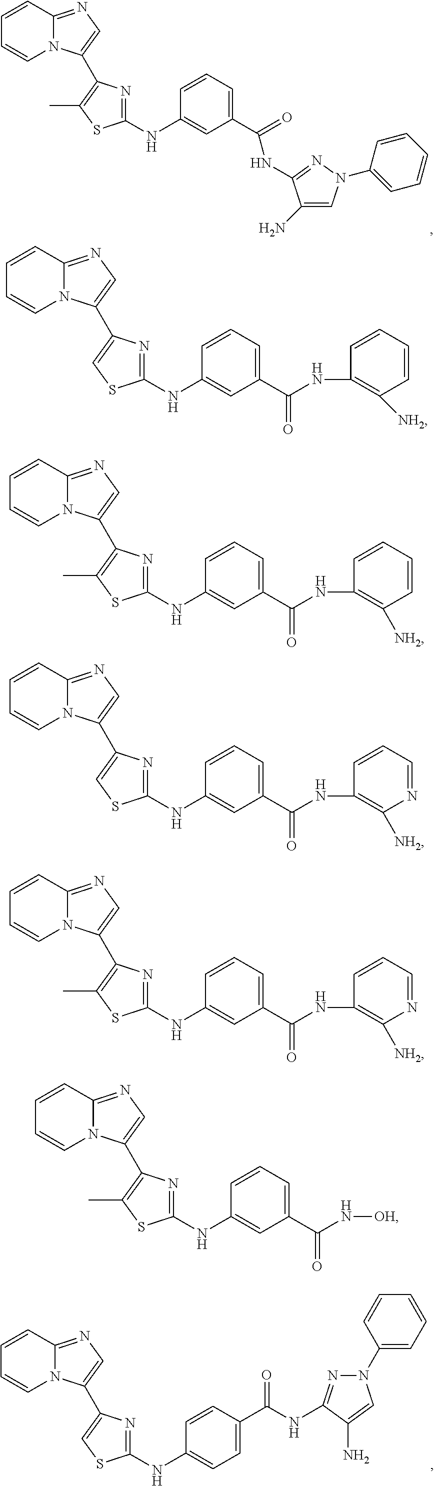 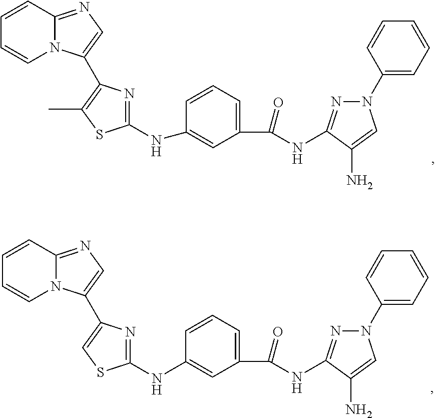
and pharmaceutically acceptable salts thereof.

6. The compound of claim 4 which is selected from the group consisting of: 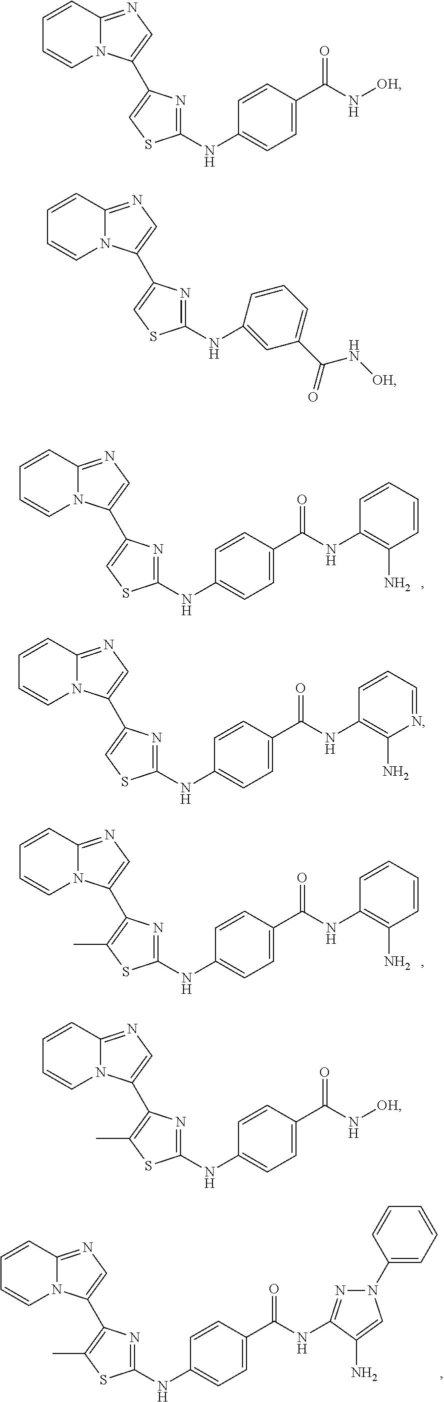
and pharmaceutically acceptable salts thereof.

7. The compound of claim 3, wherein R¹ is methyl; R², R³, R⁴ and R⁵ are H; R⁶ is H, alkyl or haloalkyl; each R⁷ is independently fluoro, chloro, bromo, or methyl; n is 0, 1, or 2; and R⁸ is hydroxyl, aryl or heteroaryl, wherein aryl or heteroaryl are substituted with —NH₂ or —OH at a ring position adjacent to attachment of the —CONH-moiety and R⁸ is optionally substituted with one or more groups R¹⁰ selected from amino, halo, alkyl, cycloalkyl, heterocyclyl, aryl, and heteroaryl.

8. The compound of claim 7 which is selected from the group consisting of: 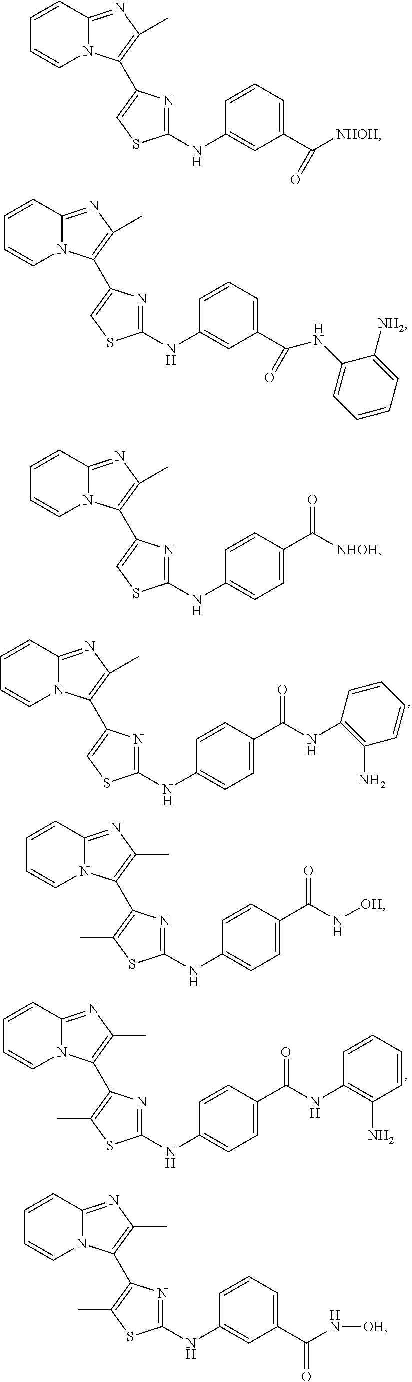 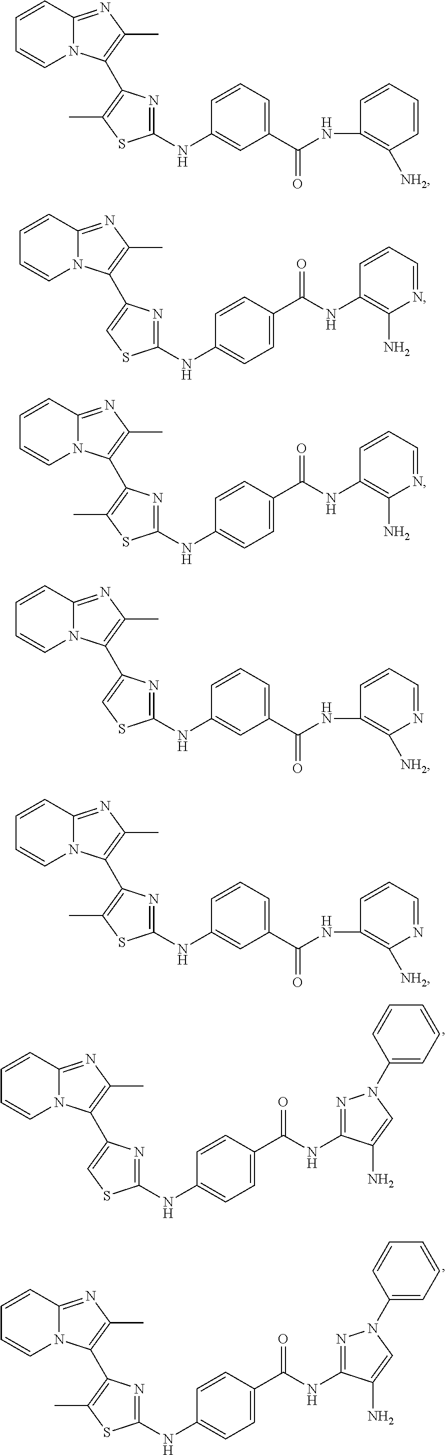 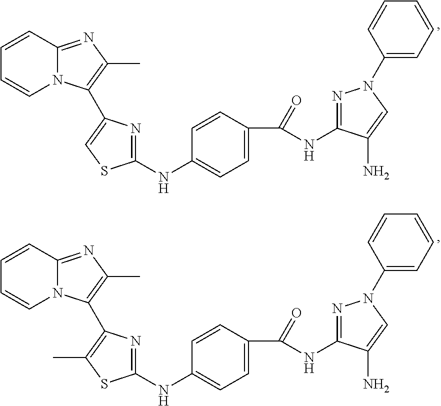
and pharmaceutically acceptable salts thereof.

9. The compound of claim 7 which is selected from the group consisting of: 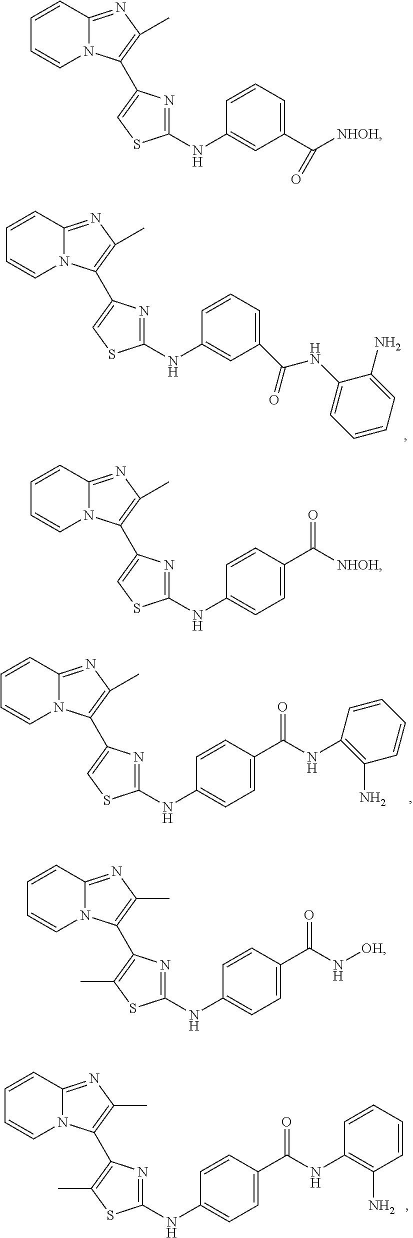
and pharmaceutically acceptable salts thereof.

10. The compound of claim 3, wherein at least two of R¹, R², R³, R⁴ and R⁵ are H, and each non-hydrogen R¹, R², R³, R⁴ and R⁵ is selected from chloro, fluoro, bromo, methyl, ethyl, propyl, methoxy, ethoxy, carboxy, cyano, methoxymethyl, ethoxyethyl, propoxyethyl, methoxyethoxy, trifluoromethyl, hydroxyethoxy, dimethylamino, diethylamino, dimethylaminomethyl, dimethylaminoethyl, diethylaminomethyl, dimethylaminoethoxy, trifluoromethoxymethyl, trifluoroethoxymethyl, 3-oxetanoxy, trifluoroethylaminomethyl, N-methyl-N-methoxyethyl-aminoethyl, cyclopropanylmethyl, cyclobutoxy, 1-cyclopropanylethoxy, cyclopropanylmethylaminomethyl, 4-methylpiperazin-1-carbonyl, isoindolin-2-yl, N-methoxyethylcarbamoyl, N-(morpholin-4-yl)-ethylcarbamoyl, dimethylaminoethylamino, methylcarboxy, N,N-dimethylaminoethylcarbamoyl, benzyl, phenylethyl, trifluoromethylphenylethyl, phenoxymethyl, fluorophenoxymethyl, phenylethylaminomethyl, benzylaminomethyl, triazinylmethyl, piperidinylmethyl, piperidinyloxy, trifluoromethylpiperidinylmethyl, pyridinyloxymethyl, pyridinylmethoxy, tetrahydropyrazinyloxy, methylpiperazinylmethyl, pyrrolidin-1-yl, pyrrolidin-2-yl, pyrrolidin-3-yl, pyrrolidin-1-ylmethyl, pyrrolidin-2-ylmethyl, pyrrolidin-3-ylmethyl, pyrrolidin-1-ylethoxy, pyrrolidin-2-ylethoxy, pyrrolidin-3-ylethoxy, imidazol-1-ylmethyl, imidazol-2-ylmethyl, imidazol-4-ylmethyl, imidazolidin-1-yl, imidazolidin-2-yl, imidazolidin-4-yl, imidazolidin-1-ylmethyl, imidazolidin-2-ylmethyl, imidazolidin-4-ylmethyl, imidazolin-1-yl, imidazolin-2-yl, imidazolin-4-yl, pyrazolidin-1-yl, pyrazolidin-3-yl, pyrazolidin-4-yl, pyrazolin-1-yl, pyrazolin-3-yl, pyrazolin-4-yl, piperidin-1-yl, piperidin-2-yl, piperidin-3-yl, piperidin-4-yl, piperidin-1-ylmethyl, piperidin-2-ylmethyl, piperidin-3-ylmethyl, piperidin-4-ylmethyl, piperazin-1-yl, piperazin-2-yl, piperazin-3-yl, morpholin-2-yl, morpholin-3-yl, morpholin-4-yl, morpholin-2-ylmethyl, morpholin-3-ylmethyl, morpholin-4-ylmethyl, morpholin-2-ylethoxy, morpholin-3-ylethoxy and morpholin-4-ylethoxy;
R⁶ is H, alkyl or haloalkyl;
R⁷ is independently fluoro, chloro, bromo, or methyl;
n is 0, 1, or 2; and
R⁸ is hydroxyl, aryl or heteroaryl, wherein aryl or heteroaryl are substituted with —NH₂ or —OH at a ring position adjacent to attachment of the —CONH-moiety and R⁸ is optionally further substituted with one or more groups R¹⁰ selected from amino, halo, alkyl, cycloalkyl, heterocyclyl, aryl, and heteroaryl.

11. The compound of claim 10 which is selected from the group consisting of: 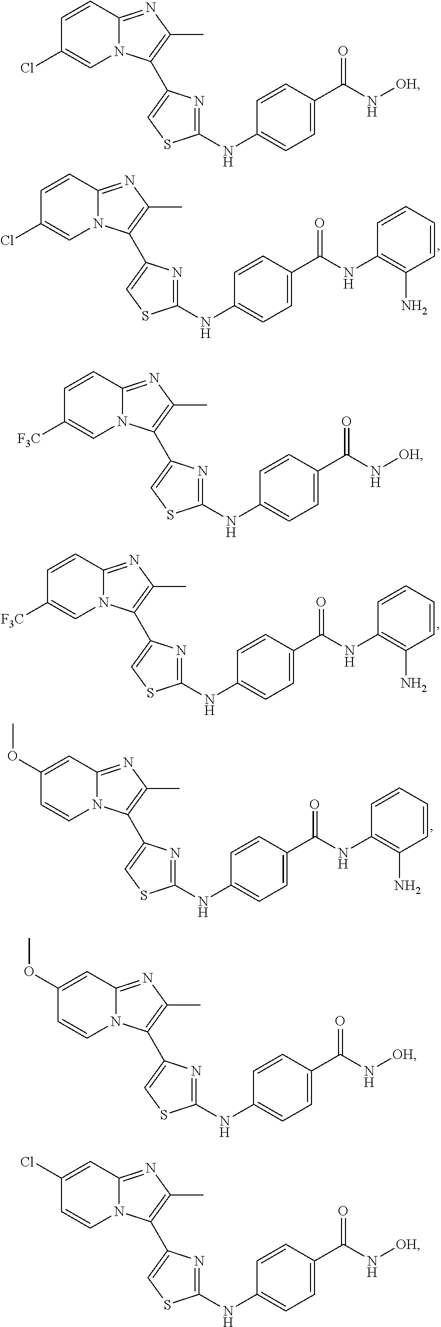 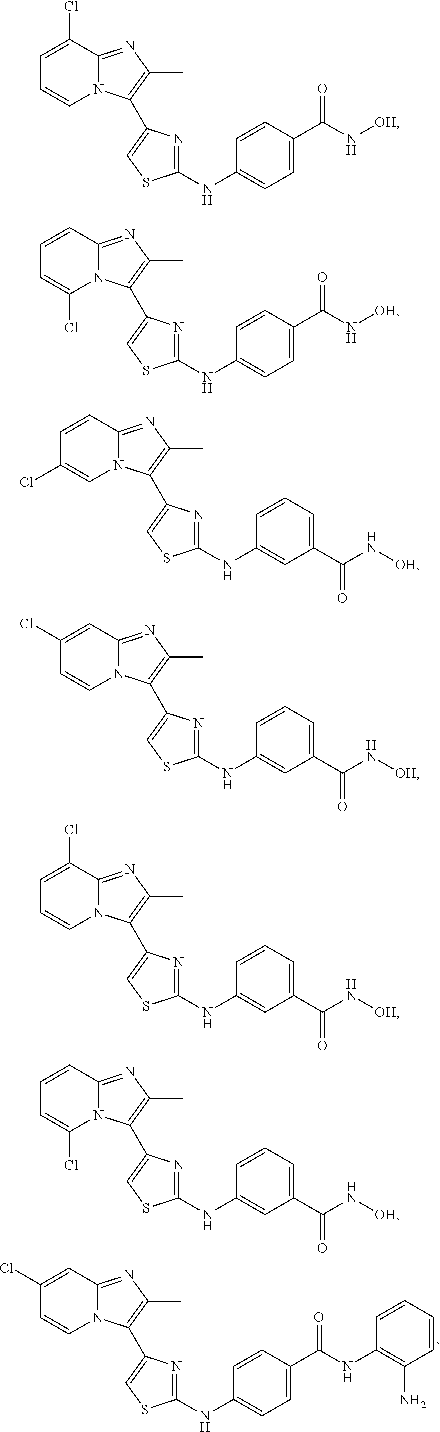 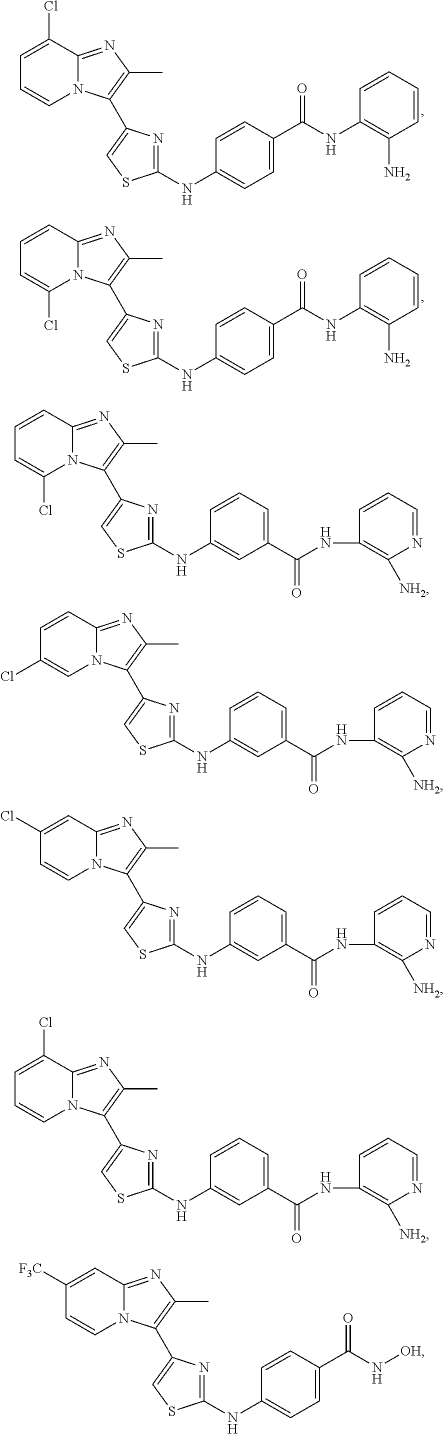 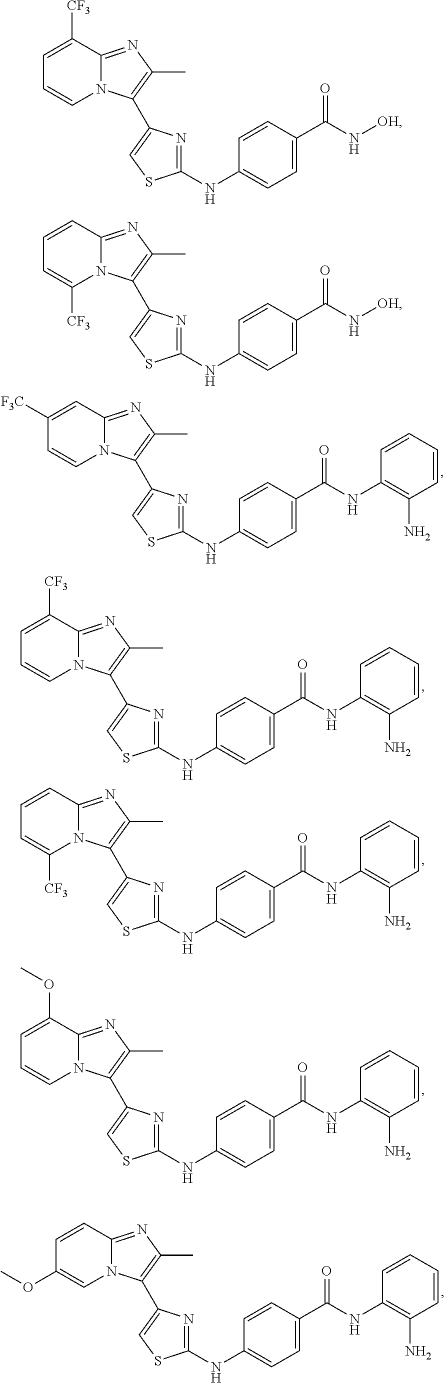 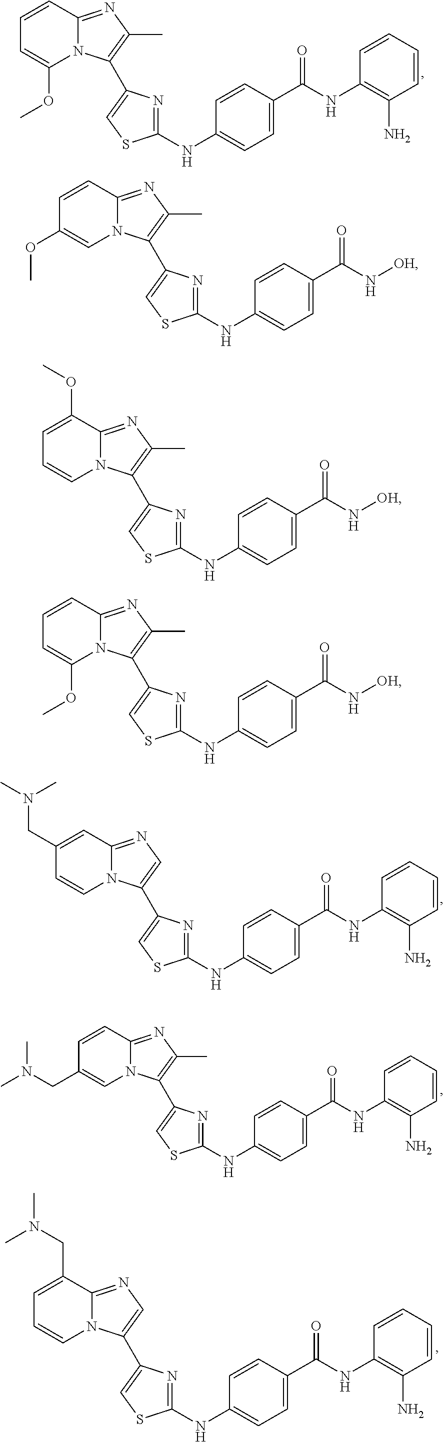 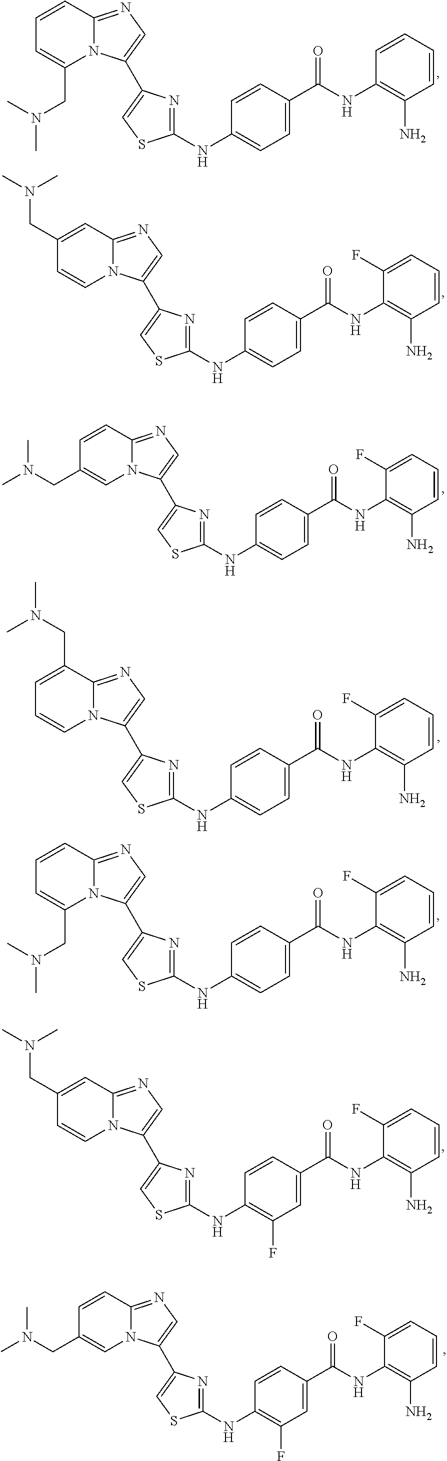 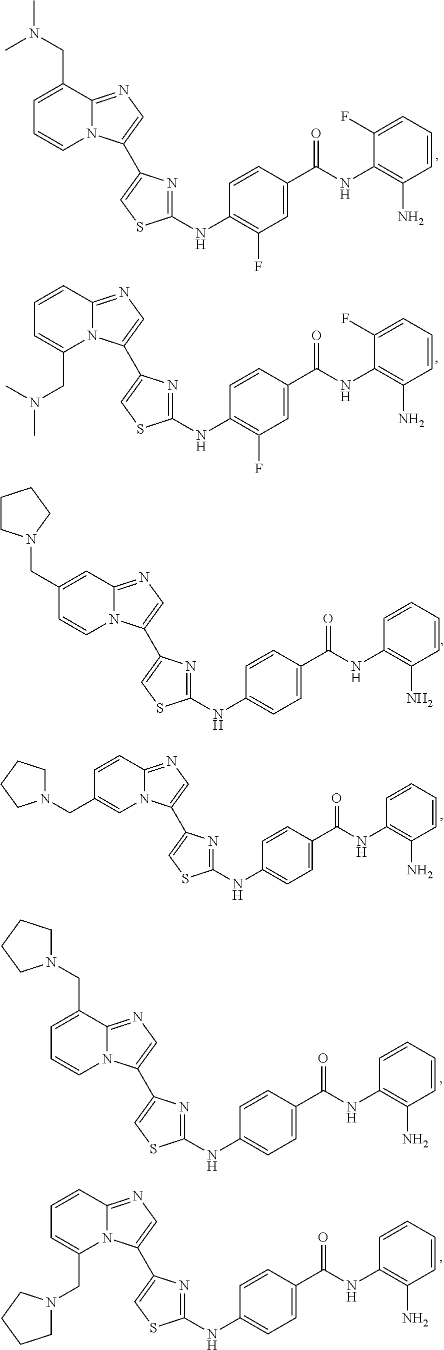 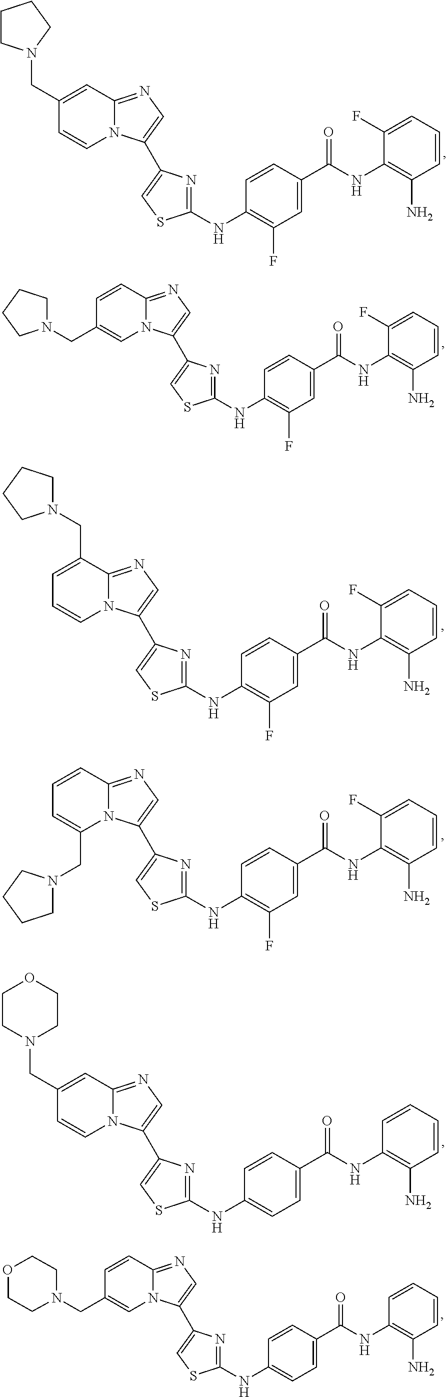 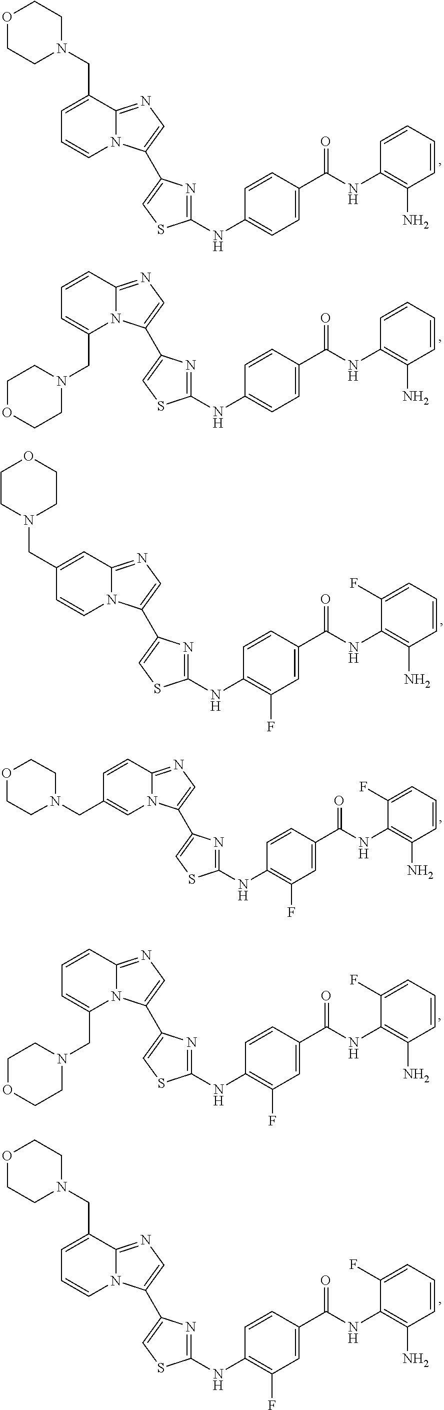 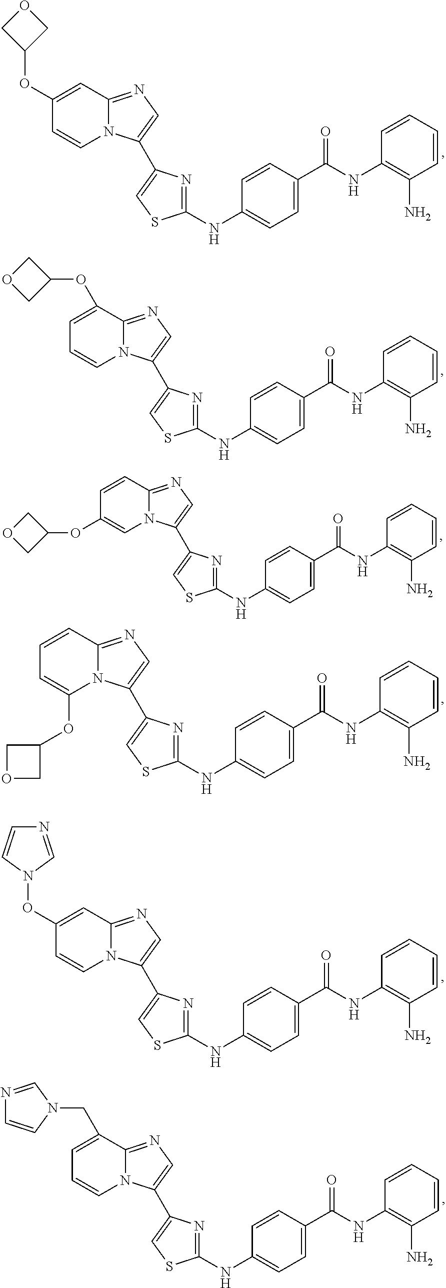 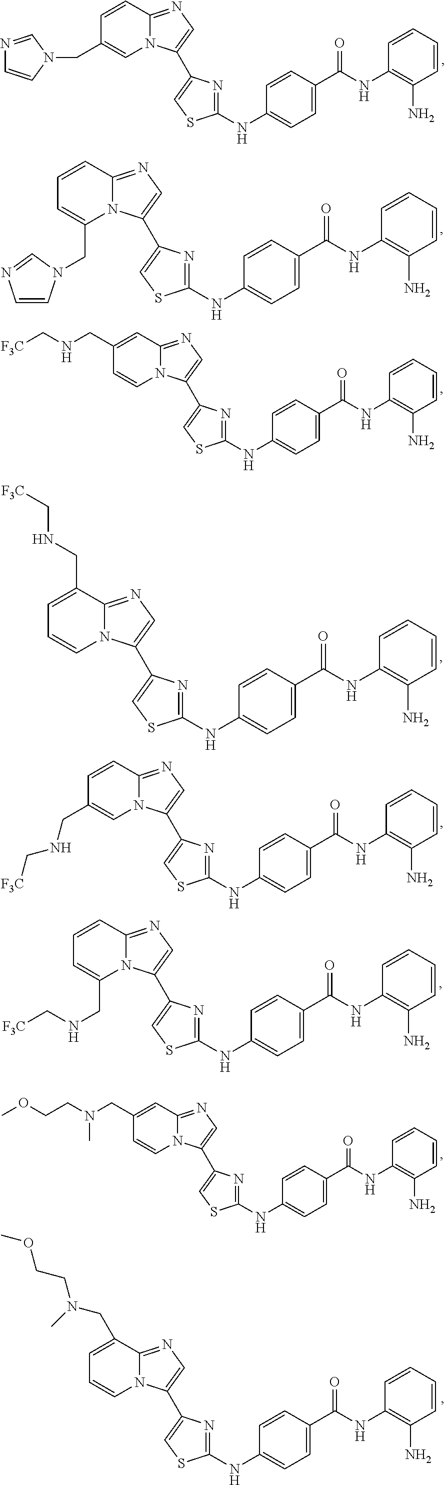 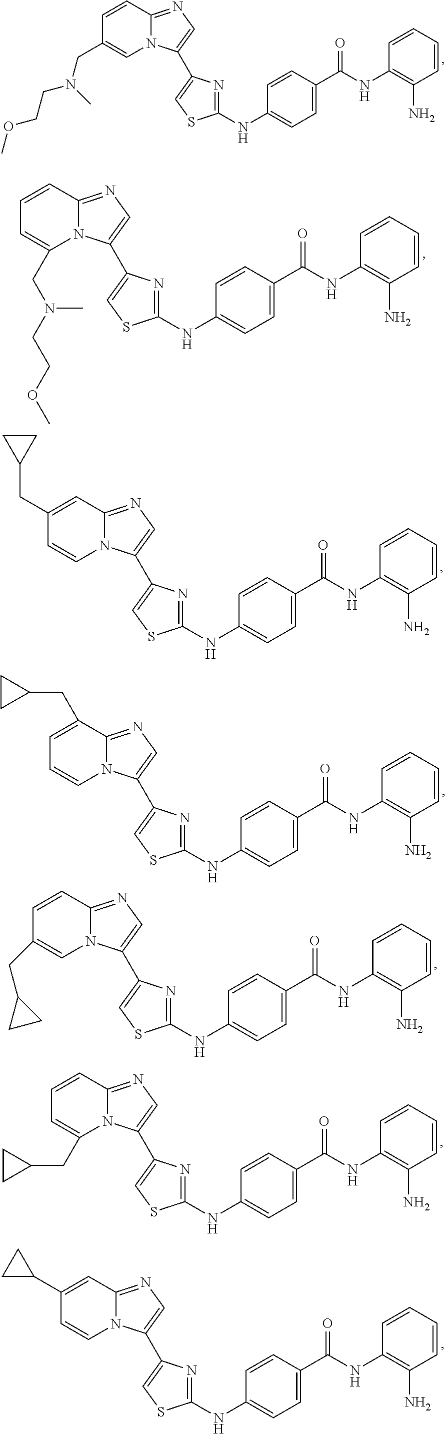 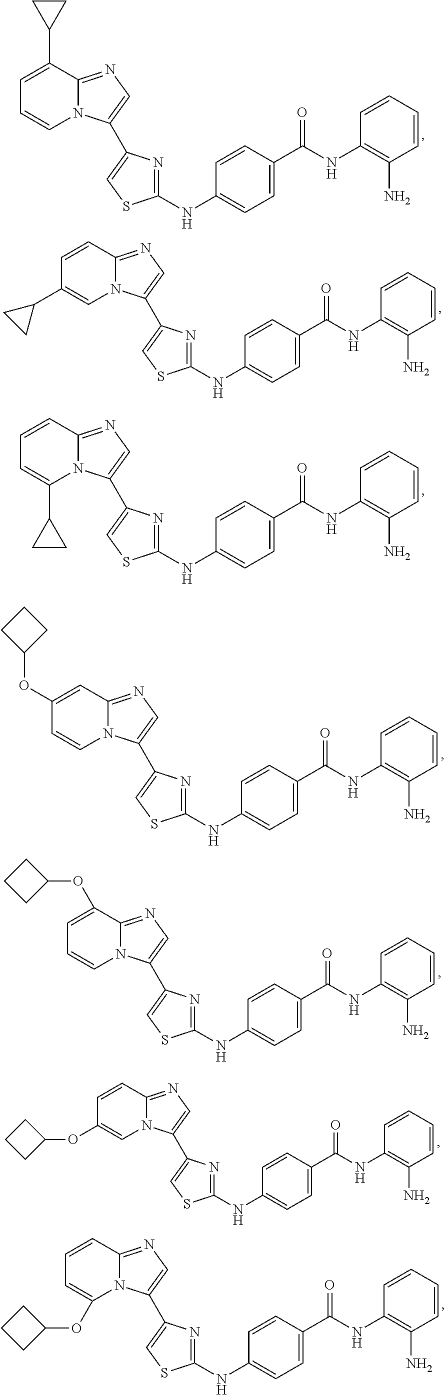 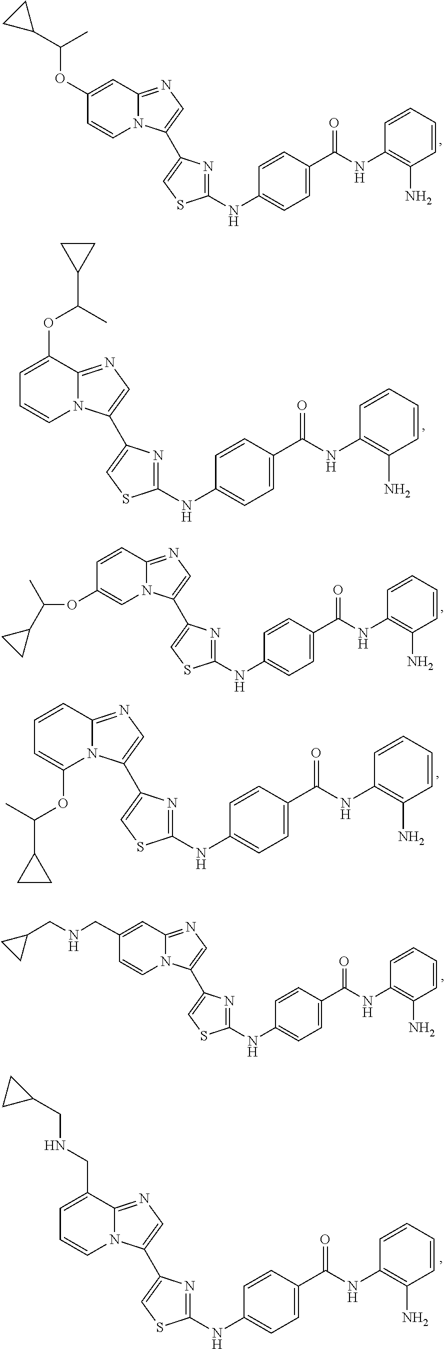 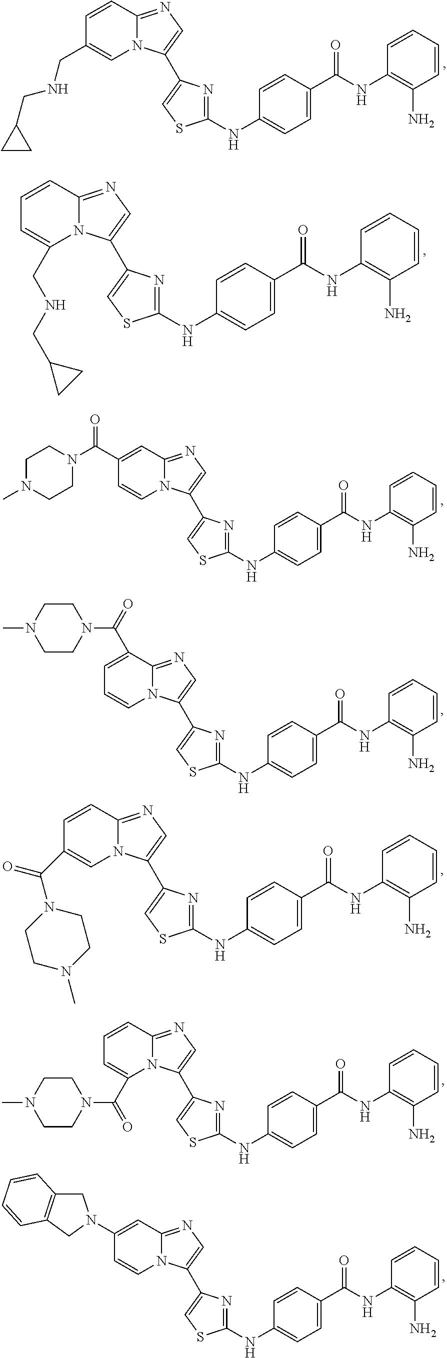 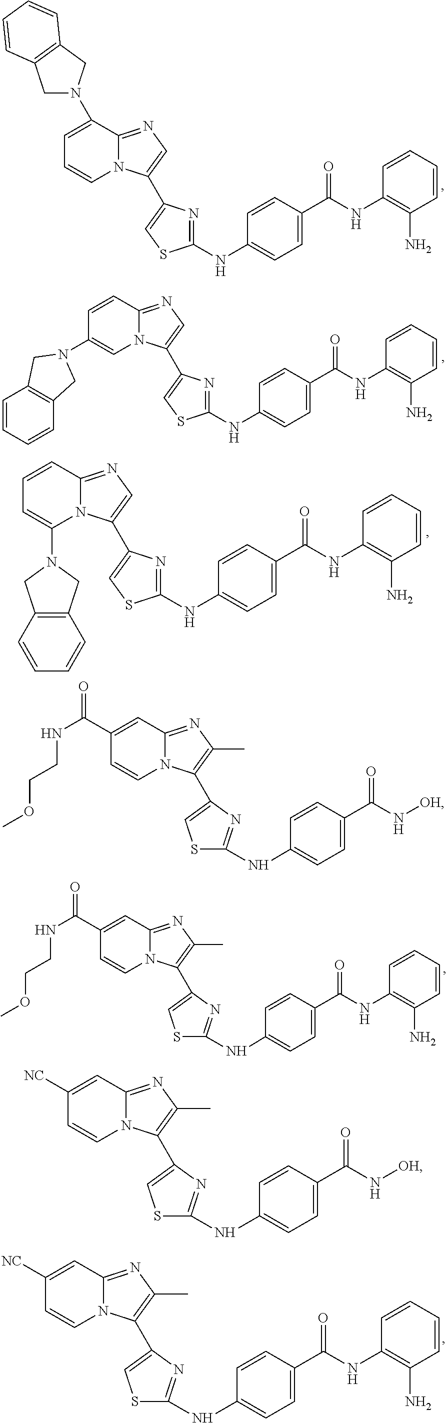 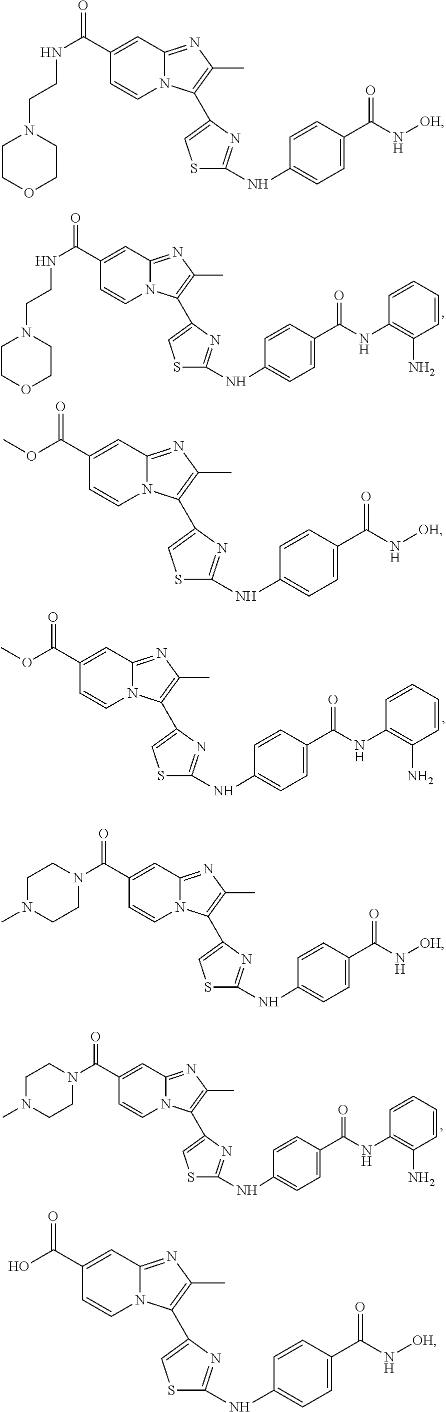 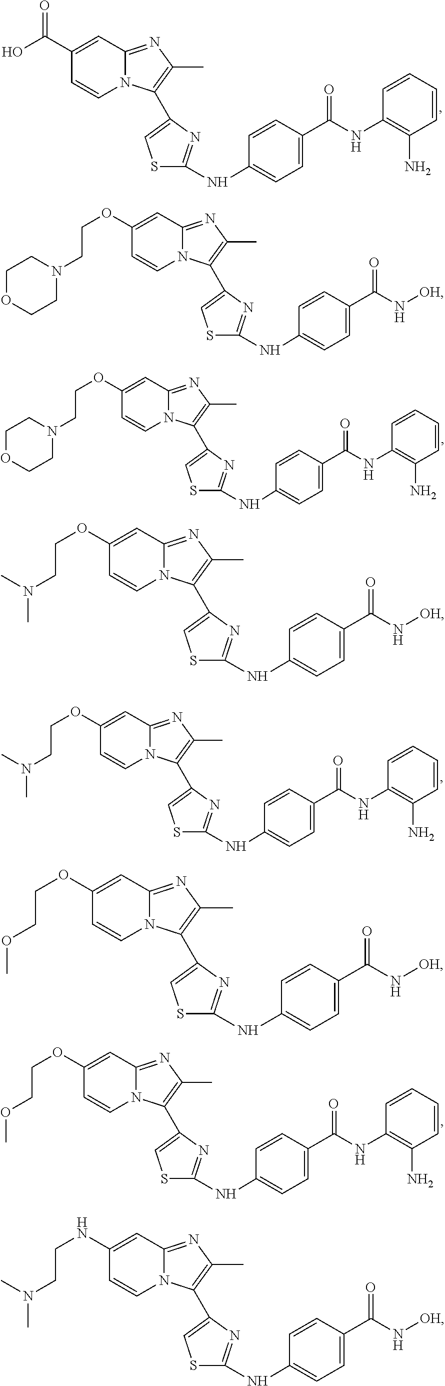 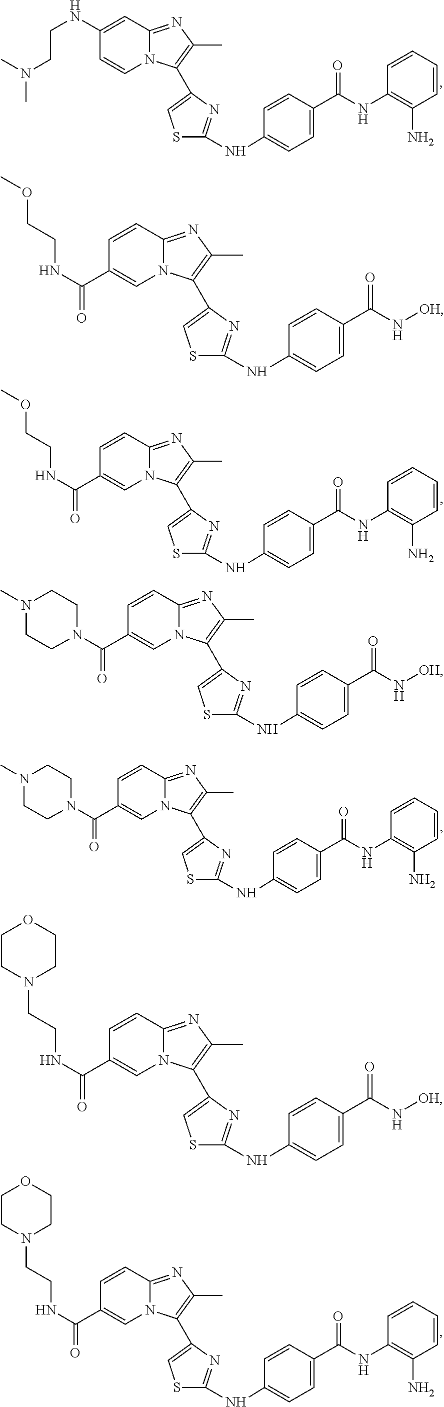 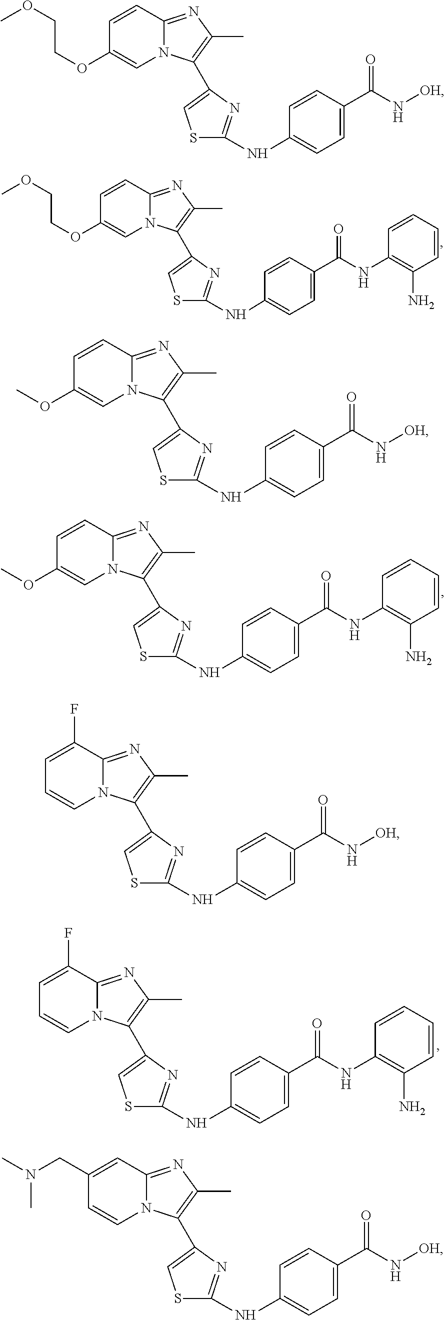 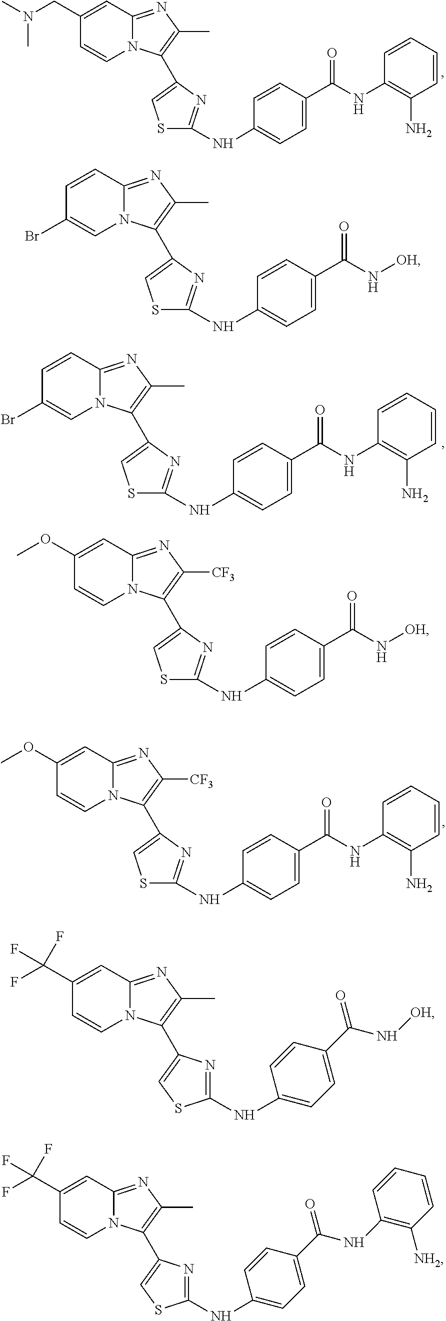 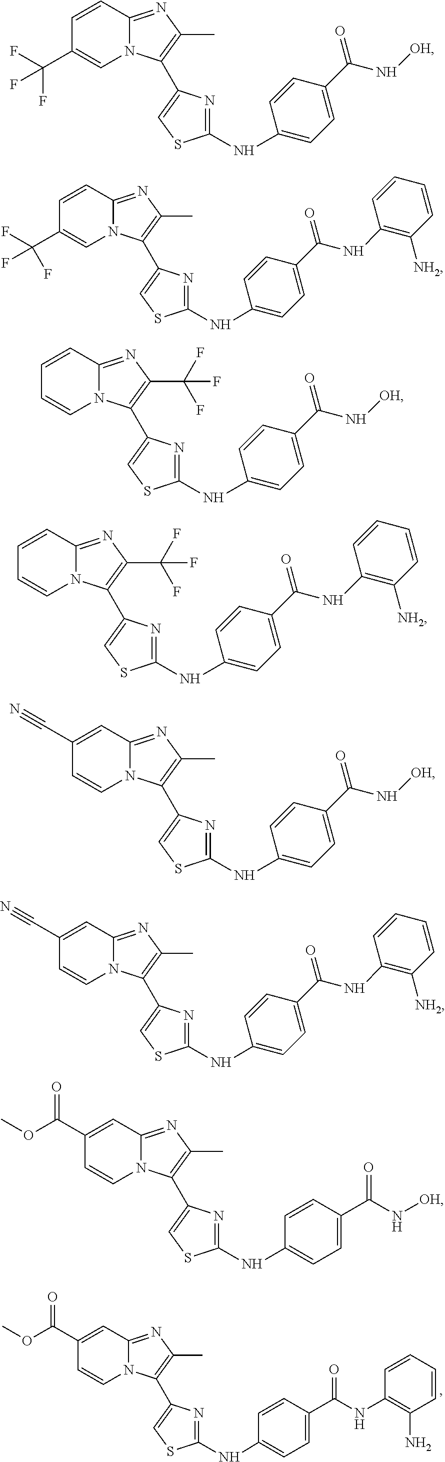 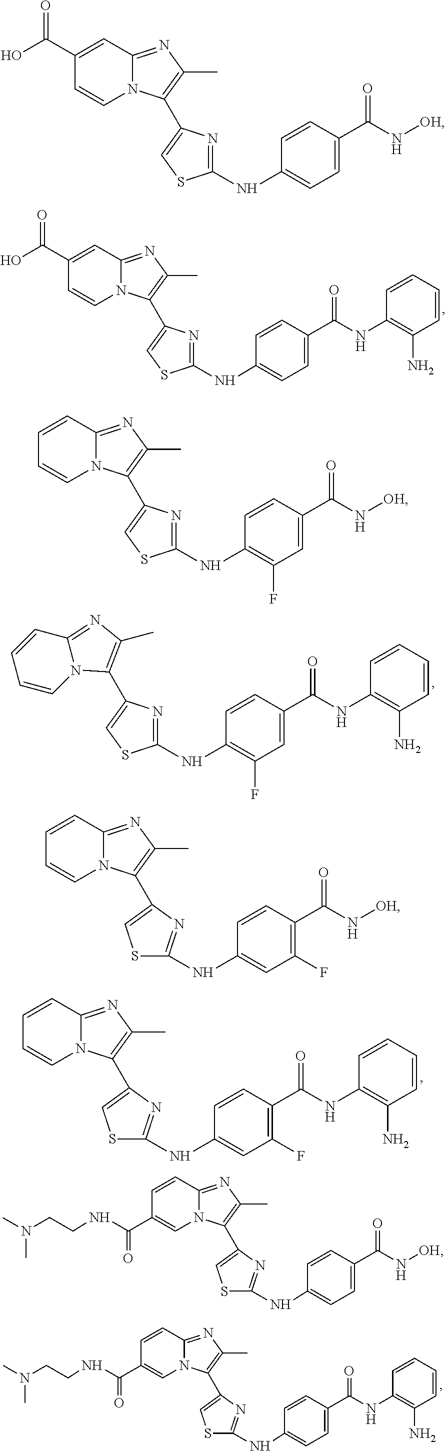 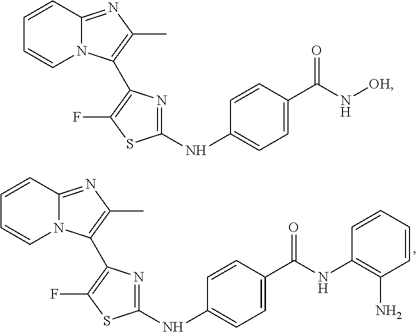
and pharmaceutically acceptable salts thereof.

12. The compound of claim 10 which is selected from the group consisting of: 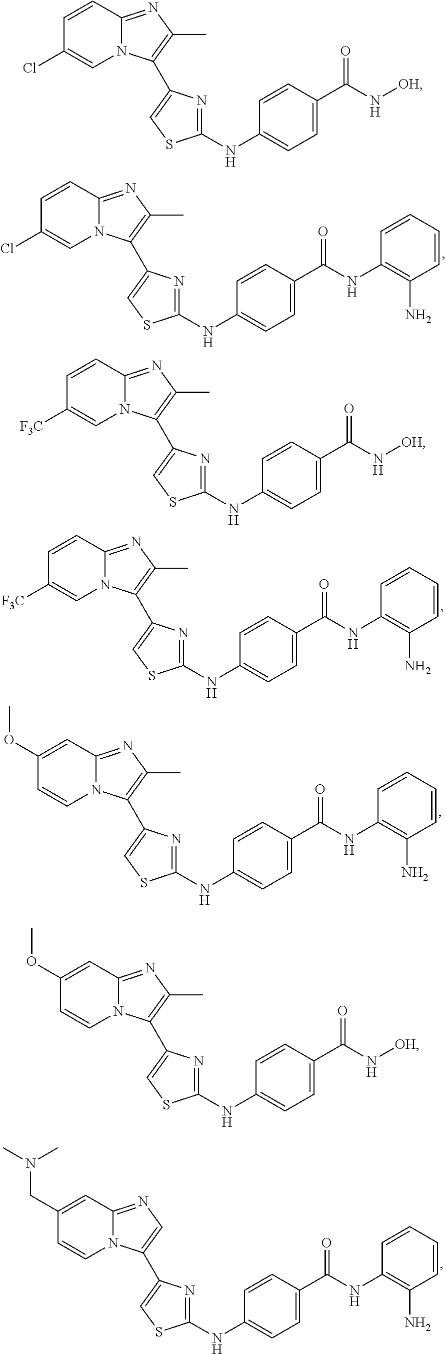 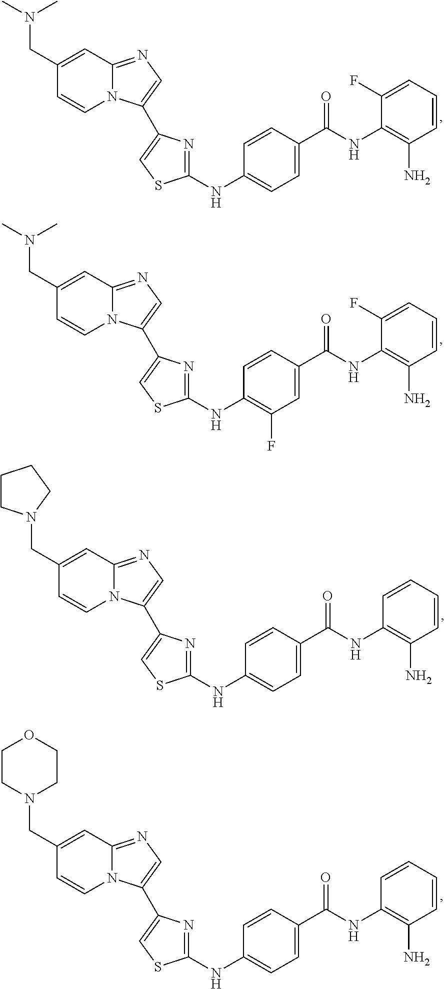
and pharmaceutically acceptable salts thereof.

13. The compound of claim 1 selected from those of Formula (I-b) and pharmaceutically acceptable salts thereof:

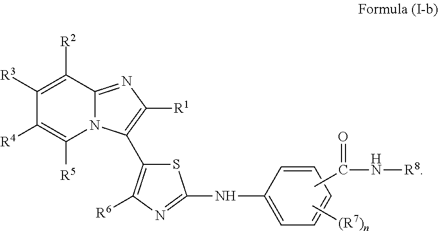

14. The compound of claim 13, wherein R¹, R², R³, R⁴ and R⁵ are H; R⁶ is H, alkyl or haloalkyl; each R⁷ is independently fluoro, chloro, bromo, or methyl; n is 0, 1, or 2; and R⁸ is hydroxyl, aryl or heteroaryl, wherein aryl or heteroaryl are substituted with —NH₂ or —OH at a ring position adjacent to attachment of the —CONH-moiety and R⁸ is optionally substituted with one or more groups R¹⁰ selected from amino, halo, alkyl, cycloalkyl, heterocyclyl, aryl, and heteroaryl.

15. The compound of claim 14 which is selected from the group consisting of: 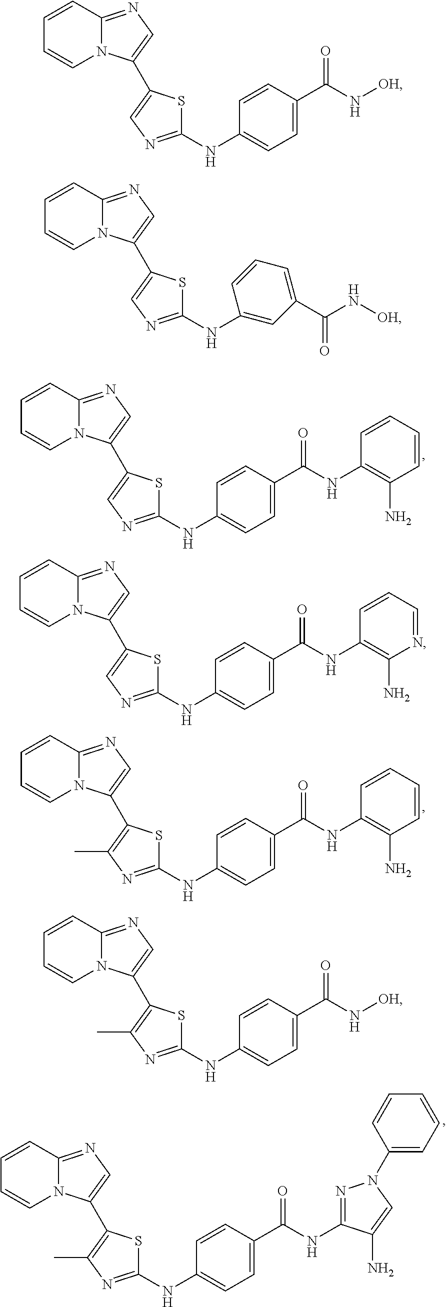 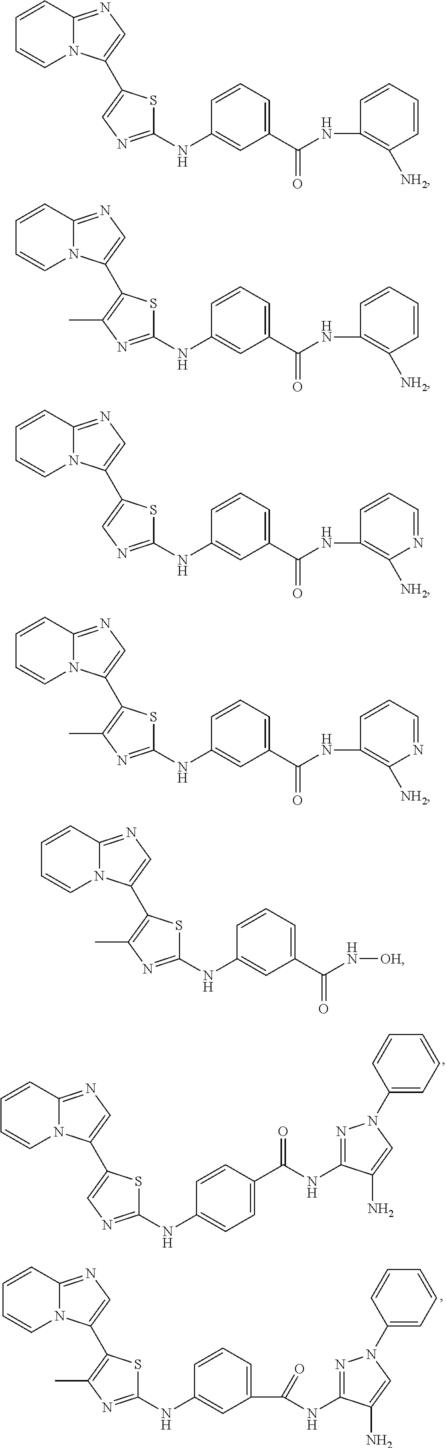 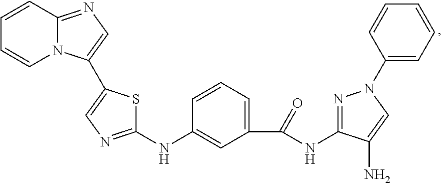
and pharmaceutically acceptable salts thereof.

16. The compound of claim 14 which is selected from the group consisting of: 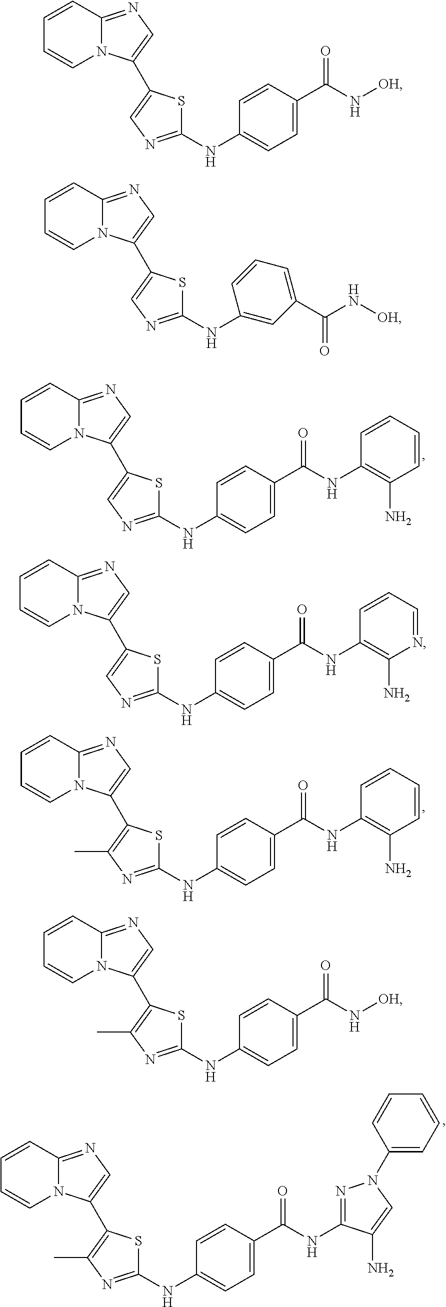
and pharmaceutically acceptable salts thereof.

17. The compound of claim 13, wherein R¹ is methyl; R², R³, R⁴ and R⁵ are H; R⁶ is H, alkyl or haloalkyl; each R⁷ is independently fluoro, chloro, bromo, or methyl; n is 0, 1, or 2; and R⁸ is hydroxyl, aryl or heteroaryl, wherein aryl or heteroaryl are substituted with —NH₂ or —OH at a ring position adjacent to attachment of the —CONH-moiety and R⁸ is optionally substituted with one or more groups R¹⁰ selected from amino, halo, alkyl, cycloalkyl, heterocyclyl, aryl, and heteroaryl.

18. The compound of claim 17 which is selected from the group consisting of: 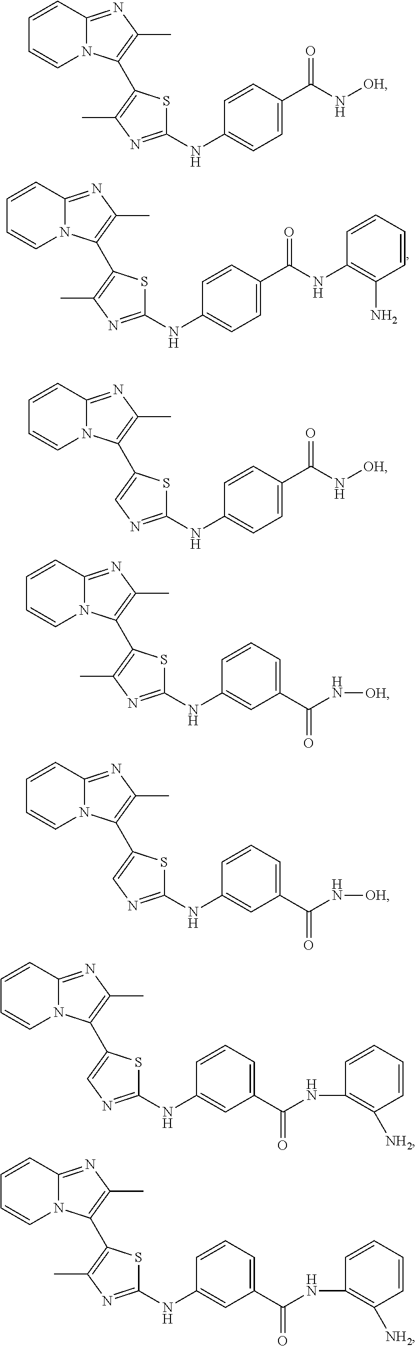 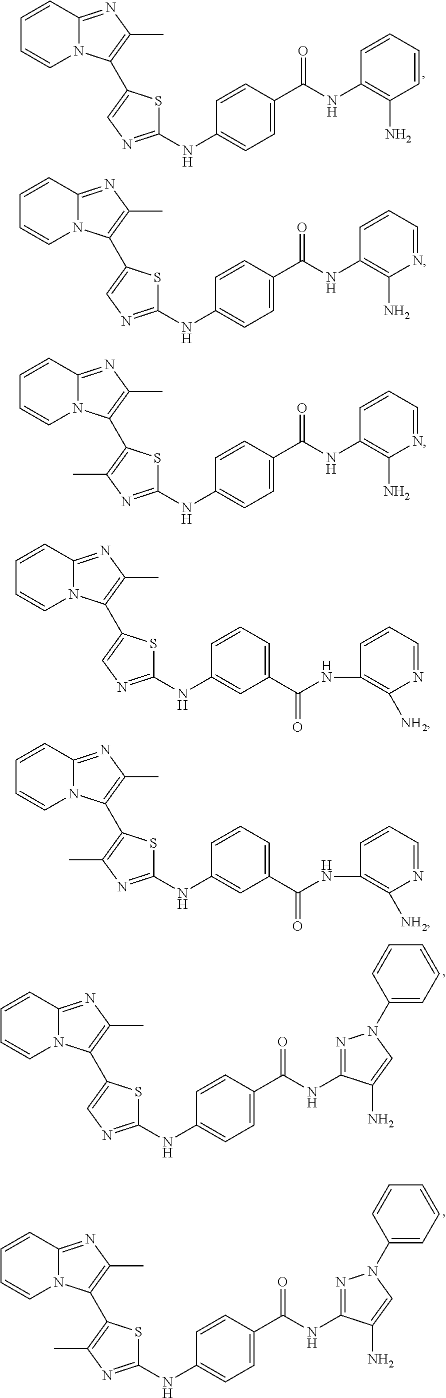 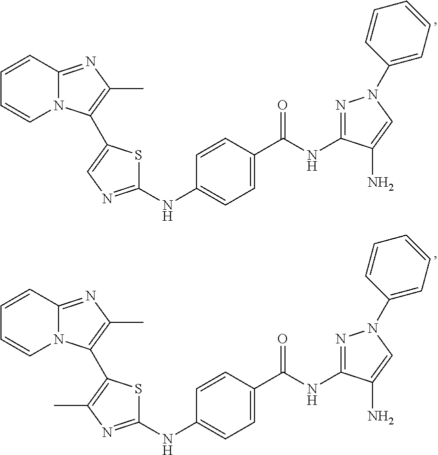
and pharmaceutically acceptable salts thereof.

19. The compound of claim 17 which is selected from the group consisting of: 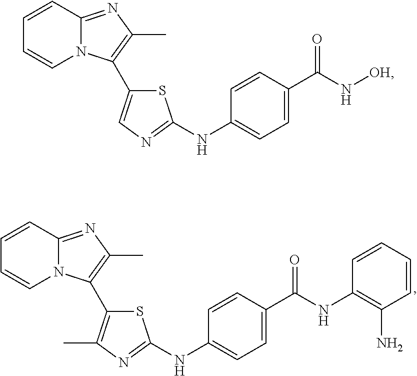
and pharmaceutically acceptable salts thereof.

20. The compound of claim 13, wherein at least two of R¹, R², R³, R⁴ and R⁵ are H and each non-hydrogen R¹, R², R³, R⁴ and R⁵ is selected from chloro, fluoro, bromo, methyl, ethyl, propyl, methoxy, ethoxy, carboxy, cyano, methoxymethyl, ethoxyethyl, propoxyethyl, methoxyethoxy, trifluoromethyl, hydroxyethoxy, dimethylamino, diethylamino, dimethylaminomethyl, dimethylamino ethyl, diethylaminomethyl, dimethylaminoethoxy, trifluoromethoxymethyl, trifluoroethoxymethyl, 3-oxetanoxy, trifluoroethylaminomethyl, N-methyl-N-methoxyethyl-aminoethyl, cyclopropanylmethyl, cyclobutoxy, 1-cyclopropanylethoxy, cyclopropanylmethylaminomethyl, 4-methylpiperazin-1-carbonyl, isoindolin-2-yl, N-methoxyethylcarbamoyl, N-(morpholin-4-yl)-ethylcarbamoyl, dimethylaminoethylamino, methylcarboxy, N,N-dimethylaminoethylcarbamoyl, benzyl, phenylethyl, trifluoromethylphenylethyl, phenoxymethyl, fluorophenoxymethyl, phenylethylaminomethyl, benzylaminomethyl, triazinylmethyl, piperidinylmethyl, piperidinyloxy, trifluoromethylpiperidinylmethyl, pyridinyloxymethyl, pyridinylmethoxy, tetrahydropyrazinyloxy, methylpiperazinylmethyl, pyrrolidin-1-yl, pyrrolidin-2-yl, pyrrolidin-3-yl, pyrrolidin-1-ylmethyl, pyrrolidin-2-ylmethyl, pyrrolidin-3-ylmethyl, pyrrolidin-1-ylethoxy, pyrrolidin-2-ylethoxy, pyrrolidin-3-ylethoxy, imidazol-1-ylmethyl, imidazol-2-ylmethyl, imidazol-4-ylmethyl, imidazolidin-1-yl, imidazolidin-2-yl, imidazolidin-4-yl, imidazolidin-1-ylmethyl, imidazolidin-2-ylmethyl, imidazolidin-4-ylmethyl, imidazolin-1-yl, imidazolin-2-yl, imidazolin-4-yl, pyrazolidin-1-yl, pyrazolidin-3-yl, pyrazolidin-4-yl, pyrazolin-1-yl, pyrazolin-3-yl, pyrazolin-4-yl, piperidin-1-yl, piperidin-2-yl, piperidin-3-yl, piperidin-4-yl, piperidin-1-ylmethyl, piperidin-2-ylmethyl, piperidin-3-ylmethyl, piperidin-4-ylmethyl, piperazin-1-yl, piperazin-2-yl, piperazin-3-yl, morpholin-2-yl, morpholin-3-yl, morpholin-4-yl, morpholin-2-ylmethyl, morpholin-3-ylmethyl, morpholin-4-ylmethyl, morpholin-2-ylethoxy, morpholin-3-ylethoxy and morpholin-4-ylethoxy;
R⁶ is H, alkyl or haloalkyl;
R⁷ is independently fluoro, chloro, bromo, or methyl;
n is 0, 1, or 2; and
R⁸ is hydroxyl, aryl or heteroaryl, wherein aryl or heteroaryl are substituted with —NH₂ or —OH and R⁸ is optionally substituted with one or more groups R¹⁰ selected from amino, halo, alkyl, cycloalkyl, heterocyclyl, aryl, and heteroaryl.

21. The compound of claim 20 which is selected from the group consisting of: 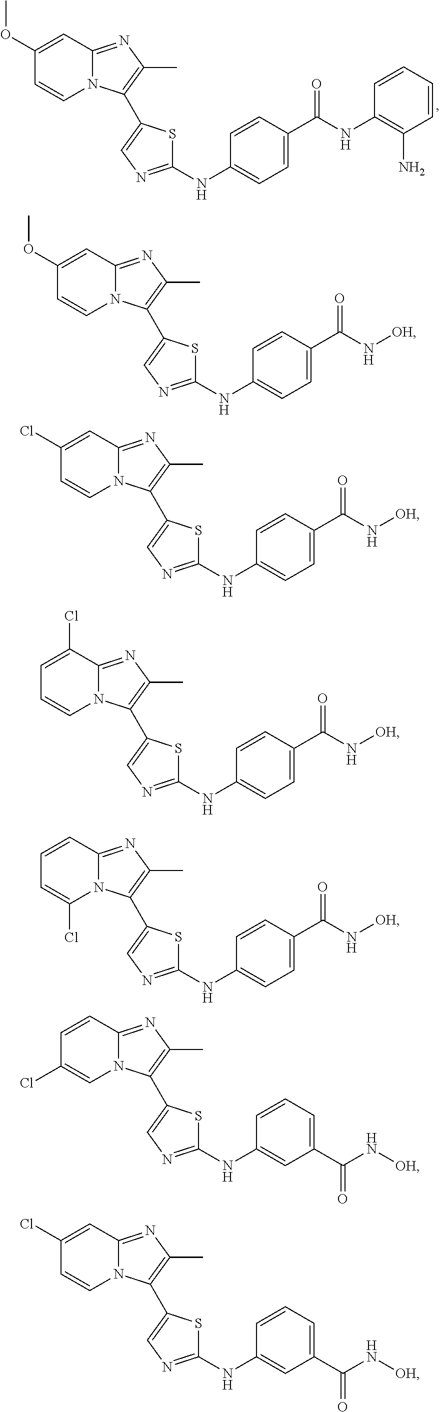 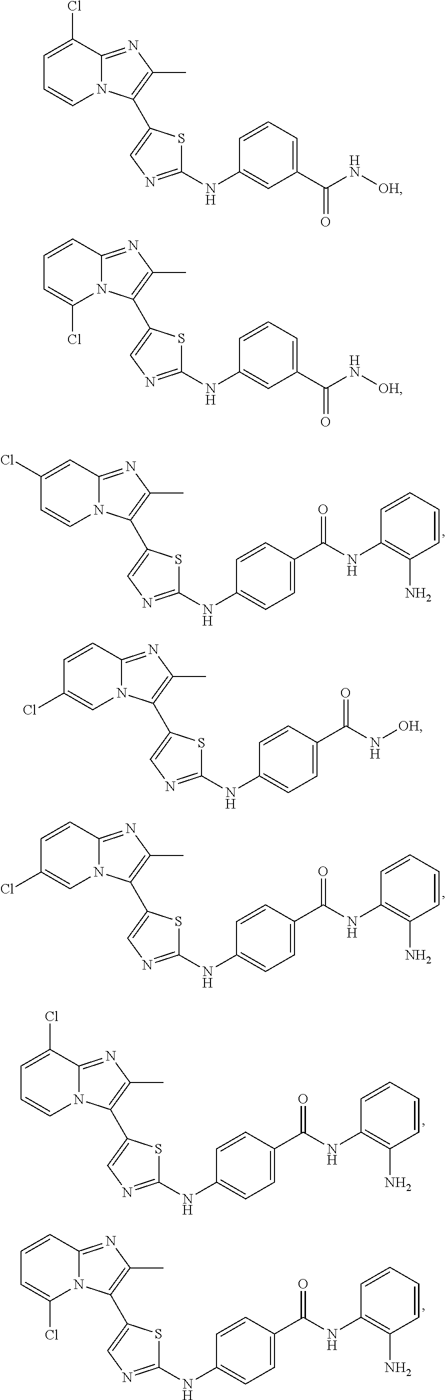 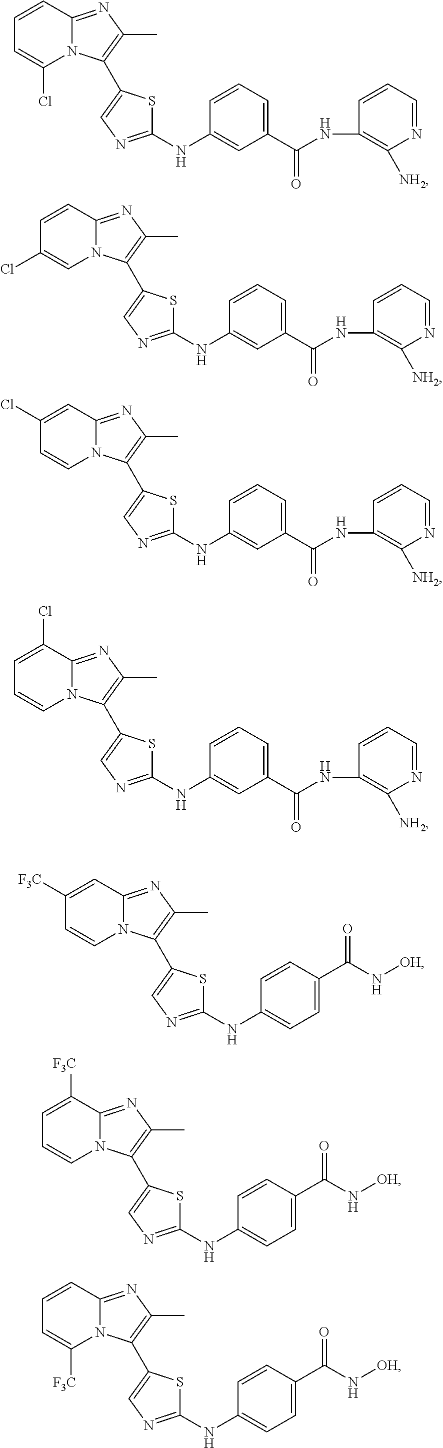 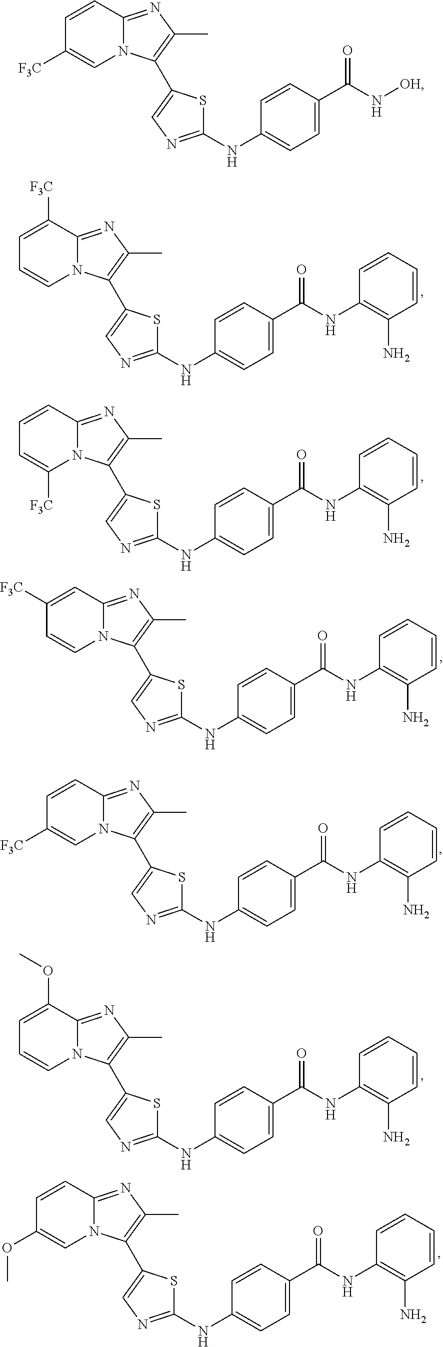 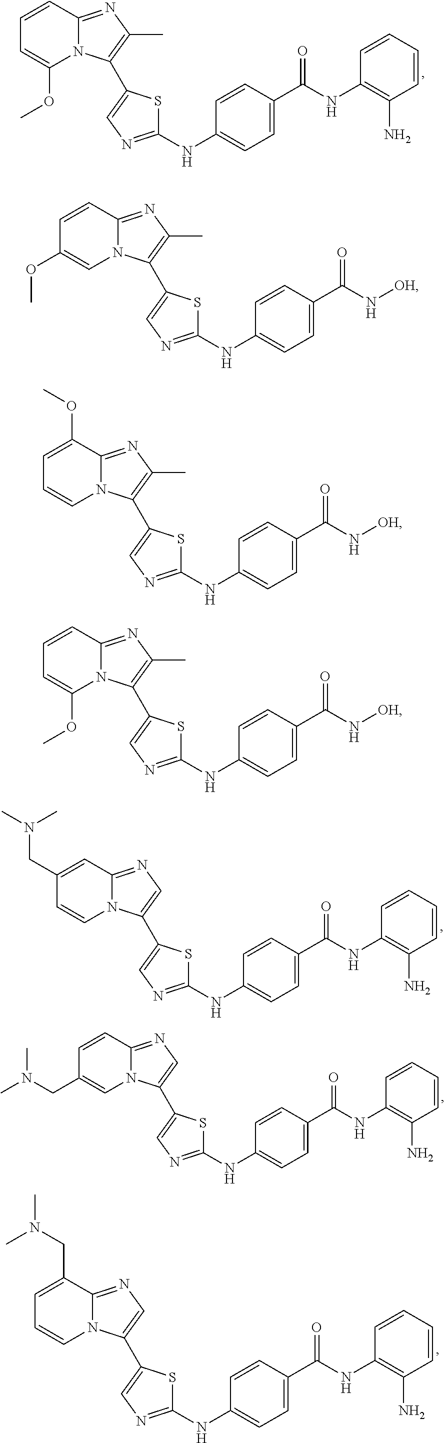 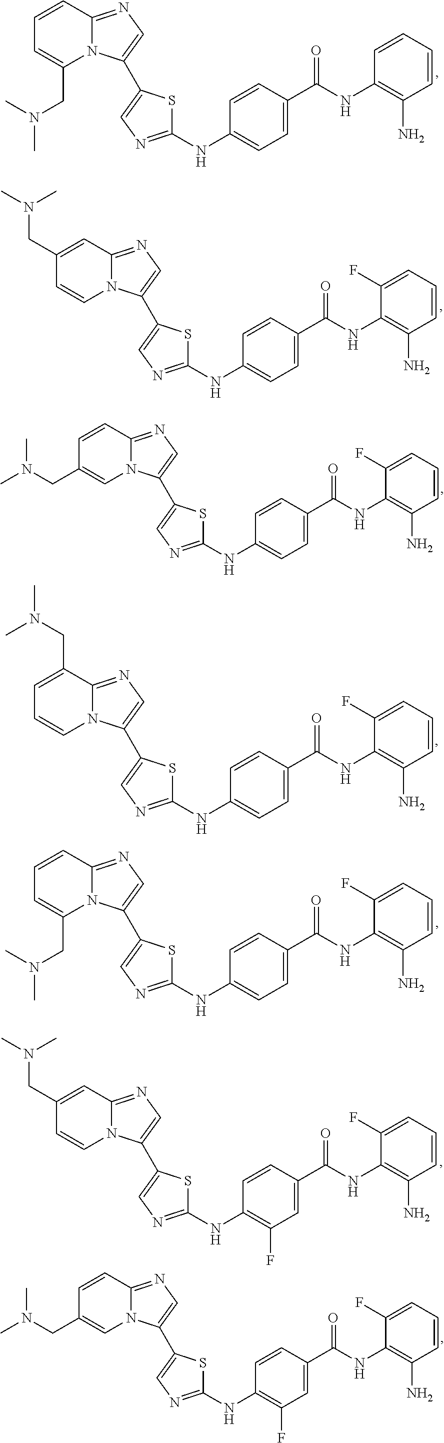 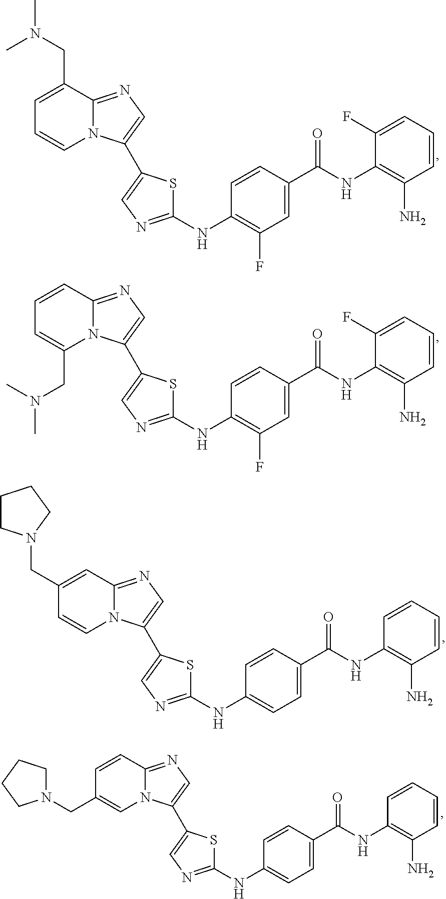 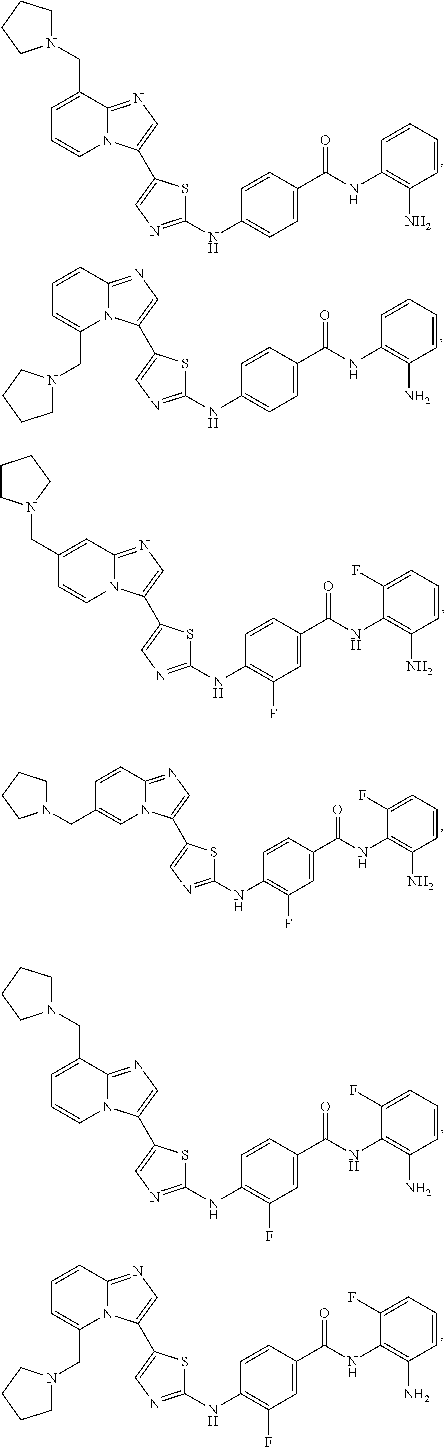 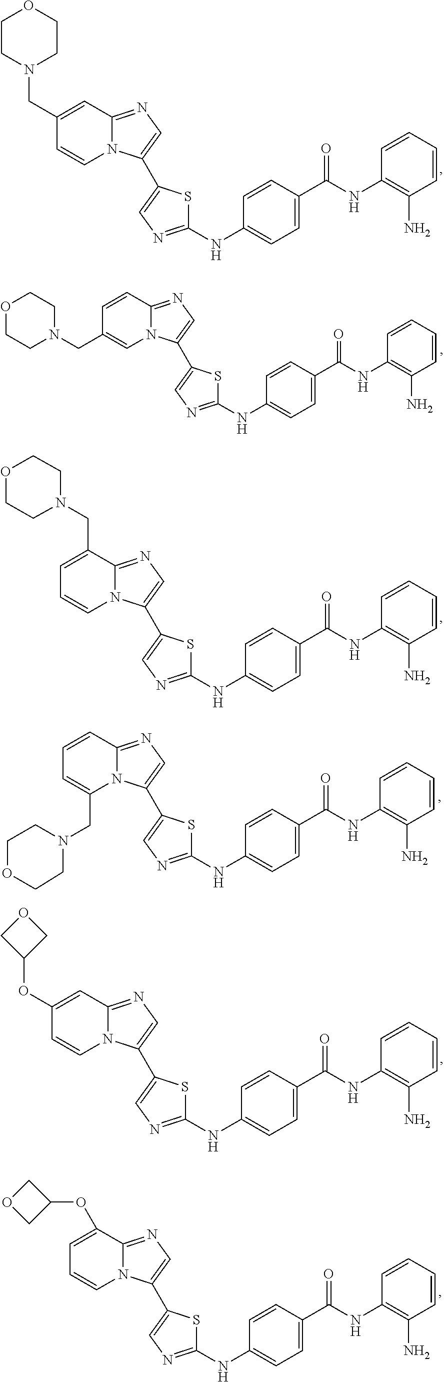 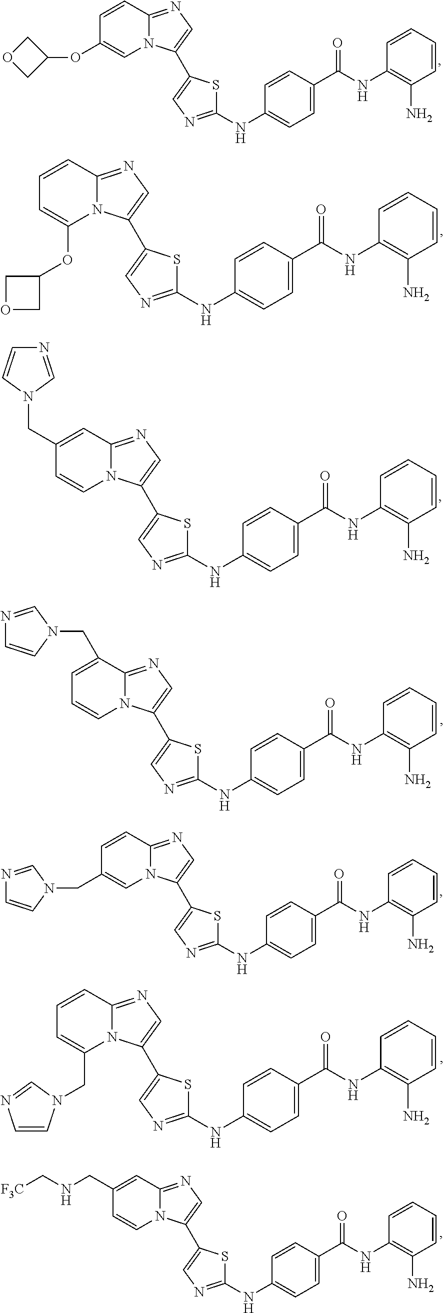 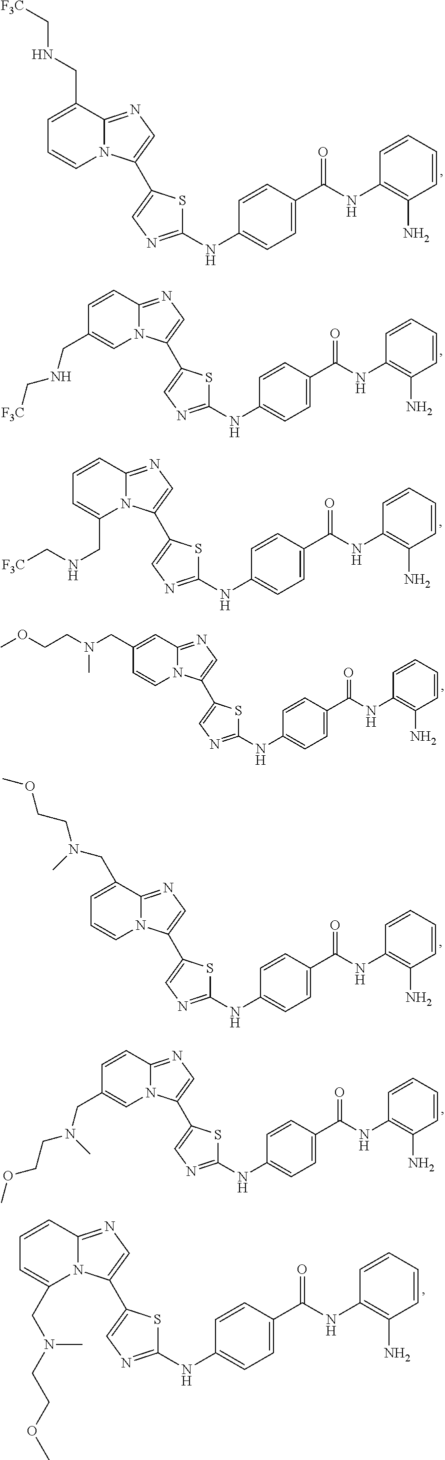 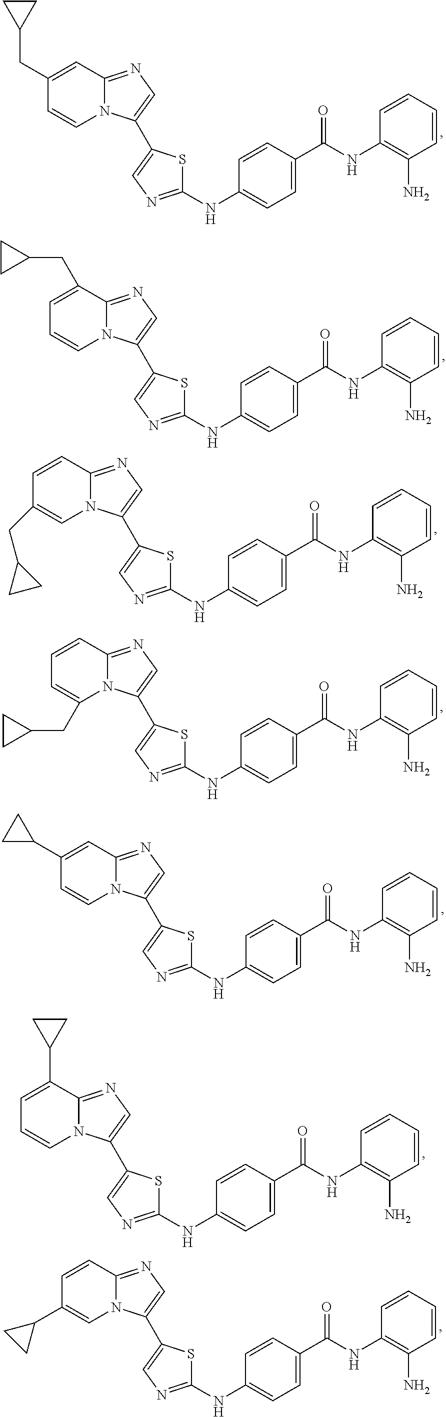 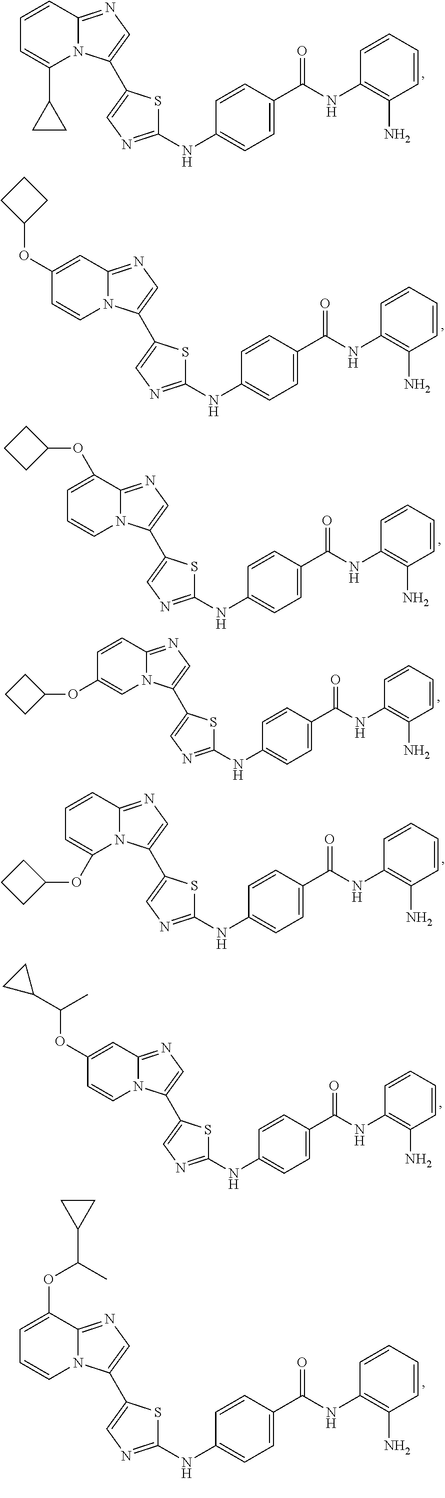 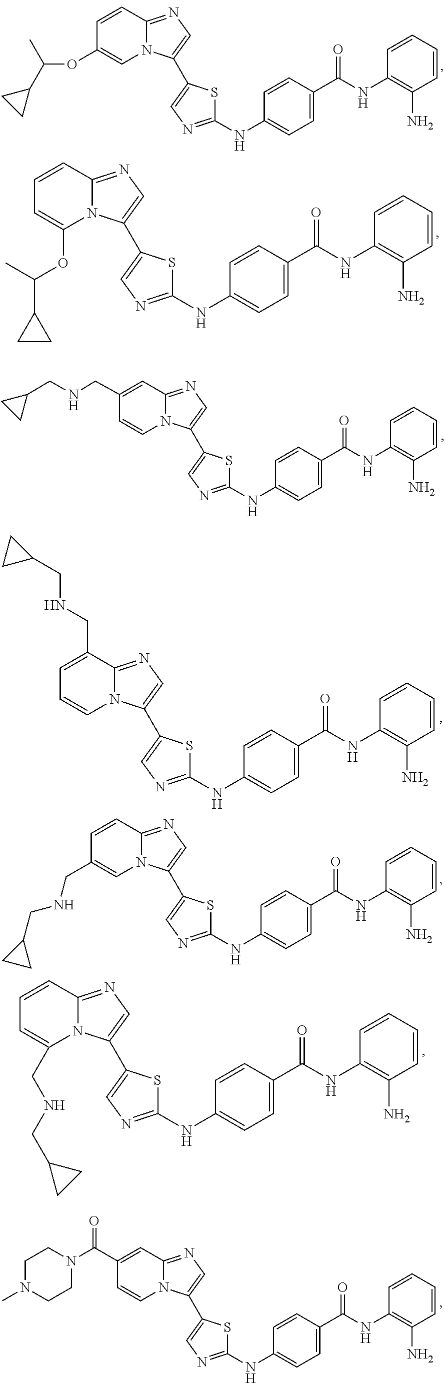 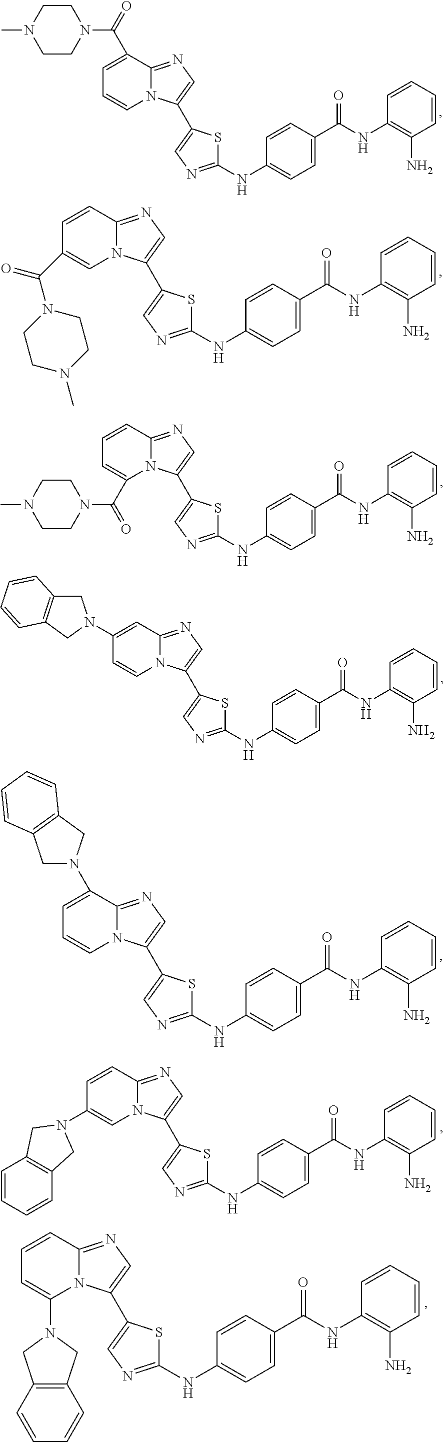
and pharmaceutically acceptable salts thereof.

22. The compound of claim 20 which is selected from the group consisting of: 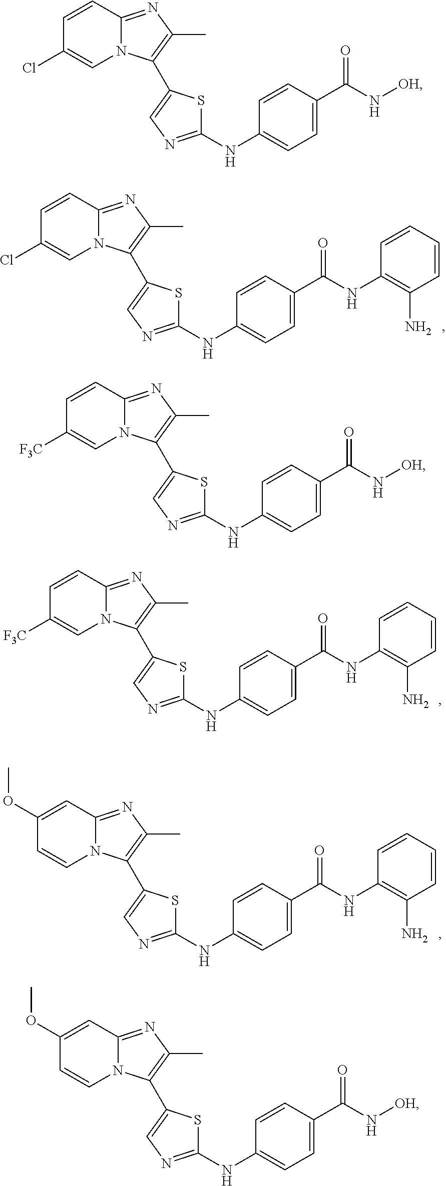
or a pharmaceutically acceptable salt thereof.

23. The compound of claim 1 which is selected from those of Formula (I-c) and pharmaceutically acceptable salts thereof:

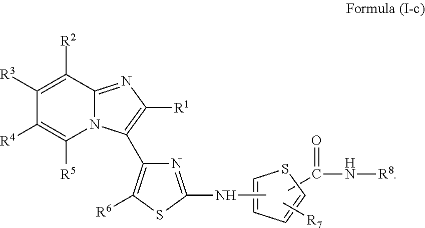

24. The compound of claim 23, wherein R¹, R², R³, R⁴ and R⁵ are H; R⁶ is H, alkyl or haloalkyl; R⁷ is fluoro, chloro, bromo, or methyl; n is 0 or 1; and R⁸ is hydroxyl, aryl or heteroaryl, wherein aryl or heteroaryl are substituted with —NH₂ or —OH at a ring position adjacent to attachment of the —CONH-moiety and R⁸ is optionally substituted with one or more groups R¹⁰ selected from amino, halo, alkyl, cycloalkyl, heterocyclyl, aryl, and heteroaryl.

25. The compound of claim 24 which is selected from the group consisting of: 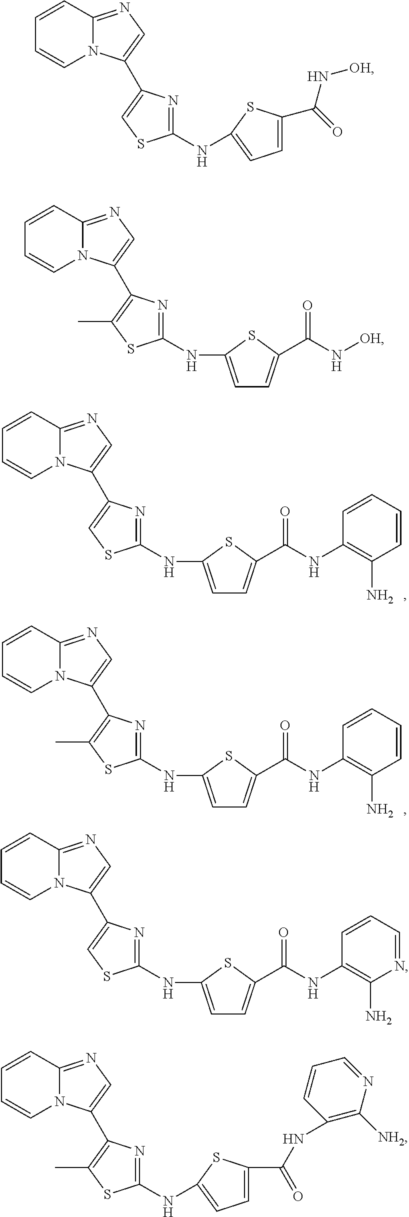 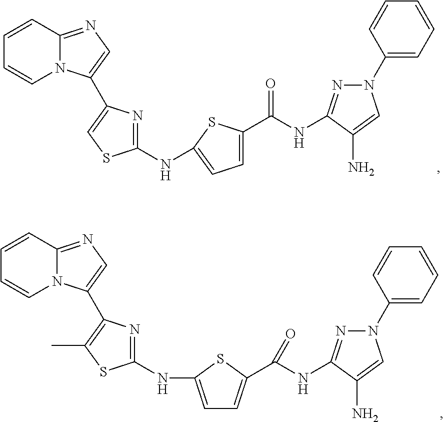
and pharmaceutically acceptable salts thereof.

26. The compound of claim 24 which is selected from the group consisting of: 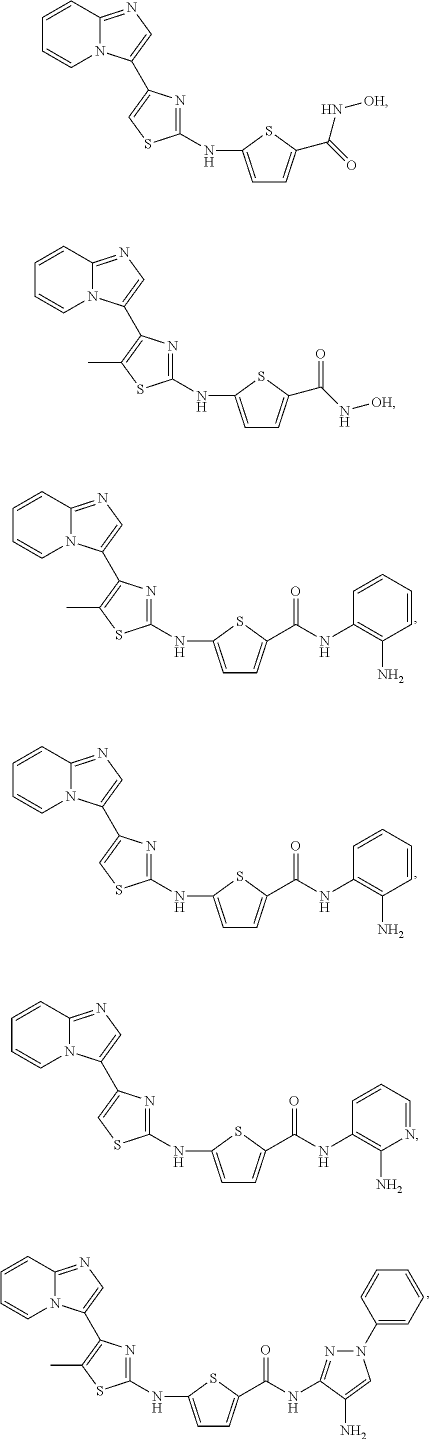
and pharmaceutically acceptable salts thereof.

27. The compound of claim 23, wherein R¹ is methyl; R², R³, R⁴ and R⁵ are H; R⁶ is H, alkyl or haloalkyl; R⁷ is fluoro, chloro, bromo, or methyl; n is 0 or 1; and R⁸ is hydroxyl, aryl or heteroaryl, wherein aryl or heteroaryl are substituted with —NH₂ or —OH at a ring position adjacent to attachment of the —CONH-moiety and R⁸ is optionally substituted with one or more groups R¹⁰ selected from amino, halo, alkyl, cycloalkyl, heterocyclyl, aryl, and heteroaryl.

28. The compound of claim 27 which is selected from the group consisting of: 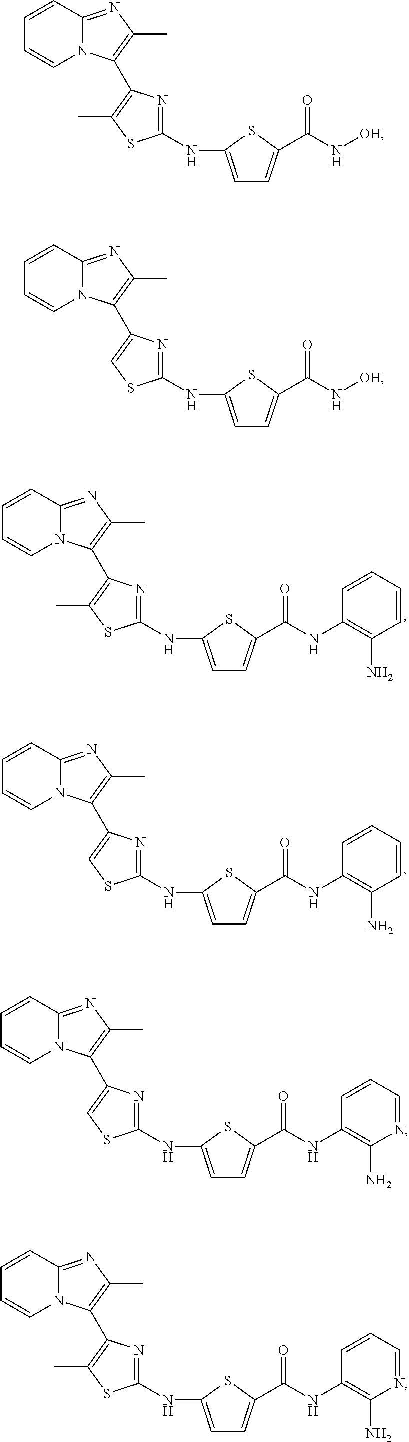 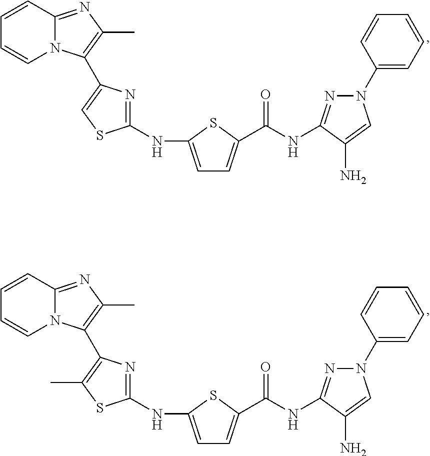
and pharmaceutically acceptable salts thereof.

29. The compound of claim 27 which is selected from the group consisting of: 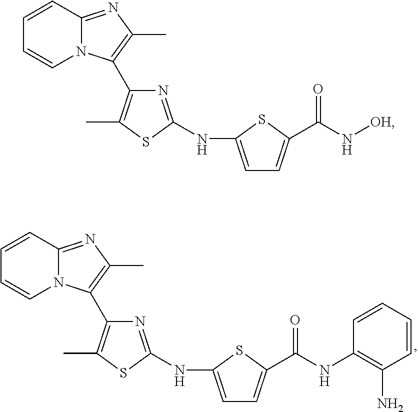
and pharmaceutically acceptable salts thereof.

30. The compound of claim 23, wherein at least two of R¹, R², R³, R⁴ and R⁵ are H and each non-hydrogen R¹, R², R³, R⁴ and R⁵ is selected from chloro, fluoro, bromo, methyl, ethyl, propyl, methoxy, ethoxy, carboxy, cyano, methoxymethyl, ethoxyethyl, propoxyethyl, methoxyethoxy, trifluoromethyl, hydroxyethoxy, dimethylamino, diethylamino, dimethylaminomethyl, dimethylamino ethyl, diethylaminomethyl, dimethylaminoethoxy, trifluoromethoxymethyl, trifluoroethoxymethyl, 3-oxetanoxy, trifluoroethylaminomethyl, N-methyl-N-methoxyethyl-aminoethyl, cyclopropanylmethyl, cyclobutoxy, 1-cyclopropanylethoxy, cyclopropanylmethylaminomethyl, 4-methylpiperazin-1-carbonyl, isoindolin-2-yl, N-methoxyethylcarbamoyl, N-(morpholin-4-yl)-ethylcarbamoyl, dimethylaminoethylamino, methylcarboxy, N,N-dimethylaminoethylcarbamoyl, benzyl, phenylethyl, trifluoromethylphenylethyl, phenoxymethyl, fluorophenoxymethyl, phenylethylaminomethyl, benzylaminomethyl, triazinylmethyl, piperidinylmethyl, piperidinyloxy, trifluoromethylpiperidinylmethyl, pyridinyloxymethyl, pyridinylmethoxy, tetrahydropyrazinyloxy, methylpiperazinylmethyl, pyrrolidin-1-yl, pyrrolidin-2-yl, pyrrolidin-3-yl, pyrrolidin-1-ylmethyl, pyrrolidin-2-ylmethyl, pyrrolidin-3-ylmethyl, pyrrolidin-1-ylethoxy, pyrrolidin-2-ylethoxy, pyrrolidin-3-ylethoxy, imidazol-1-ylmethyl, imidazol-2-ylmethyl, imidazol-4-ylmethyl, imidazolidin-1-yl, imidazolidin-2-yl, imidazolidin-4-yl, imidazolidin-1-ylmethyl, imidazolidin-2-ylmethyl, imidazolidin-4-ylmethyl, imidazolin-1-yl, imidazolin-2-yl, imidazolin-4-yl, pyrazolidin-1-yl, pyrazolidin-3-yl, pyrazolidin-4-yl, pyrazolin-1-yl, pyrazolin-3-yl, pyrazolin-4-yl, piperidin-1-yl, piperidin-2-yl, piperidin-3-yl, piperidin-4-yl, piperidin-1-ylmethyl, piperidin-2-ylmethyl, piperidin-3-ylmethyl, piperidin-4-ylmethyl, piperazin-1-yl, piperazin-2-yl, piperazin-3-yl, morpholin-2-yl, morpholin-3-yl, morpholin-4-yl, morpholin-2-ylmethyl, morpholin-3-ylmethyl, morpholin-4-ylmethyl, morpholin-2-ylethoxy, morpholin-3-ylethoxy and morpholin-4-ylethoxy;
R⁶ is H, alkyl or haloalkyl;
R⁷ is fluoro, chloro, bromo, or methyl;
n is 0 or 1; and
R⁸ is hydroxyl, aryl or heteroaryl, wherein aryl or heteroaryl are substituted with —NH₂ or —OH and R⁸ is optionally substituted with one or more R¹⁰ selected from amino, halo, alkyl, cycloalkyl, heterocyclyl, aryl, and heteroaryl.

31. The compound of claim 30 which is selected from the group consisting of: 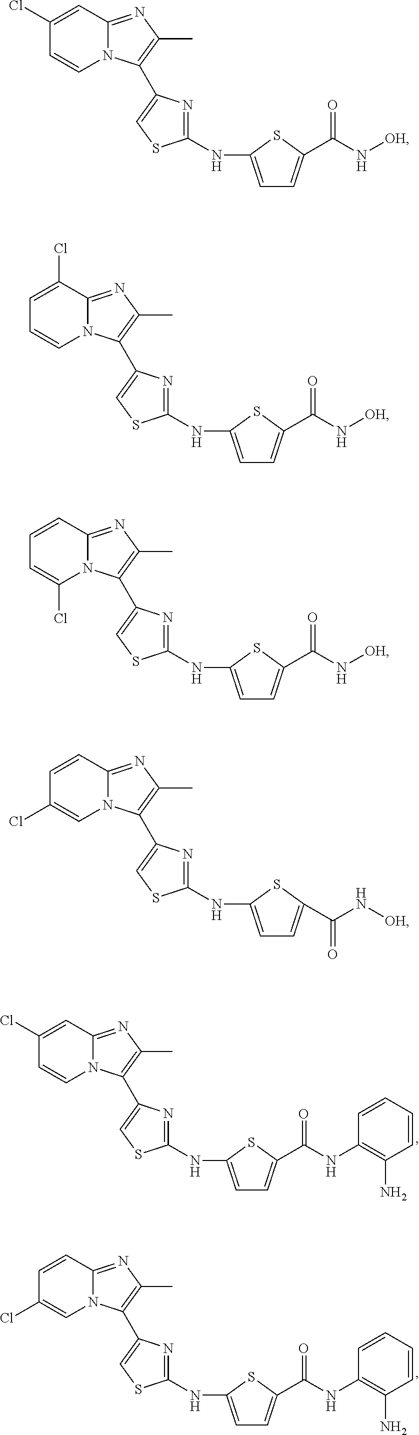 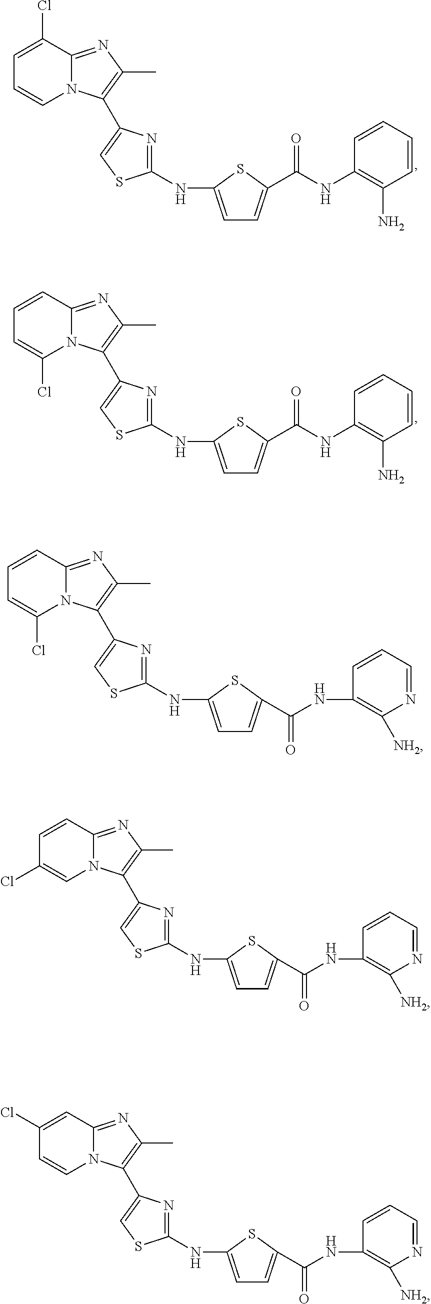 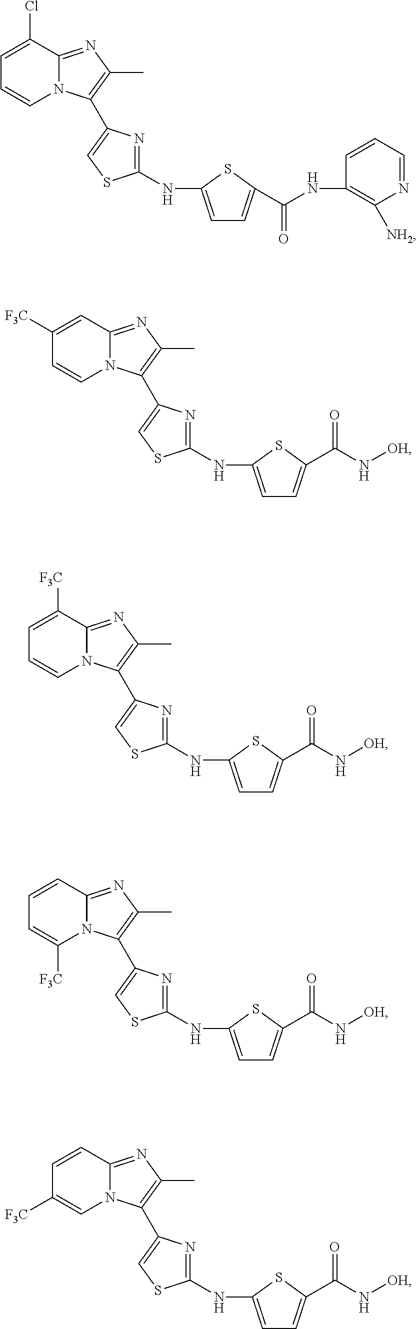 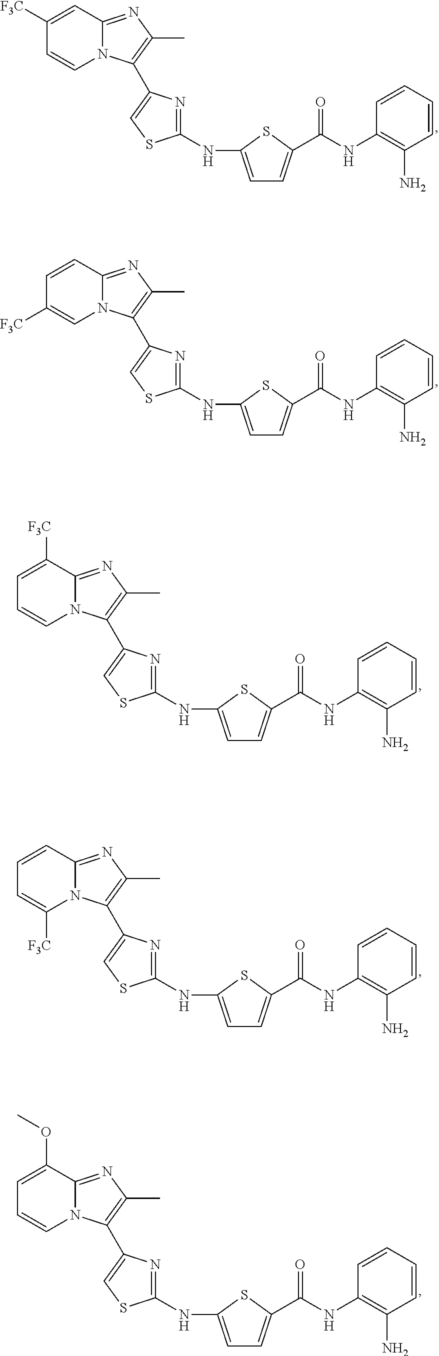 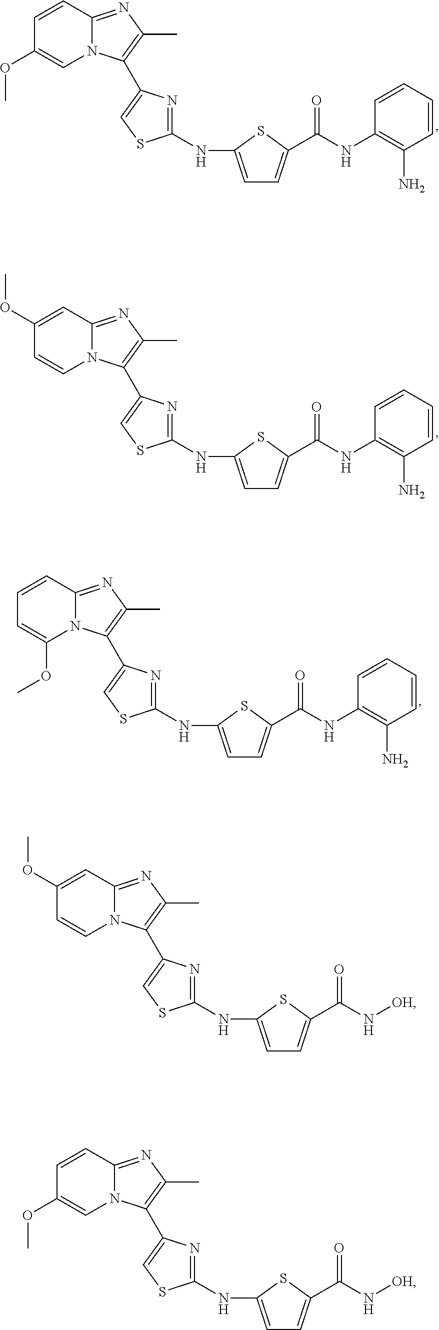 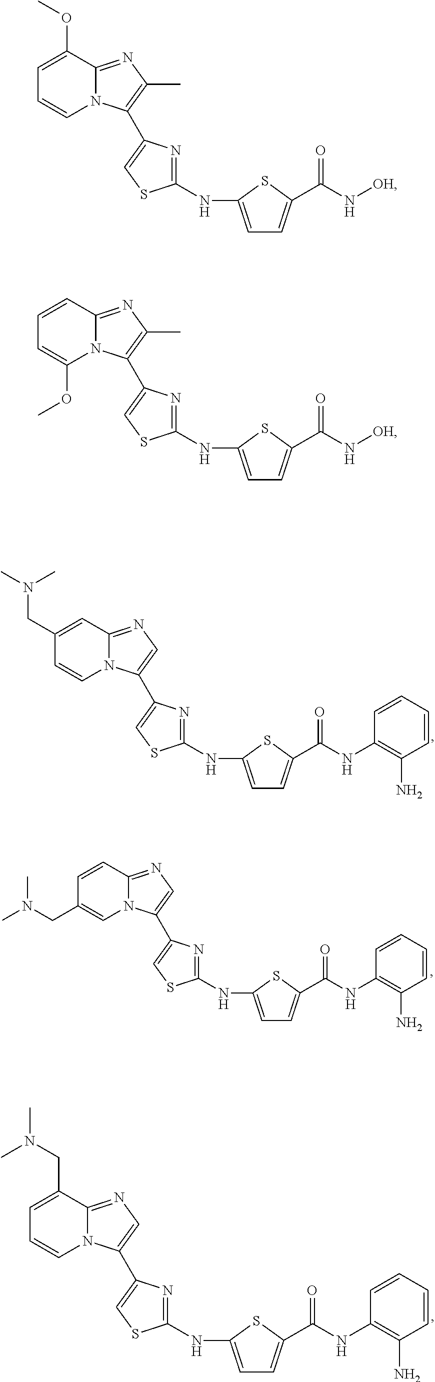 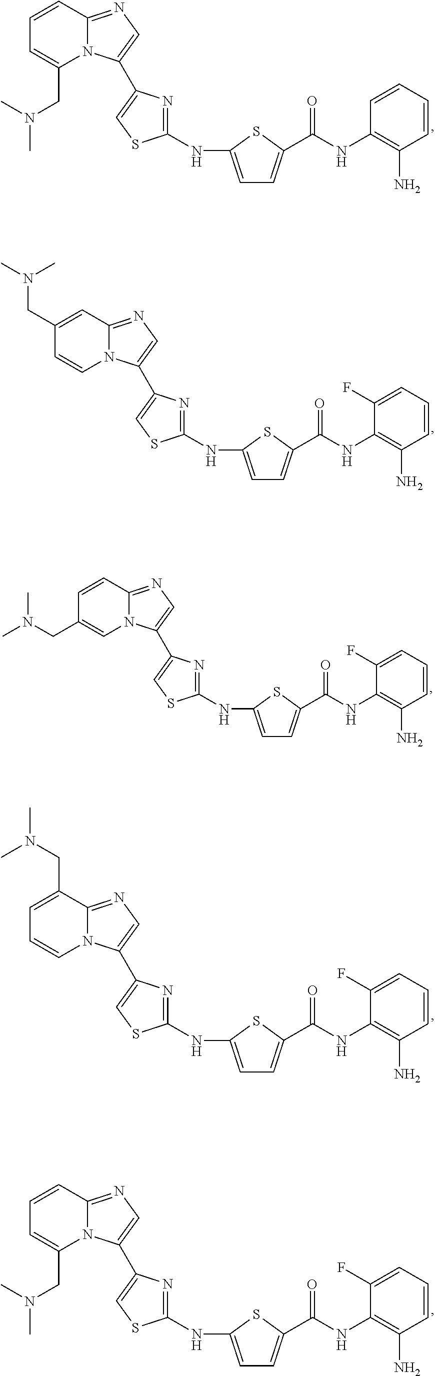 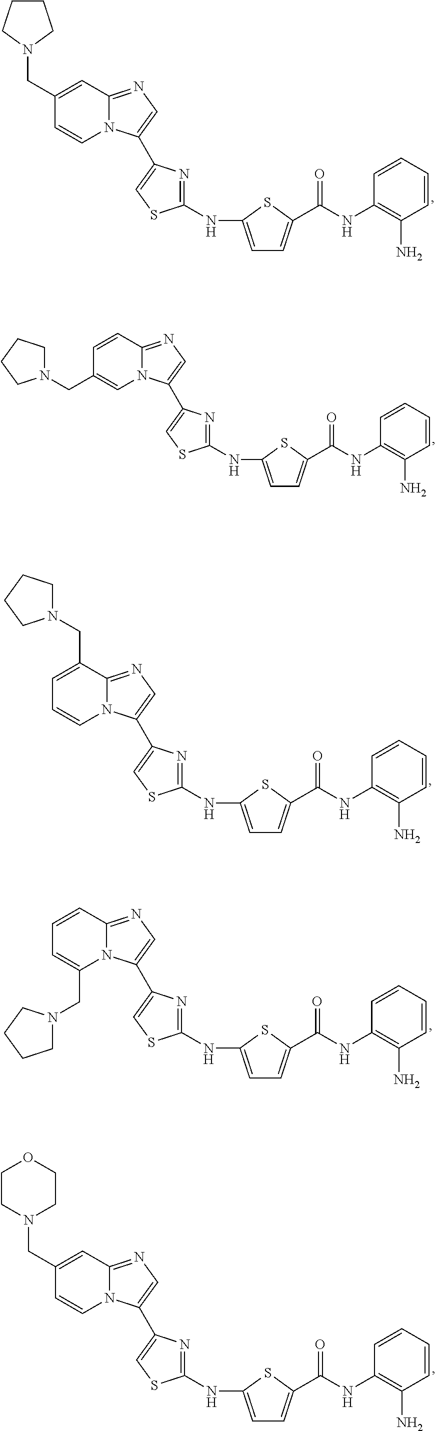 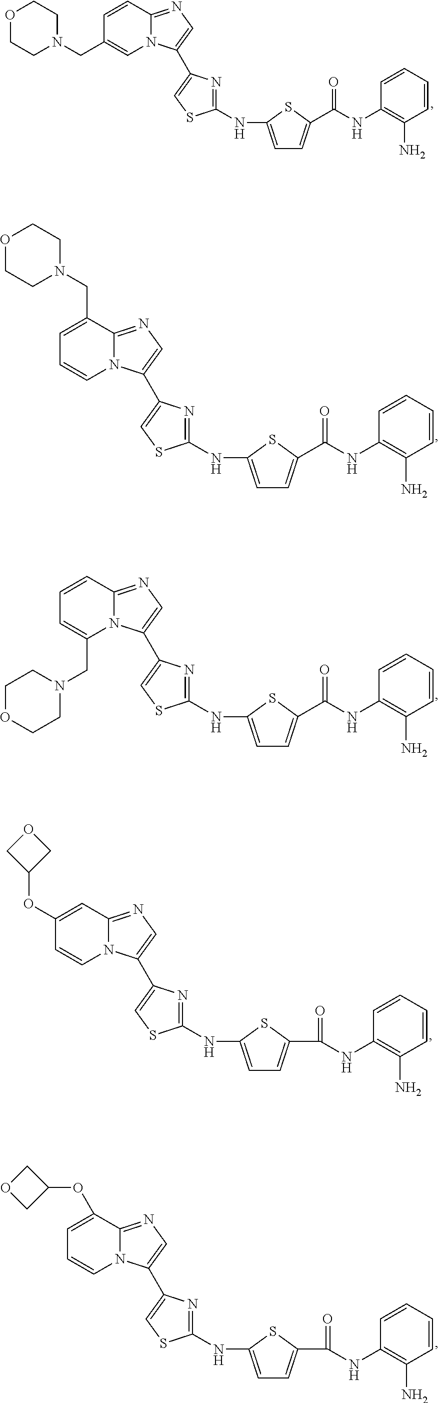 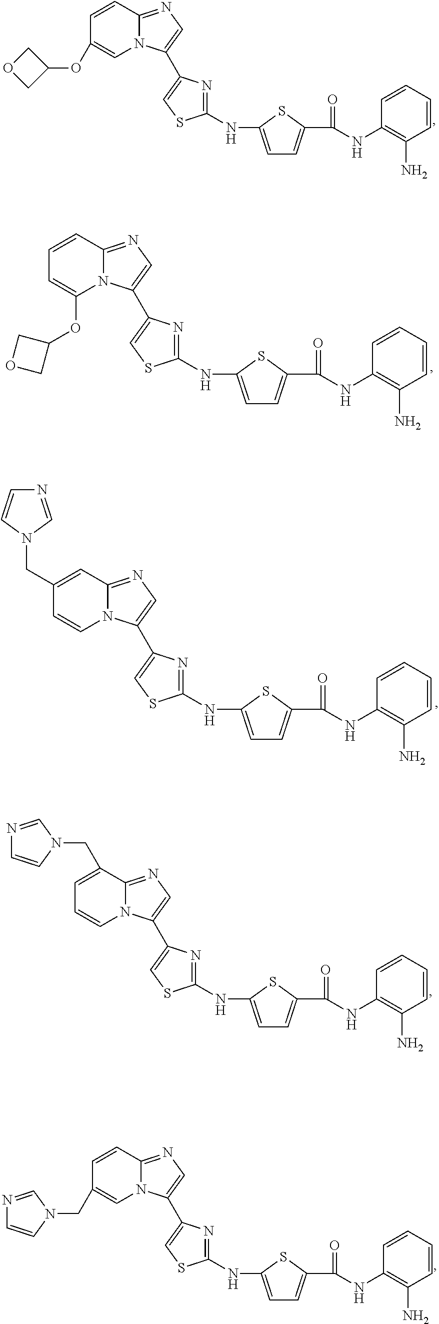 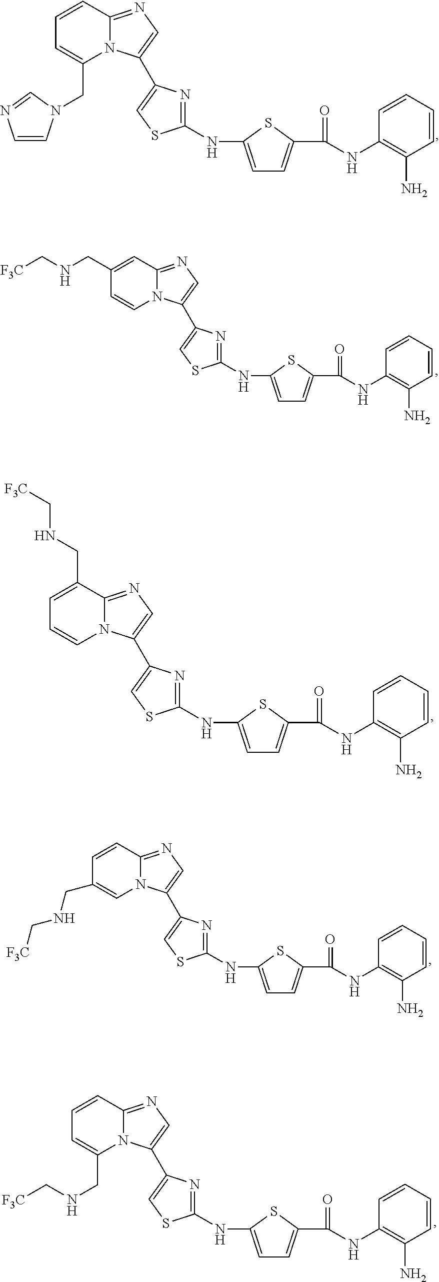 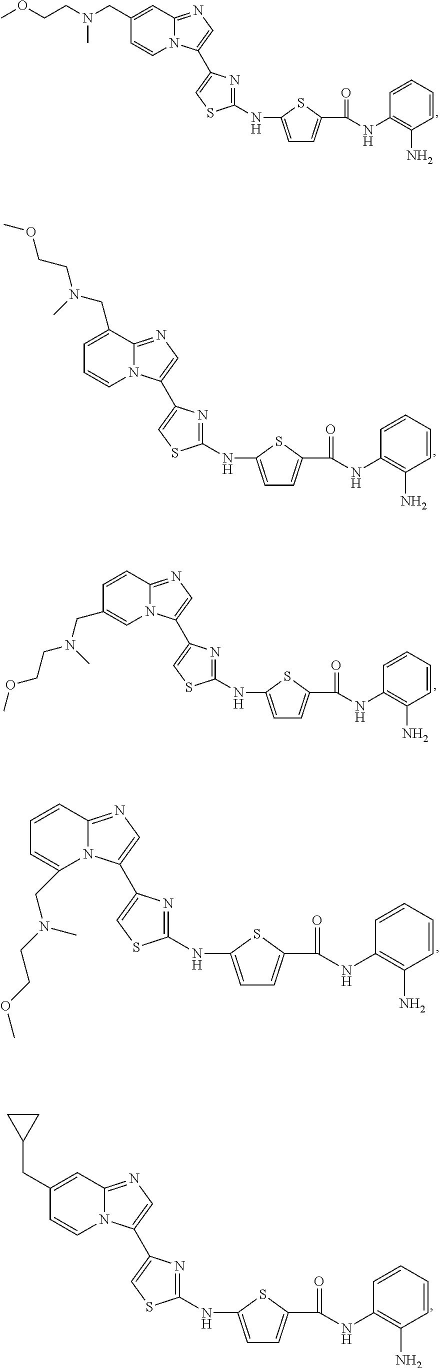 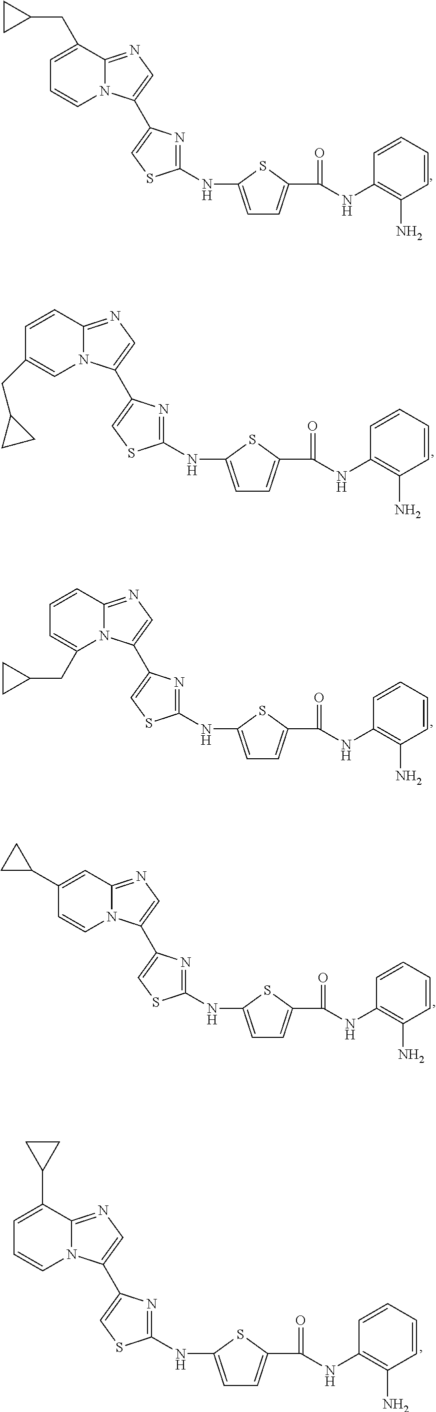 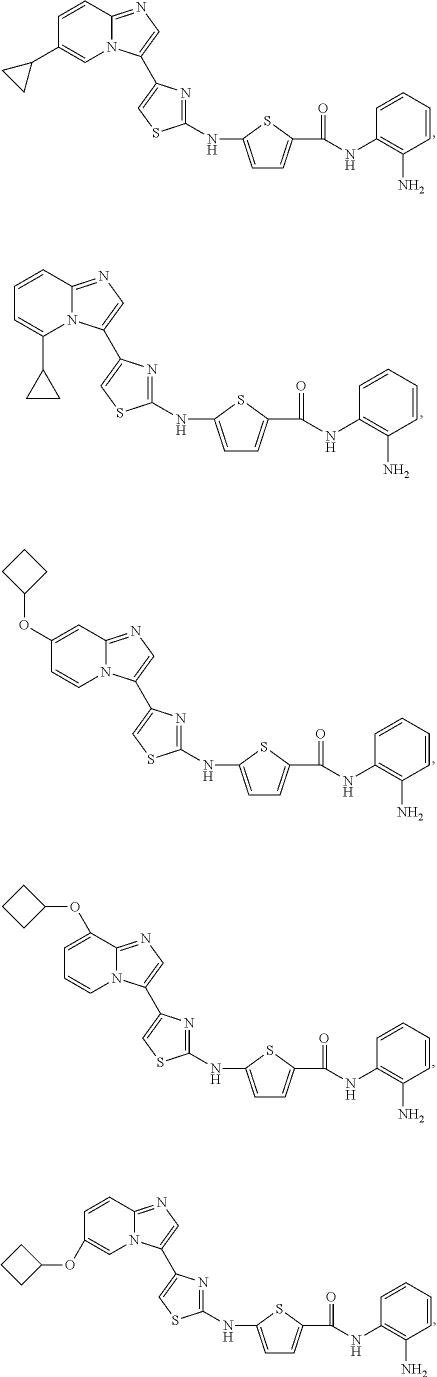 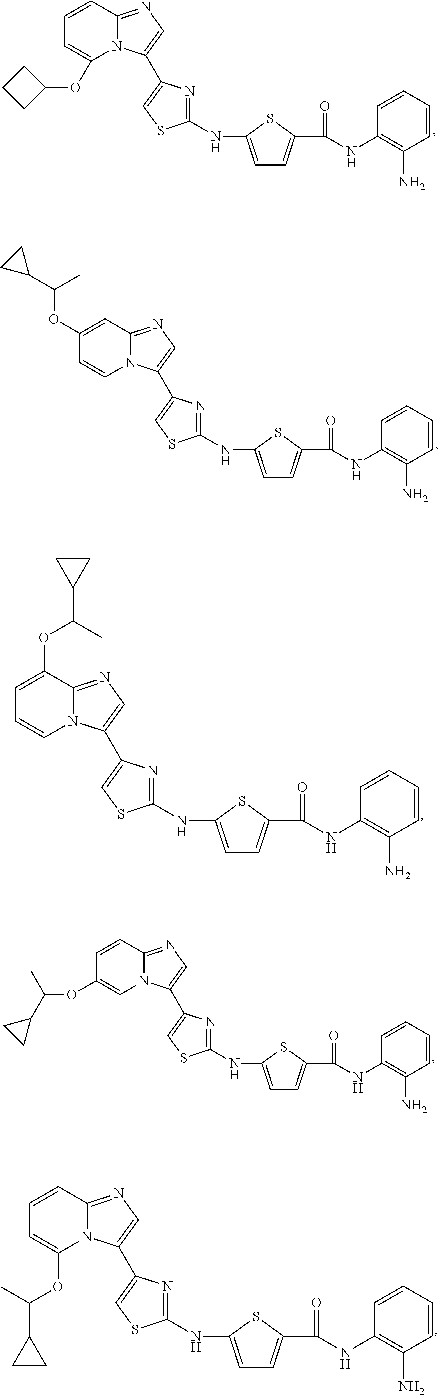 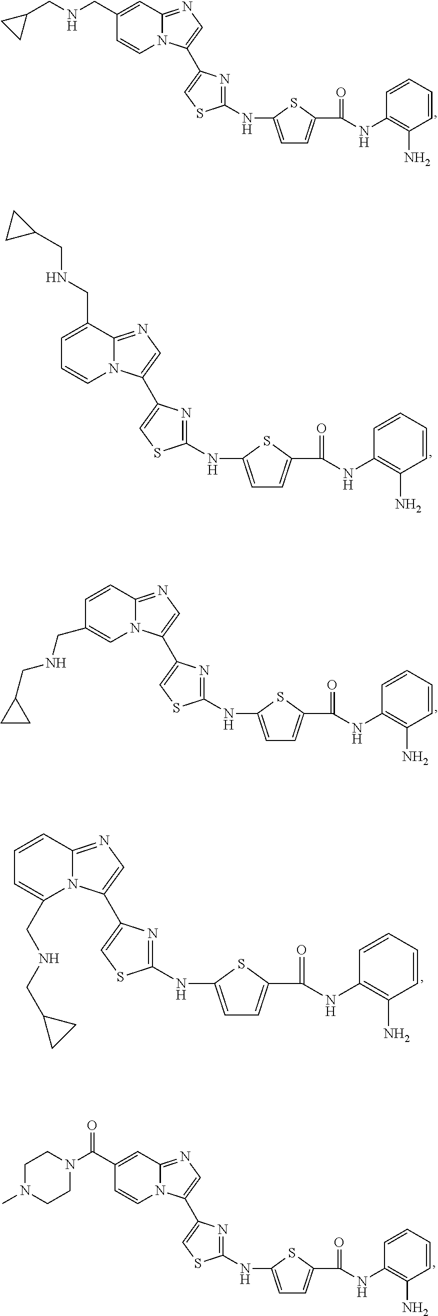 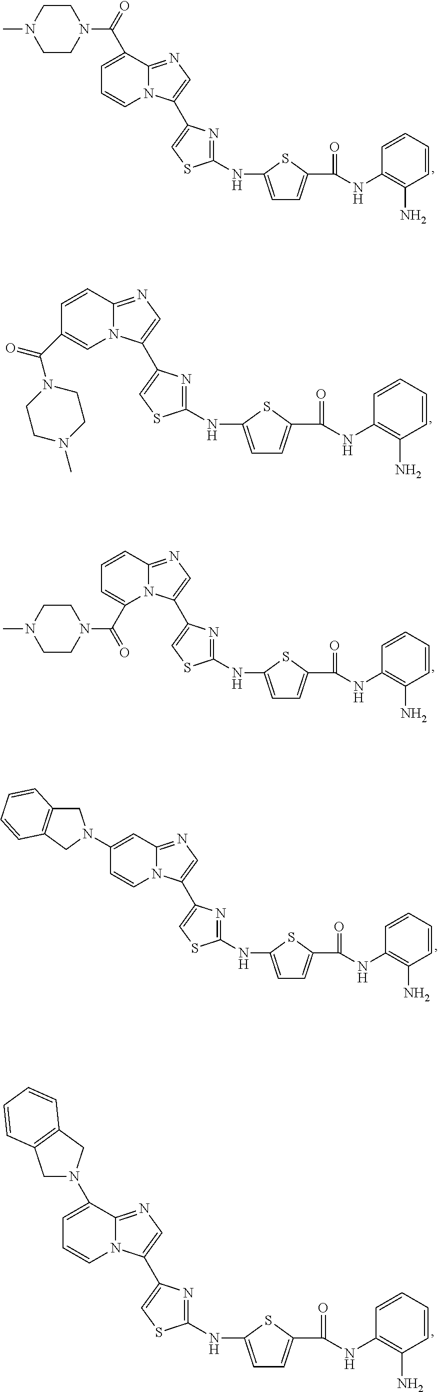 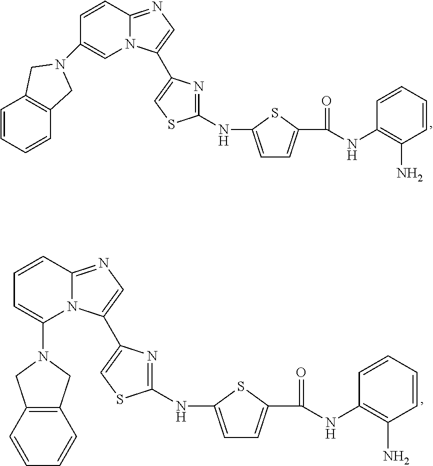
and pharmaceutically acceptable salts thereof.

32. The compound of claim 31 which is selected from the group consisting of: 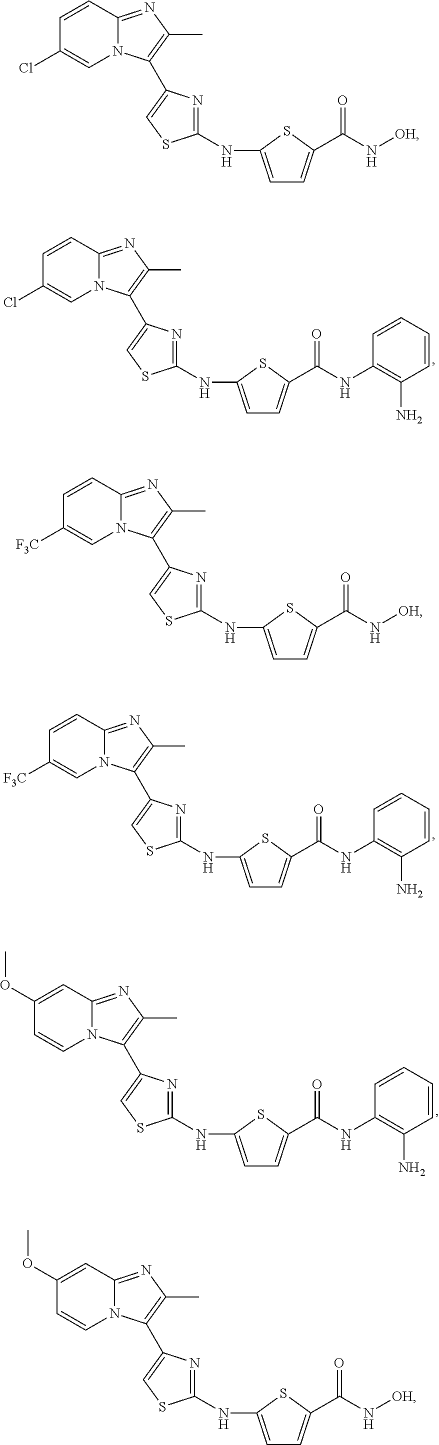
and pharmaceutically acceptable salts thereof.

33. The compound of claim 1 which is selected from those of Formula (I-d) and pharmaceutically acceptable salts thereof:

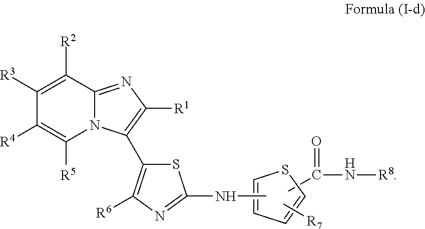

34. The compound of claim 33, wherein R¹, R², R³, R⁴ and R⁵ are H; R⁶ is H, alkyl or haloalkyl; R⁷ is fluoro, chloro, bromo, or methyl; n is 0 or 1; and R⁸ is hydroxyl, aryl or heteroaryl, wherein aryl or heteroaryl are substituted with —NH₂ or —OH at a ring position adjacent to attachment of the —CONH-moiety and R⁸ is optionally substituted with one or more groups R¹⁰ selected from amino, halo, alkyl, cycloalkyl, heterocyclyl, aryl, and heteroaryl.

35. The compound of claim 34 which is selected from the group consisting of: 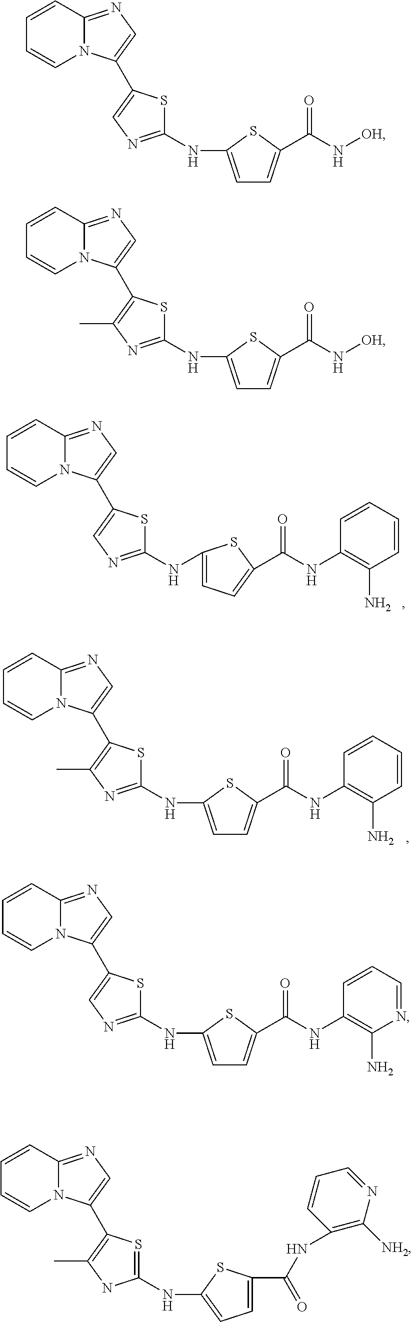 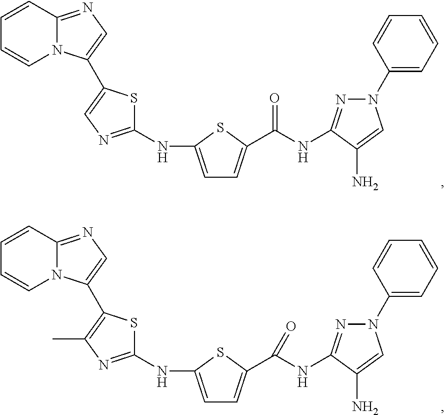
and pharmaceutically acceptable salts thereof.

36. The compound of claim 35 which is selected from the group consisting of: 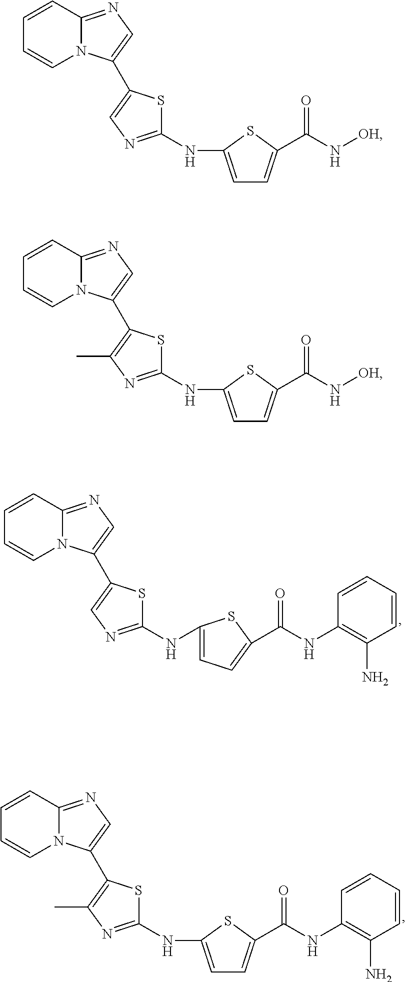
and pharmaceutically acceptable salts thereof.

37. The compound of claim 33, wherein R¹ is methyl; R², R³, R⁴ and R⁵ are H; R⁶ is H, alkyl or haloalkyl; R⁷ is fluoro, chloro, bromo, or methyl; n is 0 or 1; and R⁸ is hydroxyl, aryl or heteroaryl, wherein aryl or heteroaryl are substituted with —NH₂ or —OH at a ring position adjacent to attachment of the —CONH-moiety and R⁸ is optionally substituted with one or more groups R¹⁰ selected from amino, halo, alkyl, cycloalkyl, heterocyclyl, aryl, and heteroaryl.

38. The compound of claim 37 which is selected from the group consisting of: 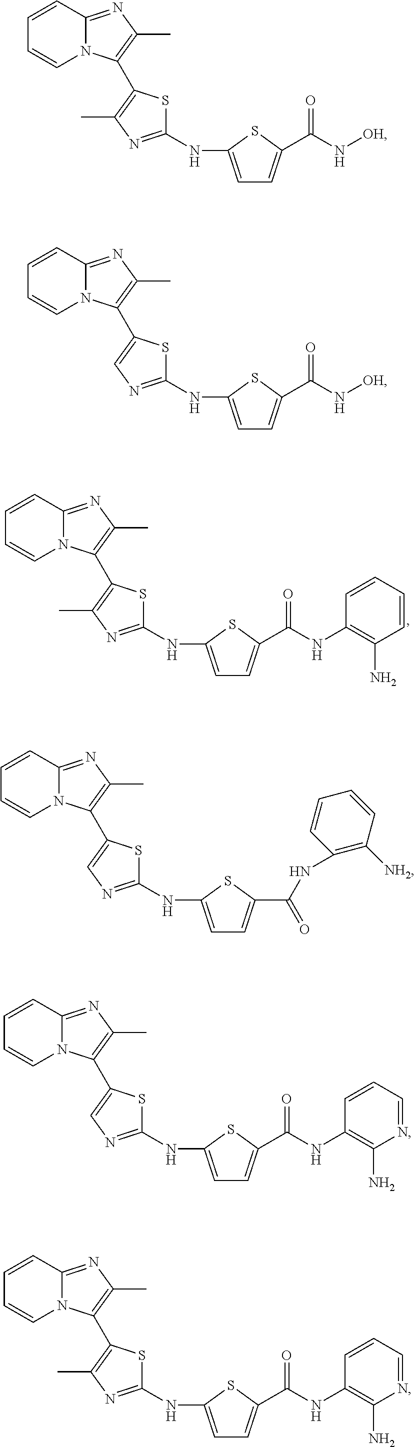 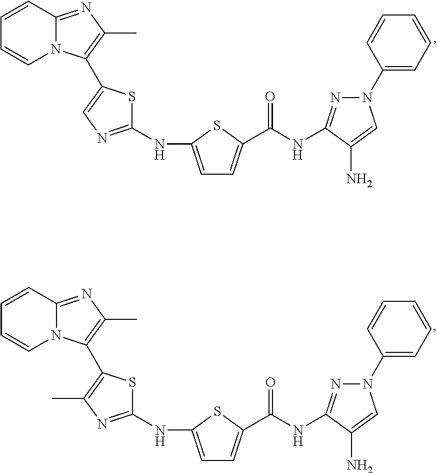
and pharmaceutically acceptable salts thereof.

39. The compound of claim 38 which is selected from the group consisting of: 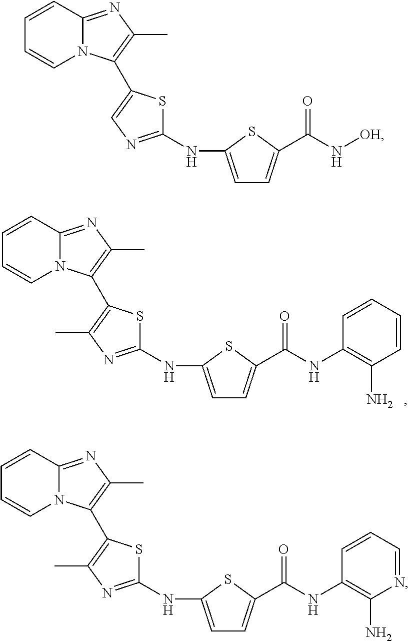
and pharmaceutically acceptable salts thereof.

40. The compound of claim 33, wherein at least two of R¹, R², R³, R⁴ and R⁵ are H and each non-hydrogen R¹, R², R³, R⁴ and R⁵ is selected from chloro, fluoro, bromo, methyl, ethyl, propyl, methoxy, ethoxy, carboxy, cyano, methoxymethyl, ethoxyethyl, propoxyethyl, methoxyethoxy, trifluoromethyl, hydroxyethoxy, dimethylamino, diethylamino, dimethylaminomethyl, dimethylaminoethyl, diethylaminomethyl, dimethylaminoethoxy, trifluoromethoxymethyl, trifluoroethoxymethyl, 3-oxetanoxy, trifluoroethylaminomethyl, N-methyl-N-methoxyethyl-aminoethyl, cyclopropanylmethyl, cyclobutoxy, 1-cyclopropanylethoxy, cyclopropanylmethylaminomethyl, 4-methylpiperazin-1-carbonyl, isoindolin-2-yl, N-methoxyethylcarbamoyl, N-(morpholin-4-yl)-ethylcarbamoyl, dimethylaminoethylamino, methylcarboxy, N,N-dimethylaminoethylcarbamoyl, benzyl, phenylethyl, trifluoromethylphenylethyl, phenoxymethyl, fluorophenoxymethyl, phenylethylaminomethyl, benzylaminomethyl, triazinylmethyl, piperidinylmethyl, piperidinyloxy, trifluoromethylpiperidinylmethyl, pyridinyloxymethyl, pyridinylmethoxy, tetrahydropyrazinyloxy, methylpiperazinylmethyl, pyrrolidin-1-yl, pyrrolidin-2-yl, pyrrolidin-3-yl, pyrrolidin-1-ylmethyl, pyrrolidin-2-ylmethyl, pyrrolidin-3-ylmethyl, pyrrolidin-1-ylethoxy, pyrrolidin-2-ylethoxy, pyrrolidin-3-ylethoxy, imidazol-1-ylmethyl, imidazol-2-ylmethyl, imidazol-4-ylmethyl, imidazolidin-1-yl, imidazolidin-2-yl, imidazolidin-4-yl, imidazolidin-1-ylmethyl, imidazolidin-2-ylmethyl, imidazolidin-4-ylmethyl, imidazolin-1-yl, imidazolin-2-yl, imidazolin-4-yl, pyrazolidin-1-yl, pyrazolidin-3-yl, pyrazolidin-4-yl, pyrazolin-1-yl, pyrazolin-3-yl, pyrazolin-4-yl, piperidin-1-yl, piperidin-2-yl, piperidin-3-yl, piperidin-4-yl, piperidin-1-ylmethyl, piperidin-2-ylmethyl, piperidin-3-ylmethyl, piperidin-4-ylmethyl, piperazin-1-yl, piperazin-2-yl, piperazin-3-yl, morpholin-2-yl, morpholin-3-yl, morpholin-4-yl, morpholin-2-ylmethyl, morpholin-3-ylmethyl, morpholin-4-ylmethyl, morpholin-2-ylethoxy, morpholin-3-ylethoxy and morpholin-4-ylethoxy;
R⁶ is H, alkyl or haloalkyl; and
R⁸ is hydroxyl, aryl or heteroaryl, wherein aryl or heteroaryl are substituted with —NH₂ or —OH and R⁸ is optionally substituted with one or more groups R¹⁰ selected from amino, halo, alkyl, cycloalkyl, heterocyclyl, aryl, and heteroaryl.

41. The compound of claim 40 which is selected from the group consisting of: 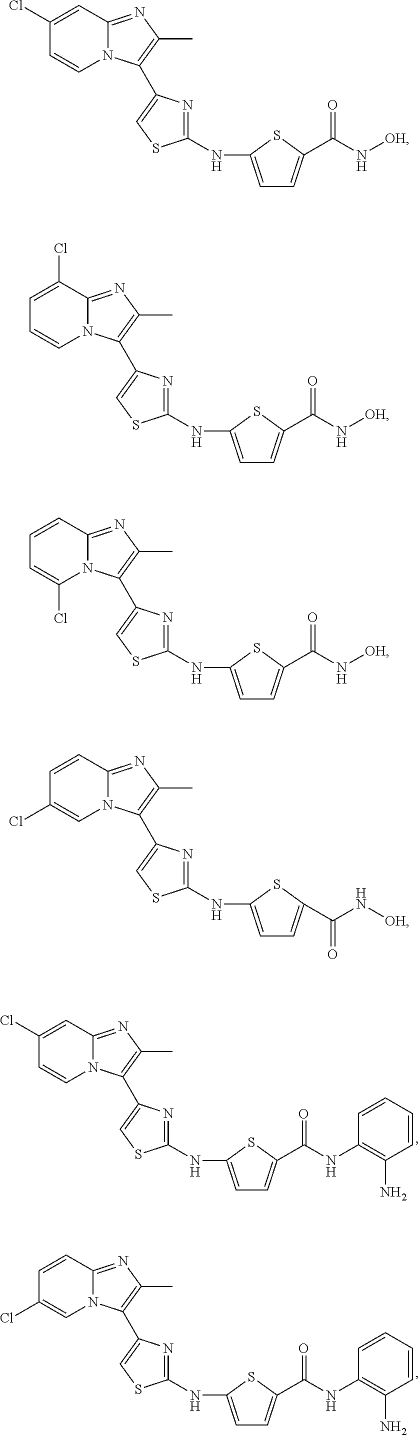 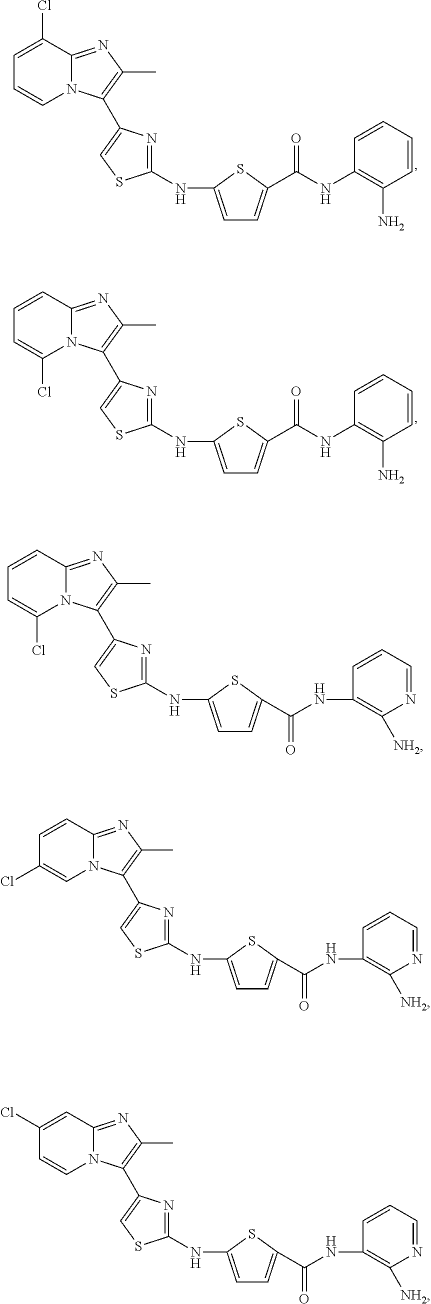 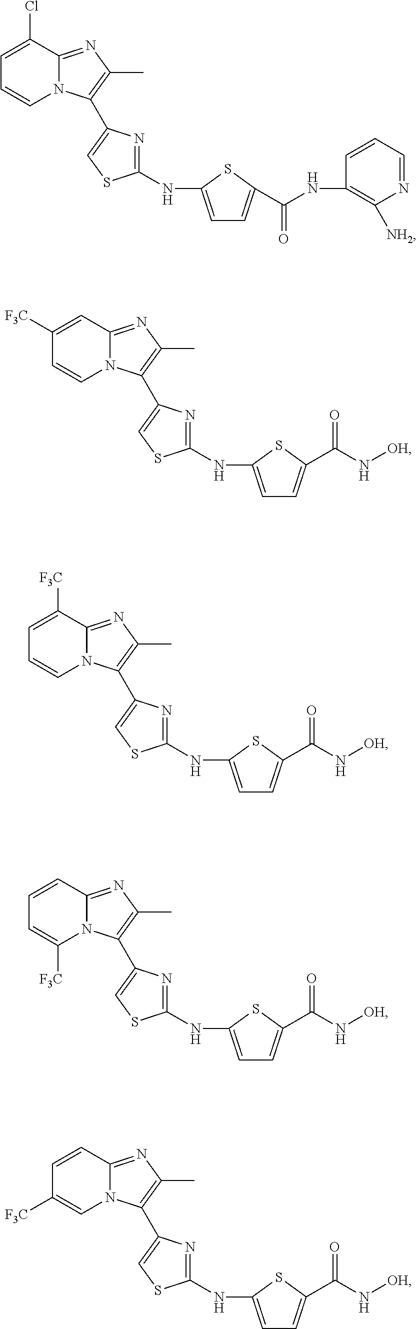 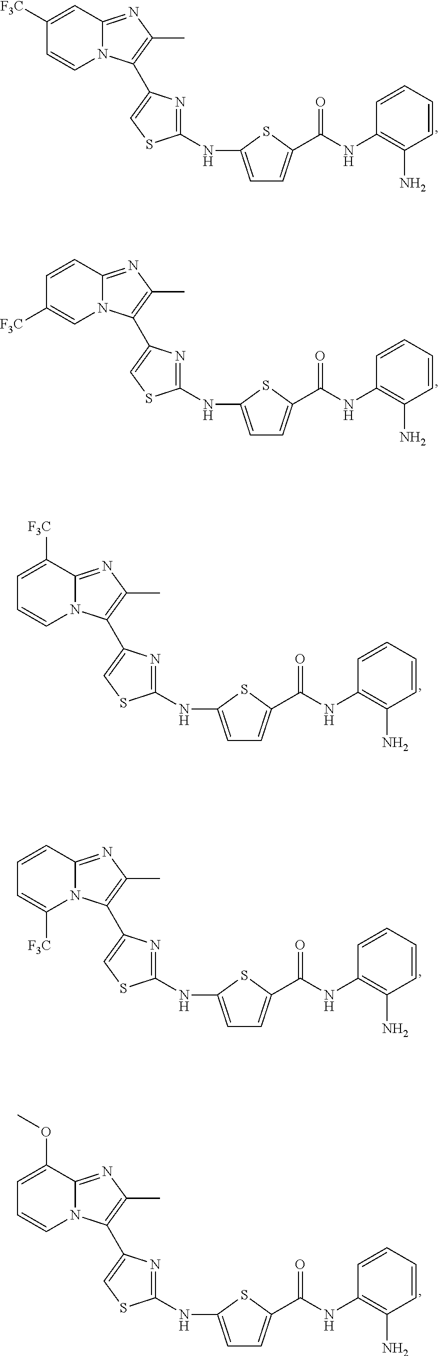 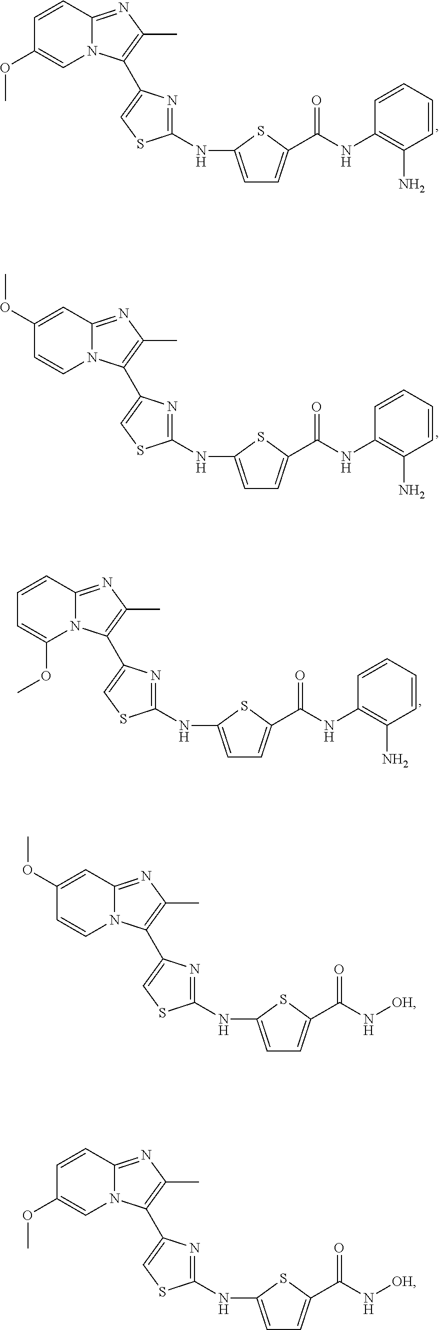 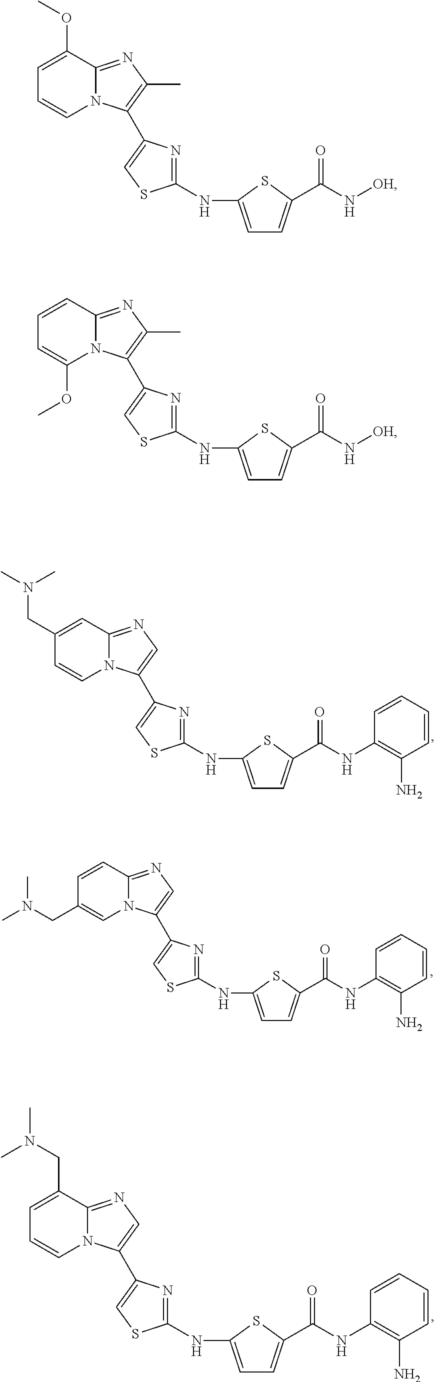 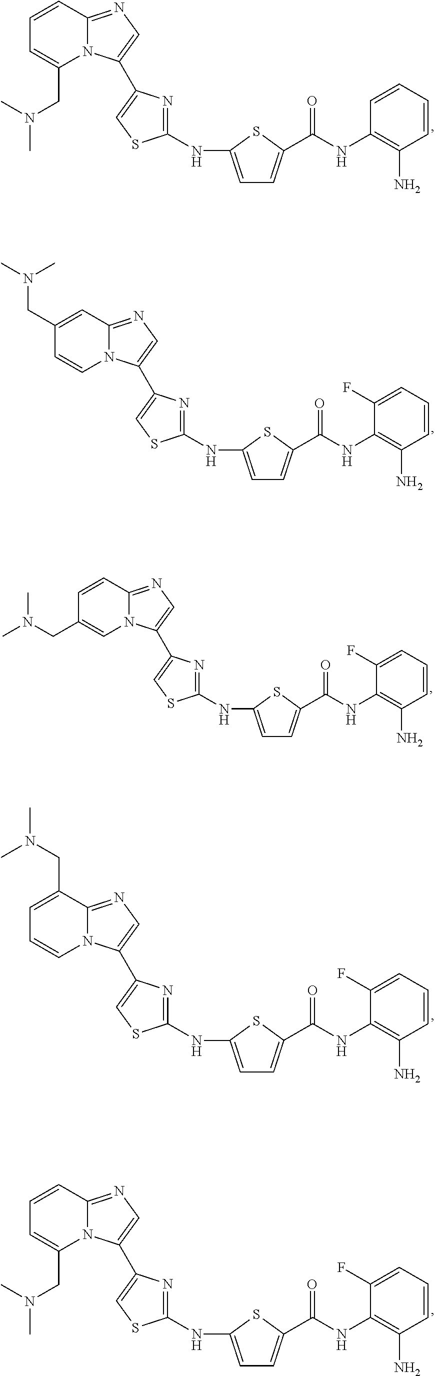 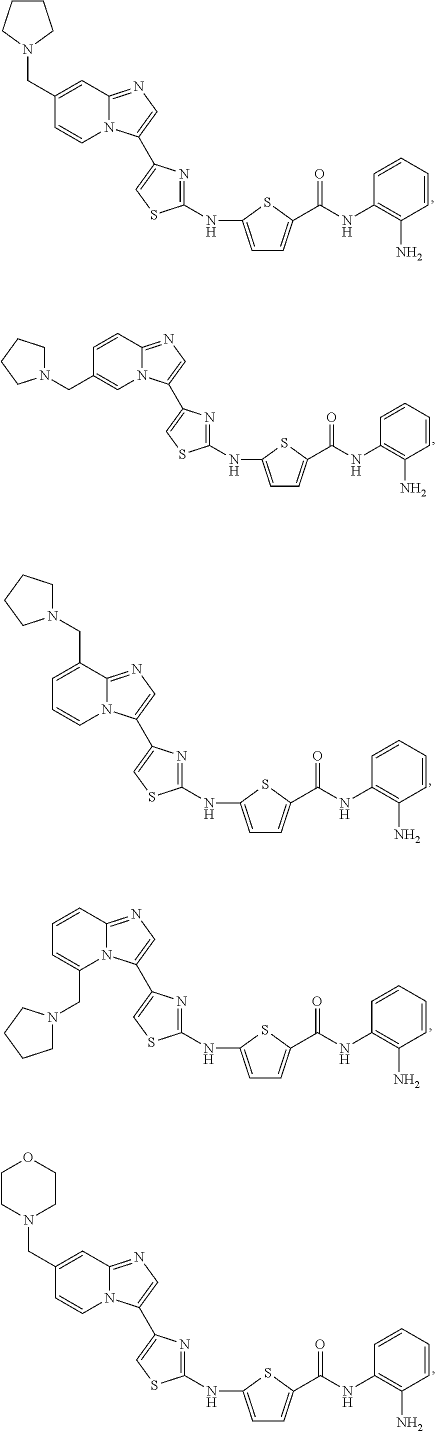 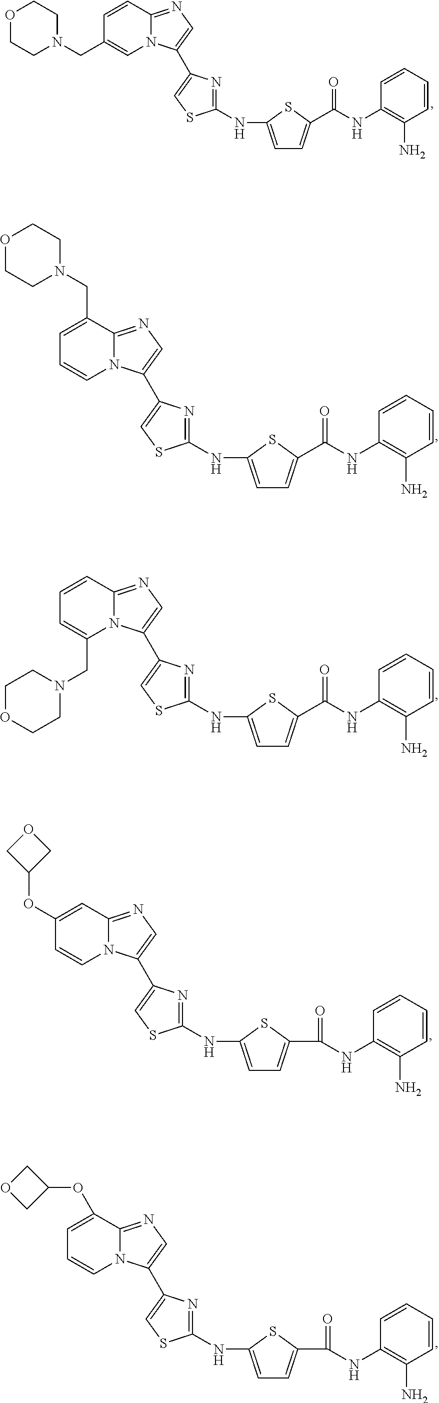 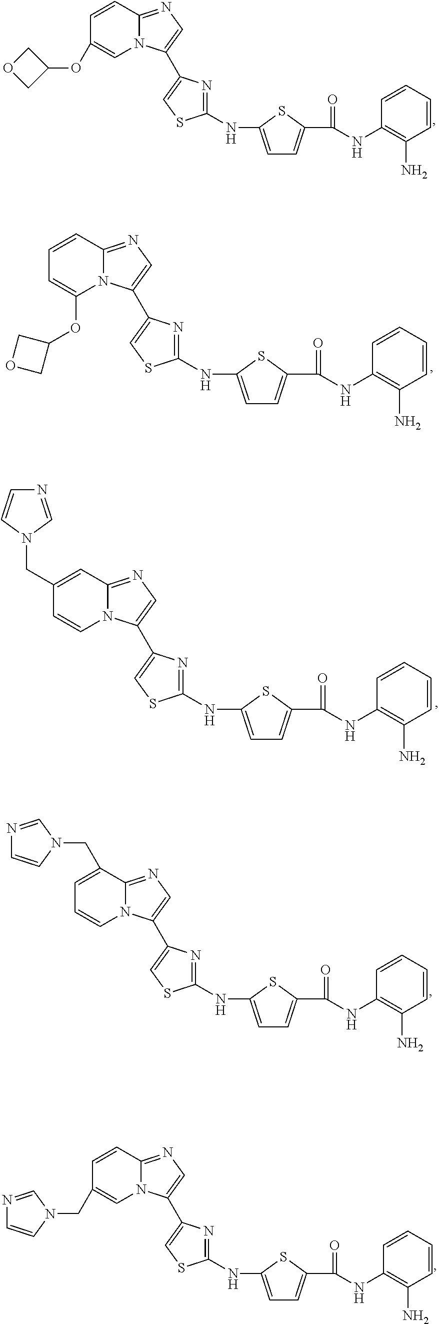 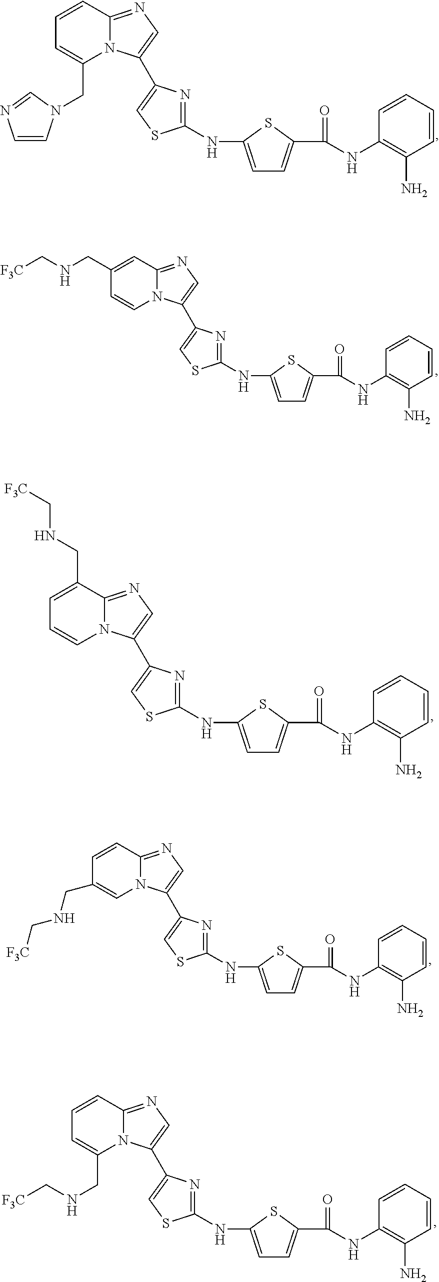 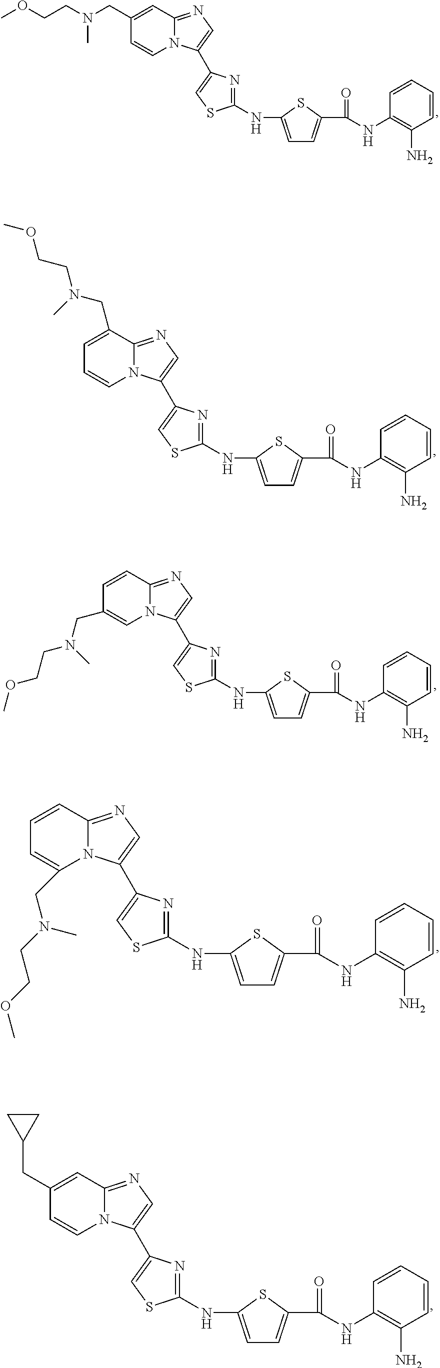 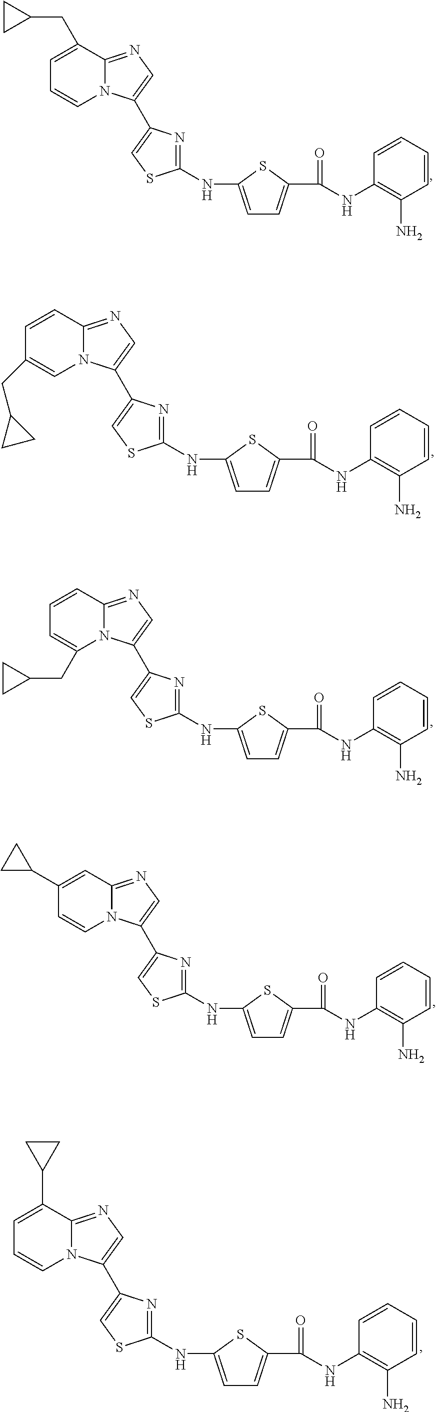 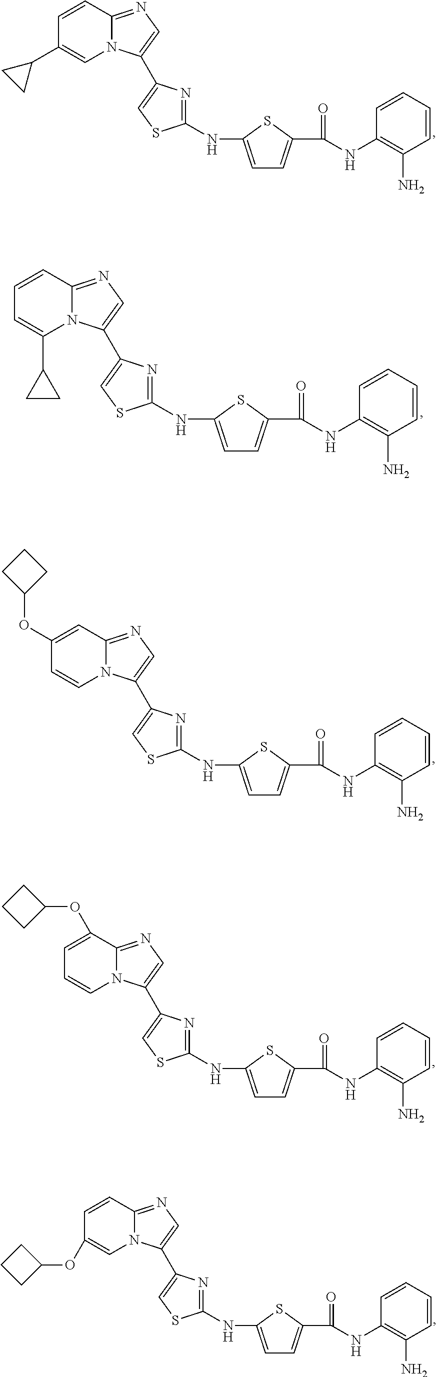 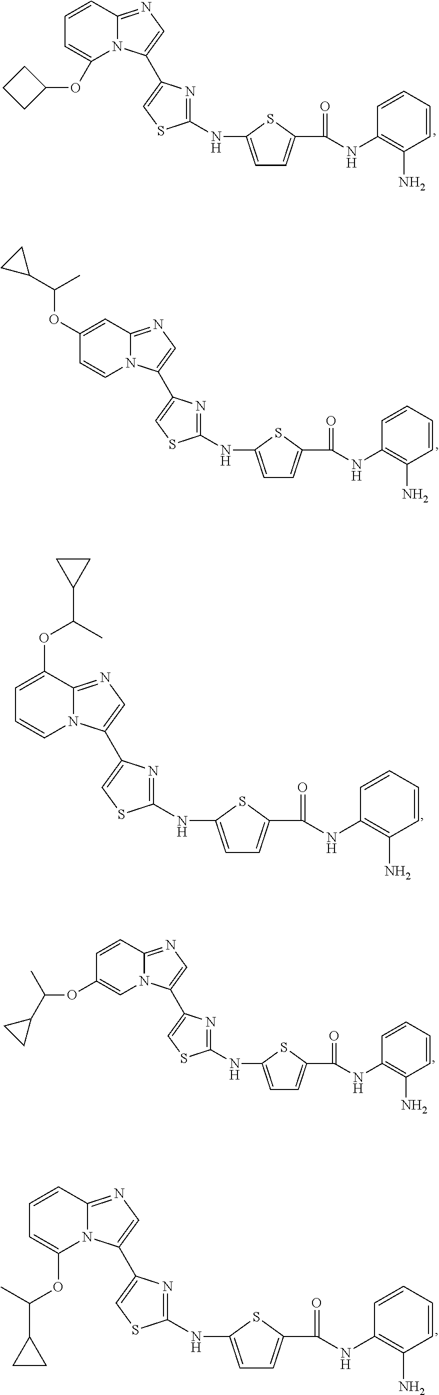 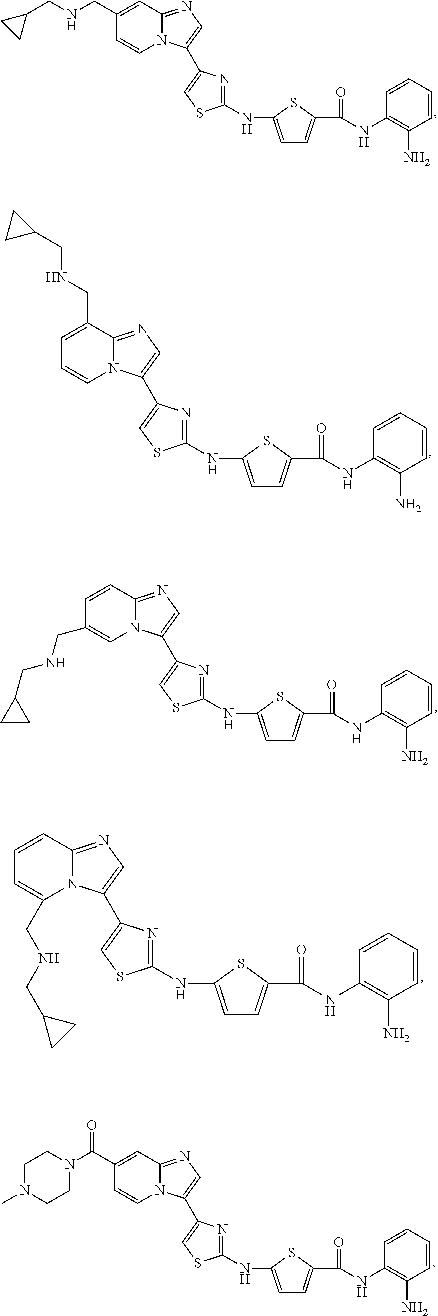 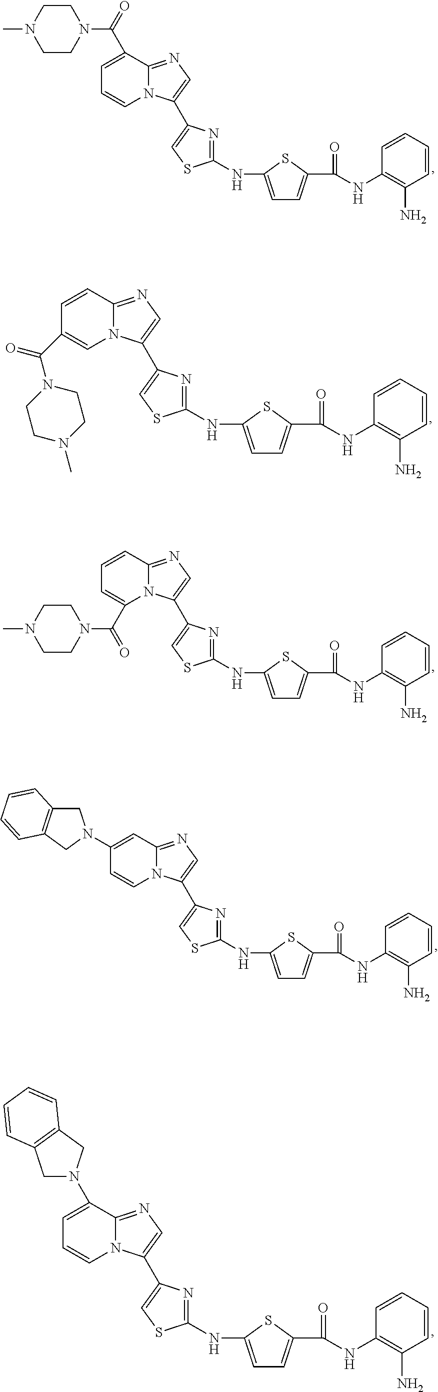 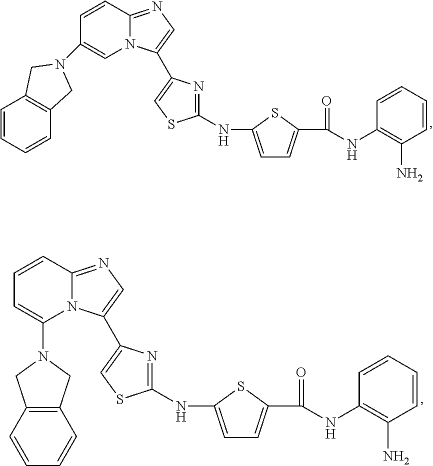
and pharmaceutically acceptable salts thereof.

42. The compound of claim 41 which is selected from the group consisting of: 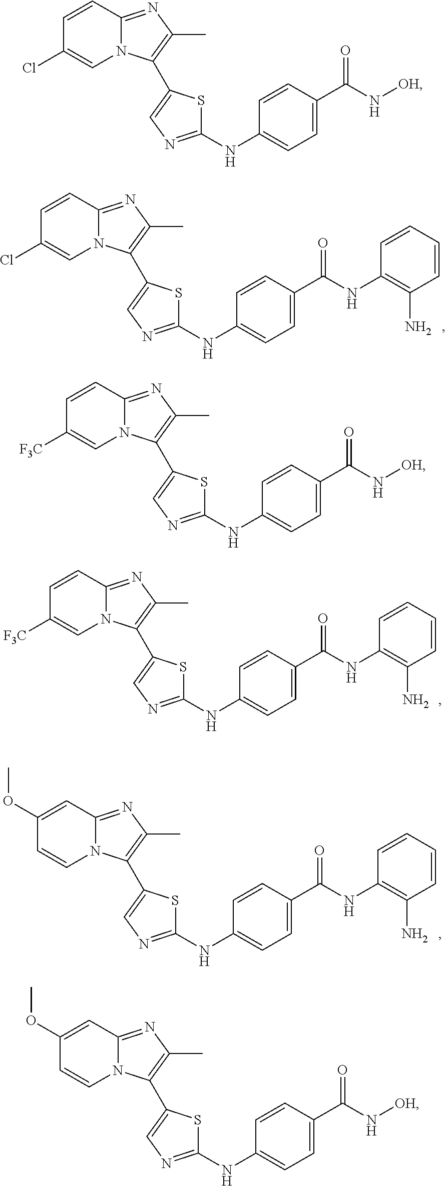
and pharmaceutically acceptable salts thereof.

43. The compound of claim 1 selected from those of Formula (I-e) and pharmaceutically acceptable salts thereof: 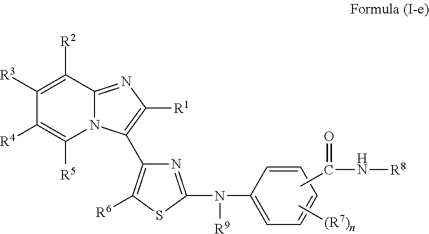
wherein R⁹ is selected from alkyl, haloalkyl, aminoalkyl, cycloalkyl, heterocyclyl, aryl, and heteroaryl, wherein R⁹ is optionally substituted by one or more groups selected from halo, nitro, cyano, hydroxy, amino, azido, carboxy and mercapto.

44. The compound of claim 43, wherein at least two of R¹, R², R³, R⁴ and R⁵ are H and each non-hydrogen R¹, R², R³, R⁴ and R⁵ is independently selected from chloro, fluoro, bromo, methyl, ethyl, propyl, methoxy, ethoxy, carboxy, cyano, methoxymethyl, ethoxyethyl, propoxyethyl, methoxyethoxy, trifluoromethyl, hydroxyethoxy, dimethylamino, diethylamino, dimethylaminomethyl, dimethylaminoethyl, diethylaminomethyl, dimethylaminoethoxy, trifluoromethoxymethyl, trifluoroethoxymethyl, 3-oxetanoxy, trifluoroethylaminomethyl, N-methyl-N-methoxyethyl-aminoethyl, cyclopropanylmethyl, cyclobutoxy, 1-cyclopropanylethoxy, cyclopropanylmethylaminomethyl, 4-methylpiperazin-1-carbonyl, isoindolin-2-yl, N-methoxyethylcarbamoyl, N-(morpholin-4-yl)-ethylcarbamoyl, dimethylaminoethylamino, methylcarboxy, N,N-dimethylaminoethylcarbamoyl, benzyl, phenylethyl, trifluoromethylphenylethyl, phenoxymethyl, fluorophenoxymethyl, phenylethylaminomethyl, benzylaminomethyl, triazinylmethyl, piperidinylmethyl, piperidinyloxy, trifluoromethylpiperidinylmethyl, pyridinyloxymethyl, pyridinylmethoxy, tetrahydropyrazinyloxy, methylpiperazinylmethyl, pyrrolidin-1-yl, pyrrolidin-2-yl, pyrrolidin-3-yl, pyrrolidin-1-ylmethyl, pyrrolidin-2-ylmethyl, pyrrolidin-3-ylmethyl, pyrrolidin-1-ylethoxy, pyrrolidin-2-ylethoxy, pyrrolidin-3-ylethoxy, imidazol-1-ylmethyl, imidazol-2-ylmethyl, imidazol-4-ylmethyl, imidazolidin-1-yl, imidazolidin-2-yl, imidazolidin-4-yl, imidazolidin-1-ylmethyl, imidazolidin-2-ylmethyl, imidazolidin-4-ylmethyl, imidazolin-1-yl, imidazolin-2-yl, imidazolin-4-yl, pyrazolidin-1-yl, pyrazolidin-3-yl, pyrazolidin-4-yl, pyrazolin-1-yl, pyrazolin-3-yl, pyrazolin-4-yl, piperidin-1-yl, piperidin-2-yl, piperidin-3-yl, piperidin-4-yl, piperidin-1-ylmethyl, piperidin-2-ylmethyl, piperidin-3-ylmethyl, piperidin-4-ylmethyl, piperazin-1-yl, piperazin-2-yl, piperazin-3-yl, morpholin-2-yl, morpholin-3-yl, morpholin-4-yl, morpholin-2-ylmethyl, morpholin-3-ylmethyl, morpholin-4-ylmethyl, morpholin-2-ylethoxy, morpholin-3-ylethoxy and morpholin-4-ylethoxy; R⁶ is H, alkyl or haloalkyl; R⁷ is independently fluoro, chloro, bromo, or methyl; n is 0, 1, or 2; R⁸ is hydroxyl, aryl or heteroaryl, wherein aryl or heteroaryl are substituted with —NH₂ or —OH at a ring position adjacent to attachment of the —CONH-moiety and R⁸ is optionally substituted with one or more groups R¹⁰ selected from amino, halo, alkyl, cycloalkyl, heterocyclyl, aryl, and heteroaryl; and R⁹ is selected from alkyl, haloalkyl and aminoalkyl.

45. The compound of claim 43 which is selected from the group consisting of:

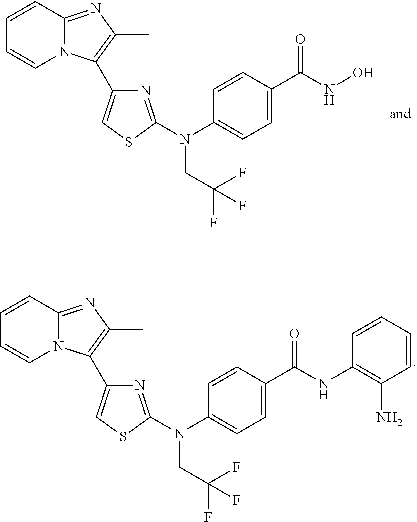

46. A compound selected from those of Formula (II) and pharmaceutically acceptable salts thereof: 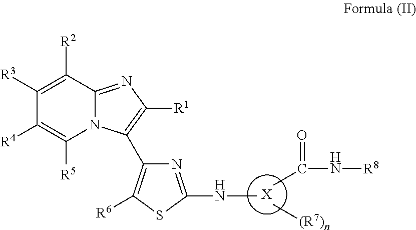
wherein
R¹ is selected from the group consisting of H, methyl, ethyl, trifluoromethyl, dimethylaminomethyl, morpholinylmethyl and pyrrolidinylmethyl;
at least two of R², R³, R⁴ and R⁵ are H, and the others are independently selected from the group consisting of H, hydroxyl, methyl, methoxy, chloro, fluoro, trifluoromethyl, dimethylaminomethyl, morpholinylmethyl and pyrrolidinylmethyl;
R⁶ is H or methyl;
X is phenyl, 5-membered heteroaryl, or 6-membered heteroaryl, each optionally substituted with halo, wherein the heteroaryl contains one or more heteroatoms selected from N, S and O;
R⁷ is halo and n is 0 or 1; and
R⁸ is hydroxyl, aryl or heteroaryl, wherein aryl or heteroaryl are substituted with —NH₂ at a ring position adjacent to attachment of the —CONH-moiety and R⁸ is optionally substituted with one or more groups R¹⁰ selected from amino, halo, alkyl, cycloalkyl, heterocyclyl, aryl, and heteroaryl.

47. A pharmaceutical composition comprising an effective amount of one or more compounds according to claim 1 and a pharmaceutically-acceptable carrier.

48. The pharmaceutical composition according to claim 47, further comprising one or more anti-cancer agents.

49. The pharmaceutical composition according to claim 47, wherein the one or more anti-cancer agents are selected from the group consisting of cyclophosphamide, dacarbazine, cisplatin, methotrexate, mercaptopurine, thioguanine, fluorouracil, cytarabine, vinblastine, paclitaxel, doxorubicin, bleomycin, mitomycin, prednisone, tamoxifen, flutamide, asparaginase, rituximab, trastuzumab, imatinib, retinoic acid, colony-stimulating factor, amifostine, lenalidomide, HDAC inhibitor, CDK inhibitor, camptothecin and topotecan.

50. A method of inhibiting or treating a disease arising from abnormal cell proliferation and/or differentiation in an animal, comprising administering to said animal a therapeutically effective amount of one or more compounds according to claim 1.

51. The method according to claim 50, wherein the animal is human.

52. The method according to claim 50, wherein the disease is mediated by a histone deacetylase or CDK.

53. The method according to claim 50, wherein the disease is selected from the group consisting of a cell proliferative disease, autosomal dominant disorder, genetic related metabolic disorder, fibrosis, autoimmune disease, diabetes, neurological disease, and Alzheimer's disease.

54. The method according to claim 50, wherein the disease is cancer or pulmonary fibrosis.

55. The method according to claim 50, wherein the disease is cancer selected from the group consisting of bladder cancer, breast cancer, colon cancer, rectal cancer, endometrial cancer, kidney cancer, leukemia, lung cancer, melanoma, non-Hodgkin's lymphoma, pancreatic cancer, prostate cancer, skin cancer and thyroid cancer.
